SEQUENCE LISTING (385 sequences)

SEQ ID NO: 1 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 15: note = (2R,3S)-3-benzylpyrrolidine-2-carboxylic acid
XXXGXXXEWXFQSGX

SEQ ID NO: 2 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-(4-pyridyl)propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXEWXXQXGX

SEQ ID NO: 3 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 15: note = (2R,4R)-4-[(4-fluorophenyl)methyl]pyrrolidine-2-car boxylic acid
XXXGXXXEWXXQSGX

SEQ ID NO: 4 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXEWXXQXGX

SEQ ID NO: 5 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXSWXSQXGX

SEQ ID NO: 6 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXAWXXQXGX

SEQ ID NO: 7 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-2,3-diaminopropanoic acid
  SITE 15: note = (2R,4R)-4-[(4-fluorophenyl)methyl]pyrrolidine-2-car boxylic acid
XXXGXXXEWXXQXGX

SEQ ID NO: 8 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = l-Ornithine
  SITE 15: note = (2R,4R)-4-[(4-fluorophenyl)methyl]pyrrolidine-2-car boxylic acid
XXXGXXXEWXXQXGX

SEQ ID NO: 9 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 15: note = (2R,4R)-4-[(4-fluorophenyl)methyl]pyrrolidine-2-car boxylic acid
XXXGXXXEWXXQKGX

SEQ ID NO: 10 - protein, 15 aa, synthetic construct
  SITE 1: note = (2S)-3-[4-(2-acetamidoethoxy)phenyl]-2-amino-propan oic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXEWXXQXGX

SEQ ID NO: 11 - protein, 15 aa, synthetic construct
  SITE 1: note = (2S)-3-[4-(2-acetamidoethoxy)phenyl]-2-amino-propan oic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-2,3-diaminopropanoic acid
  SITE 15: note = (2R,4R)-4-[(4-fluorophenyl)methyl]pyrrolidine-2-car boxylic acid
XXXGXXXEWXXQXGX

SEQ ID NO: 12 - protein, 15 aa, synthetic construct
  SITE 1: note = (2S)-3-[6-(2-acetamidoethoxy)-3-pyridyl]-2-amino-pr opanoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXEWXXQXGX

SEQ ID NO: 13 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(1-bicyclo[1.1.1]pentanyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXEWXXQXGX

SEQ ID NO: 14 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXEWXXQXGX

SEQ ID NO: 15 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-(4-pyridyl)propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXEWXXQXGX

SEQ ID NO: 16 - protein, 15 aa, synthetic construct
  SITE 1: note = (2S)-3-[4-(2-acetamidoethoxy)phenyl]-2-amino-propan oic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXSWXXQXGX

SEQ ID NO: 17 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXDWXXQXGX

SEQ ID NO: 18 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2S)-2-amino-3-hydroxy-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXEWXXQXGX

SEQ ID NO: 19 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = (2R)-2-amino-3-hydroxy-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXEWXXQXGX

SEQ ID NO: 20 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 3-aminoazetidine-3-carboxylic acid
  SITE 10: note = 2-amino-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXEWXXQXGX

SEQ ID NO: 21 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 3-aminoazetidine-3-carboxylic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXEWXXQXGX

SEQ ID NO: 22 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = (2S)-2-amino-3-hydroxy-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXEWXXQXGX

SEQ ID NO: 23 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-3-hydroxy-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXEWXXQXGX

SEQ ID NO: 24 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-2-amino-3-morpholino-propanoic acid
  SITE 15: note = (2R,4R)-4-[(4-fluorophenyl)methyl]pyrrolidine-2-car boxylic acid
XXXGXXXEWXXQXGX

SEQ ID NO: 25 - protein, 15 aa, synthetic construct
  SITE 1: note = (2S)-3-[4-(2-acetamidoethoxy)phenyl]-2-amino-propan oic acid
  SITE 2: note = (2S)-2-amino-3-(1-bicyclo[1.1.1]pentanyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXEWXXQXGX

SEQ ID NO: 26 - protein, 15 aa, synthetic construct
  SITE 1: note = 4-[5-[(2S)-2-amino-2-carboxy-ethyl]-2-pyridyl]benzo ic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXEWXXQXGX

SEQ ID NO: 27 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 15: note = (2R,3S)-3-(pyrimidin-5-ylmethyl)pyrrolidine-2-carbo xylic acid
XXXGXXXEWXFQSGX

SEQ ID NO: 28 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 15: note = (2R,3S)-3-benzylpyrrolidine-2-carboxylic acid
XXXGXXXEWXXQSGX

SEQ ID NO: 29 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,3S)-3-benzylpyrrolidine-2-carboxylic acid
XXXGXXXEWXXQXGX

SEQ ID NO: 30 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 15: note = (2R,4R)-4-benzylpyrrolidine-2-carboxylic acid
XXXGXXXEWXXQSGX

SEQ ID NO: 31 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-benzylpyrrolidine-2-carboxylic acid
XXXGXXXEWXXQXGX

SEQ ID NO: 32 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 15: note = (2R,4R)-4-[(4-fluorophenyl)methyl]pyrrolidine-2-car boxylic acid
XXXGXXXEWXRQSGX

SEQ ID NO: 33 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXEWXRQXGX

SEQ ID NO: 34 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-(3-pyridyl)propanoic acid
  SITE 15: note = (2R,4R)-4-[(4-fluorophenyl)methyl]pyrrolidine-2-car boxylic acid
XXXGXXXEWXXQSGX

SEQ ID NO: 35 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 15: note = (2R,4R)-4-[(4-fluorophenyl)methyl]pyrrolidine-2-car boxylic acid
XXXGXXXEWXFQRGX

SEQ ID NO: 36 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-(2-pyridyl)propanoic acid
  SITE 15: note = (2R,4R)-4-[(4-fluorophenyl)methyl]pyrrolidine-2-car boxylic acid
XXXGXXXEWXXQSGX

SEQ ID NO: 37 - protein, 15 aa, synthetic construct
  SITE 1: note = (2S)-3-[4-(4-acetylpiperazin-1-yl)phenyl]-2-amino-p ropanoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXEWXXQXGX

SEQ ID NO: 38 - protein, 15 aa, synthetic construct
  SITE 1: note = 2-[4-[(2S)-2-amino-2-carboxy-ethyl]phenyl]pyrimidin e-5-carboxylic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXEWXXQXGX

SEQ ID NO: 39 - protein, 15 aa, synthetic construct
  SITE 1: note = (2S)-3-[6-(4-acetylpiperazin-1-yl)-3-pyridyl]-2-ami no-propanoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXEWXXQXGX

SEQ ID NO: 40 - protein, 15 aa, synthetic construct
  SITE 1: note = (2S)-3-[2-(4-acetylpiperazin-1-yl)pyrimidin-5-yl]-2 -amino-propanoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXEWXXQXGX

SEQ ID NO: 41 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXAWXXQXGX

SEQ ID NO: 42 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 43 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(1-bicyclo[1.1.1]pentanyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXEWXXQXGX

SEQ ID NO: 44 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(1-bicyclo[1.1.1]pentanyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 45 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(1-bicyclo[1.1.1]pentanyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-(4-pyridyl)propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXEWXXQXGX

SEQ ID NO: 46 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(1-bicyclo[1.1.1]pentanyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-2,3-diaminopropanoic acid
  SITE 15: note = (2R,4R)-4-(4-pyridylmethyl)pyrrolidine-2-carboxylic acid
XXXGXXXEWXXQXGX

SEQ ID NO: 47 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-(4-pyridyl)propanoic acid
  SITE 13: note = (2S)-2,3-diaminopropanoic acid
  SITE 15: note = (2R,4R)-4-(4-pyridylmethyl)pyrrolidine-2-carboxylic acid
XXXGXXXEWXXQXGX

SEQ ID NO: 48 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(1-bicyclo[1.1.1]pentanyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-(4-pyridyl)propanoic acid
  SITE 13: note = (2S)-2,3-diaminopropanoic acid
  SITE 15: note = (2R,4R)-4-(4-pyridylmethyl)pyrrolidine-2-carboxylic acid
XXXGXXXEWXXQXGX

SEQ ID NO: 49 - protein, 15 aa, synthetic construct
  SITE 1: note = (2S)-3-[4-(2-acetamidoethoxy)phenyl]-2-amino-propan oic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXEWXXQXGX

SEQ ID NO: 50 - protein, 15 aa, synthetic construct
  SITE 1: note = (2S)-3-[4-(2-acetamidoethoxy)phenyl]-2-amino-propan oic acid
  SITE 2: note = (2S)-2-amino-3-(1-bicyclo[1.1.1]pentanyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXEWXXQXGX

SEQ ID NO: 51 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,3S)-3-[(4-bromophenyl)methyl]pyrrolidine-2-carb oxylic acid
XXXGXXXEWXXQXGX

SEQ ID NO: 52 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-5-ureido-pentanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXEWXXQXGX

SEQ ID NO: 53 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,3S)-3-[(4-pyrimidin-5-ylphenyl)methyl]pyrrolidi ne-2-carboxylic acid
XXXGXXXEWXXQXGX

SEQ ID NO: 54 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,3S)-3-[[4-[2-(methylamino)pyrimidin-5-yl]phenyl ]methyl]pyrrolidine-2-carboxylic acid
XXXGXXXEWXXQXGX

SEQ ID NO: 55 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,3S)-3-[[4-(4-pyridyl)phenyl]methyl]pyrrolidine- 2-carboxylic acid
XXXGXXXEWXXQXGX

SEQ ID NO: 56 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,3S)-3-[[4-(3-pyridyl)phenyl]methyl]pyrrolidine- 2-carboxylic acid
XXXGXXXEWXXQXGX

SEQ ID NO: 57 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,3S)-3-[[4-(5-aminopyrazin-2-yl)phenyl]methyl]py rrolidine-2-carboxylic acid
XXXGXXXEWXXQXGX

SEQ ID NO: 58 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,3S)-3-[(4-pyridazin-4-ylphenyl)methyl]pyrrolidi ne-2-carboxylic acid
XXXGXXXEWXXQXGX

SEQ ID NO: 59 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,3S)-3-[[4-(2-aminopyrimidin-5-yl)phenyl]methyl] pyrrolidine-2-carboxylic acid
XXXGXXXEWXXQXGX

SEQ ID NO: 60 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,3S)-3-[[4-(2-methoxypyrimidin-5-yl)phenyl]methy l]pyrrolidine-2-carboxylic acid
XXXGXXXEWXXQXGX

SEQ ID NO: 61 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,3S)-3-[[4-(2-pyridyl)phenyl]methyl]pyrrolidine- 2-carboxylic acid
XXXGXXXEWXXQXGX

SEQ ID NO: 62 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,3S)-3-(4-(3-methylisoxazol-4-yl)benzyl)pyrrolid ine-2-carboxylic acid
XXXGXXXEWXXQXGX

SEQ ID NO: 63 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,3S)-3-(4-(1-methyl-1H-imidazol-2-yl)benzyl)pyrr olidine-2-carboxylic acid
XXXGXXXEWXXQXGX

SEQ ID NO: 64 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,3S)-3-(4-(1-methyl-1H-imidazol-4-yl)benzyl)pyrr olidine-2-carboxylic acid
XXXGXXXEWXXQXGX

SEQ ID NO: 65 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 4-aminopiperidine-4-carboxylic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXEWXXQXGX

SEQ ID NO: 66 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = (2S)-2,6-diamino-2-methyl-hexanoic acid
  SITE 10: note = 2-amino-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXEWXXQXGX

SEQ ID NO: 67 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2S)-2,6-diamino-2-methyl-hexanoic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXEWXXQXGX

SEQ ID NO: 68 - protein, 15 aa, synthetic construct
  SITE 1: note = trans-4-[4-[(2S)-2-amino-2-carboxy-ethyl]phenyl]cyc lohexanecarboxylic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 3-aminoazetidine-3-carboxylic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXEWXXQXGX

SEQ ID NO: 69 - protein, 15 aa, synthetic construct
  SITE 1: note = cis-4-[4-[(2S)-2-amino-2-carboxy-ethyl]phenyl]cyclo hexanecarboxylic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 3-aminoazetidine-3-carboxylic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXEWXXQXGX

SEQ ID NO: 70 - protein, 14 aa, synthetic construct
  SITE 1: note = (2S)-3-[4-(2-acetamidoethoxy)phenyl]-2-amino-propan oic acid
  SITE 2: note = (2S)-2-amino-3-(4-pyridyl)propanoic acid
  SITE 3: note = 4-[(2S)-2-amino-2-carboxy-ethyl]benzoic acid
  SITE 4: note = (2S,4S)-4-fluoropyrrolidine-2-carboxylic acid
  SITE 5: note = (2S,3S)-2-amino-3-(4-fluoro-1H-indol-3-yl)butanoic acid
  SITE 6: note = 2-amino-2-methyl-propanoic acid
  SITE 9: note = 2-amino-2-methyl-propanoic acid
  SITE 10: note = (2S)-2-amino-3-pyrimidin-5-yl-propanoic acid
  SITE 12: note = (2S)-2,4-diaminobutanoic acid
  SITE 14: note = (2R,3S)-3-(pyrimidin-5-ylmethyl)pyrrolidine-2-carbo xylic acid
XXXXXXEWXXQXGX

SEQ ID NO: 71 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,3S)-3-(pyrimidin-5-ylmethyl)pyrrolidine-2-carbo xylic acid
XXXGXXXEWXXQXGX

SEQ ID NO: 72 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-(3-pyridyl)propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXEWXXQXGX

SEQ ID NO: 73 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-(2-pyridyl)propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXEWXXQXGX

SEQ ID NO: 74 - protein, 14 aa, synthetic construct
  SITE 1: note = (2S)-3-[4-(2-acetamidoethoxy)phenyl]-2-amino-propan oic acid
  SITE 2: note = (2S)-2-amino-3-(4-pyridyl)propanoic acid
  SITE 3: note = 4-[(2S)-2-amino-2-carboxy-ethyl]benzoic acid
  SITE 4: note = (2S,4S)-4-fluoropyrrolidine-2-carboxylic acid
  SITE 5: note = (2S,3S)-2-amino-3-(4-fluoro-1H-indol-3-yl)butanoic acid
  SITE 6: note = 2-amino-2-methyl-propanoic acid
  SITE 9: note = 2-amino-2-methyl-propanoic acid
  SITE 12: note = (2S)-2,4-diaminobutanoic acid
  SITE 14: note = (2R,4R)-4-[(4-fluorophenyl)methyl]pyrrolidine-2-car boxylic acid
XXXXXXEWXRQXGX

SEQ ID NO: 75 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 15: note = (2R,4R)-4-[(4-fluorophenyl)methyl]pyrrolidine-2-car boxylic acid
XXXGXXXEWXXQRGX

SEQ ID NO: 76 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-(4-pyridyl)propanoic acid
  SITE 15: note = (2R,4R)-4-[(4-fluorophenyl)methyl]pyrrolidine-2-car boxylic acid
XXXGXXXEWXXQRGX

SEQ ID NO: 77 - protein, 14 aa, synthetic construct
  SITE 1: note = (2S)-3-[4-(2-acetamidoethoxy)phenyl]-2-amino-propan oic acid
  SITE 2: note = (2S)-2-amino-3-(4-pyridyl)propanoic acid
  SITE 3: note = 4-[(2S)-2-amino-2-carboxy-ethyl]benzoic acid
  SITE 4: note = (2S,4S)-4-fluoropyrrolidine-2-carboxylic acid
  SITE 5: note = (2S,3S)-2-amino-3-(4-fluoro-1H-indol-3-yl)butanoic acid
  SITE 6: note = 2-amino-2-methyl-propanoic acid
  SITE 9: note = 2-amino-2-methyl-propanoic acid
  SITE 10: note = (2S)-2-amino-3-pyrimidin-5-yl-propanoic acid
  SITE 14: note = (2R,3S)-3-benzylpyrrolidine-2-carboxylic acid
XXXXXXEWXXQSGX

SEQ ID NO: 78 - protein, 14 aa, synthetic construct
  SITE 1: note = (2S)-3-[4-(2-acetamidoethoxy)phenyl]-2-amino-propan oic acid
  SITE 2: note = (2S)-2-amino-3-(4-pyridyl)propanoic acid
  SITE 3: note = 4-[(2S)-2-amino-2-carboxy-ethyl]benzoic acid
  SITE 4: note = (2S,4S)-4-fluoropyrrolidine-2-carboxylic acid
  SITE 5: note = (2S,3S)-2-amino-3-(4-fluoro-1H-indol-3-yl)butanoic acid
  SITE 6: note = 2-amino-2-methyl-propanoic acid
  SITE 9: note = 2-amino-2-methyl-propanoic acid
  SITE 10: note = (2S)-2-amino-3-pyrimidin-5-yl-propanoic acid
  SITE 12: note = (2S)-2,4-diaminobutanoic acid
  SITE 14: note = (2R,3S)-3-benzylpyrrolidine-2-carboxylic acid
XXXXXXEWXXQXGX

SEQ ID NO: 79 - protein, 14 aa, synthetic construct
  SITE 1: note = (2S)-3-[4-(2-acetamidoethoxy)phenyl]-2-amino-propan oic acid
  SITE 2: note = (2S)-2-amino-3-(4-pyridyl)propanoic acid
  SITE 3: note = 4-[(2S)-2-amino-2-carboxy-ethyl]benzoic acid
  SITE 4: note = (2S,4S)-4-fluoropyrrolidine-2-carboxylic acid
  SITE 5: note = (2S,3S)-2-amino-3-(4-fluoro-1H-indol-3-yl)butanoic acid
  SITE 6: note = 2-amino-2-methyl-propanoic acid
  SITE 9: note = 2-amino-2-methyl-propanoic acid
  SITE 10: note = (2S)-2-amino-3-pyrimidin-5-yl-propanoic acid
  SITE 14: note = (2R,4R)-4-benzylpyrrolidine-2-carboxylic acid
XXXXXXEWXXQSGX

SEQ ID NO: 80 - protein, 14 aa, synthetic construct
  SITE 1: note = (2S)-3-[4-(2-acetamidoethoxy)phenyl]-2-amino-propan oic acid
  SITE 2: note = (2S)-2-amino-3-(4-pyridyl)propanoic acid
  SITE 3: note = 4-[(2S)-2-amino-2-carboxy-ethyl]benzoic acid
  SITE 4: note = (2S,4S)-4-fluoropyrrolidine-2-carboxylic acid
  SITE 5: note = (2S,3S)-2-amino-3-(4-fluoro-1H-indol-3-yl)butanoic acid
  SITE 6: note = 2-amino-2-methyl-propanoic acid
  SITE 9: note = 2-amino-2-methyl-propanoic acid
  SITE 10: note = (2S)-2-amino-3-pyrimidin-5-yl-propanoic acid
  SITE 12: note = (2S)-2,4-diaminobutanoic acid
  SITE 14: note = (2R,4R)-4-benzylpyrrolidine-2-carboxylic acid
XXXXXXEWXXQXGX

SEQ ID NO: 81 - protein, 15 aa, synthetic construct
  SITE 1: note = 2-[4-[(2S)-2-amino-2-carboxy-ethyl]phenyl]pyrimidin e-5-carboxylic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 3-aminoazetidine-3-carboxylic acid
  SITE 11: note = (2S)-2-amino-3-(4-pyridyl)propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXEWXXQXGX

SEQ ID NO: 82 - protein, 15 aa, synthetic construct
  SITE 1: note = 2-[4-[(2S)-2-amino-2-carboxy-ethyl]phenyl]pyrimidin e-5-carboxylic acid
  SITE 2: note = (2S)-2-amino-3-(1-bicyclo[1.1.1]pentanyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXEWXXQXGX

SEQ ID NO: 83 - protein, 15 aa, synthetic construct
  SITE 1: note = 2-[4-[(2S)-2-amino-2-carboxy-ethyl]phenyl]pyrimidin e-5-carboxylic acid
  SITE 2: note = (2S)-2-amino-3-(1-bicyclo[1.1.1]pentanyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXSWXXQXGX

SEQ ID NO: 84 - protein, 15 aa, synthetic construct
  SITE 1: note = 2-[4-[(2S)-2-amino-2-carboxy-ethyl]phenyl]pyrimidin e-5-carboxylic acid
  SITE 2: note = (2S)-2-amino-3-(1-bicyclo[1.1.1]pentanyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-(4-pyridyl)propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXSWXXQXGX

SEQ ID NO: 85 - protein, 15 aa, synthetic construct
  SITE 1: note = 2-[4-[(2S)-2-amino-2-carboxy-ethyl]phenyl]pyrimidin e-5-carboxylic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-(4-pyridyl)propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXAWXXQXGX

SEQ ID NO: 86 - protein, 15 aa, synthetic construct
  SITE 1: note = 2-[4-[(2S)-2-amino-2-carboxy-ethyl]phenyl]pyrimidin e-5-carboxylic acid
  SITE 2: note = (2S)-2-amino-3-(1-bicyclo[1.1.1]pentanyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-(4-pyridyl)propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXEWXXQXGX

SEQ ID NO: 87 - protein, 15 aa, synthetic construct
  SITE 1: note = 2-[4-[(2S)-2-amino-2-carboxy-ethyl]phenyl]pyrimidin e-5-carboxylic acid
  SITE 2: note = (2S)-2-amino-3-(1-bicyclo[1.1.1]pentanyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXAWXXQXGX

SEQ ID NO: 88 - protein, 15 aa, synthetic construct
  SITE 1: note = 2-[4-[(2S)-2-amino-2-carboxy-ethyl]phenyl]pyrimidin e-5-carboxylic acid
  SITE 2: note = (2S)-2-amino-3-(1-bicyclo[1.1.1]pentanyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-(4-pyridyl)propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXAWXXQXGX

SEQ ID NO: 89 - protein, 15 aa, synthetic construct
  SITE 1: note = 2-[4-[(2S)-2-amino-2-carboxy-ethyl]phenyl]pyrimidin e-5-carboxylic acid
  SITE 2: note = (2S)-2-amino-3-(1-bicyclo[1.1.1]pentanyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-(4-pyridyl)propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 90 - protein, 15 aa, synthetic construct
  SITE 1: note = (2S)-3-[4-(4-acetylpiperazin-1-yl)phenyl]-2-amino-p ropanoic acid
  SITE 2: note = (2S)-2-amino-3-(1-bicyclo[1.1.1]pentanyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-(4-pyridyl)propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXEWXXQXGX

SEQ ID NO: 91 - protein, 15 aa, synthetic construct
  SITE 1: note = 4-[5-[(2S)-2-amino-2-carboxy-ethyl]pyrimidin-2-yl]b enzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXEWXXQXGX

SEQ ID NO: 92 - protein, 15 aa, synthetic construct
  SITE 1: note = 4-[5-[(2S)-2-amino-2-carboxy-ethyl]pyrimidin-2-yl]b enzoic acid
  SITE 2: note = (2S)-2-amino-3-(1-bicyclo[1.1.1]pentanyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXEWXXQXGX

SEQ ID NO: 93 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(1-bicyclo[1.1.1]pentanyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2,3-diaminopropanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXEWXXQXGX

SEQ ID NO: 94 - protein, 15 aa, synthetic construct
  SITE 1: note = 2-[4-[(2S)-2-amino-2-carboxy-ethyl]phenyl]pyrimidin e-5-carboxylic acid
  SITE 2: note = (2S)-2-amino-3-(1-bicyclo[1.1.1]pentanyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXAWXSQXGX

SEQ ID NO: 95 - protein, 15 aa, synthetic construct
  SITE 1: note = (2S)-3-[4-(2-acetamidoethoxy)phenyl]-2-amino-propan oic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-(4-pyridyl)propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXEWXXQXGX

SEQ ID NO: 96 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(1-bicyclo[1.1.1]pentanyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2,3-diaminopropanoic acid
  SITE 15: note = (2R,4R)-4-(4-pyridylmethyl)pyrrolidine-2-carboxylic acid
XXXGXXXEWXXQSGX

SEQ ID NO: 97 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(1-bicyclo[1.1.1]pentanyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = (2S)-2-amino-3-hydroxy-2-methyl-propanoic acid
  SITE 10: note = 2-amino-2-methyl-propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXSWXSQXGX

SEQ ID NO: 98 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(1-bicyclo[1.1.1]pentanyl)propanoic acid
  SITE 3: note = 5-[(2S)-2-amino-2-carboxy-ethyl]pyridine-2-carboxyl ic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXEWXXQXGX

SEQ ID NO: 99 - protein, 15 aa, synthetic construct
  SITE 1: note = 3-[4-[(2S)-2-amino-2-carboxy-ethyl]phenyl]bicyclo[1 .1.1]pentane-1-carboxylic acid
  SITE 2: note = (2S)-2-amino-3-(1-bicyclo[1.1.1]pentanyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXEWXXQXGX

SEQ ID NO: 100 - protein, 15 aa, synthetic construct
  SITE 1: note = 4-[5-[(2S)-2-amino-2-carboxy-ethyl]-2-pyridyl]benzo ic acid
  SITE 2: note = (2S)-2-amino-3-(1-bicyclo[1.1.1]pentanyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2,3-diaminopropanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXEWXXQXGX

SEQ ID NO: 101 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrazin-2-yl-propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXEWXXQXGX

SEQ ID NO: 102 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyridazin-3-yl-propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXEWXXQXGX

SEQ ID NO: 103 - protein, 15 aa, synthetic construct
  SITE 1: note = cis-4-[4-[(2S)-2-amino-2-carboxy-ethyl]phenyl]cyclo hexanecarboxylic acid
  SITE 2: note = (2S)-2-amino-3-(1-bicyclo[1.1.1]pentanyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = (2S)-2-amino-3-hydroxy-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXEWXXQXGX

SEQ ID NO: 104 - protein, 15 aa, synthetic construct
  SITE 1: note = cis-4-[4-[(2S)-2-amino-2-carboxy-ethyl]phenyl]cyclo hexanecarboxylic acid
  SITE 2: note = (2S)-2-amino-3-(1-bicyclo[1.1.1]pentanyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = (2S)-2-amino-3-hydroxy-2-methyl-propanoic acid
  SITE 10: note = (2S)-2,6-diamino-2-methyl-hexanoic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXEWXXQXGX

SEQ ID NO: 105 - protein, 15 aa, synthetic construct
  SITE 1: note = cis-4-[4-[(2S)-2-amino-2-carboxy-ethyl]phenyl]cyclo hexanecarboxylic acid
  SITE 2: note = (2S)-2-amino-3-(1-bicyclo[1.1.1]pentanyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 4-aminopiperidine-4-carboxylic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXEWXSQXGX

SEQ ID NO: 106 - protein, 15 aa, synthetic construct
  SITE 1: note = cis-4-[4-[(2S)-2-amino-2-carboxy-ethyl]phenyl]cyclo hexanecarboxylic acid
  SITE 2: note = (2S)-2-amino-3-(1-bicyclo[1.1.1]pentanyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXSWXSQXGX

SEQ ID NO: 107 - protein, 15 aa, synthetic construct
  SITE 1: note = cis-4-[4-[(2S)-2-amino-2-carboxy-ethyl]phenyl]cyclo hexanecarboxylic acid
  SITE 2: note = (2S)-2-amino-3-(1-bicyclo[1.1.1]pentanyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = (2S)-2-amino-3-hydroxy-2-methyl-propanoic acid
  SITE 10: note = 2-amino-2-methyl-propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXEWXSQXGX

SEQ ID NO: 108 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = l-Ornithine
  SITE 15: note = (2R,4R)-4-(4-pyridylmethyl)pyrrolidine-2-carboxylic acid
XXXGXXXSWXXQXGX

SEQ ID NO: 109 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(1-bicyclo[1.1.1]pentanyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = l-Ornithine
  SITE 15: note = (2R,4R)-4-(4-pyridylmethyl)pyrrolidine-2-carboxylic acid
XXXGXXXSWXXQXGX

SEQ ID NO: 110 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(1-bicyclo[1.1.1]pentanyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = l-Ornithine
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXSWXXQXGX

SEQ ID NO: 111 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-3-hydroxy-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 112 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(1-bicyclo[1.1.1]pentanyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-3-hydroxy-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 113 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-2-yl-propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXEWXXQXGX

SEQ ID NO: 114 - protein, 15 aa, synthetic construct
  SITE 1: note = trans-4-[4-[(2S)-2-amino-2-carboxy-ethyl]phenyl]cyc lohexanecarboxylic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXEWXXQXGX

SEQ ID NO: 115 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyridazin-4-yl-propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXEWXXQXGX

SEQ ID NO: 116 - protein, 15 aa, synthetic construct
  SITE 1: note = 4-[4-[(2S)-2-amino-2-carboxy-ethyl]phenyl]benzoic acid
  SITE 2: note = (2S)-2-amino-3-pyridazin-4-yl-propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXEWXXQXGX

SEQ ID NO: 117 - protein, 15 aa, synthetic construct
  SITE 1: note = cis-4-[4-[(2S)-2-amino-2-carboxy-ethyl]phenyl]cyclo hexanecarboxylic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrazin-2-yl-propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXEWXXQXGX

SEQ ID NO: 118 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-tetrahydropyran-4-yl-propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXEWXXQXGX

SEQ ID NO: 119 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-(4-piperidyl)propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXEWXXQXGX

SEQ ID NO: 120 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-piperazin-1-yl-propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXEWXXQXGX

SEQ ID NO: 121 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-(4-piperidyl)propanoic acid
  SITE 15: note = (2R,4R)-4-[(4-fluorophenyl)methyl]pyrrolidine-2-car boxylic acid
XXXGXXXEWXXQSGX

SEQ ID NO: 122 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-piperazin-1-yl-propanoic acid
  SITE 15: note = (2R,4R)-4-[(4-fluorophenyl)methyl]pyrrolidine-2-car boxylic acid
XXXGXXXEWXXQSGX

SEQ ID NO: 123 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-morpholino-propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXEWXXQXGX

SEQ ID NO: 124 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-morpholino-propanoic acid
  SITE 15: note = (2R,4R)-4-[(4-fluorophenyl)methyl]pyrrolidine-2-car boxylic acid
XXXGXXXEWXXQSGX

SEQ ID NO: 125 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-morpholino-propanoic acid
  SITE 15: note = (2R,4R)-4-[(4-fluorophenyl)methyl]pyrrolidine-2-car boxylic acid
XXXGXXXEWXXQSGX

SEQ ID NO: 126 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-morpholino-propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXEWXXQXGX

SEQ ID NO: 127 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-hydroxypyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXSWXXQXGX

SEQ ID NO: 128 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXSWXXQXGX

SEQ ID NO: 129 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = (2S)-2,4-diamino-2-methyl-butanoic acid
  SITE 10: note = 2-amino-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXSWXXQXGX

SEQ ID NO: 130 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2S)-2,4-diamino-2-methyl-butanoic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXSWXXQXGX

SEQ ID NO: 131 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = (2R)-2,4-diamino-2-methyl-butanoic acid
  SITE 10: note = 2-amino-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXSWXXQXGX

SEQ ID NO: 132 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2S)-2,6-diamino-2-methyl-hexanoic acid
  SITE 11: note = (2S)-2-amino-3-pyrimidin-5-yl-propanoic acid
  SITE 13: note = (2S)-2,4-diaminobutanoic acid
  SITE 14: note = 2-(methylamino)acetic acid
  SITE 15: note = (2R,4R)-4-[(4-fluorophenyl)methyl]pyrrolidine-2-car boxylic acid
XXXGXXXEWXXQXXX

SEQ ID NO: 133 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 8: note = l-homoserine
  SITE 10: note = (2S)-2,6-diamino-2-methyl-hexanoic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXXWXXQXGX

SEQ ID NO: 134 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2S)-2,6-diamino-2-methyl-hexanoic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXAWXXQXGX

SEQ ID NO: 135 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2S)-2,6-diamino-2-methyl-hexanoic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXVWXXQXGX

SEQ ID NO: 136 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 8: note = l-alloisoleucine
  SITE 10: note = (2S)-2,6-diamino-2-methyl-hexanoic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXXWXXQXGX

SEQ ID NO: 137 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2S)-2,6-diamino-2-methyl-hexanoic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXTWXXQXGX

SEQ ID NO: 138 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 8: note = l-allothreonine
  SITE 10: note = (2S)-2,6-diamino-2-methyl-hexanoic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXXWXXQXGX

SEQ ID NO: 139 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-chloro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXSWXXQXGX

SEQ ID NO: 140 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(1-naphthyl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXSWXXQXGX

SEQ ID NO: 141 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2S)-2-amino-2-methyl-butanedioic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXSWXXQXGX

SEQ ID NO: 142 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2,4-diamino-2-methyl-butanoic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXSWXXQXGX

SEQ ID NO: 143 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-[(4-bromophenyl)methyl]pyrrolidine-2-carb oxylic acid
XXXGXXXEWXXQXGX

SEQ ID NO: 144 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(1-bicyclo[1.1.1]pentanyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2S)-2,6-diamino-2-methyl-hexanoic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXSWXXQXGX

SEQ ID NO: 145 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(1-bicyclo[1.1.1]pentanyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2,6-diamino-2-methyl-hexanoic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXSWXXQXGX

SEQ ID NO: 146 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(1-bicyclo[1.1.1]pentanyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2S)-2,6-diamino-2-methyl-hexanoic acid
  SITE 11: note = (2S)-2-amino-3-tetrahydropyran-4-yl-propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXSWXXQXGX

SEQ ID NO: 147 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(1-bicyclo[1.1.1]pentanyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 8: note = l-homoserine
  SITE 10: note = (2S)-2,6-diamino-2-methyl-hexanoic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXXWXXQXGX

SEQ ID NO: 148 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-chloro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXEWXXQXGX

SEQ ID NO: 149 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(1-naphthyl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXEWXXQXGX

SEQ ID NO: 150 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-2,3-diaminopropanoic acid
  SITE 15: note = (2R,3S)-3-[(4-bromophenyl)methyl]pyrrolidine-2-carb oxylic acid
XXXGXXXEWXXQXGX

SEQ ID NO: 151 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = l-Ornithine
  SITE 15: note = (2R,3S)-3-[(4-bromophenyl)methyl]pyrrolidine-2-carb oxylic acid
XXXGXXXEWXXQXGX

SEQ ID NO: 152 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 15: note = (2R,3S)-3-[(4-bromophenyl)methyl]pyrrolidine-2-carb oxylic acid
XXXGXXXEWXXQKGX

SEQ ID NO: 153 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(1-naphthyl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,3S)-3-[(4-bromophenyl)methyl]pyrrolidine-2-carb oxylic acid
XXXGXXXEWXXQXGX

SEQ ID NO: 154 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 155 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(1-bicyclo[1.1.1]pentanyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 156 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = (2S)-2-amino-3-pyrimidin-5-yl-propanoic acid
  SITE 15: note = (2R,4R)-4-(4-pyridylmethyl)pyrrolidine-2-carboxylic acid
XXXGXXXSWXXQSGX

SEQ ID NO: 157 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(1-bicyclo[1.1.1]pentanyl)propanoic acid
  SITE 3: note = 4-[(2S)-2-amino-2-carboxy-ethyl]benzoic acid
  SITE 5: note = (2S,4S)-4-hydroxypyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = (2S)-2-amino-3-pyrimidin-5-yl-propanoic acid
  SITE 15: note = (2R,4R)-4-(4-pyridylmethyl)pyrrolidine-2-carboxylic acid
XXXGXXXSWXXQSGX

SEQ ID NO: 158 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(1-bicyclo[1.1.1]pentanyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = (2S)-2-amino-3-pyrimidin-5-yl-propanoic acid
  SITE 15: note = (2R,4R)-4-(4-pyridylmethyl)pyrrolidine-2-carboxylic acid
XXXGXXXSWXXQSGX

SEQ ID NO: 159 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-hydroxypyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 160 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(1-bicyclo[1.1.1]pentanyl)propanoic acid
  SITE 3: note = 4-[(2S)-2-amino-2-carboxy-ethyl]benzoic acid
  SITE 5: note = (2S,4S)-4-hydroxypyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 161 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-hydroxypyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = (2S)-2-amino-3-pyrimidin-5-yl-propanoic acid
  SITE 15: note = (2R,4R)-4-(4-pyridylmethyl)pyrrolidine-2-carboxylic acid
XXXGXXXSWXXQSGX

SEQ ID NO: 162 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 9: note = (2S)-2-amino-3-(1H-pyrrolo[3,2-b]pyridin-3-yl)propa noic acid
  SITE 10: note = 2-amino-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSXXXQXGX

SEQ ID NO: 163 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(cyclobutyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 164 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(cyclobutyl)propanoic acid
  SITE 3: note = 4-[(2S)-2-amino-2-carboxy-ethyl]benzoic acid
  SITE 5: note = (2S,4S)-4-hydroxypyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 165 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(1-bicyclo[1.1.1]pentanyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2S)-2,6-diamino-2-methyl-hexanoic acid
  SITE 11: note = (2S)-2-amino-3-morpholino-propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXEWXXQXGX

SEQ ID NO: 166 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(1-bicyclo[1.1.1]pentanyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2S)-2,6-diamino-2-methyl-hexanoic acid
  SITE 11: note = (2S)-2-amino-3-(4-piperidyl)propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXEWXXQXGX

SEQ ID NO: 167 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(1-bicyclo[1.1.1]pentanyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-morpholino-propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXSWXXQXGX

SEQ ID NO: 168 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(1-bicyclo[1.1.1]pentanyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-piperazin-1-yl-propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXSWXXQXGX

SEQ ID NO: 169 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(1-bicyclo[1.1.1]pentanyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-(4-piperidyl)propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXSWXXQXGX

SEQ ID NO: 170 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(1-bicyclo[1.1.1]pentanyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-3-hydroxy-2-methyl-propanoic acid
  SITE 11: note = (2S)-2-amino-3-morpholino-propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXSWXXQXGX

SEQ ID NO: 171 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(1-bicyclo[1.1.1]pentanyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-3-hydroxy-2-methyl-propanoic acid
  SITE 11: note = (2S)-2-amino-3-piperazin-1-yl-propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXSWXXQXGX

SEQ ID NO: 172 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(1-bicyclo[1.1.1]pentanyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-3-hydroxy-2-methyl-propanoic acid
  SITE 11: note = (2S)-2-amino-3-(4-piperidyl)propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXSWXXQXGX

SEQ ID NO: 173 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(1-bicyclo[1.1.1]pentanyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2S)-2,6-diamino-2-methyl-hexanoic acid
  SITE 11: note = (2S)-2-amino-3-morpholino-propanoic acid
  SITE 15: note = (2R,4R)-4-[(4-fluorophenyl)methyl]pyrrolidine-2-car boxylic acid
XXXGXXXSWXXQSGX

SEQ ID NO: 174 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(1-bicyclo[1.1.1]pentanyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2S)-2,6-diamino-2-methyl-hexanoic acid
  SITE 11: note = (2S)-2-amino-3-morpholino-propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXSWXXQXGX

SEQ ID NO: 175 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(1-bicyclo[1.1.1]pentanyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2S)-2,6-diamino-2-methyl-hexanoic acid
  SITE 11: note = (2S)-2-amino-3-piperazin-1-yl-propanoic acid
  SITE 15: note = (2R,4R)-4-[(4-fluorophenyl)methyl]pyrrolidine-2-car boxylic acid
XXXGXXXSWXXQSGX

SEQ ID NO: 176 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(1-bicyclo[1.1.1]pentanyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-(4-piperidyl)propanoic acid
  SITE 15: note = (2R,4R)-4-(4-pyridylmethyl)pyrrolidine-2-carboxylic acid
XXXGXXXEWXXQSGX

SEQ ID NO: 177 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(1-bicyclo[1.1.1]pentanyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-3-hydroxy-2-methyl-propanoic acid
  SITE 11: note = (2S)-2-amino-3-morpholino-propanoic acid
  SITE 15: note = (2R,4R)-4-(4-pyridylmethyl)pyrrolidine-2-carboxylic acid
XXXGXXXEWXXQSGX

SEQ ID NO: 178 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(1-bicyclo[1.1.1]pentanyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-3-hydroxy-2-methyl-propanoic acid
  SITE 11: note = (2S)-2-amino-3-(4-piperidyl)propanoic acid
  SITE 15: note = (2R,4R)-4-(4-pyridylmethyl)pyrrolidine-2-carboxylic acid
XXXGXXXEWXXQSGX

SEQ ID NO: 179 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(1-bicyclo[1.1.1]pentanyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-morpholino-propanoic acid
  SITE 15: note = (2R,4R)-4-(4-pyridylmethyl)pyrrolidine-2-carboxylic acid
XXXGXXXSWXXQSGX

SEQ ID NO: 180 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(1-bicyclo[1.1.1]pentanyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-(4-piperidyl)propanoic acid
  SITE 15: note = (2R,4R)-4-(4-pyridylmethyl)pyrrolidine-2-carboxylic acid
XXXGXXXSWXXQSGX

SEQ ID NO: 181 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(1-bicyclo[1.1.1]pentanyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-3-hydroxy-2-methyl-propanoic acid
  SITE 11: note = (2S)-2-amino-3-(4-piperidyl)propanoic acid
  SITE 15: note = (2R,4R)-4-(4-pyridylmethyl)pyrrolidine-2-carboxylic acid
XXXGXXXSWXXQSGX

SEQ ID NO: 182 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(1-bicyclo[1.1.1]pentanyl)propanoic acid
  SITE 3: note = 4-[(2S)-2-amino-2-carboxy-ethyl]benzoic acid
  SITE 5: note = (2S,4S)-4-hydroxypyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 8: note = l-allothreonine
  SITE 10: note = (2R)-2-amino-2-methyl-butanedioic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXXWXXQXGX

SEQ ID NO: 183 - protein, 15 aa, synthetic construct
  SITE 1: note = (2S)-3-[4-(4-acetylpiperazin-1-yl)phenyl]-2-amino-p ropanoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXSWXXQXGX

SEQ ID NO: 184 - protein, 15 aa, synthetic construct
  SITE 1: note = (2S)-3-[4-(4-acetylpiperazin-1-yl)phenyl]-2-amino-p ropanoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2S)-2-amino-2-methyl-butanedioic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXSWXXQXGX

SEQ ID NO: 185 - protein, 15 aa, synthetic construct
  SITE 1: note = 2-[4-[(2S)-2-amino-2-carboxy-ethyl]phenyl]pyrimidin e-5-carboxylic acid
  SITE 2: note = (2S)-2-amino-3-(1-bicyclo[1.1.1]pentanyl)propanoic acid
  SITE 3: note = 4-[(2S)-2-amino-2-carboxy-ethyl]benzoic acid
  SITE 5: note = (2S,4S)-4-hydroxypyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 186 - protein, 15 aa, synthetic construct
  SITE 1: note = 2-[4-[(2S)-2-amino-2-carboxy-ethyl]phenyl]pyrimidin e-5-carboxylic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 187 - protein, 15 aa, synthetic construct
  SITE 1: note = 2-[4-[(2S)-2-amino-2-carboxy-ethyl]phenyl]pyrimidin e-5-carboxylic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2S)-2-amino-2-methyl-butanedioic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 188 - protein, 15 aa, synthetic construct
  SITE 1: note = 2-[4-[(2S)-2-amino-2-carboxy-ethyl]phenyl]pyrimidin e-5-carboxylic acid
  SITE 2: note = (2S)-2-amino-3-(1-bicyclo[1.1.1]pentanyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2S)-2-amino-2-methyl-butanedioic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 189 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2S)-2-amino-2-methyl-butanedioic acid
  SITE 11: note = (2S)-2-amino-3-tetrahydropyran-4-yl-propanoic acid
  SITE 15: note = (2R,4R)-4-(4-pyridylmethyl)pyrrolidine-2-carboxylic acid
XXXGXXXSWXXQSGX

SEQ ID NO: 190 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(1-bicyclo[1.1.1]pentanyl)propanoic acid
  SITE 3: note = 4-[(2S)-2-amino-2-carboxy-ethyl]benzoic acid
  SITE 5: note = (2S,4S)-4-hydroxypyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-3-hydroxy-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 191 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(1-bicyclo[1.1.1]pentanyl)propanoic acid
  SITE 3: note = 4-[(2S)-2-amino-2-carboxy-ethyl]benzoic acid
  SITE 5: note = (2S,4S)-4-hydroxypyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2S)-2-amino-2-methyl-butanedioic acid
  SITE 11: note = (2S)-2-amino-3-tetrahydropyran-4-yl-propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 192 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(1-bicyclo[1.1.1]pentanyl)propanoic acid
  SITE 3: note = 4-[(2S)-2-amino-2-carboxy-ethyl]benzoic acid
  SITE 5: note = (2S,4S)-4-hydroxypyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-3-hydroxy-2-methyl-propanoic acid
  SITE 11: note = (2S)-2-amino-3-tetrahydropyran-4-yl-propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 193 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(1-bicyclo[1.1.1]pentanyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2S)-2-amino-2-methyl-butanedioic acid
  SITE 11: note = (2S)-2-amino-3-tetrahydropyran-4-yl-propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 194 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-hydroxypyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 195 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-hydroxypyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-3-hydroxy-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 196 - protein, 15 aa, synthetic construct
  SITE 1: note = (2S)-3-[4-(4-acetylpiperazin-1-yl)phenyl]-2-amino-p ropanoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-hydroxypyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXSWXXQXGX

SEQ ID NO: 197 - protein, 15 aa, synthetic construct
  SITE 1: note = 2-[4-[(2S)-2-amino-2-carboxy-ethyl]phenyl]pyrimidin e-5-carboxylic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-hydroxypyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 198 - protein, 15 aa, synthetic construct
  SITE 1: note = 2-[4-[(2S)-2-amino-2-carboxy-ethyl]phenyl]pyrimidin e-5-carboxylic acid
  SITE 2: note = (2S)-2-amino-3-(1-bicyclo[1.1.1]pentanyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 199 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-hydroxypyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2S)-2-amino-2-methyl-butanedioic acid
  SITE 11: note = (2S)-2-amino-3-tetrahydropyran-4-yl-propanoic acid
  SITE 15: note = (2R,4R)-4-(4-pyridylmethyl)pyrrolidine-2-carboxylic acid
XXXGXXXSWXXQSGX

SEQ ID NO: 200 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2S)-2-amino-2-methyl-butanedioic acid
  SITE 11: note = (2S)-2-amino-3-tetrahydropyran-4-yl-propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 201 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-hydroxypyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2S)-2-amino-2-methyl-butanedioic acid
  SITE 11: note = (2S)-2-amino-3-tetrahydropyran-4-yl-propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 202 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(1-bicyclo[1.1.1]pentanyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2S)-2-amino-2-methyl-butanedioic acid
  SITE 11: note = l-allothreonine
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 203 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(1-bicyclo[1.1.1]pentanyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2S)-2-amino-2-methyl-butanedioic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSWXVQXGX

SEQ ID NO: 204 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(1-bicyclo[1.1.1]pentanyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-3-hydroxy-2-methyl-propanoic acid
  SITE 11: note = (2S)-2-amino-3-tetrahydropyran-4-yl-propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 205 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-2,4-diaminobutanoic acid
  SITE 14: note = 2-(methylamino)acetic acid
  SITE 15: note = (2R,4R)-4-(4-pyridylmethyl)pyrrolidine-2-carboxylic acid
XXXGXXXSWXXQXXX

SEQ ID NO: 206 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-3-hydroxy-2-methyl-propanoic acid
  SITE 11: note = (2S)-2-amino-3-pyrimidin-5-yl-propanoic acid
  SITE 13: note = (2S)-2,4-diaminobutanoic acid
  SITE 14: note = 2-(methylamino)acetic acid
  SITE 15: note = (2R,4R)-4-(4-pyridylmethyl)pyrrolidine-2-carboxylic acid
XXXGXXXSWXXQXXX

SEQ ID NO: 207 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-hydroxypyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-3-hydroxy-2-methyl-propanoic acid
  SITE 11: note = (2S)-2-amino-3-tetrahydropyran-4-yl-propanoic acid
  SITE 13: note = (2S)-2,4-diaminobutanoic acid
  SITE 14: note = 2-(methylamino)acetic acid
  SITE 15: note = (2R,4R)-4-(4-pyridylmethyl)pyrrolidine-2-carboxylic acid
XXXGXXXSWXXQXXX

SEQ ID NO: 208 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2S)-2-amino-2-methyl-butanedioic acid
  SITE 11: note = (2S)-2-amino-3-tetrahydropyran-4-yl-propanoic acid
  SITE 13: note = (2S)-2,4-diaminobutanoic acid
  SITE 14: note = 2-(methylamino)acetic acid
  SITE 15: note = (2R,4R)-4-(4-pyridylmethyl)pyrrolidine-2-carboxylic acid
XXXGXXXSWXXQXXX

SEQ ID NO: 209 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-hydroxypyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2S)-2-amino-2-methyl-butanedioic acid
  SITE 11: note = (2S)-2-amino-3-pyrimidin-5-yl-propanoic acid
  SITE 13: note = (2S)-2,4-diaminobutanoic acid
  SITE 14: note = 2-(methylamino)acetic acid
  SITE 15: note = (2R,4R)-4-(4-pyridylmethyl)pyrrolidine-2-carboxylic acid
XXXGXXXSWXXQXXX

SEQ ID NO: 210 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-hydroxypyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2S)-2-amino-2-methyl-butanedioic acid
  SITE 11: note = (2S)-2-amino-3-tetrahydropyran-4-yl-propanoic acid
  SITE 13: note = (2S)-2,4-diaminobutanoic acid
  SITE 14: note = 2-(methylamino)acetic acid
  SITE 15: note = (2R,4R)-4-(4-pyridylmethyl)pyrrolidine-2-carboxylic acid
XXXGXXXSWXXQXXX

SEQ ID NO: 211 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-tetrahydropyran-4-yl-propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 212 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-hydroxypyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-tetrahydropyran-4-yl-propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 213 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-hydroxypyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-3-hydroxy-2-methyl-propanoic acid
  SITE 11: note = (2S)-2-amino-3-tetrahydropyran-4-yl-propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 214 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-2,4-diaminobutanoic acid
  VARIANT 15: note = Any amino acid
XXXGXXXEWXXQXGX

SEQ ID NO: 215 - protein, 15 aa, synthetic construct
  SITE 1: note = (2S)-2-amino-3-[4-(4-carbamoylphenyl)phenyl]propano ic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-3-hydroxy-2-methyl-propanoic acid
  SITE 11: note = (2S)-2-amino-3-tetrahydropyran-4-yl-propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 216 - protein, 15 aa, synthetic construct
  SITE 1: note = (2S)-2-amino-3-[4-(4-carbamoylphenyl)phenyl]propano ic acid
  SITE 2: note = (2S)-2-amino-3-(1-bicyclo[1.1.1]pentanyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-3-hydroxy-2-methyl-propanoic acid
  SITE 11: note = (2S)-2-amino-3-tetrahydropyran-4-yl-propanoic acid
  SITE 13: note = (2S)-2,4-diaminobutanoic acid
  SITE 15: note = (2R,3S)-3-[(4-fluorophenyl)methyl]pyrrolidine-2-car boxylic acid
XXXGXXXSWXXQXGX

SEQ ID NO: 217 - protein, 15 aa, synthetic construct
  SITE 1: note = (2S)-2-amino-3-[4-(4-carbamoylphenyl)phenyl]propano ic acid
  SITE 2: note = (2S)-2-amino-3-(1-bicyclo[1.1.1]pentanyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2,4-diamino-2-methyl-4-oxo-butanoic acid
  SITE 11: note = (2S)-2-amino-3-tetrahydropyran-4-yl-propanoic acid
  SITE 13: note = (2S)-2,4-diaminobutanoic acid
  SITE 15: note = (2R,3S)-3-[(4-fluorophenyl)methyl]pyrrolidine-2-car boxylic acid
XXXGXXXSWXXQXGX

SEQ ID NO: 218 - protein, 15 aa, synthetic construct
  SITE 1: note = (2S)-3-[4-(2-acetamidoethoxy)phenyl]-2-amino-propan oic acid
  SITE 2: note = (2S)-2-amino-3-(4-pyridyl)propanoic acid
  SITE 3: note = (2S)-2-amino-3-(4-carbamoylphenyl)propanoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-4-amino-2-(methylamino)butanoic acid
  SITE 15: note = (2R,4R)-4-(4-pyridylmethyl)pyrrolidine-2-carboxylic acid
XXXGXXXSWXXQXGX

SEQ ID NO: 219 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = L-homotyrosine
  SITE 13: note = (2S)-2,4-diaminobutanoic acid
  SITE 15: note = (2R,3S)-3-(4-pyridylmethyl)pyrrolidine-2-carboxylic acid
XXXGXXXSWXXQXGX

SEQ ID NO: 220 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = L-homotyrosine
  SITE 13: note = (2S)-2,4-diaminobutanoic acid
  SITE 15: note = (2R,3S)-3-(4-pyridylmethyl)pyrrolidine-2-carboxylic acid
XXXGXXXSWXXQXGX

SEQ ID NO: 221 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = L-homophenylalanine
  SITE 13: note = (2S)-2,4-diaminobutanoic acid
  SITE 15: note = (2R,3S)-3-(4-pyridylmethyl)pyrrolidine-2-carboxylic acid
XXXGXXXSWXXQXGX

SEQ ID NO: 222 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = (2S)-2-amino-4-(4-pyridyl)butanoic acid
  SITE 13: note = (2S)-2,4-diaminobutanoic acid
  SITE 15: note = (2R,3S)-3-(4-pyridylmethyl)pyrrolidine-2-carboxylic acid
XXXGXXXSWXXQXGX

SEQ ID NO: 223 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = (2S)-2-amino-4-(4-pyridyl)butanoic acid
  SITE 13: note = (2S)-2-amino-3-ureido-propanoic acid
  SITE 15: note = (2R,3S)-3-(4-pyridylmethyl)pyrrolidine-2-carboxylic acid
XXXGXXXSWXXQXGX

SEQ ID NO: 224 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-3-methoxy-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,3S)-3-(4-pyridylmethyl)pyrrolidine-2-carboxylic acid
XXXGXXXSWXXQXGX

SEQ ID NO: 225 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 9: note = (2S)-2-amino-3-(4-chloro-1H-indol-3-yl)propanoic acid
  SITE 10: note = (2R)-2-amino-2-methyl-butanedioic acid
  SITE 11: note = (2S)-2-amino-3-pyrimidin-5-yl-propanoic acid
  SITE 13: note = (2S)-2,4-diaminobutanoic acid
  SITE 15: note = (2R,3S)-3-(4-pyridylmethyl)pyrrolidine-2-carboxylic acid
XXXGXXXSXXXQXGX

SEQ ID NO: 226 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 9: note = (2S)-2-amino-3-(4-chloro-1H-indol-3-yl)propanoic acid
  SITE 10: note = (2R)-2-amino-2-methyl-butanedioic acid
  SITE 11: note = (2S)-2-amino-3-pyrimidin-5-yl-propanoic acid
  SITE 13: note = (2S)-2-amino-3-ureido-propanoic acid
  SITE 15: note = (2R,3S)-3-(4-pyridylmethyl)pyrrolidine-2-carboxylic acid
XXXGXXXSXXXQXGX

SEQ ID NO: 227 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = (2S,3R)-2-amino-3-methoxy-butanoic acid
  SITE 13: note = (2S)-2,4-diaminobutanoic acid
  SITE 15: note = (2R,3S)-3-(4-pyridylmethyl)pyrrolidine-2-carboxylic acid
XXXGXXXSWXXQXGX

SEQ ID NO: 228 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = (2S,3R)-2-amino-3-methoxy-butanoic acid
  SITE 13: note = (2S)-2-amino-3-ureido-propanoic acid
  SITE 15: note = (2R,3S)-3-(4-pyridylmethyl)pyrrolidine-2-carboxylic acid
XXXGXXXSWXXQXGX

SEQ ID NO: 229 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = (2S,3R)-2-amino-3-benzyloxy-butanoic acid
  SITE 13: note = (2S)-2,4-diaminobutanoic acid
  SITE 15: note = (2R,3S)-3-(4-pyridylmethyl)pyrrolidine-2-carboxylic acid
XXXGXXXSWXXQXGX

SEQ ID NO: 230 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = (2S)-2-amino-3-pyrimidin-5-yl-propanoic acid
  SITE 13: note = (2S)-2-amino-3-ureido-propanoic acid
  SITE 15: note = (2R,3S)-3-(4-pyridylmethyl)pyrrolidine-2-carboxylic acid
XXXGXXXSWXXQXGX

SEQ ID NO: 231 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = (2S)-2-aminopentanoic acid
  SITE 13: note = (2S)-2,4-diaminobutanoic acid
  SITE 15: note = (2R,3S)-3-(4-pyridylmethyl)pyrrolidine-2-carboxylic acid
XXXGXXXSWXXQXGX

SEQ ID NO: 232 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = (2S)-2-amino-5,5,5-trifluoro-pentanoic acid
  SITE 13: note = (2S)-2,4-diaminobutanoic acid
  SITE 15: note = (2R,3S)-3-(4-pyridylmethyl)pyrrolidine-2-carboxylic acid
XXXGXXXSWXXQXGX

SEQ ID NO: 233 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2R)-2-amino-3-pyrimidin-5-yl-propanoic acid
  SITE 13: note = (2S)-2,4-diaminobutanoic acid
  SITE 15: note = (2R,3S)-3-(4-pyridylmethyl)pyrrolidine-2-carboxylic acid
XXXGXXXSWXXQXGX

SEQ ID NO: 234 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = D-aspartic acid
  SITE 13: note = (2S)-2,4-diaminobutanoic acid
  SITE 15: note = (2R,3S)-3-(4-pyridylmethyl)pyrrolidine-2-carboxylic acid
XXXGXXXSWXDQXGX

SEQ ID NO: 235 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = (2S)-2-amino-3-pyrimidin-5-yl-propanoic acid
  SITE 13: note = (2S)-4-amino-2-(methylamino)butanoic acid
  SITE 15: note = (2R,3S)-3-(4-pyridylmethyl)pyrrolidine-2-carboxylic acid
XXXGXXXSWXXQXGX

SEQ ID NO: 236 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = (2R)-2-amino-3-(4-fluorophenyl)propanoic acid
  SITE 13: note = (2S)-2,4-diaminobutanoic acid
  SITE 15: note = (2R,3S)-3-(4-pyridylmethyl)pyrrolidine-2-carboxylic acid
XXXGXXXSWXXQXGX

SEQ ID NO: 237 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = (2R)-2-amino-3-cyclohexyl-propanoic acid
  SITE 13: note = (2S)-2,4-diaminobutanoic acid
  SITE 15: note = (2R,3S)-3-(4-pyridylmethyl)pyrrolidine-2-carboxylic acid
XXXGXXXSWXXQXGX

SEQ ID NO: 238 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = D-leucine
  SITE 13: note = (2S)-2,4-diaminobutanoic acid
  SITE 15: note = (2R,3S)-3-(4-pyridylmethyl)pyrrolidine-2-carboxylic acid
XXXGXXXSWXLQXGX

SEQ ID NO: 239 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = (2R)-2-amino-3-cyclobutyl-propanoic acid
  SITE 13: note = (2S)-2,4-diaminobutanoic acid
  SITE 15: note = (2R,3S)-3-(4-pyridylmethyl)pyrrolidine-2-carboxylic acid
XXXGXXXSWXXQXGX

SEQ ID NO: 240 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = (2R)-2-amino-3-(3-pyridyl)propanoic acid
  SITE 13: note = (2S)-2,4-diaminobutanoic acid
  SITE 15: note = (2R,3S)-3-(4-pyridylmethyl)pyrrolidine-2-carboxylic acid
XXXGXXXSWXXQXGX

SEQ ID NO: 241 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-4-amino-2-(methylamino)butanoic acid
  SITE 15: note = (2R,3S)-3-(4-pyridylmethyl)pyrrolidine-2-carboxylic acid
XXXGXXXSWXXQXGX

SEQ ID NO: 242 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = D-serine
  SITE 13: note = (2S)-2,4-diaminobutanoic acid
  SITE 15: note = (2R,4R)-4-(4-pyridylmethyl)pyrrolidine-2-carboxylic acid
XXXGXXXSWXSQXGX

SEQ ID NO: 243 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = D-phenylalanine
  SITE 13: note = (2S)-2,4-diaminobutanoic acid
  SITE 15: note = (2R,4R)-4-(4-pyridylmethyl)pyrrolidine-2-carboxylic acid
XXXGXXXSWXFQXGX

SEQ ID NO: 244 - protein, 15 aa, synthetic construct
  SITE 1: note = (2S)-2-amino-3-(4-phenylphenyl)propanoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = D-lysine
  SITE 13: note = (2S)-2-amino-3-ureido-propanoic acid
  SITE 15: note = (2R,4R)-4-(4-pyridylmethyl)pyrrolidine-2-carboxylic acid
XXXGXXXSWXKQXGX

SEQ ID NO: 245 - protein, 15 aa, synthetic construct
  SITE 1: note = (2S)-2-amino-3-(4-phenylphenyl)propanoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = D-ornithine
  SITE 13: note = (2S)-2-amino-3-ureido-propanoic acid
  SITE 15: note = (2R,4R)-4-(4-pyridylmethyl)pyrrolidine-2-carboxylic acid
XXXGXXXSWXXQXGX

SEQ ID NO: 246 - protein, 15 aa, synthetic construct
  SITE 1: note = (2S)-2-amino-3-(4-phenylphenyl)propanoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = (2R)-2,3-diaminopropanoic acid
  SITE 13: note = (2S)-2-amino-3-ureido-propanoic acid
  SITE 15: note = (2R,4R)-4-(4-pyridylmethyl)pyrrolidine-2-carboxylic acid
XXXGXXXSWXXQXGX

SEQ ID NO: 247 - protein, 15 aa, synthetic construct
  SITE 1: note = (2S)-2-amino-3-(4-phenylphenyl)propanoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = (2R)-2,4-diaminobutanoic acid
  SITE 13: note = (2S)-2-amino-3-ureido-propanoic acid
  SITE 15: note = (2R,4R)-4-(4-pyridylmethyl)pyrrolidine-2-carboxylic acid
XXXGXXXSWXXQXGX

SEQ ID NO: 248 - protein, 15 aa, synthetic construct
  SITE 1: note = (2S)-2-amino-3-(4-phenylphenyl)propanoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = (2R)-2,4-diaminobutanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 249 - protein, 15 aa, synthetic construct
  SITE 1: note = (2S)-2-amino-3-(4-phenylphenyl)propanoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = D-glutamic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSWXEQXGX

SEQ ID NO: 250 - protein, 15 aa, synthetic construct
  SITE 1: note = (2S)-2-amino-3-(4-phenylphenyl)propanoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = D-glutamic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSWXEQXGX

SEQ ID NO: 251 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 9: note = (2S)-2-amino-3-(4-chloro-1H-indol-3-yl)propanoic acid
  SITE 10: note = (2R)-2-amino-2-methyl-butanedioic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSXXXQXGX

SEQ ID NO: 252 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 9: note = (2S)-2-amino-3-(4-fluoro-1H-indol-3-yl)propanoic acid
  SITE 10: note = (2R)-2-amino-2-methyl-butanedioic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSXXXQXGX

SEQ ID NO: 253 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = (2S)-2-aminopentanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 254 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = (2S,3S)-2-amino-3-methoxy-butanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 255 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = (2S,3R)-2-amino-3-methoxy-butanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 256 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = L-homotyrosine
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 257 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-3-phenyl-propanoic acid
  SITE 11: note = l-allothreonine
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 258 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-3-phenyl-propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSWXEQXGX

SEQ ID NO: 259 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-3-phenyl-propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSWXDQXGX

SEQ ID NO: 260 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-3-methoxy-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 261 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-3-methoxy-2-methyl-propanoic acid
  SITE 11: note = l-allothreonine
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 262 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-4-amino-2-(methylamino)butanoic acid
  SITE 15: note = (2R,4R)-4-(4-pyridylmethyl)pyrrolidine-2-carboxylic acid
XXXGXXXSWXXQXGX

SEQ ID NO: 263 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = (2S,3S)-2-amino-3-hydroxy-3-(4-pyridyl)propanoic acid
  SITE 13: note = (2S)-2,4-diaminobutanoic acid
  SITE 15: note = (2R,3S)-3-[(4-fluorophenyl)methyl]pyrrolidine-2-car boxylic acid
XXXGXXXSWXXQXGX

SEQ ID NO: 264 - protein, 15 aa, synthetic construct
  SITE 1: note = (2S)-2-amino-3-(4-phenylphenyl)propanoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-hydroxypyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 265 - protein, 15 aa, synthetic construct
  SITE 1: note = (2S)-2-amino-3-(4-phenylphenyl)propanoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = D-aspartic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSWXDQXGX

SEQ ID NO: 266 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-4-methylsulfonyl-butanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 267 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-(2-carbamoylpyrimidin-5-yl)propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 268 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = 5-[(2S)-2-amino-2-carboxy-ethyl]pyrimidine-2-carbox ylic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 269 - protein, 15 aa, synthetic construct
  SITE 1: note = (2S)-2-amino-3-(4-phenylphenyl)propanoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = 5-[(2S)-2-amino-2-carboxy-ethyl]pyrimidine-2-carbox ylic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 270 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-2-amino-3-ureido-propanoic acid
  SITE 15: note = (2R,4R)-4-(4-pyridylmethyl)pyrrolidine-2-carboxylic acid
XXXGXXXSWXXQXGX

SEQ ID NO: 271 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (S)-3-(4-acetylpiperazin-1-yl)-2-aminopropanoic acid
  SITE 15: note = (2R,4R)-4-[(4-fluorophenyl)methyl]pyrrolidine-2-car boxylic acid
XXXGXXXEWXXQSGX

SEQ ID NO: 272 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S,3S)-2-amino-3-hydroxy-3-(4-pyridyl)propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXSWXXQXGX

SEQ ID NO: 273 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S,3R)-2-amino-3-hydroxy-3-(4-pyridyl)propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXSWXXQXGX

SEQ ID NO: 274 - protein, 15 aa, synthetic construct
  SITE 1: note = 4-[4-[(2S)-2-amino-2-carboxy-ethyl]phenyl]benzoic acid
  SITE 2: note = (2S,3R)-2-amino-3-hydroxy-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXSWXXQXGX

SEQ ID NO: 275 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(1R,2S)-2-amino-2-carboxy-1-hydroxy-ethyl]benzoi c acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXSWXXQXGX

SEQ ID NO: 276 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXEWXHQXGX

SEQ ID NO: 277 - protein, 15 aa, synthetic construct
  SITE 1: note = (2S)-2-amino-3-(4-fluorophenyl)propanoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = (2S)-2-amino-3-pyrimidin-5-yl-propanoic acid
  SITE 13: note = (2S)-2,4-diaminobutanoic acid
  SITE 15: note = (2R,3S)-3-[(4-fluorophenyl)methyl]pyrrolidine-2-car boxylic acid
XXXGXXXSWXXQXGX

SEQ ID NO: 278 - protein, 15 aa, synthetic construct
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = (2S)-2-amino-3-pyrimidin-5-yl-propanoic acid
  SITE 13: note = (2S)-2,4-diaminobutanoic acid
  SITE 15: note = (2R,3S)-3-[(4-fluorophenyl)methyl]pyrrolidine-2-car boxylic acid
YXXGXXXSWXXQXGX

SEQ ID NO: 279 - protein, 15 aa, synthetic construct
  SITE 1: note = (S)-2-amino-3-(4-(1-methyl-1H-imidazol-4-yl)phenyl) propanoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = (2S)-2-amino-3-pyrimidin-5-yl-propanoic acid
  SITE 13: note = (2S)-2,4-diaminobutanoic acid
  SITE 15: note = (2R,3S)-3-[(4-fluorophenyl)methyl]pyrrolidine-2-car boxylic acid
XXXGXXXSWXXQXGX

SEQ ID NO: 280 - protein, 15 aa, synthetic construct
  SITE 1: note = (S)-2-amino-3-(4-(1-methyl-1H-imidazol-2-yl)phenyl) propanoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = (2S)-2-amino-3-pyrimidin-5-yl-propanoic acid
  SITE 13: note = (2S)-2,4-diaminobutanoic acid
  SITE 15: note = (2R,3S)-3-[(4-fluorophenyl)methyl]pyrrolidine-2-car boxylic acid
XXXGXXXSWXXQXGX

SEQ ID NO: 281 - protein, 15 aa, synthetic construct
  SITE 1: note = (S)-2-amino-3-(4-(2-methoxypyrimidin-5-yl)phenyl)pr opanoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = (2S)-2-amino-3-pyrimidin-5-yl-propanoic acid
  SITE 13: note = (2S)-2,4-diaminobutanoic acid
  SITE 15: note = (2R,3S)-3-[(4-fluorophenyl)methyl]pyrrolidine-2-car boxylic acid
XXXGXXXSWXXQXGX

SEQ ID NO: 282 - protein, 15 aa, synthetic construct
  SITE 1: note = (S)-2-amino-3-(4-(pyrimidin-5-yl)phenyl)propanoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = (2S)-2-amino-3-pyrimidin-5-yl-propanoic acid
  SITE 13: note = (2S)-2,4-diaminobutanoic acid
  SITE 15: note = (2R,3S)-3-[(4-fluorophenyl)methyl]pyrrolidine-2-car boxylic acid
XXXGXXXSWXXQXGX

SEQ ID NO: 283 - protein, 15 aa, synthetic construct
  SITE 1: note = (S)-2-amino-3-(4-(pyridin-4-yl)phenyl)propanoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = (2S)-2-amino-3-pyrimidin-5-yl-propanoic acid
  SITE 13: note = (2S)-2,4-diaminobutanoic acid
  SITE 15: note = (2R,3S)-3-[(4-fluorophenyl)methyl]pyrrolidine-2-car boxylic acid
XXXGXXXSWXXQXGX

SEQ ID NO: 284 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(1-bicyclo[1.1.1]pentanyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = (2S)-2-amino-3-pyrimidin-4-yl-propanoic acid
  SITE 13: note = (2S)-2,4-diaminobutanoic acid
  SITE 15: note = (2R,3S)-3-(4-pyridylmethyl)pyrrolidine-2-carboxylic acid
XXXGXXXSWXXQXGX

SEQ ID NO: 285 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(1-bicyclo[1.1.1]pentanyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = (2S)-2-amino-3-pyrimidin-4-yl-propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 286 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = (2S)-2-amino-3-morpholino-propanoic acid
  SITE 13: note = (2S)-2,4-diaminobutanoic acid
  SITE 15: note = (2R,3S)-3-[(4-fluorophenyl)methyl]pyrrolidine-2-car boxylic acid
XXXGXXXSWXXQXGX

SEQ ID NO: 287 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = (2S)-2-amino-3-piperazin-1-yl-propanoic acid
  SITE 13: note = (2S)-2,4-diaminobutanoic acid
  SITE 15: note = (2R,3S)-3-[(4-fluorophenyl)methyl]pyrrolidine-2-car boxylic acid
XXXGXXXSWXXQXGX

SEQ ID NO: 288 - protein, 15 aa, synthetic construct
  SITE 1: note = (2S)-2-amino-3-(4-phenylphenyl)propanoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(1-naphthyl)butanoic acid
  SITE 7: note = 2-amino-2-methyl-propanoic acid
  SITE 10: note = (2R)-2-amino-2-methyl-butanedioic acid
  SITE 11: note = l-allothreonine
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 289 - protein, 15 aa, synthetic construct
  SITE 1: note = (2S)-2-amino-3-(4-phenylphenyl)propanoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-chloro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = l-allothreonine
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 290 - protein, 15 aa, synthetic construct
  SITE 1: note = (2S)-2-amino-3-(4-phenylphenyl)propanoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(1-naphthyl)butanoic acid
  SITE 7: note = 2-amino-2-methyl-propanoic acid
  SITE 10: note = (2R)-2-amino-2-methyl-butanedioic acid
  SITE 11: note = (2S)-2-amino-3-tetrahydropyran-4-yl-propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 291 - protein, 15 aa, synthetic construct
  SITE 1: note = (2S)-2-amino-3-(4-phenylphenyl)propanoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(1-naphthyl)butanoic acid
  SITE 7: note = 2-amino-2-methyl-propanoic acid
  SITE 10: note = (2R)-2-amino-2-methyl-butanedioic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 292 - protein, 15 aa, synthetic construct
  SITE 1: note = (2S)-2-amino-3-(4-phenylphenyl)propanoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-chloro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 293 - protein, 15 aa, synthetic construct
  SITE 1: note = (2S)-2-amino-3-(4-phenylphenyl)propanoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = l-allothreonine
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 294 - protein, 15 aa, synthetic construct
  SITE 1: note = (2S)-2-amino-3-(4-phenylphenyl)propanoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = (2S)-2-amino-3-tetrahydropyran-4-yl-propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 295 - protein, 15 aa, synthetic construct
  SITE 1: note = (2S)-2-amino-3-[4-[4-(dimethylcarbamoyl)phenyl]phen yl]propanoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = (2S)-2-amino-3-tetrahydropyran-4-yl-propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 296 - protein, 15 aa, synthetic construct
  SITE 1: note = (2S)-2-amino-3-[4-(4-carbamoylphenyl)phenyl]propano ic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = (2S)-2-amino-3-tetrahydropyran-4-yl-propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 297 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = [(4S)-4-amino-5-oxo-pentyl]-trimethyl-ammonium
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXSWXXQXGX

SEQ ID NO: 298 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = [(5S)-5-amino-5-carboxy-pentyl]-trimethyl-ammonium
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXSWXXQXGX

SEQ ID NO: 299 - protein, 15 aa, synthetic construct
  SITE 1: note = (2S)-2-amino-3-(4-bromophenyl)propanoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = (2S)-2-amino-3-pyrimidin-5-yl-propanoic acid
  SITE 13: note = (2S)-2,4-diaminobutanoic acid
  SITE 15: note = (2R,3S)-3-[(4-fluorophenyl)methyl]pyrrolidine-2-car boxylic acid
XXXGXXXSWXXQXGX

SEQ ID NO: 300 - protein, 15 aa, synthetic construct
  SITE 1: note = (S)-2-amino-3-(4-(2-pivalamidoethoxy)phenyl)propano ic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 301 - protein, 15 aa, synthetic construct
  SITE 1: note = (S)-2-amino-3-(4-(2-(cyclohexanecarboxamido)ethoxy) phenyl)propanoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 302 - protein, 15 aa, synthetic construct
  SITE 1: note = (S)-3-(4-(2-(1-acetylpiperidine-4-carboxamido)ethox y)phenyl)-2-aminopropanoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 303 - protein, 15 aa, synthetic construct
  SITE 1: note = (S)-2-amino-3-(4-(2-(pyrazine-2-carboxamido)ethoxy) phenyl)propanoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 304 - protein, 15 aa, synthetic construct
  SITE 1: note = (S)-2-amino-3-(4-(2-(pyrimidine-2-carboxamido)ethox y)phenyl)propanoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 305 - protein, 15 aa, synthetic construct
  SITE 1: note = (S)-2-amino-3-(4-(2-(pyrimidine-4-carboxamido)ethox y)phenyl)propanoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 306 - protein, 15 aa, synthetic construct
  SITE 1: note = (S)-2-amino-3-(4-(2-(4-(piperazin-1-yl)benzamido)et hoxy)phenyl)propanoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 307 - protein, 15 aa, synthetic construct
  SITE 1: note = (S)-2-amino-3-(4-(2-(isonicotinamido)ethoxy)phenyl) propanoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 308 - protein, 15 aa, synthetic construct
  SITE 1: note = (S)-2-amino-3-(4-(2-benzamidoethoxy)phenyl)propanoi c acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 309 - protein, 15 aa, synthetic construct
  SITE 1: note = (2S)-3-[4-(2-acetamidoethoxy)phenyl]-2-amino-propan oic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-(3-chlorophenyl)propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXEWXXQXGX

SEQ ID NO: 310 - protein, 15 aa, synthetic construct
  SITE 1: note = (2S)-3-[4-(2-acetamidoethoxy)phenyl]-2-amino-propan oic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXEWXYQXGX

SEQ ID NO: 311 - protein, 15 aa, synthetic construct
  SITE 1: note = (2S)-2-amino-3-(4-tert-butylphenyl)propanoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXEWXXQXGX

SEQ ID NO: 312 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-4-yl-propanoic acid
  SITE 13: note = (2S)-2,4-diaminobutanoic acid
  SITE 15: note = (2R,3S)-3-[(4-fluorophenyl)methyl]pyrrolidine-2-car boxylic acid
XXXGXXXEWXXQXGX

SEQ ID NO: 313 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = (2S)-2-amino-3-tetrahydropyran-4-yl-propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXSWXXQXGX

SEQ ID NO: 314 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = [(3S)-3-amino-4-oxo-butyl]-trimethyl-ammonium
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXSWXXQXGX

SEQ ID NO: 315 - protein, 15 aa, synthetic construct
  SITE 1: note = (2S)-3-[4-(2-acetamidoethoxy)phenyl]-2-amino-propan oic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (S)-2-amino-3-(5-methylisoxazol-3-yl)propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXEWXXQXGX

SEQ ID NO: 316 - protein, 15 aa, synthetic construct
  SITE 1: note = (2S)-3-[4-(2-acetamidoethoxy)phenyl]-2-amino-propan oic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (S)-2-amino-3-(oxazol-2-yl)propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXEWXXQXGX

SEQ ID NO: 317 - protein, 15 aa, synthetic construct
  SITE 1: note = (2S)-3-[4-(2-acetamidoethoxy)phenyl]-2-amino-propan oic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXEWXHQXGX

SEQ ID NO: 318 - protein, 15 aa, synthetic construct
  SITE 1: note = (2S)-3-[4-(2-acetamidoethoxy)phenyl]-2-amino-propan oic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-4-(4-pyridyl)butanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXEWXXQXGX

SEQ ID NO: 319 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = (2S)-2-amino-3-morpholino-propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXSWXXQXGX

SEQ ID NO: 320 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = (2S)-2-amino-3-(4-piperidyl)propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXSWXXQXGX

SEQ ID NO: 321 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = (2S)-2-amino-3-pyrazin-2-yl-propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXSWXXQXGX

SEQ ID NO: 322 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = (2S)-2-amino-3-pyridazin-4-yl-propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXSWXXQXGX

SEQ ID NO: 323 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = (2S)-2-amino-3-(3-pyridyl)propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXSWXXQXGX

SEQ ID NO: 324 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = (2S)-2-amino-3-(4-pyridyl)propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXSWXXQXGX

SEQ ID NO: 325 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-hydroxypyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,3S)-3-(4-pyridylmethyl)pyrrolidine-2-carboxylic acid
XXXGXXXSWXXQXGX

SEQ ID NO: 326 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,3S)-3-(4-pyridylmethyl)pyrrolidine-2-carboxylic acid
XXXGXXXSWXXQXGX

SEQ ID NO: 327 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-[(4-fluoro-1-naphthyl)methyl]pyrrolidine- 2-carboxylic acid
XXXGXXXSWXXQXGX

SEQ ID NO: 328 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = (2S)-2-amino-3-pyrimidin-5-yl-propanoic acid
  SITE 13: note = (2S)-2,4-diaminobutanoic acid
  SITE 15: note = (2R,3S)-3-(4-pyridylmethyl)pyrrolidine-2-carboxylic acid
XXXGXXXSWXXQXGX

SEQ ID NO: 329 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-[(4-chlorophenyl)methyl]pyrrolidine-2-car boxylic acid
XXXGXXXSWXXQXGX

SEQ ID NO: 330 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = (2S)-2-amino-3-pyrimidin-5-yl-propanoic acid
  SITE 13: note = (2S)-2,4-diaminobutanoic acid
  SITE 15: note = (2R,3S)-3-[(4-chlorophenyl)methyl]pyrrolidine-2-car boxylic acid
XXXGXXXSWXXQXGX

SEQ ID NO: 331 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = (2S)-2-amino-3-pyrimidin-5-yl-propanoic acid
  SITE 13: note = (2S)-2,4-diaminobutanoic acid
  SITE 15: note = (2R,3S)-3-[(4-fluorophenyl)methyl]pyrrolidine-2-car boxylic acid
XXXGXXXSWXXQXGX

SEQ ID NO: 332 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = (2S)-2-amino-3-pyrimidin-4-yl-propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXSWXXQXGX

SEQ ID NO: 333 - protein, 15 aa, synthetic construct
  SITE 1: note = (2S)-2-amino-3-(4-phenylphenyl)propanoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 334 - protein, 15 aa, synthetic construct
  SITE 1: note = (1R,4s)-4-(4-((S)-2-amino-2-carboxyethyl)phenyl)cyc lohexane-1-carboxylic acid
  SITE 2: note = (2S)-2-amino-3-(1-bicyclo[1.1.1]pentanyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = l-allothreonine
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 335 - protein, 15 aa, synthetic construct
  SITE 1: note = (1R,4s)-4-(4-((S)-2-amino-2-carboxyethyl)phenyl)cyc lohexane-1-carboxylic acid
  SITE 2: note = (2S)-2-amino-3-(1-bicyclo[1.1.1]pentanyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 336 - protein, 15 aa, synthetic construct
  SITE 1: note = (2S)-3-[4-(2-acetamidoethoxy)phenyl]-2-amino-propan oic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXEWXFQXGX

SEQ ID NO: 337 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = l-allothreonine
  SITE 13: note = (2S)-2,4-diaminobutanoic acid
  SITE 14: note = 2-(methylamino)acetic acid
  SITE 15: note = (2R,4R)-4-(4-pyridylmethyl)pyrrolidine-2-carboxylic acid
XXXGXXXSWXXQXXX

SEQ ID NO: 338 - protein, 15 aa, synthetic construct
  SITE 1: note = (2S)-2-amino-3-[4-[4-(dimethylcarbamoyl)phenyl]phen yl]propanoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-3-hydroxy-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 339 - protein, 15 aa, synthetic construct
  SITE 1: note = (2S)-2-amino-3-[4-(4-carbamoylphenyl)phenyl]propano ic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-3-hydroxy-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 340 - protein, 15 aa, synthetic construct
  SITE 1: note = (2S)-2-amino-3-(4-phenylphenyl)propanoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-3-hydroxy-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 341 - protein, 15 aa, synthetic construct
  SITE 1: note = (2S)-2-amino-3-[4-[4-(dimethylcarbamoyl)phenyl]phen yl]propanoic acid
  SITE 2: note = (2S)-2-amino-3-(1-bicyclo[1.1.1]pentanyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 342 - protein, 15 aa, synthetic construct
  SITE 1: note = (2S)-2-amino-3-[4-(4-carbamoylphenyl)phenyl]propano ic acid
  SITE 2: note = (2S)-2-amino-3-(1-bicyclo[1.1.1]pentanyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 343 - protein, 15 aa, synthetic construct
  SITE 1: note = (2S)-2-amino-3-(4-phenylphenyl)propanoic acid
  SITE 2: note = (2S)-2-amino-3-(1-bicyclo[1.1.1]pentanyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 344 - protein, 15 aa, synthetic construct
  SITE 1: note = (2S)-2-amino-3-[4-(4-chlorophenyl)phenyl]propanoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 345 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-4-yl-propanoic acid
  SITE 13: note = (2S)-2,4-diaminobutanoic acid
  SITE 15: note = (2R,4R)-4-[(4-fluorophenyl)methyl]pyrrolidine-2-car boxylic acid
XXXGXXXEWXXQXGX

SEQ ID NO: 346 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,3S)-3-[(4-fluorophenyl)methyl]pyrrolidine-2-car boxylic acid
XXXGXXXEWXXQXGX

SEQ ID NO: 347 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-3-hydroxy-2-methyl-propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSWXLQXGX

SEQ ID NO: 348 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(1-bicyclo[1.1.1]pentanyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = (S)-2-amino-3-cyclobutylpropanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 349 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(1-bicyclo[1.1.1]pentanyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-butanedioic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSWXLQXGX

SEQ ID NO: 350 - protein, 15 aa, synthetic construct
  SITE 1: note = (2S)-3-[4-(4-acetylpiperazin-1-yl)phenyl]-2-amino-p ropanoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-3-hydroxy-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 351 - protein, 15 aa, synthetic construct
  SITE 1: note = 2-[4-[(2S)-2-amino-2-carboxy-ethyl]phenyl]pyrimidin e-5-carboxylic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = (2S)-2-amino-3-tetrahydropyran-4-yl-propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 352 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 8: note = l-allothreonine
  SITE 10: note = (2R)-2-amino-3-hydroxy-2-methyl-propanoic acid
  SITE 11: note = l-allothreonine
  SITE 13: note = (2S)-2,4-diaminobutanoic acid
  SITE 15: note = (2R,4R)-4-(4-pyridylmethyl)pyrrolidine-2-carboxylic acid
XXXGXXXXWXXQXGX

SEQ ID NO: 353 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 8: note = l-allothreonine
  SITE 10: note = (2R)-2-amino-2-methyl-butanedioic acid
  SITE 11: note = (2S)-2-amino-3-tetrahydropyran-4-yl-propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXXWXXQXGX

SEQ ID NO: 354 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]-3-chloro-benzoic acid
  SITE 5: note = (2S,4S)-4-fluoropyrrolidine-2-carboxylic acid
  SITE 6: note = (2S,3S)-2-amino-3-(1-naphthyl)butanoic acid
  SITE 7: note = 2-amino-2-methyl-propanoic acid
  SITE 10: note = (2R)-2-amino-2-methyl-butanedioic acid
  SITE 11: note = l-allothreonine
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 355 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]-3-chloro-benzoic acid
  SITE 5: note = (2S,4S)-4-fluoropyrrolidine-2-carboxylic acid
  SITE 6: note = (2S,3S)-2-amino-3-(4-chloro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = l-allothreonine
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 356 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]-3-chloro-benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = l-allothreonine
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 357 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(1-naphthyl)butanoic acid
  SITE 7: note = 2-amino-2-methyl-propanoic acid
  SITE 10: note = (2R)-2-amino-2-methyl-butanedioic acid
  SITE 11: note = l-allothreonine
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 358 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-chloro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = l-allothreonine
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 359 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = l-allothreonine
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 360 - protein, 15 aa, synthetic construct
  SITE 1: note = 2-[4-[(2S)-2-amino-2-carboxy-ethyl]phenyl]pyrimidin e-5-carboxylic acid
  SITE 2: note = (2S)-2-amino-3-(4-pyridyl)propanoic acid
  SITE 3: note = 4-[(2S)-2-amino-2-carboxy-ethyl]-3-chloro-benzoic acid
  SITE 5: note = (2S,4S)-4-fluoropyrrolidine-2-carboxylic acid
  SITE 6: note = (2S,3S)-2-amino-3-(1-naphthyl)butanoic acid
  SITE 7: note = 2-amino-2-methyl-propanoic acid
  SITE 10: note = (2R)-2-amino-2-methyl-butanedioic acid
  SITE 11: note = (2S)-2-amino-3-tetrahydropyran-4-yl-propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 361 - protein, 15 aa, synthetic construct
  SITE 1: note = 2-[4-[(2S)-2-amino-2-carboxy-ethyl]phenyl]pyrimidin e-5-carboxylic acid
  SITE 2: note = (2S)-2-amino-3-(4-pyridyl)propanoic acid
  SITE 3: note = (S)-4-(2-amino-2-carboxyethyl)-2-chlorobenzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = (2S)-2-amino-3-tetrahydropyran-4-yl-propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 362 - protein, 15 aa, synthetic construct
  SITE 1: note = (2S)-3-[4-(2-acetamidoethoxy)phenyl]-2-amino-propan oic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-chloro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = (2S)-2-amino-3-tetrahydropyran-4-yl-propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 363 - protein, 15 aa, synthetic construct
  SITE 1: note = (2S)-3-[4-(2-acetamidoethoxy)phenyl]-2-amino-propan oic acid
  SITE 2: note = (2S)-2-amino-3-(4-pyridyl)propanoic acid
  SITE 3: note = 4-[(2S)-2-amino-2-carboxy-ethyl]-3-chloro-benzoic acid
  SITE 5: note = (2S,4S)-4-fluoropyrrolidine-2-carboxylic acid
  SITE 6: note = (2S,3S)-2-amino-3-(1-naphthyl)butanoic acid
  SITE 7: note = 2-amino-2-methyl-propanoic acid
  SITE 10: note = (2R)-2-amino-2-methyl-butanedioic acid
  SITE 11: note = (2S)-2-amino-3-tetrahydropyran-4-yl-propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 364 - protein, 15 aa, synthetic construct
  SITE 1: note = 4-[4-[(2S)-2-amino-2-carboxy-ethyl]phenyl]benzoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-3-hydroxy-2-methyl-propanoic acid
  SITE 11: note = (2S)-2-amino-3-tetrahydropyran-4-yl-propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 365 - protein, 15 aa, synthetic construct
  SITE 1: note = (2S)-3-[4-(4-acetylpiperazin-1-yl)phenyl]-2-amino-p ropanoic acid
  SITE 2: note = (2S)-2-amino-3-(4-pyridyl)propanoic acid
  SITE 3: note = 4-[(2S)-2-amino-2-carboxy-ethyl]-3-chloro-benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 366 - protein, 15 aa, synthetic construct
  SITE 1: note = (2S)-3-[4-(4-acetylpiperazin-1-yl)phenyl]-2-amino-p ropanoic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 367 - protein, 15 aa, synthetic construct
  SITE 1: note = (2S)-3-[4-(2-acetamidoethoxy)phenyl]-2-amino-propan oic acid
  SITE 2: note = (2S)-2-amino-3-(4-pyridyl)propanoic acid
  SITE 3: note = 4-[(2S)-2-amino-2-carboxy-ethyl]-3-chloro-benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 368 - protein, 15 aa, synthetic construct
  SITE 1: note = (2S)-3-[4-(2-acetamidoethoxy)phenyl]-2-amino-propan oic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 369 - protein, 15 aa, synthetic construct
  SITE 1: note = (2S)-3-[4-(2-acetamidoethoxy)phenyl]-2-amino-propan oic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = (2S)-2-amino-3-pyrimidin-5-yl-propanoic acid
  SITE 13: note = (2S)-2,4-diaminobutanoic acid
  SITE 15: note = (2R,3S)-3-[(4-fluorophenyl)methyl]pyrrolidine-2-car boxylic acid
XXXGXXXSWXXQXGX

SEQ ID NO: 370 - protein, 15 aa, synthetic construct
  SITE 1: note = (2S)-3-[4-(2-acetamidoethoxy)phenyl]-2-amino-propan oic acid
  SITE 2: note = (2S)-2-amino-3-(1-bicyclo[1.1.1]pentanyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = (2S)-2-amino-3-tetrahydropyran-4-yl-propanoic acid
  SITE 13: note = (2S)-2,4-diaminobutanoic acid
  SITE 15: note = (2R,3S)-3-[(4-fluorophenyl)methyl]pyrrolidine-2-car boxylic acid
XXXGXXXSWXXQXGX

SEQ ID NO: 371 - protein, 15 aa, synthetic construct
  SITE 1: note = (2S)-3-[4-(2-acetamidoethoxy)phenyl]-2-amino-propan oic acid
  SITE 2: note = (2S)-2-amino-3-(1-bicyclo[1.1.1]pentanyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-3-hydroxy-2-methyl-propanoic acid
  SITE 11: note = (2S)-2-amino-3-tetrahydropyran-4-yl-propanoic acid
  SITE 13: note = (2S)-2,4-diaminobutanoic acid
  SITE 15: note = (2R,3S)-3-[(4-fluorophenyl)methyl]pyrrolidine-2-car boxylic acid
XXXGXXXSWXXQXGX

SEQ ID NO: 372 - protein, 15 aa, synthetic construct
  SITE 1: note = (2S)-3-[4-(2-acetamidoethoxy)phenyl]-2-amino-propan oic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = (2S)-2-amino-3-tetrahydropyran-4-yl-propanoic acid
  SITE 13: note = (2S)-2,4-diaminobutanoic acid
  SITE 15: note = (2R,3S)-3-[(4-fluorophenyl)methyl]pyrrolidine-2-car boxylic acid
XXXGXXXSWXXQXGX

SEQ ID NO: 373 - protein, 15 aa, synthetic construct
  SITE 1: note = (2S)-3-[4-(2-acetamidoethoxy)phenyl]-2-amino-propan oic acid
  SITE 2: note = (2S)-2-amino-3-(1-bicyclo[1.1.1]pentanyl)propanoic acid
  SITE 3: note = (2S)-2-amino-3-(4-carbamoylphenyl)propanoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = (2S)-2-amino-3-tetrahydropyran-4-yl-propanoic acid
  SITE 13: note = (2S)-2,4-diaminobutanoic acid
  SITE 15: note = (2R,3S)-3-[(4-fluorophenyl)methyl]pyrrolidine-2-car boxylic acid
XXXGXXXSWXXQXGX

SEQ ID NO: 374 - protein, 15 aa, synthetic construct
  SITE 1: note = (2S)-3-[4-(2-acetamidoethoxy)phenyl]-2-amino-propan oic acid
  SITE 2: note = (2S)-2-amino-3-(1-bicyclo[1.1.1]pentanyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = (2S)-2-amino-3-tetrahydropyran-4-yl-propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 375 - protein, 15 aa, synthetic construct
  SITE 1: note = (2S)-3-[4-(2-acetamidoethoxy)phenyl]-2-amino-propan oic acid
  SITE 2: note = (2S)-2-amino-3-(1-bicyclo[1.1.1]pentanyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-3-hydroxy-2-methyl-propanoic acid
  SITE 11: note = (2S)-2-amino-3-tetrahydropyran-4-yl-propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 376 - protein, 15 aa, synthetic construct
  SITE 1: note = (2S)-3-[4-(2-acetamidoethoxy)phenyl]-2-amino-propan oic acid
  SITE 2: note = (2S)-2-amino-3-(1-bicyclo[1.1.1]pentanyl)propanoic acid
  SITE 3: note = (2S)-2-amino-3-(4-carbamoylphenyl)propanoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = (2S)-2-amino-3-tetrahydropyran-4-yl-propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 377 - protein, 15 aa, synthetic construct
  SITE 1: note = (2S)-3-[4-(2-acetamidoethoxy)phenyl]-2-amino-propan oic acid
  SITE 2: note = (2S)-2-amino-3-(4-pyridyl)propanoic acid
  SITE 3: note = (2S)-2-amino-3-(4-carbamoylphenyl)propanoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = (2S)-2-amino-3-tetrahydropyran-4-yl-propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 378 - protein, 15 aa, synthetic construct
  SITE 1: note = (2S)-3-[4-(2-acetamidoethoxy)phenyl]-2-amino-propan oic acid
  SITE 2: note = (2S)-2-amino-3-(4-pyridyl)propanoic acid
  SITE 3: note = (2S)-2-amino-3-(4-carbamoylphenyl)propanoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-3-hydroxy-2-methyl-propanoic acid
  SITE 11: note = (2S)-2-amino-3-tetrahydropyran-4-yl-propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 379 - protein, 15 aa, synthetic construct
  SITE 1: note = (2S)-2-amino-3-[4-(4-carbamoylphenyl)phenyl]propano ic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = (2S)-2-amino-3-pyrimidin-5-yl-propanoic acid
  SITE 13: note = (2S)-2,4-diaminobutanoic acid
  SITE 15: note = (2R,3S)-3-[(4-fluorophenyl)methyl]pyrrolidine-2-car boxylic acid
XXXGXXXSWXXQXGX

SEQ ID NO: 380 - protein, 15 aa, synthetic construct
  SITE 1: note = (2S)-2-amino-3-[4-(4-carbamoylphenyl)phenyl]propano ic acid
  SITE 2: note = (2S)-2-amino-3-(4-pyridyl)propanoic acid
  SITE 3: note = (2S)-2-amino-3-(4-carbamoylphenyl)propanoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = (2S)-2-amino-3-pyrimidin-5-yl-propanoic acid
  SITE 13: note = (2S)-2,4-diaminobutanoic acid
  SITE 15: note = (2R,3S)-3-[(4-fluorophenyl)methyl]pyrrolidine-2-car boxylic acid
XXXGXXXSWXXQXGX

SEQ ID NO: 381 - protein, 15 aa, synthetic construct
  SITE 1: note = (2S)-3-[4-(2-acetamidoethoxy)phenyl]-2-amino-propan oic acid
  SITE 2: note = (2S)-2-amino-3-(4-pyridyl)propanoic acid
  SITE 3: note = (2S)-2-amino-3-(4-carbamoylphenyl)propanoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = O-ethyl-L-allothreonine
  SITE 13: note = (2S)-4-amino-2-(methylamino)butanoic acid
  SITE 15: note = (2R,4R)-4-(4-pyridylmethyl)pyrrolidine-2-carboxylic acid
XXXGXXXSWXXQXGX

SEQ ID NO: 382 - protein, 15 aa, synthetic construct
  SITE 1: note = (2S)-3-[4-(2-acetamidoethoxy)phenyl]-2-amino-propan oic acid
  SITE 2: note = (2S)-2-amino-3-(4-pyridyl)propanoic acid
  SITE 3: note = (2S)-2-amino-3-(4-carbamoylphenyl)propanoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = O-ethyl-L-threonine
  SITE 13: note = (2S)-4-amino-2-(methylamino)butanoic acid
  SITE 15: note = (2R,4R)-4-(4-pyridylmethyl)pyrrolidine-2-carboxylic acid
XXXGXXXSWXXQXGX

SEQ ID NO: 383 - protein, 15 aa, synthetic construct
  SITE 1: note = (2S)-3-[4-(2-acetamidoethoxy)phenyl]-2-amino-propan oic acid
  SITE 2: note = (2S)-2-amino-3-(4-pyridyl)propanoic acid
  SITE 3: note = (2S,3S)-2-amino-3-(4-carbamoylphenyl)-3-hydroxyprop anoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-4-amino-2-(methylamino)butanoic acid
  SITE 15: note = (2R,4R)-4-(4-pyridylmethyl)pyrrolidine-2-carboxylic acid
XXXGXXXSWXXQXGX

SEQ ID NO: 384 - protein, 15 aa, synthetic construct
  SITE 1: note = (2S)-3-[4-(2-acetamidoethoxy)phenyl]-2-amino-propan oic acid
  SITE 2: note = (2S)-2-amino-3-(4-pyridyl)propanoic acid
  SITE 3: note = (2S)-2-amino-3-(4-carbamoylphenyl)propanoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = 2-amino-2-methyl-propanoic acid
  SITE 11: note = (2S)-2-amino-3-pyrimidin-5-yl-propanoic acid
  SITE 13: note = (2S)-2,4-diaminobutanoic acid
  SITE 15: note = (2R,4R)-4-(4-pyridylmethyl)pyrrolidine-2-carboxylic acid
XXXGXXXSWXXQXGX

SEQ ID NO: 385 - protein, 15 aa, synthetic construct
  SITE 1: note = (S)-2-amino-3-(4-(2-aminopyridin-4-yl)phenyl)propan oic acid
  SITE 2: note = (2S)-2-amino-3-(4-pyridyl)propanoic acid
  SITE 3: note = 4-[(2S)-2-amino-2-carboxy-ethyl]benzoic acid
  SITE 5: note = (2S,4S)-4-fluoropyrrolidine-2-carboxylic acid
  SITE 6: note = (2S,3S)-2-amino-3-(4-fluoro-1H-indol-3-yl)butanoic acid
  SITE 7: note = 2-amino-2-methyl-propanoic acid
  SITE 10: note = (2R)-2-amino-2-methyl-butanedioic acid
  SITE 11: note = (2S)-2-amino-3-pyrimidin-5-yl-propanoic acid
  SITE 13: note = (2S)-2,4-diaminobutanoic acid
  SITE 15: note = (2R,3S)-3-[(4-fluorophenyl)methyl]pyrrolidine-2-car boxylic acid
XXXGXXXSWXXQXGX

(19) United States
(12) Patent Application Publication
(10) Pub. No.: US 2026/0242419 A1
(43) Pub. Date: Aug. 20, 2026

(54) A CYCLIC PEPTIDE FOR TRAPPING INTERLEUKIN-1 BETA

(71) Applicant: Merck Sharp & Dohme LLC, Rahway, NJ (US)

(72) Inventors: Yan Guo, Westfield, NJ (US); Jennifer Hanisak, Stewartsville, NJ (US); Jennifer L. Hickey, Fords, NJ (US); Ahmet Kekec, Redwood City, CA (US); Angela D. Kerekes, Marlborough, MA (US); Michael Man-Chu Lo, Bendminster, NJ (US); Victor W. Mak, San Mateo, CA (US); Christopher W. Plummer, Westfield, NJ (US); Steven M. Silverman, Jersey City, NJ (US); Stefania Colarusso, Rome (IT); Emanuela Nizi, Ariccia (IT); Francesca Pavone, Rome (IT)

(73) Assignee: Merck Sharp & Dohme LLC, Rahway, NJ (US)

(21) Appl. No.: 19/471,956

(22) PCT Filed: Apr. 11, 2024
(86) PCT No.: PCT/US2024/023955

§ 371 Date: Oct. 2, 2025

Related U.S. Application Data
(60) Provisional application No. 63/496,260, filed on Apr. 14, 2023.

(51) Int. Cl. C07K 7/64; A61K 38/00

(52) U.S. Cl. CPC C07K 7/64; A61K 38/00

(57) ABSTRACT

Provided are compounds of the Formula (I), or their pharmaceutically acceptable salts, wherein X¹, X², X³, X⁴, R¹-R⁴, R^{5a}, R^{5b}, R^{6a}, R^{6b}, R^{7a}, R^{7b}, R^{8a}, R^{8b}, R⁹-R⁷ and the subscripts v and w are as herein described can trap IL-1β and are expected to have utility as therapeutic agents, for example, for treating cardiovascular disease. The disclosure also provides pharmaceutical compositions which comprise the compounds disclosed herein or pharmaceutically acceptable salts thereof. The disclosure also relates to methods for use of the compounds or their pharmaceutically acceptable salts in the therapy and prophylaxis of cardiovascular disease and inflammatory disorders and for preparing pharmaceuticals for this purpose.

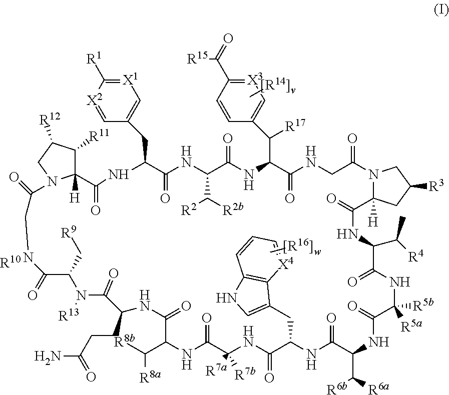

### CROSS-REFERENCE TO RELATED APPLICATIONS

This application claims the benefit of U.S. Provisional Patent Application Ser. No. 63/496,260 filed Apr. 14, 2023, the entire contents of which are incorporated by reference herein.

### REFERENCE TO SEQUENCE LISTING SUBMITTED ELECTRONICALLY

The contents of the electronic sequence listing (25677-WO-PCT_SL.xml; Size: 1,859,444 bytes; and Date of Creation: Apr. 4, 2024) are herein incorporated by reference in their entirety.

### FIELD OF THE INVENTION

The present disclosure relates to certain cyclic peptides that trap interleukin-1β (IL-1β), pharmaceutical compositions comprising such peptides, and methods for using the compounds for treating, inhibiting, or ameliorating one or more cardiovascular disease states that could benefit from trapping IL-1β, including atherosclerosis.

### BACKGROUND OF THE INVENTION

Atherosclerosis is a disease of the arteries characterized by the accumulation of cholesterol plaques on the interior wall of the artery. Progression of atherosclerosis can result in hardening or narrowing of the arteries and increases the risk of plaque ruptures. These ruptures release cholesterol globules and other material into the bloodstream which may result in blockage of blood flow to the brain, heart, or other organ. Medically, these are known as Major Adverse Cardiac Events (MACE).

Risk factors for the development and progression of Atherosclerotic Cardiovascular Disease (ASCVD) include high cholesterol, high blood pressure, diet high in saturated fat, smoking, obesity, diabetes, lack of exercise, and elevated levels of C-reactive protein (CRP), a marker of inflammation.

The first line of treatment to prevent the progression of ASCVD is a healthy diet and exercise, however, compliance is generally poor. Pharmacological treatments for ASCVD have largely focused on cholesterol-lowering medications such as statins, cholesterol absorption inhibitors, and low-density lipoprotein (LDL) receptor inhibitors. These medications are highly effective at reducing the buildup of fatty acid deposits and improving arterial health. Other medications that are prescribed for ASCVD which do not ameliorate the disease state include blood thinners, such as aspirin, to prevent clumping of platelets in narrow arteries, and blood pressure medications to reduce the risk and severity of heart attacks. Surgical options for more aggressive intervention in advanced cases of ASCVD include angioplasty, stent placement, endarterectomy (surgical removal of plaques), and bypass surgery.

While cholesterol-lowering medications have served as an important standard of care for slowing the progression of atherosclerosis, clinical data support an additional critical role for inflammation in the progression of ASCVD that has remained untreated. Biomarkers of inflammation such as CRP are associated with increased risk of cardiovascular events, independent of cholesterol levels. The Canakinumab Anti-inflammatory Thrombosis Outcomes Study (CANTOS) was the first clinical trial to show that reducing vascular inflammation in the absence of concomitant lipid lowering reduces the rates of cardiovascular events. N Engl J Med 2017; 377:1119-1131. Canakinumab is an anti-interleukin-1 beta (IL-1β) human monoclonal antibody approved for clinical use in rheumatologic disorders. IL-1β is a proinflammatory cytokine that induces IL-6 and thereby elevates the downstream inflammatory biomarker high sensitivity CRP (hsCRP). Therefore, CANTOS provides proof of concept that therapies targeting IL-1β could reduce rates of MACE in certain patients in a manner that is complimentary and potentially additive to the LDL-lowering standard of care.

There is a need for additional, non-surgical therapeutic approaches beyond the standard of care cholesterol-lowering medications for slowing the progression of atherosclerosis and decreasing the risk of MACE. In addition, patients suffering from inflammatory disorders would benefit from orally administered agents which block the same cytokine, IL-1β, as canakinumab.

### SUMMARY OF THE DISCLOSURE

The present disclosure provides certain cyclic peptides that reduce inflammation by binding to the IL-1β cytokine and prevent engagement with the IL-1 receptor, resulting in inhibition of downstream pro-inflammatory signaling. These cyclic peptides can be valuable pharmaceutically active compounds for the treatment of cardiovascular diseases and inflammatory disorders. In one aspect, the present disclosure provides compounds of Formula (I)

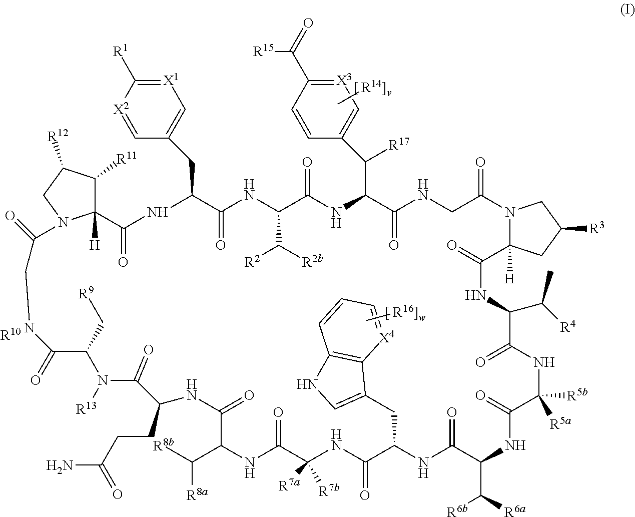

and their pharmaceutically acceptable salts.

The compounds can trap IL-1β and thereby affect the downstream pro-inflammatory signaling pathway which may be associated with cardiovascular disorders. Accordingly, in another aspect, the present disclosure provides a method for treating a cardiovascular disorder (e.g., atherosclerosis, vascular inflammation) comprising administering a therapeutically effective amount of the compound of the disclosure to a subject in need thereof. In some embodiments, the administration comprises an oral administration of the compound.

The disclosure furthermore provides processes for preparing compounds of the disclosure and pharmaceutical compositions which comprise compounds of the disclosure and a pharmaceutically acceptable carrier.

### DETAILED DESCRIPTION OF THE INVENTION

### Compounds of the Disclosure

In one embodiment, the present disclosure provides a compound having the Formula (I) as shown above, wherein:
R¹ is R^{1e}—C(O)NH—CH₂CH₂—O—, C₁-C₄ alkyl, halo, or C¹:
   R^{1e}— is:
      (a) C₁-C₄ alkyl; or
      (b) C^{Y1}; wherein C^{Y1} is:
         (i) C₃-C₆ cycloalkyl;
         (ii) phenyl; or
         (iii) a 5- to 6-membered monocyclic heteroaryl, wherein said heteroaryl contains 1 to 2 heteroatoms independently selected from the group consisting of N, O, and S;
         wherein C^{Y1} is unsubstituted or substituted by 1 to 3 R^{Y1} substituents selected from the group consisting of C₁-C₃ alkyl, halo, and piperazinyl;
   C¹ is:
      (i) a 5- to 6-membered monocyclic aryl or heteroaryl, wherein said heteroaryl contains 1 to 2 heteroatoms independently selected from the group consisting of N, O, and S;
      (ii) a 3- to 6-membered monocyclic or a 5- to 8-membered bicyclic cycloalkyl; or
      (iii) a 5- to 6-membered monocyclic, saturated heterocycloalkyl containing 1 to 2 heteroatoms independently selected from the group consisting of N, O, and S;
         wherein C¹ is unsubstituted or substituted by 1 to 3 R^{C1} substituents independently selected from the group consisting of halo, C₁-C₃ alkyl, C₁-C₃ fluoroalkyl, carboxy, C₁-C₃ alkoxy, C₂-C₃ acyl, —C(O)NH₂, and —C(O)N(CH₃)₂;
R² is:
   (i) a 5- to 6-membered monocyclic aryl or heteroaryl, wherein said heteroaryl contains 1 to 2 heteroatoms independently selected from the group consisting of N, O, and S; and
   (ii) a 3- to 8-membered mono- or bicyclic cycloalkyl;
      wherein R² is unsubstituted or substituted by 1 to 3 R^{2a} substituents independently selected from the group consisting of halo, amino, hydroxy, C₁-C₃ alkyl, C₁-C₃ fluoroalkyl, and C₁-C₃ alkoxy;
R^{2b} is H or hydroxy;
R³ is F or hydroxy;
R⁴ is:
   (i) naphthyl; or
   (ii) a 9- to 10-membered heteroaryl, wherein said heteroaryl contains 1 to 2 heteroatoms independently selected from the group consisting of N, O, and S;
   wherein R⁴ is unsubstituted or substituted by 1 to 2 R^{4a} substituents independently selected from the group consisting of halo;
R^{5a} is H, C₁-C₃ alkyl, H₂N(CH₂)ₘ—, or HOCH₂—;
R^{5b} is H, C₁-C₃ alkyl, H₂N(CH₂)ₘ—, or HOCH₂—;
or, alternatively R^{5a} and R^{5b}, together with the carbon atom to which they attached, form a C₃-C₆ cycloalkyl or a 4- to 6-membered saturated heterocycloalkyl containing one N atom;
each of R^{6a} and R^{6b} is independently H, —(CH₂)ₙ₁CH₃, —(CH₂)ₙ₂—OH, or —(CH₂)ₙ₂CO₂H;
R^{7a} is H, C₁-C₃ alkyl, HOCH₂—, H₂N(CH₂)ₚ—, HO₂CCH₂—, H₂NC(O)CH₂—, CH₃OCH₂—, or PhCH₂—;
R^{7b} is H, C₁-C₃ alkyl, HOCH₂—, H₂N(CH₂)ₚ—, HO₂CCH₂—, H₂NC(O)CH₂—, CH₃OCH₂—, or PhCH₂—;
   or, alternatively R^{7a} and R^{7b}, together with the carbon atom to which they are attached, form a 4- to 6-membered saturated heterocycloalkyl containing one N atom;
R^{8a} is HO—(CH₂)_{q}—, CH₃—O—(CH₂)_{q}—, CH₃CH₂—O—(CH₂)_{q}—, PhCH₂—O—(CH₂)_{q}—, C₁-C₃ alkyl, C₁-C₃ fluoroalkyl, H₂N—(CH₂)ᵣ—, (CH₃)₃N—(CH₂)ᵣ—, H₂NC(NH)N(H)—(CH₂)ᵣ—, H₂NC(O)N(H)—(CH₂)ᵣ—, HO₂C—(CH₂)ᵣ—, (CH₃)SO₂—(CH₂)ᵣ—, C^{8a}, or C^{8a}—CH₂—;
   wherein C^{8a} is:
   (i) a 5- to 6-membered monocyclic aryl or heteroaryl, wherein said heteroaryl contains 1 to 2 heteroatoms independently selected from the group consisting of N, O, and S;
   (ii) a 5- to 6-membered monocyclic, saturated heterocycloalkyl, wherein said heterocycloalkyl contains 1 to 2 heteroatoms independently selected from the group consisting of N, O, and S; or
   (iii) a C₃-C₆ cycloalkyl;
      wherein C^{8a} is unsubstituted or substituted by 1 to 3 R^{C8a} substituents independently selected from the group consisting of halo, amino, hydroxy, C₁-C₃ alkyl, C₁-C₃ fluoroalkyl, C₁-C₃ alkoxy, H₂N—(CH₂)ₛ—, H₂NC(O)—(CH₂)ₛ—, H₂C═CH—CH₂O—, and phenyl;
R^{8b} is H, methyl, or hydroxy;
R⁹ is HO—(CH₂)ₜ—, H₂N—(CH₂)ᵤ—, H₂NC(NH)N(H)—(CH₂)ᵤ—, H₂NC(O)N(H)—(CH₂)ᵤ—, or C⁹;
   C⁹ is a 5- to 6-membered saturated heterocycloalkyl containing 1 to 2 heteroatoms independently selected from the group consisting of N, O and S;
   wherein C⁹ is unsubstituted or substituted by 1 to 2 R^{C9} moieties independently selected from the group consisting of halo, C₁-C₃ alkyl, C₁-C₃ fluoroalkyl, and C₁-C₃ alkoxy:
R¹⁰ is H or methyl;
R¹¹ is H, —CH₂—C¹¹, or —CH₂—C¹¹—C^{a};
   C¹¹ is:
      (i) phenyl; or
      (ii) a 5- to 6-membered monocyclic heteroaryl, wherein said heteroaryl contains 1 to 2 heteroatoms independently selected from the group consisting of N, O, and S; wherein C¹¹ is unsubstituted or substituted by 1 to 3 R^{C11} substituents independently selected from the group consisting of halo, hydroxy, amino, C₁-C₃ alkyl, C₁-C₃ fluoroalkyl, carboxy, C₁-C₃ alkoxy, and C₂-C₃ acyl;
   C^{a} is a 5- to 6-membered monocyclic heteroaryl, wherein said heteroaryl contains 1 to 2 heteroatoms independently selected from the group consisting of N, O, and S:
      wherein C^{a} is unsubstituted or substituted by 1 to 3 R^{Ca} substituents independently selected from the group consisting of halo, hydroxy, amino, C₁-C₃ alkyl, C₁-C₃ fluoroalkyl, carboxy, C₁-C₃ alkoxy, and C₂-C₃ acyl;
R¹² is H or —CH₂C¹²;
   C¹² is:
      (i) phenyl; or
      (ii) a 5- to 6-membered monocyclic heteroaryl, wherein said heteroaryl contains 1 to 2 heteroatoms independently selected from the group consisting of N, O, and S;
      wherein C¹² is unsubstituted or substituted by 1 to 3 R^{C12} substituents independently selected from the group consisting of halo, hydroxy, amino, C₁-C₃ alkyl, C₁-C₃ fluoroalkyl, carboxy, C₁-C₃ alkoxy, and C₂-C₃ acyl;
R¹³ is H or methyl;
R¹⁴ is halo;
R¹⁵ is —OH or —NH₂;
R¹⁶ is halo, hydroxy, C₁-C₃ alkyl, C₁-C₃ fluoroalkyl, or C₁-C₃ alkoxy;
R¹⁷ is H, hydroxy, or methyl;
each occurrence of subscript m is independently 1, 2, 3, or 4;
subscript n1 is 0, 1, 2, or 3;
subscript n2 is 0, 1, or 2;
each occurrence of subscript p is independently 2, 3 or 4;
subscript q is 0, 1 or 2;
subscript r is 0, 1, 2, or 3;
each occurrence of subscript s is independently 1 or 2;
subscript t is 0, 1, or 2;
subscript u is 0, 1, 2, or 3;
subscript v is 0, 1 or 2;
subscript w is 0, 1, or 2;
X¹, and X², are independently C(H) or N; and
X³ and X⁴ are independently C(H), C(Cl), C(F) or N; or
a pharmaceutically acceptable salt thereof.

In some embodiments, the present disclosure provides a compound of Formula (I), the group

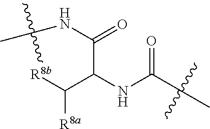

is

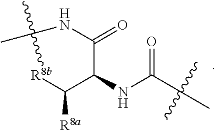

In certain embodiments, the present disclosure provides a compound of Formula (I), wherein:
X¹ and X² are C(H); and
R¹ is:
   C¹, wherein C¹ is phenyl or bicyclo[1.1.1]pentanyl;
      wherein C¹ is substituted by carboxy; or
   R^{1e}—C(O)NH—CH₂CH₂—O—,
      wherein R^{1e} is C₁-C₄ alkyl.

In specific embodiments, the present disclosure provides a compound of Formula (I), wherein R^{2b} is H.

In certain embodiments, the present disclosure provides a compound of Formula (I), wherein R¹⁷ is H.

In one embodiment, the present disclosure provides a compound of Formula (I) having the Formula (IA)

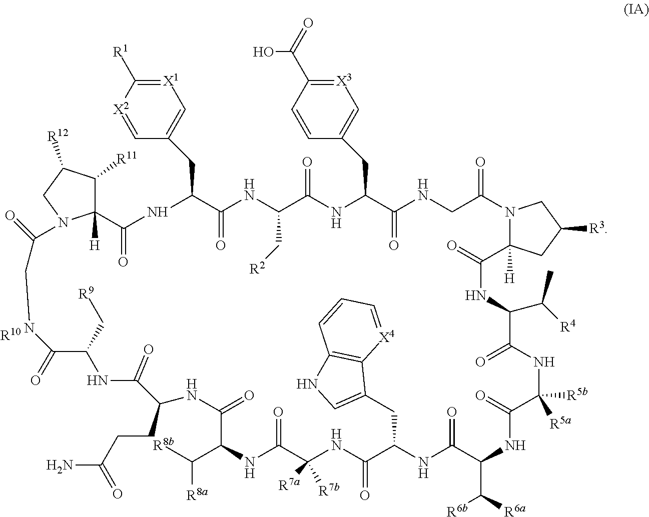

In specific embodiments, the present disclosure provides a compound having the Formula (IA), wherein:
R¹ is CH₃C(O)NH—CH₂CH₂—O— or C¹;
   C¹ is:
      (i) a 5- to 6-membered monocyclic aryl or heteroaryl, wherein said heteroaryl contains 1 to 2 heteroatoms independently selected from the group consisting of N, O, and S;
      (ii) a 3- to 6-membered monocyclic or a 5- to 8-membered bicyclic cycloalkyl; or
      (iii) a 5- to 6-membered monocyclic, saturated heterocycloalkyl containing 1 to 2 heteroatoms independently selected from the group consisting of N, O, and S; wherein C¹ is unsubstituted or substituted by 1 to 3 R^{C1} substituents independently selected from the group consisting of halo, C₁-C₃ alkyl, C₁-C₃ fluoroalkyl, carboxy, C₁-C₃ alkoxy, and C₂-C₃ acyl;
R² is:
   (i) a 5- to 6-membered monocyclic aryl or heteroaryl, wherein said heteroaryl contains 1 to 2 heteroatoms independently selected from the group consisting of N, O, and S; and
   (ii) a 3- to 8-membered mono- or bicyclic cycloalkyl;
      wherein R² is unsubstituted or substituted by 1 to 3 R^{2a} substituents independently selected from the group consisting of halo, amino, hydroxy, C₁-C₃ alkyl, C₁-C₃ fluoroalkyl, and C₁-C₃ alkoxy;
R³ is F or hydroxy;
R⁴ is
   (i) naphthyl; or
   (ii) a 9- to 10-membered heteroaryl, wherein said heteroaryl contains 1 to 2 heteroatoms independently selected from the group consisting of N, O, and S;
   wherein R⁴ is unsubstituted or substituted by 1 to 2 R^{4a} substituents independently selected from the group consisting of halo;
R^{5a} is H, C₁-C₃ alkyl, H₂N(CH₂)ₘ—, or HOCH₂—;
R^{5b} is H, C₁-C₃ alkyl, H₂N(CH₂)ₘ—, or HOCH₂—;
or, alternatively R^{5a} and R^{5b}, together with the carbon atom to which they attached, form a 4- to 6-membered saturated heterocycloalkyl containing one N atom;
each of R^{6a} and R^{6b} is independently H, —(CH₂)ₙ₁CH₃, —(CH₂)ₙ₂—OH, or —(CH₂)ₙ₂CO₂H;
R^{7a} is H, C₁-C₃ alkyl, HOCH₂—, H₂N(CH₂)ₚ—, or HO₂CCH₂—;
R^{7b} is H, C₁-C₃ alkyl, HOCH₂—, H₂N(CH₂)ₚ—, or HO₂CCH₂—;
   or, alternatively R^{7a} and R^{7b}, together with the carbon atom to which they are attached, form a 4- to 6-membered saturated heterocycloalkvl containing one N atom;
R^{8a} is HO—(CH₂)_{q}—, C₁-C₃ alkyl, H₂N—(CH₂)ᵣ—, H₂NC(NH)N(H)—(CH₂)ᵣ—, H₂NC(O)N(H)—(CH₂)ᵣ—, or C^{8a},
   wherein C^{8a} is
   (i) a 5- to 6-membered monocyclic aryl or heteroaryl, wherein said heteroaryl contains 1 to 2 heteroatoms independently selected from the group consisting of N, O, and S; or
   (ii) a 5- to 6-membered monocyclic, saturated heterocycloalkyl, wherein said heterocycloalkyl contains 1 to 2 heteroatoms independently selected from the group consisting of N, O, and S;
      wherein C^{8a} is unsubstituted or substituted by 1 to 3 R^{C8a} substituents independently selected from the group consisting of halo, amino, hydroxy, C₁-C₃ alkyl, C₁-C₃ fluoroalkyl, C₁-C₃ alkoxy, H₂N—(CH₂)ₛ—, H₂NC(O)—(CH₂)ₛ—, H₂C═CH—CH₂O—, and phenyl;
R^{8b} is H or CH₃;
R⁹ is HO—(CH₂)ₜ—, H₂N—(CH₂)ᵤ—, H₂NC(NH)N(H)—(CH₂)ᵤ—, or C₉;
   C⁹ is a 5- to 6-membered saturated heterocycloalkyl containing 1 to 2 heteroatoms independently selected from the group consisting of N, O and S;
      wherein C⁹ is unsubstituted or substituted by 1 to 2 R^{C9} moieties independently selected from the group consisting of halo, C₁-C₃ alkyl, C₁-C₃ fluoroalkyl, and C₁-C₃ alkoxy;
R¹⁰ is H or methyl;
R¹¹ is H, —CH₂—C₁₁, or —CH₂—C¹¹—C^{a};
   C¹¹ is:
      (i) phenyl; or
      (ii) a 5- to 6-membered monocyclic heteroaryl, wherein said heteroaryl contains 1 to 2 heteroatoms independently selected from the group consisting of N, O, and S;
         wherein C¹¹ is unsubstituted or substituted by 1 to 3 R^{C11} substituents independently selected from the group consisting of halo, hydroxy, amino, C₁-C₃ alkyl, C₁-C₃ fluoroalkyl, carboxy, C₁-C₃ alkoxy, and C₂-C₃ acyl;
   C^{a} is a 5- to 6-membered monocyclic heteroaryl, wherein said heteroaryl contains 1 to 2 heteroatoms independently selected from the group consisting of N, O, and S;
      wherein C^{a} is unsubstituted or substituted by 1 to 3 R^{Ca} substituents independently selected from the group consisting of halo, hydroxy, amino, C₁-C₃ alkyl, C₁-C₃ fluoroalkyl, carboxy, C₁-C₃ alkoxy, and C₂-C₃ acyl;
R¹² is H or —CH₂C¹²;
   C¹² is:
      (i) phenyl; or
      (ii) a 5- to 6-membered monocyclic heteroaryl, wherein said heteroaryl contains 1 to 2 heteroatoms independently selected from the group consisting of N, O, and S;
      wherein C¹² is unsubstituted or substituted by 1 to 3 R^{C12} substituents independently selected from the group consisting of halo, hydroxy, amino, C₁-C₃ alkyl, C₁-C₃ fluoroalkyl, carboxy, C₁-C₃ alkoxy, and C₂-C₃ acyl;
each occurrence of subscript m is independently 1, 2, 3, or 4;
subscript n1 is 0, 1, 2, or 3;
subscript n2 is 0, 1, or 2;
each occurrence of subscript p is independently 2, 3 or 4;
subscript q is 0, 1 or 2;
subscript r is 0, 1, 2, or 3;
each occurrence of subscript s is independently 1 or 2;
subscript t is 0, 1, or 2;
subscript u is 0, 1, 2, or 3; and
X¹, X², X³ and X⁴ are independently C(H) or N; or
a pharmaceutically acceptable salt thereof.

In another embodiment, the present disclosure provides a compound Formula (I) or (IA), wherein:
C¹ is phenyl, pyrimidinyl, or piperazinyl, wherein C¹ is unsubstituted or substituted by 1 to 2 R^{C1} substituents;
R² is pyridyl or bicyclo[1.1.1]pentanyl, wherein R² is unsubstituted or substituted by 1 to 2 R^{2a} substituents;
R⁴ is indolyl or naphthyl, wherein R⁴ is unsubstituted or substituted by 1 R^{4a} substituent;
C^{8a} is phenyl, pyridyl, pyrimidinyl, pyridazinyl, tetrahydropyranyl, piperidinyl, morpholinyl, or piperazinyl; wherein C^{8a} is unsubstituted or substituted by 1 to 2 R^{C8a};
C⁹ is morpholinyl, wherein C⁹ is unsubstituted or substituted by 1 R^{C9};
R¹¹ is
   —CH₂—C¹¹, wherein C¹¹ is phenyl, pyridyl, pyrimidinyl, or pyrazinyl, wherein C¹¹ is unsubstituted or substituted by 1 R^{C8};
   —CH₂—C¹¹—C^{a}, wherein:
      C¹¹ is phenyl, wherein C¹¹ is unsubstituted or substituted by 1 R^{C11}; and
      C^{a} is pyridyl, pyrimidinyl, pyrazinyl, pyridazinyl, isoxazolyl, or imidazolyl; wherein C^{a} is unsubstituted or substituted by 1 R^{Ca}; and
R¹² is —CH₂C¹², wherein C¹² is phenyl or pyridyl, wherein C¹² is unsubstituted or substituted by 1 R^{C12}.

In another embodiment, the present disclosure provides a compound of Formula (I) or (IA), wherein:
X¹ and X² are C(H); and
R¹ is phenyl substituted by carboxy.

In another embodiment, the present disclosure provides a compound of Formula (I) or (IA), wherein R² is the 5- to 6-membered monocyclic aryl or heteroaryl, unsubstituted or substituted by 1 to 3 R^{2a} substituents; and X³ is C(H). For instance, in one specific embodiment, R² is unsubstituted or substituted pyridyl.

In another embodiment, the present disclosure provides a compound of Formula (I) or (IA), wherein R² is unsubstituted bicyclo[1.1.1]pentanyl.

In another embodiment, the present disclosure provides a compound of Formula (I) or (IA), wherein R³ is fluoro.

In another embodiment, the present disclosure provides a compound of Formula (I) or (IA), wherein R⁴ is 4-fluoroindolyl.

In another embodiment, the present disclosure provides a compound of Formula (I) or (IA), wherein R^{5a} and R^{5b} bare methyl.

In another embodiment, the present disclosure provides a compound of Formula (I) or (IA), wherein:
R^{6a} is H, —(CH₂)ₙ₁CH₃, —(CH₂)ₙ₂—OH, or —(CH₂)ₙ₂CO₂H;
R^{6b} is H or methyl;
subscript n1 is 1, 2, or 3; and
subscript n2 is 0, 1, or 2.

In another embodiment, the present disclosure provides a compound of Formula (I) or (IA), wherein:
R^{6a} is —OH or —CH₂CO₂H; and
R^{6b} is H.

In another embodiment, the present disclosure provides a compound of Formula (I) or (IA), wherein X⁴ is C(H).

In another embodiment, the present disclosure provides a compound of Formula (I) or (IA), wherein R^{7a} and R^{7b} are methyl.

In another embodiment, the present disclosure provides a compound of Formula (I) or (IA), wherein:
R^{8a} is phenyl, pyridyl pyrimidinyl, pyrazinyl, pyridazinyl, piperidinyl, tetrahydropyranyl, or morpholinyl, substituted or unsubstituted by 1 to 3 R^{C8a} substituents; and
R^{8b} is H.

In another embodiment, the present disclosure provides a compound of Formula (I) or (IA), wherein R^{8a} is unsubstituted pyridyl, pyrimidinyl or pyrazinyl.

In another embodiment, the present disclosure provides a compound of Formula (I) or (IA), wherein R⁹ is H₂N—(CH₂)ᵤ— and subscript u is 1 or 2.

In another embodiment, the present disclosure provides a compound of Formula (I) or (IA), wherein R¹⁰ is H.

In another embodiment, the present disclosure provides a compound of Formula (I) or (IA), wherein:
R¹¹ is H; and
R¹² is —CH₂C¹², wherein C¹² is phenyl or pyridyl, wherein C¹² is unsubstituted or substituted by 1 R^{C12}.

In another embodiment, the present disclosure provides a compound of Formula (I) or (IA), wherein:
R¹¹ is:
   —CH₂—C¹¹, wherein C¹¹ is phenyl, pyridyl, pyrimidinyl, or pyrazinyl, wherein C¹¹ is unsubstituted or substituted by 1 R^{C11}; or
   —CH₂—C¹¹—C^{a}, wherein:
      C¹¹ is phenyl, wherein C¹¹ is unsubstituted or substituted by 1 R^{C11}; and
      C^{a} is pyridyl, pyrimidinyl, pyrazinyl, pyridazinyl, isoxazolyl, or imidazolyl;
         wherein C^{a} is unsubstituted or substituted by 1 R^{Ca}; and
R¹² is H.

In another embodiment, the present disclosure provides a compound of Formula (I), wherein:
X¹ X², X³ and X⁴ are C(H);
R¹ is phenyl substituted by carboxy;
R² is the 5- to 6-membered monocyclic aryl or heteroaryl, unsubstituted or substituted by 1 to 3 R^{2a} substituents;
R³ is fluoro;
R⁴ is 4-fluoroindolyl;
R^{5a} and R^{5b} bare methyl;
R^{6a} is —OH or —CH₂CO₂H;
R^{6b} is H;
R^{7a} and R^{7b} are methyl;
R^{8a} is phenyl, pyridyl, pyrimidinyl, pyrazinyl, pyridazinyl, piperidinyl, tetrahydropyranyl, or morpholinyl, unsubstituted or substituted by 1 to 3 R^{C8a} substituents;
R^{8b} is H
R⁹ is H₂N—(CH₂)ᵤ—;
R¹⁰ is H; and
subscript u is 1 or 2.

In a specific aspect of this embodiment, the present disclosure provides a compound of Formula (I) or (IA), wherein:
R¹¹ is H; and
R¹² is —CH₂C¹², wherein C¹² is phenyl or pyridyl, wherein C¹² is unsubstituted or substituted by 1 R^{C12}.

In another specific aspect of this embodiment, the present disclosure provides a compound of Formula (I) or (IA), wherein:
R¹¹ is:
   —CH₂—C¹¹, wherein C¹¹ is phenyl, pyridyl, pyrimidinyl, or pyrazinyl, wherein C¹¹ is unsubstituted or substituted by 1 R^{C11}; or
   —CH₂—C¹¹—C^{a}, wherein:
      C¹¹ is phenyl, wherein C¹¹ is unsubstituted or substituted by 1 R^{C11}; and
      C^{a} is pyridyl, pyrimidinyl, pyrazinyl, pyridazinyl, isoxazolyl, or imidazolyl; wherein C^{a} is unsubstituted or substituted by 1 R^{Ca}; and
R¹² is H.

In another embodiment, the present disclosure provides a compound of Formula (I) or (IA), wherein:
R¹ is CH₃C(O)NH—CH₂CH₂—O—, 5-CO₂H-pyrimidin-2-yl, 4-CH₃C(O)-piperazin-1-yl, 4-CO₂H-cyclohex-4-yl, 4-CO₂H-phenyl, or bicyclo[1.1.1]pentane-1-carboxylic acid;
R² is pyrid-4-yl, pyridazine-4-yl, bicyclo[1.1.1]pentan-1-yl, or cyclobutyl;
R⁴ is 4-fluoroindoly-3-yl, 4-chloroindoly-3-yl, or naphth-1-yl;
R^{5a} is CH3, HOCH₂—, H₂NCH₂CH₂CH₂CH₂—, or H₂NCH₂CH₂—;
R^{5b} is CH₃, HOCH₂—, or H₂NCH₂CH₂—; or, alternatively R^{5a} and R^{5b}, together with the carbon atom to which they are attached, form an azetidinyl ring;
R^{6a} is —CH₂CO₂H, —OH, —H, —CO₂H, —CH₂OH, CH₃, or —CH₂CH₃;
R^{6b} is H, or CH₃—;
R^{7a} is CH₃, HOCH₂—, H₂NCH₂CH₂—, H₂NCH₂CH₂CH₂CH₂—, or —CH₂CO₂H;
R^{7b} is CH₃, HOCH₂—, H₂NCH₂CH₂—, H₂NCH₂CH₂CH₂CH₂—, or —CH₂CO₂H;
or, alternatively R^{7a} and R^{7b}, together with the carbon atom to which they are attached, form an azetidinyl or a piperidinyl ring;
R^{8a} is amino, hydroxy, methyl, H₂NC(NH)N(H)CH₂CH₂—, H₂NC(O)—N(H)CH₂CH₂—, H₂N CH₂CH₂—, phenyl, pyrid-4-yl, pyrid-3-yl, pyrid-2-yl, pyrimidin-5-yl, pyrimidin-2-yl, pyrazin-2-yl, pyridazin-3-yl, pyridazin-4-yl, piperidin-4-yl, tetrahydropyran-4-yl, or morpholin-4-yl;
R^{8b} is H or CH₃;
R⁹ is HO—, H₂N—, H₂NCH₂—, H₂NCH₂CH₂—, H₂NCH₂CH₂CH₂—, H₂NC(NH)N(H)CH₂CH₂—, or morpholin-4-yl;
R¹⁰ is H or methyl;
R¹¹ is H, —CH₂Ph, —CH₂-(4-bromophenyl), —CH₂-(pyrimidin-5-yl), —CH₂-4-(pyrimidin-5-yl)phenyl, —CH₂-4-(2-aminopyrimidin-5-yl)phenyl, —CH₂-4-(pyrid-4-yl)phenyl, CH₂-4-(pyrid-3-yl)phenyl, —CH₂-4-(5-aminopyrazin-2-yl)phenyl, —CH₂-4-(2-aminopyrimidin-5-yl)phenyl, —CH₂-4-(2-methoxypyrimidin-5-yl)phenyl, —CH₂-4-(pyrid-2-yl)phenyl, —CH₂-4-[(3-methyl)-isoxazol-4-yl]phenyl, —CH₂-4-[(1-methyl)imidazol-2-yl]phenyl, or —CH₂-4-[(1-methyl)imidazol-4-yl]phenyl, and
R¹² is H, —CH₂Ph, —CH₂-(4-F phenyl), or —CH₂-(4-pyrid-4-yl).

In some embodiments, the present disclosure provides a compound of Formula (I or (IA)), wherein:
R¹ is CH₃C(O)NH—CH₂CH₂—O—, 5-CO₂H-pyrimidin-2-yl, 4-CH₃C(O)-piperazin-1-yl, 4-CO₂H-cyclohex-4-yl, 4-CO₂H-phenyl, or bicyclo[1.1.1]pentane-1-carboxylic acid;
R² is pyrid-4-yl, pyridazine-4-yl, bicyclo[1.1.1]pentan-1-yl, or cyclobutyl;
R⁴ is 4-fluoroindoly-3-yl, 4-chloroindoly-3-yl, or naphth-1-yl;
R^{5a} is CH₃, HOCH₂—, H₂NCH₂CH₂CH₂CH₂—, or H₂NCH₂CH₂—;
R^{5b} is CH₃, HOCH₂—, or H₂NCH₂CH₂—;
or, alternatively R^{5a} and R^{5b}, together with the carbon atom to which they are attached, form an azetidinyl ring;
R^{6a} is —CH₂CO₂H, —OH, —H, —CO₂H, —CH₂OH, CH₃, or —CH₂CH₃;
R^{6b} is H, or CH₃—;
R^{7a} is CH₃, HOCH₂—, H₂NCH₂CH₂—, H₂NCH₂CH₂CH₂CH₂—, or —CH₂CO₂H;
R^{7b} is CH₃, HOCH₂—, H₂NCH₂CH₂—, H₂NCH₂CH₂CH₂CH₂—, or —CH₂CO₂H;
or, alternatively R^{7a} and R^{7b}, together with the carbon atom to which they are attached, form an azetidinyl or a piperidinyl ring;
R^{8a} is amino, hydroxy, methyl, H₂NC(NH)N(H)CH₂CH₂—, H₂NC(O)—N(H)CH₂CH₂—, H₂N CH₂CH₂—, phenyl, pyrid-4-yl, pyrid-3-yl, pyrid-2-yl, pyrimidin-5-yl, pyrimidin-2-yl, pyrazin-2-yl, pyridazin-3-yl, pyridazin-4-yl, piperidin-4-yl, tetrahydropyran-4-yl, or morpholin-4-yl;
R^{8b} is H or CH₃;
R⁹ is HO—, H₂N—, H₂NCH₂—, H₂NCH₂CH₂—, H₂NCH₂CH₂CH₂—, H₂NC(NH)N(H)CH₂CH₂—, or morpholin-4-yl;
R¹⁰ is H or methyl;
R¹¹ is H, —CH₂Ph, —CH₂-(4-bromophenyl), —CH₂-(pyrimidin-5-yl), —CH₂-4-(pyrimidin-5-yl)phenyl, —CH₂-4-(2-aminopyrimidin-5-yl)phenyl, —CH₂-4-(pyrid-4-yl)phenyl, CH₂-4-(pyrid-3-yl)phenyl, —CH₂-4-(5-aminopyrazin-2-yl)phenyl, —CH₂-4-(2-aminopyrimidin-5-yl)phenyl, —CH₂-4-(2-methoxypyrimidin-5-yl)phenyl. —CH₂-4-(pyrid-2-yl)phenyl, —CH₂-4-[(3-methyl)-isoxazol-4-yl]phenyl, —CH₂-4-[(1-methyl)imidazol-2-yl]phenyl, or —CH₂-4-[(1-methvl)imidazol-4-yl]phenyl, and
R¹² is H, —CH₂Ph, —CH₂-(4-F phenyl), or —CH₂-(4-pyrid-4-yl).

In one embodiment, the present disclosure provides a compound of Formula (I) having the Formula (IB)

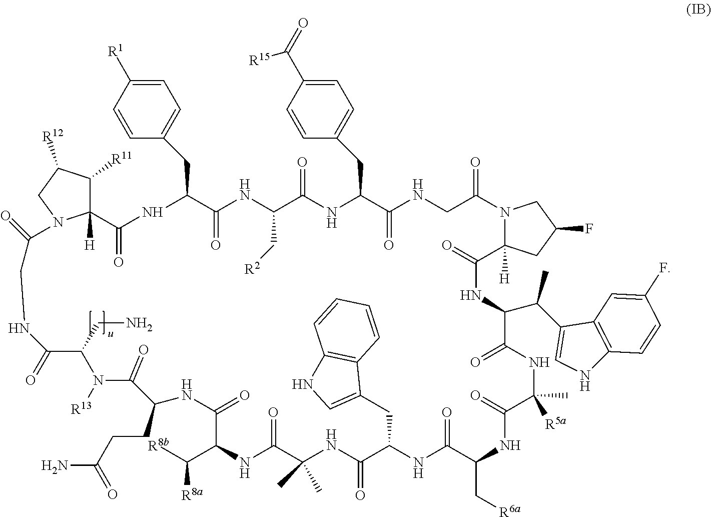

In specific embodiments, the present disclosure provides a compound having the Formula (IB), wherein:
R^{5a} is methyl or HOCH₂—;
R^{6a} is H, —OH, or —CH₂CO₂H;
R^{8a} is:
   (i) C⁸, wherein C⁸ is unsubstituted pyridyl, pyrimidinyl or pyrazinyl; or
   (ii) CH₃CH₂—O—;
R^{8b} is H, methyl;
R¹¹ is H or —CH₂Ph
R¹² is:
   (i) H or
   (ii) —CH₂C¹², wherein C¹² is phenyl or pyridyl
      wherein C¹² is unsubstituted or substituted by 1 halo; and subscript u is 1 or 2.

In certain embodiments, the present disclosure provides a compound of Formula (I), wherein the compound is selected from the group consisting of SEQ ID NOS: SEQ ID NOS: 1-213 and 215-385 as set forth in Table 1.

In specific embodiments, the present disclosure provides a compound of Formula (I), wherein the compound is selected from the group consisting of (SEQ ID NOS 22, 29, 41, 42, 44, 48, 51, 67, 72, 99, 101, 218, and 381, respectively, in order of appearance):

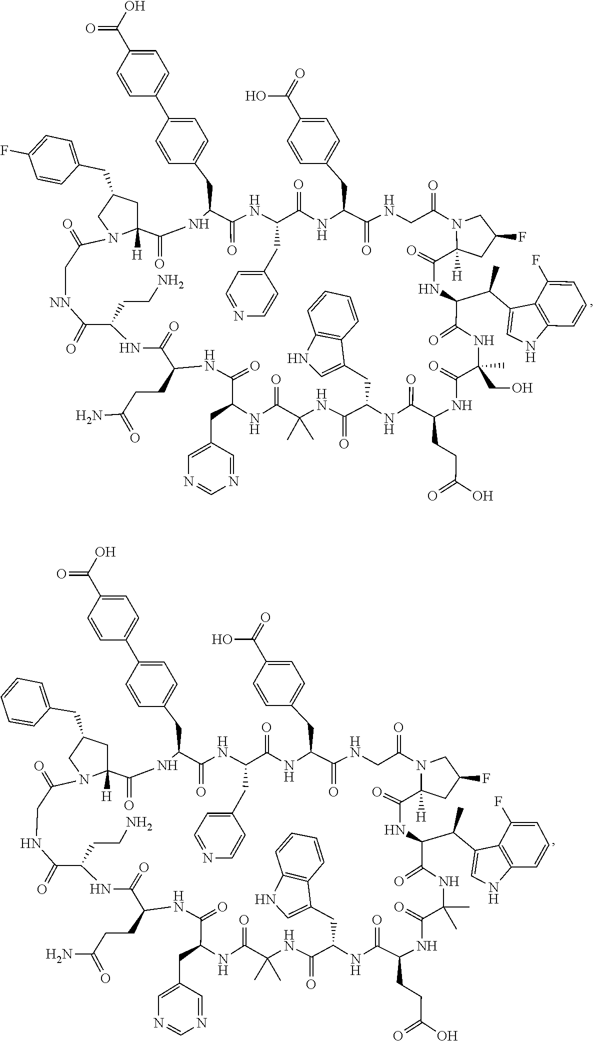

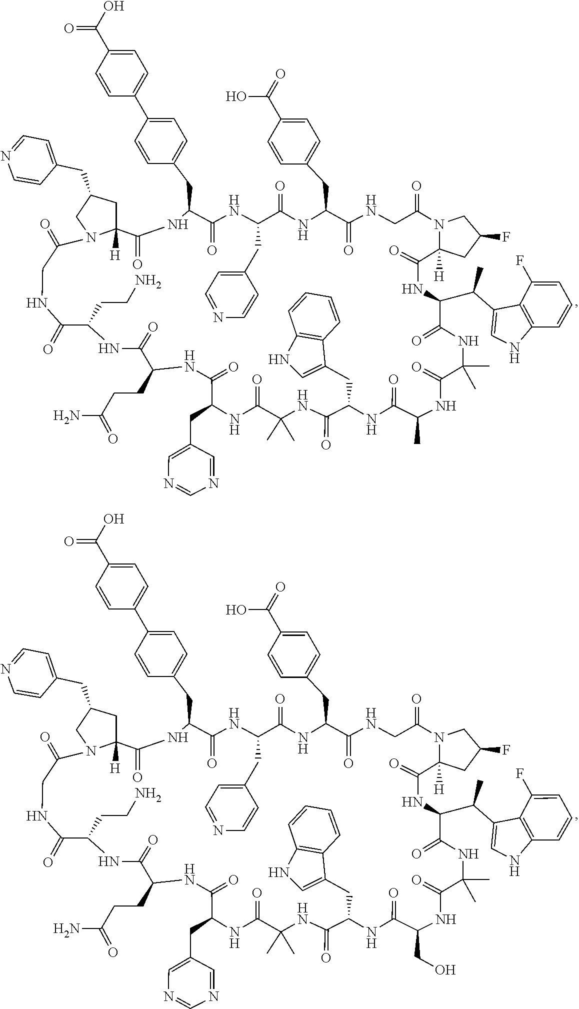

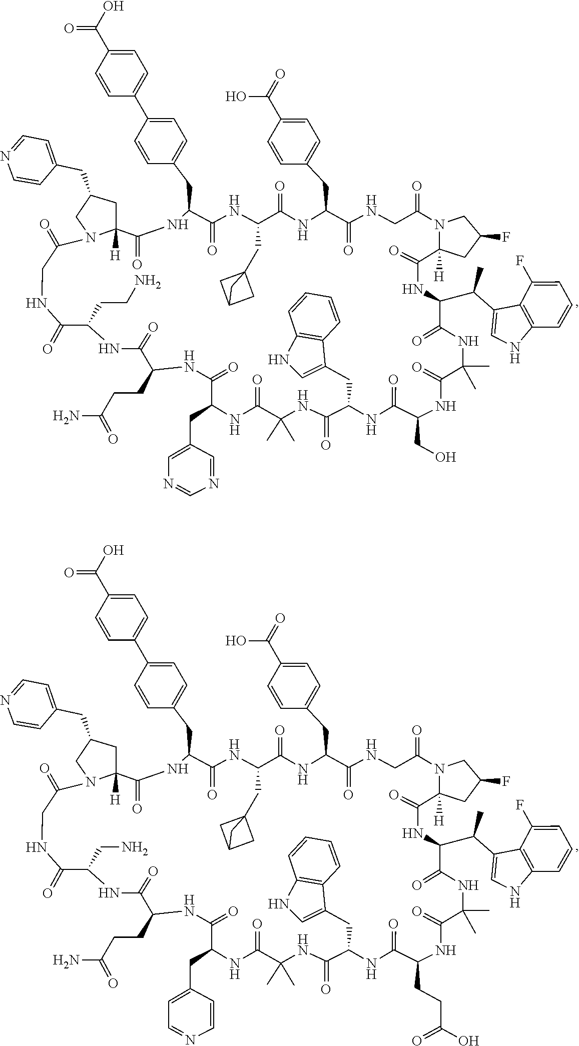

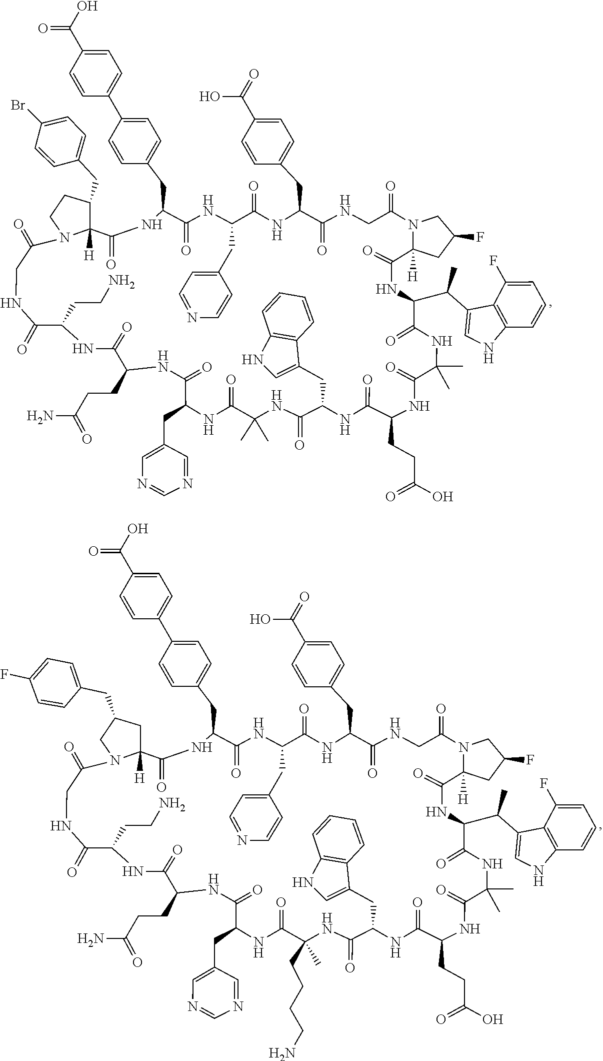

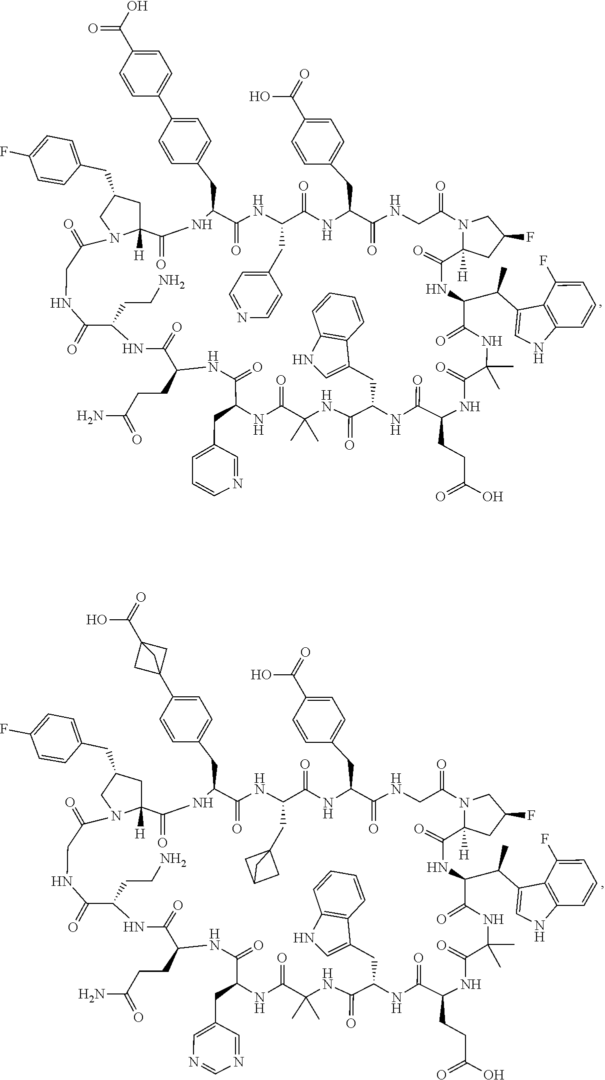

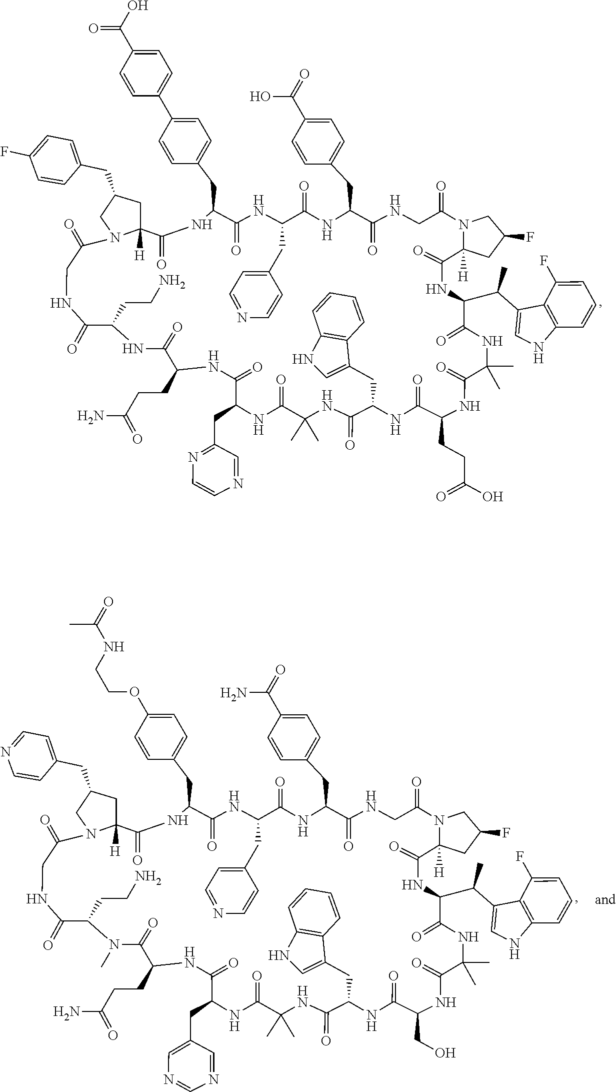

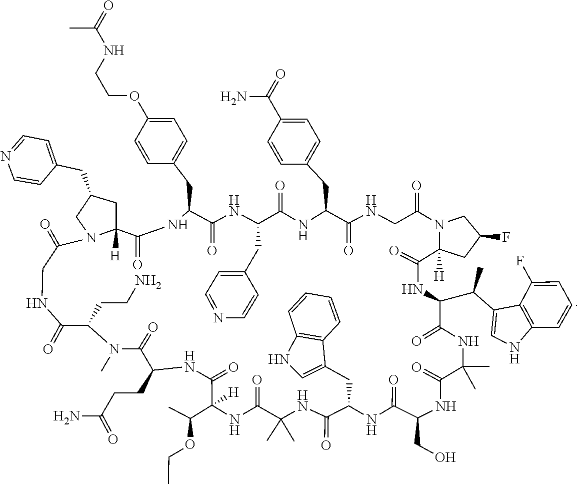 [SMILES: CCO[C@@H](C)[C@@H]1NC(=O)C(C)(C)NC(=O)[C@H](Cc2c[nH]c3ccccc23)NC(=O)[C@H](CO)NC(=O)C(C)(C)NC(=O)[C@H]([C@@H](C)c2c[nH]c3cccc(F)c23)NC(=O)[C@@H]2C[C@H](F)CN2C(=O)CNC(=O)[C@H](Cc2ccc(C(N)=O)cc2)NC(=O)[C@H](Cc2ccncc2)NC(=O)[C@H](Cc2ccc(OCCNC(C)=O)cc2)NC(=O)[C@H]2C[C@@H](Cc3ccncc3)CN2C(=O)CNC(=O)[C@H](CCN)N(C)C(=O)[C@H](CCC(N)=O)NC1=O]

While not being bound by any specific theory, the Applicants believe that the compounds of the disclosure trap interleukin-1β, prevent signaling through the IL-1 receptor and hence reduce the downstream markers IL-6 and CRP. Hence the compounds can be useful to treat the inflammatory components of cardiovascular diseases such as ASCVD and heart failure with preserved ejection fraction (HFpEF). The compounds can also be useful to treat inflammatory disorders such as hidradenitis suppurativa (acne inversa), inflammatory bowel disease, and osteoarthritis.

### Definitions

Unless defined otherwise, all technical and scientific terms used herein have the same meaning as is commonly understood by one of skill in the art to which this invention belongs.

Reference to the compounds of structural Formula (I) includes the compounds of other generic structural Formulas and embodiments that fall within the scope of Formula (I), including but not limited to the compounds of Formulas (IA) or (IB).

As used throughout this disclosure, “a compound of the disclosure”, “a compound of the present disclosure” and “a compound disclosed herein” are used interchangeably are to be understood to include the disclosed cyclic peptides and compounds of Formula (I). The compounds of Formula (I) can form salts which are also within the scope of the present disclosure. Reference to a compound of the disclosure (or compound of Formula (I)) herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a compound of Formula (I) contains both a basic moiety, such as, but not limited to an amino group, pyrrolidine or imidazole, and an acidic moiety, such as, but not limited to a carboxylic acid, zwitterions (“inner salts”) may be formed and are included within the term “salt(s)” as used herein. In one embodiment, the salt is a pharmaceutically acceptable (i.e., non-toxic, physiologically acceptable) salt. In another embodiment, the salt is other than a pharmaceutically acceptable salt. Salts of the compounds of Formula (I) may be formed, for example, by reacting a compound of Formula (I) with an amount of acid or base, such as an equivalent amount, in a medium such as one in which the salt precipitates or in an aqueous medium followed by lyophilization.

“Acyl” means an alkyl-C(O)— group, wherein alkyl is as defined below. The bond to the parent group is through the carbon atom of the carbonyl group.

“Alkyl”, as well as other groups having the prefix “alk”, such as alkoxy, and the like, means carbon chains which may be linear or branched, or combinations thereof, containing the indicated number of carbon atoms. For instance, a C₁-C₆ alkyl means an alkyl group having one (i.e., methyl) up to 6 carbon atoms (i.e., hexyl). In particular embodiments, linear alkyl groups have 1-6 carbon atoms and branched alkyl groups have 3-7 carbon atoms. Examples of alkyl groups include methyl, ethyl, propyl, isopropyl, butyl, sec- and tert-butyl, pentyl, hexyl, heptyl, octyl, nonyl and the like.

“Alkoxy” and “alkyl-O—” are used interchangeably and refer to an alkyl group linked to oxygen.

“Amino” means a H₂N— group. The bond to the parent group is through the nitrogen atom.

“Amino acid” refers to naturally-occurring α-amino acids and their stereoisomers, as well as unnatural amino acids (such as β-amino acids and substituted amino acids) and their stereoisomers. In the sequences given for the peptides (compounds) according to the present disclosure, the amino acid residues have their conventional meaning. Thus, “G” is glycine, “W” is tryptophan, “A” is alanine, “S” is serine, and so on. It is to be understood that “D” isomers are designated by a “d” before the one letter code or amino acid name, such that for example dA is the D isomer of L-alanine. Amino acid residues not encompassed by the foregoing have the definitions provided in the Table in the Examples section below.

“Aryl”, as used herein, represents a monocyclic 6-membered or bicyclic 10-membered ring system, wherein at least one ring is aromatic, and all the ring atoms are carbon.

“Bicyclic ring system” refers to two joined rings. The rings may be fused, i.e., share two adjacent atoms, or “spirocyclic”, i.e., share only a single atom.

“Carboxy” means a HO₂C— group. The bond to the parent group is through the carbon atom of the carbonyl component.

“Cycloalkyl” means a saturated cyclic hydrocarbon radical. In particular embodiments, the cycloalkyl group has 3-12 carbon atoms, forming 1-3 carbocyclic rings that. The rings may be fused, or “spirocyclic”, i.e., share only a single atom, or “bridged”, i.e., share three or more atoms with two bridgehead atoms being connected by a bridge containing at least one atom. Examples of cycloalkyl include cyclopropyl, cyclobutyl, cyclopentyl, cyclohexyl, cycloheptyl, adamantyl, bicyclo[1.1.1]pentanyl,and the like.

“Fluoroalkyl” includes mono-substituted as well as multiple fluoro-substituted alkyl groups, up to perfluoro substituted alkyl. For example, fluoromethyl, 1,1-difluoroethyl, trifluoromethyl or 1,1,1,2,2-pentafluorobutyl are included.

“Halogen” or “halo”, unless otherwise indicated, includes fluorine (fluoro), chlorine (chloro), bromine (bromo) and iodine (iodo). In one embodiment, halo is fluoro (—F) or chloro (—Cl).

“Heterocycloalkyl” means a non-aromatic monocyclic, bicyclic or tricyclic ring system comprising about 3 to about 10 ring atoms, preferably about 5 to about 10 ring atoms, in which one or more of the atoms in the ring system is an element other than carbon, for example nitrogen, oxygen or sulfur, alone or in combination. The rings of bi- and tricyclic ring may be fused, or “spirocyclic”, i.e., share only a single atom, or “bridged”, i.e., share three or more atoms with two bridgehead atoms being connected by a bridge containing at least one atom. There are no adjacent oxygen and/or sulfur atoms present in the ring system. In some embodiments, heterocycloalkyls contain about 5 to about 6 ring atoms. The prefix aza, oxa or thia before the heterocyclyl root name means that at least a nitrogen, oxygen or sulfur atom respectively is present as a ring atom. In some embodiments, the nitrogen or sulfur atom of the heterocycloalkyl can be optionally oxidized to the corresponding N-oxide, S-oxide or S,S-dioxide. Non-limiting examples of suitable monocyclic heterocyclyl rings include piperidyl, pyrrolidinyl, piperazinyl, morpholinyl, thiomorpholinyl, thiazolidinyl, 1,4-dioxanyl, tetrahydrofuranyl, tetrahydropyranyl, tetrahydrothiophenyl, and the like.

“Heteroaryl” refers to aromatic monocyclic, bicyclic and tricyclic ring structures in which one or more atoms in the ring, the heteroatom(s), is an element other than carbon. Heteroatoms are typically O, S, or N atoms. Examples of heteroaromatic groups include pyridinyl, pyrimidinyl, pyrrolyl, pyridazinyl, isoxazolyl, thiazolyl, oxazolyl, indolyl, benzoxazolyl, benzothiazolyl, and imidazolyl.

When any variable (e.g., R^{C1}) occurs more than one time in any constituent or in Formula (I) or other generic formulas herein, its definition on each occurrence is independent of its definition at every other occurrence. Combinations of substituents and/or variables are permissible only if such combinations result in stable compounds. In choosing compounds of the present disclosure, one of ordinary skill in the art will recognize that the various substituents, e.g., R^{C9}, are to be chosen in conformity with well-known principles of chemical structure connectivity and stability. Unless expressly stated to the contrary, substitution by a named substituent is permitted on any atom in a ring (e.g., aryl, a heteroaryl ring, or a saturated heteroaryl ring) provided such ring substitution is chemically allowed and results in a stable compound. A “stable” compound is a compound which can be prepared and isolated and whose structure and properties remain or can be caused to remain essentially unchanged for a period of time sufficient to allow use of the compound for the purposes described herein (e.g., therapeutic or prophylactic administration to a subject).

The term “substituted” shall be deemed to include multiple degrees of substitution by a named substituent. Where multiple substituent moieties are disclosed or claimed, the substituted compound can be independently substituted by one or more of the disclosed or claimed substituent moieties, singly or plurally. By independently substituted, it is meant that the (two or more) substituents can be the same or different.

Unless expressly depicted or described otherwise, variables depicted in a structural formula with a “floating” bond, are permitted on any available carbon atom in the ring to which the variable is attached. When a moiety is noted as being “optionally substituted” in Formula (I) or any embodiment thereof, it means that Formula (I) or the embodiment thereof encompasses compounds that contain the noted substituent (or substituents) on the moiety and also compounds that do not contain the noted substituent (or substituents) on the moiety.

The wavy line [symbol] , as used herein, indicates a point of attachment to the rest of the compound.

Some of the compounds described herein may exist as tautomers which have different points of attachment of hydrogen accompanied by one or more double bond shifts. For example, a ketone and its enol form are keto-enol tautomers. The individual tautomers as well as mixtures thereof are encompassed with compounds of the present disclosure.

In the compounds of the disclosure, the atoms may exhibit their natural isotopic abundances, or one or more of the atoms may be artificially enriched in a particular isotope having the same atomic number, but an atomic mass or mass number different from the atomic mass or mass number predominantly found in nature. The present disclosure as described and claimed herein is meant to include all suitable isotopic variations of the compounds of the disclosure and embodiments thereof. For example, different isotopic forms of hydrogen (H) include protium (H) and deuterium (²H, also denoted herein as D). Protium is the predominant hydrogen isotope found in nature. Enriching for deuterium may afford certain therapeutic advantages, such as increasing in vivo half-life or reducing dosage requirements or may provide a compound useful as a standard for characterization of biological samples. Isotopically-enriched compounds of the disclosure, can be prepared without undue experimentation by conventional techniques well known to those skilled in the art or by processes analogous to those described in the Schemes and Examples herein using appropriate isotopically-enriched reagents and/or intermediates.

The term “pharmaceutically acceptable salts” refers to salts prepared from pharmaceutically acceptable non-toxic bases or acids. When the compound of the present disclosure is acidic (or has a functional group which may be anionic), its corresponding salt can be conveniently prepared from pharmaceutically acceptable non-toxic bases, including inorganic bases and organic bases. Examples of suitable inorganic cations include, but are not limited to, alkali metal ions such as Li⁺, Na⁺, and K⁺, alkaline earth metal cations such as Ca2⁺, and Mg2⁺, and other cations such as Al3⁺ and Zn⁺. Examples of suitable organic cations include, but are not limited to, ammonium ion (i.e., NH₄⁺) and substituted ammonium ions. Examples of suitable substituted ammonium ions are those derived from methylamine, ethylamine, diethylamine, triethylamine and ethylenediamine. When a compound of the present disclosure is basic, its corresponding salt can be conveniently prepared from pharmaceutically acceptable non-toxic acids, including inorganic acids and organic acids. Example of such acid addition salts include salts formed from hydrohalic acids (e.g., hydrochloric, hydrobromic, hydroiodic), formic acid, acetic acid, capric acid, and citric acids. Salts containing acetate, formate, caprate, chloride, or sodium salts are typical for use with the compounds of the present disclosure. In some embodiments, salts of compounds of the present disclosure can be formed by exchange well-known to those of ordinary skill in the art, such as by anion exchange, e.g., replacement of trifluoroacetate ions with chloride ions.

Furthermore, compounds of the present disclosure may exist in amorphous form and/or one or more crystalline forms, and as such all amorphous and crystalline forms and mixtures thereof of the compounds of Formula (I), including the Examples, are intended to be included within the scope of the present disclosure. In addition, some of the compounds of the instant disclosure may form solvates with water (i.e., a hydrate) or common organic solvents such as, but not limited to, acetic acid or acetonitrile. Such solvates and hydrates, particularly the pharmaceutically acceptable solvates and hydrates, of the instant compounds are likewise encompassed within the scope of this disclosure, along with un-solvated and anhydrous forms.

Any pharmaceutically acceptable pro-drug modification of a compound of this disclosure which results in conversion in vivo to a compound within the scope of this disclosure is also within the scope of this disclosure.

The present disclosure also relates to processes for the preparation of the compounds of Formula (I) which are described in the following Examples and by which the compounds of the disclosure are obtainable.

“Treatment” and “treating” refer to all processes in which there may be a slowing, interrupting, arresting, controlling, or stopping of the progression of a disease or disorder described herein. The terms do not necessarily indicate a total elimination of all disease or disorder symptoms.

“Preventing,” or “prophylaxis,” as used herein, refers to reducing the likelihood of contracting disease or disorder described herein, or reducing the severity of a disease or disorder described herein.

The terms “therapeutically effective (or efficacious) amount” and similar descriptions such as “an amount efficacious for treatment” or “an effective dose” are intended to mean that amount of a compound of the disclosure that will elicit the biological or medical response of a tissue, a system, animal or human that is being sought by a researcher, veterinarian, medical doctor or other clinician. In a preferred embodiment, the term “therapeutically effective amount” means an amount of a compound of the disclosure that alleviates at least one clinical symptom in a human patient. The terms “prophylactically effective (or efficacious) amount” and similar descriptions such as “an amount efficacious for prevention” are intended to mean that amount of a compound of the disclosure that will prevent or reduce the risk of occurrence of the biological or medical event that is sought to be prevented in a tissue, a system, animal or human by a researcher, veterinarian, medical doctor or other clinician.

### Dosages of the Compounds of the Present Disclosure

The dosage regimen utilizing a compound of the present disclosure is selected in accordance with a variety of factors including type, species, age, weight, sex and medical condition of the patient; the severity of the condition to be treated; the potency of the compound chosen to be administered; the route of administration; and the renal and hepatic function of the patient. A consideration of these factors is well within the purview of the ordinarily skilled clinician for the purpose of determining the therapeutically effective or prophylactically effective dosage amount needed to prevent, counter, or arrest the progress of the condition. It is understood that a specific daily dosage amount can simultaneously be both a therapeutically effective amount, e.g., for treatment of an oncological condition, and a prophylactically effective amount, e.g., for prevention of an oncological condition.

While individual needs vary, determination of optimal ranges of effective amounts of the compound of the present disclosure is within the skill of the art. For administration to a human in the curative or prophylactic treatment of the conditions and disorders identified herein, for example, typical dosages of the compounds of the present disclosure can be about 0.05 mg/kg/day to about 50 mg/kg/day. In some embodiments, a patient is administered from about 5 mg/day to about 120 mg/day, such as from 10 mg/day, 20 mg/day, 30 mg/day, 40 mg/day, 50 mg/day, 60 mg/day, 70 mg/day, 80 mg/day, mg/day, 90 mg/day, or 100 mg/day of a compound of the present disclosure. In certain embodiments, a patient is administered from about 0.2 mg/kg to about 5 mg/kg, such as from 0.5 mg/kg, 0.75 mg/kg, 1.0 mg/kg, 1.25 mg/kg, or 1.5 mg/kg of a compound of the present disclosure. Such doses may be administered in a single dose or may be divided into multiple doses.

### Pharmaceutical Compositions

The compounds of the disclosure and their pharmaceutically acceptable salts can be administered to animals, preferably to mammals, and particularly to humans, as pharmaceuticals by themselves, in mixtures with one another or in the form of pharmaceutical compositions. The term “subject” or “patient” includes animals, preferably mammals and especially humans, who use the instant active agents for the prevention or treatment of a medical condition. Administering of the drug to the subject includes both self-administration and administration to the patient by another person. The subject may be in need of, or desire, treatment for an existing disease or medical condition, or may be in need of or desire prophylactic treatment to prevent or reduce the risk of occurrence of the disease or medical condition. As used herein, a subject “in need” of treatment of an existing condition or of prophylactic treatment encompasses both a determination of need by a medical professional as well as the desire of a patient for such treatment.

The present disclosure therefore also provides the compounds of the disclosure and their pharmaceutically acceptable salts for use as pharmaceuticals, their use for modulating the activity of the cytokine IL-1β, and in particular, their use in the therapy and prophylaxis of the below-mentioned diseases or disorders as well as their use for preparing medicaments for these purposes. In certain embodiments, the compounds of the disclosure and their pharmaceutically acceptable salts trap IL-1β.

Furthermore, the present disclosure provides pharmaceutical compositions which comprise as active component an effective dose of at least one compound of the disclosure and/or a pharmaceutically acceptable salt thereof and a customary pharmaceutically acceptable carrier, i.e., one or more pharmaceutically acceptable carrier substances and/or additives.

Thus, the present disclosure provides, for example, said compound and its pharmaceutically acceptable salts for use as pharmaceutical compositions which comprise as active component an effective dose of the compound of the disclosure and/or a pharmaceutically acceptable salt thereof and a customary pharmaceutically acceptable carrier, and the uses of said compound and/or a pharmaceutically acceptable salt thereof in the therapy or prophylaxis of the below-mentioned diseases or disorders, e.g., atherosclerosis, as well as their use for preparing medicaments for these purposes.

The pharmaceutical compositions according to the disclosure can be administered orally, for example, in the form of pills, tablets, lacquered tablets, sugar-coated tablets, granules, hard and soft gelatin capsules, aqueous, alcoholic or oily solutions, syrups, emulsions or suspensions, or rectally, for example, in the form of suppositories. Administration can also be carried out parenterally, for example, subcutaneously, intramuscularly or intravenously in the form of solutions for injection or infusion.

Other suitable administration forms are, for example, percutaneous or topical administration, for example, in the form of ointments, tinctures, sprays or transdermal therapeutic systems, or, for example, microcapsules, implants or rods. The preferred administration form depends, for example, on the disease to be treated and on its severity.

The present disclosure also provides pharmaceutical compositions comprising a compound of Formula (I). The compound of Formula (I) can be used in combination with any suitable pharmaceutical carrier or excipient. Such pharmaceutical compositions comprise a therapeutically effective amount of one or more compounds of Formula (I), and pharmaceutically acceptable excipient(s) and/or carrier(s). The specific pharmaceutic composition will suit the mode of administration. In particular aspects, the pharmaceutical acceptable carrier may be water or a buffered solution.

Excipients included in the pharmaceutical compositions have different purposes depending, for example on the nature of the drug, and the mode of administration. Examples of generally used excipients include, without limitation: saline, buffered saline, dextrose, water-for-infection, glycerol, ethanol, and combinations thereof, stabilizing agents, solubilizing agents and surfactants, buffers and preservatives, tonicity agents, bulking agents, lubricating agents (such as talc or silica, and fats, such as vegetable stearin, magnesium stearate or stearic acid), emulsifiers, suspending or viscosity agents, inert diluents, fillers (such as cellulose, dibasic calcium phosphate, vegetable fats and oils, lactose, sucrose, glucose, mannitol, sorbitol, calcium carbonate, and magnesium stearate), disintegrating agents (such as crosslinked polyvinyl pyrrolidone, sodium starch glycolate, cross-linked sodium carboxymethyl cellulose), binding agents (such as starches, gelatin, cellulose, methyl cellulose or modified cellulose such as microcrystalline cellulose, hydroxypropyl cellulose, sugars such as sucrose and lactose, or sugar alcohols such as xylitol, sorbitol or maltitol, polyvinylpyrrolidone and polyethylene glycol), wetting agents, antibacterials, chelating agents, coatings (such as a cellulose film coating, synthetic polymers, shellac, corn protein zein or other polysaccharides, and gelatin), preservatives (including vitamin A, vitamin E, vitamin C, retinyl palmitate, and selenium, cysteine, methionine, citric acid and sodium citrate, and synthetic preservatives, including methyl paraben and propyl paraben), sweeteners, perfuming agents, flavoring agents, coloring agents, absorption enhancers, administration aids, and combinations thereof.

Carriers are compounds and substances that improve and/or prolong the delivery of an active ingredient to a subject in the context of a pharmaceutical composition. Carriers may serve to prolong the in vivo activity of a drug or slow the release of the drug in a subject, using controlled-release technologies. Carriers may also decrease drug metabolism in a subject and/or reduce the toxicity of the drug. Carriers can also be used to target the delivery of the drug to particular cells or tissues in a subject. Common carriers (both hydrophilic and hydrophobic carriers) include fat emulsions, lipids, PEGylated phospholipids, PEGylated liposomes, PEGylated liposomes coated via a PEG spacer with a cyclic RGD peptide, liposomes and lipospheres, microspheres (including those made of biodegradable polymers or albumin), polymer matrices, biocompatible polymers, protein-DNA complexes, protein conjugates, erythrocytes, vesicles, nanoparticles, and side-chains for hydro-carbon stapling. The aforementioned carriers can also be used to increase cell membrane permeability of the compounds of Formula (I). In addition to their use in the pharmaceutical compositions of the present disclosure, carriers may also be used in compositions for other uses, such as research uses in vitro (e.g., for delivery to cultured cells) and/or in vivo.

Pharmaceutical compositions adapted for oral administration may be presented as discrete units such as capsules or tablets; as powders or granules; as solutions, syrups or suspensions (in aqueous or non-aqueous liquids; or as edible foams or whips; or as emulsions). Suitable excipients for tablets or hard gelatin capsules include lactose, maize starch or derivatives thereof, stearic acid or salts thereof. Suitable excipients for use with soft gelatin capsules include for example vegetable oils, waxes, fats, semi-solid, or liquid polyols etc. For the preparation of solutions and syrups, excipients which may be used include for example water, polyols and sugars. For the preparation of suspensions oils, e.g., vegetable oils, may be used to provide oil-in-water or water in oil suspensions. Excipients which promote absorption from the gastrointestinal tract, e.g., permeation enhancers, such as sodium caprate can be included. In certain situations, delayed release preparations may be advantageous and compositions which can deliver the compounds of the present disclosure in a delayed or controlled release manner may also be prepared. Prolonged gastric residence brings with it the problem of degradation by the enzymes present in the stomach and so enteric-coated capsules may also be prepared by standard techniques in the art where the active substance for release lower down in the gastro-intestinal tract.

Pharmaceutical compositions adapted for transdermal administration may be presented as discrete patches intended to remain in intimate contact with the epidermis of the recipient for a prolonged period of time. For example, the active ingredient may be delivered from the patch by iontophoresis as generally described in Pharmaceutical Research, 3(6):318 (1986).

Pharmaceutical compositions adapted for topical administration may be formulated as ointments, creams, suspensions, lotions, powders, solutions, pastes, gels, sprays, aerosols or oils. When formulated in an ointment, the active ingredient may be employed with either a paraffinic or a water-miscible ointment base. Alternatively, the active ingredient may be formulated in a cream with an oil-in-water cream base or a water-in-oil base. Pharmaceutical compositions adapted for topical administration to the eye include eye drops wherein the active ingredient is dissolved or suspended in a suitable carrier, especially an aqueous solvent. Pharmaceutical compositions adapted for topical administration in the mouth include lozenges, pastilles and mouth washes.

Pharmaceutical compositions adapted for rectal administration may be presented as suppositories or enemas.

Pharmaceutical compositions adapted for nasal administration wherein the carrier is a solid include a coarse powder having a particle size for example in the range 20 to 500 microns which is administered in the manner in which snuff is taken, i.e., by rapid inhalation through the nasal passage from a container of the powder held close up to the nose. Suitable compositions wherein the carrier is a liquid, for administration as a nasal spray or as nasal drops, include aqueous or oil solutions of the active ingredient.

Pharmaceutical compositions adapted for administration by inhalation include fine particle dusts or mists which may be generated by means of various types of metered dose pressurized aerosols, nebulizers or insufflators.

Pharmaceutical compositions adapted for vaginal administration may be presented as pessaries, tampons, creams, gels, pastes, foams or spray formulations.

Pharmaceutical compositions adapted for parenteral administration include aqueous and non-aqueous sterile injection solution which may contain anti-oxidants, buffers, bacteriostats and solutes which render the formulation substantially isotonic with the blood of the intended recipient; and aqueous and non-aqueous sterile suspensions which may include suspending agents and thickening agents. Excipients which may be used for injectable solutions include water-for-injection, alcohols, polyols, glycerin and vegetable oils, for example. The compositions may be presented in unit-dose or multi-dose containers, for example sealed ampoules and vials, and may be stored in a freeze-dried (lyophilized) condition requiring only the addition of the sterile liquid carrier, for example water or saline for injections, immediately prior to use. Extemporaneous injection solutions and suspensions may be prepared from sterile powders, granules and tablets. The pharmaceutical compositions may contain preserving agents, solubilizing agents, stabilizing agents, wetting agents, emulsifiers, sweeteners, colorants, odorants, salts (substances of the present disclosure may themselves be provided in the form of a pharmaceutically acceptable salt), buffers, coating agents or antioxidants. They may also contain therapeutically-active agents in addition to the compounds of the present disclosure.

### Methods of Using the Compounds of the Disclosure

The present application provides a method of IL-1 mediated cell signaling comprising contacting a cell with a compound of the disclosure or a pharmaceutically acceptable salt thereof. Inhibition of IL-1 mediated cell signaling can be assessed by detecting decreases in the levels of downstream biomarker IL-6 and CRP (e.g., hsCRP).

The present application also provides methods of using the compounds of the disclosure (or their pharmaceutically acceptable salts) or pharmaceutical compositions containing such compounds to treat disease conditions, including but not limited to, conditions implicated by IL-1β.

In some embodiments, the present disclosure provides a method of treating cardiovascular disease, the method comprising administering a therapeutically effective amount a compound of the disclosure (or a pharmaceutically acceptable salt thereof) or any of the foregoing pharmaceutical compositions comprising such a compound to a subject in need of such treatment. In some embodiments, the cardiovascular disease is vascular inflammation. In some embodiments, the cardiovascular disease is atherosclerosis. In some embodiments, the cardiovascular disease is heart failure with preserved ejection fraction (HFpEF). In other embodiments the cardiovascular disease is heart failure with reduced ejection fraction (HFrEF).

In some embodiments, the present disclosure provides a method of treating a chronic kidney disease, the method comprising administering a therapeutically effective amount a compound of the disclosure (or a pharmaceutically acceptable salt thereof) or any of the foregoing pharmaceutical compositions comprising such a compound to a subject in need of such treatment.

In some embodiments, the present disclosure provides a method of treating inflammatory disorders, the method comprising administering a therapeutically effective amount of a compound of the disclosure (or a pharmaceutically acceptable salt thereof) or any of the foregoing pharmaceutical compositions comprising such a compound to a subject in need of such treatment. In certain embodiments, the inflammatory disorder is selected from the group consisting of hidradenitis suppurativa (acne inversa), inflammatory bowel disease, arthritis, and nonalcoholic steatohepatitis (NASH).

In some embodiments, the inflammatory disorder is hidradenitis suppurativa (acne inversa).

In certain embodiments, the inflammatory disorder is inflammatory bowel disease, such as Crohn's disease or ulcerative colitis.

In some embodiments, the inflammatory disorder is arthritis, e.g., osteoarthritis, rheumatoid arthritis, psoriatic arthritis, or gouty arthritis.

In other embodiments, the inflammatory disorder is nonalcoholic steatohepatitis (NASH).

### Combination Therapies

One or more additional pharmacologically active agents may be administered in combination with a compound of the disclosure. An additional active agent (or agents) is intended to mean a pharmaceutically active agent (or agents) that is active in the body, including pro-drugs that convert to pharmaceutically active form after administration, which are different from the compound of Formula I, and also includes free-acid, free-base and pharmaceutically acceptable salts of said additional active agents. Generally, any suitable additional active agent or agents, including but not limited to anti-hypertensive agents, anti-atherosclerotic agents such as a lipid modifying compound, anti-diabetic agents and/or anti-obesity agents, anti-inflammatory agents, may be used in any combination with the compound of the disclosure in a single dosage formulation (a fixed dose drug combination), or may be administered to the subject in one or more separate dosage formulations which allows for concurrent or sequential administration of the active agents (co-administration of the separate active agents).

Examples of additional active agents which may be employed in treating cardiovascular disorders include but are not limited to angiotensin converting enzyme inhibitors (e.g., alacepril, benazepril, captopril, ceronapril, cilazapril, delapril, enalapril, enalaprilat, fosinopril, imidapril, lisinopril, moveltipril, perindopril, quinapril, ramipril, spirapril, temocapril, or trandolapril), angiotensin II receptor antagonists (e.g., losartan, i.e., COZAAR®, valsartan (including combinations with sacubitril), candesartan, olmesartan, telmesartan and any of these drugs used in combination with hydrochlorothiazide such as HYZAAR®); sGC activators (e.g., riociguat and vericiguat), PCSK9 inhibitors (e.g., evolocumab, alirocumab, MK-0616 and those disclosed in WO2019/246349), neutral endopeptidase inhibitors (e.g., thiorphan and phosphoramidon), aldosterone antagonists, aldosterone synthase inhibitors, renin inhibitors, endothelin receptor antagonists, phosphodiesterase-5 inhibitors (e.g., sildenafil, tadalafil and vardenafil), vasodilators, calcium channel blockers (e.g., amlodipine, nifedipine, verapamil, diltiazem, gallopamil, niludipine, nimodipins, nicardipine), potassium channel activators (e.g., nicorandil, pinacidil, cromakalim, minoxidil, aprilkalim, loprazolam), diuretics (e.g., hydrochlorothiazide), sympatholitics, beta-adrenergic blocking drugs (e.g., propranolol, atenolol, bisoprolol, carvedilol, metoprolol, or metoprolol tartate), alpha adrenergic blocking drugs (e.g., doxazocin, prazocin or alpha methyldopa) central alpha adrenergic agonists, peripheral vasodilators (e.g., hydralazine); lipid lowering agents e.g., HMG-CoA reductase inhibitors such as simvastatin and lovastatin which are marketed as ZOCOR® and MEVACOR®) in lactone pro-drug form and function as inhibitors after administration, and pharmaceutically acceptable salts of dihydroxy open ring acid HMG-CoA reductase inhibitors such as atorvastatin (particularly the calcium salt sold in LIPITOR®), rosuvastatin (particularly the calcium salt sold in CRESTOR®), pravastatin (particularly the sodium salt sold in PRAVACHOL®), fluvastatin (particularly the sodium salt sold in LESCOL®), crivastatin, and pitavastatin; a cholesterol absorption inhibitor such as ezetimibe (ZETIA®) and ezetimibe in combination with any other lipid lowering agents such as the HMG-CoA reductase inhibitors noted above and particularly with simvastatin (VYTORIN®) or with atorvastatin calcium; niacin in immediate-release or controlled release forms and/or with an HMG-CoA reductase inhibitor; niacin receptor agonists such as acipimox and acifran, as well as niacin receptor partial agonists; metabolic altering agents including insulin and insulin mimetics (e.g., insulin degludec, insulin glargine, insulin lispro), dipeptidyl peptidase-IV (DPP-4) inhibitors (e.g., sitagliptin, alogliptin, omarigliptin, linagliptin, vildagliptin); insulin sensitizers, including (i) PPARy agonists, such as the glitazones (e.g., pioglitazone, mitoglitazone, lobeglitazone, rosiglitazone, and balaglitazone), and other PPAR ligands, including (1) PPARα/γ dual agonists (e.g., chiglitazar, muraglitazar, aleglitazar, sodelglitazar, and naveglitazar); (2) PPARα agonists such as fenofibric acid derivatives (e.g., gemfibrozil, clofibrate, ciprofibrate, fenofibrate, bezafibrate), (3) selective PPAR γ modulators (SPPAR γ M's), (e.g., such as those disclosed in WO 02/060388, WO 02/08188, WO 2004/019869, WO 2004/020409, WO 2004/020408, and WO 2004/066963); and (4) PPAR γ partial agonists; (ii) biguanides, such as metformin and its pharmaceutically acceptable salts, in particular, metformin hydrochloride, and extended-release formulations thereof, such as Glumetza™, Fortamet™, and GlucophageXR™; and (iii) protein tyrosine phosphatase-1 B (PTP-1 B) inhibitors; insulin or insulin analogs (e.g., insulin detemir, insulin glulisine, insulin degludec, insulin glargine, insulin lispro and inhalable formulations of each); leptin and leptin derivatives and agonists; amylin and amylin analogs (e.g., pramlintide); sulfonylurea and non-sulfonylurea insulin secretagogues (e.g., tolbutamide, glyburide, glipizide, glimepiride, mitiglinide, meglitinides, nateglinide and repaglinide); α-glucosidase inhibitors (e.g., acarbose, voglibose and miglitol); glucagon receptor antagonists; incretin mimetics, such as GLP-1, GLP-1 analogs, derivatives, and mimetics; and GLP-1 receptor agonists (e.g., dulaglutide, semaglutide, albiglutide, exenatide, liraglutide, lixisenatide, taspoglutide, including intranasal, transdermal, and once-weekly formulations thereof); bile acid sequestering agents (e.g., colestilan, colestimide, colesevalam hydrochloride, colestipol, cholestyramine, and dialkylaminoalkyl derivatives of a cross-linked dextran), acyl CoA:cholesterol acyltransferase inhibitors, (e.g., avasimibe); antiobesity compounds; agents intended for use in inflammatory conditions, such as aspirin, non-steroidal anti-inflammatory drugs or NSAIDs, glucocorticoids, and selective cyclooxygenase-2 or COX-2 inhibitors; glucokinase activators (GKAs); inhibitors of 11 β-hydroxysteroid dehydrogenase type 1, (e.g., such as those disclosed in U.S. Pat. No. 6,730,690); inhibitors of fructose 1,6-bisphosphatase, (e.g., such as those disclosed in U.S. Pat. Nos. 6,054,587; 6,110,903; 6,284,748; 6.399,782; and 6,489,476); inhibitors of acetyl CoA carboxylase-1 or 2 (ACC1 or ACC2); AMP-activated Protein Kinase (AMPK) activators; other agonists of the G-protein-coupled receptors: (i) GPR-109, (ii) GPR-119, and (iii) GPR-40; SSTR3 antagonists (e.g., such as those disclosed in WO 2009/001836); neuromedin U receptor agonists (e.g., such as those disclosed in WO 2009/042053, including, but not limited to, neuromedin S (NMS)); SCD modulators; GPR-105 antagonists (e.g., such as those disclosed in WO 2009/000087); SGLT inhibitors (e.g., empagliflozin, dapagliflozin, canagliflozin, ertugliflozin, remogloflozin, tofogliflozin, and ipragliflozin); inhibitors of acyl coenzyme A:diacylglycerol acyltransferase 1 and 2 (DGAT-1 and DGAT-2); inhibitors of fatty acid synthase; inhibitors of acyl coenzyme A:monoacylglycerol acyltransferase 1 and 2 (MGAT-1 and MGAT-2); agonists of the TGR5 receptor (also known as GPBAR1, BG37, GPCR19, GPR131, and M-BAR); ileal bile acid transporter inhibitors; PACAP, PACAP mimetics, and PACAP receptor 3 agonists; PPAR agonists; protein tyrosine phosphatase-1 B (PTP-1 B) inhibitors; IL-1β antibodies, (e.g., gevokizumab and canakinumab); and bromocriptine mesylate and rapid-release formulations thereof; or with other drugs beneficial for the treatment of the above-mentioned conditions or disorders including the free-acid, free-base, and pharmaceutically acceptable salt forms of the above active agents where chemically possible.

Examples of additional active agents which may be employed in treating inflammatory disorders include but are not limited to steroidal and non-steroidal anti-inflammatory agents, glucocorticoids, and therapeutic hormones. In particular embodiments, in treating hidradenitis suppurativa (acne inversa), the additional active agent can be an antibiotic, an injectable steroid, a therapeutic hormone, a TNF inhibitor (e.g., infliximab, adalimumab, etanercept, golimumab, certolizumab), a pain medication (e.g., codeine, hydrocodone, morphine, pregabalin, gabapentin, Intralesional triamcinolone, a corticosteroid, naproxen, ketoprofen, diclofenac, ibuprofen, acetaminophen). In other embodiments, in treating an inflammatory bowel disease, the additional active agent can be methotrexate, a TNF inhibitor, an oral sphingosine 1-phosphate receptor modulator (e.g., fingolimod, siponimod, ozanimod, ponesimod) or a selective JAK inhibitor (e.g., tofacitinib, baricitinib, upadacitinib). In some embodiments, in treating osteoarthritis, the additional active agent can be a pain medication (examples listed above). In other embodiments, in treating gouty arthritis, the additional active agent can be colcichine, a non-steroidal anti-inflammatory agent, or a glucocorticoid.

### Methods of Preparing the Compounds of the Disclosure

The compounds described herein can be prepared according to the procedures of the following schemes and examples, using appropriate materials and are further exemplified by the following specific examples. The examples also include methods for testing such compounds in cellular assays. The compounds illustrated in the examples are not, however, to be construed as forming the only genus that is considered as the disclosure.

The examples further illustrate details for the preparation of the compounds of the present disclosure. Those skilled in the art will readily understand that known variations of the conditions and processes of the following preparative procedures can be used to prepare these compounds. For instance, in some cases, the order of carrying out the steps of reaction schemes may be varied to facilitate the reaction or to avoid unwanted reaction products. Starting materials and intermediates for the final compounds are purchased, made from known procedures, or as otherwise illustrated. The examples are provided for the purpose of further illustration only and are not intended to be limitations on the disclosure.

NMR data were obtained on a 300 MHz or 400 MHz instrument in CDCl₃, DMSO-d₆ or Methanol-d₄ with the chemical shifts reported relative to tetramethylsilane standard. Resonance signals are reported by the following abbreviations: s=singlet, d=doublet, t=triplet, q=quartet, dd=doublet of doublets, m=multiplet or overlap of nonequivalent resonances. Coupling constants (J) are reported in Hertz (Hz).

Throughout the synthetic schemes and examples, abbreviations and acronyms may be used with the following meanings unless otherwise indicated:

Abbreviations

| Abbreviation | Definition |
|---|---|
| 2Pal | (2S)-2-amino-3-(2-pyridyl)propanoic acid |
| 2PyrimAla | (2S)-2-amino-3-pyrimidin-2-yl-propanoic |
| | acid |
| 3AzaPhe4AcPip | (2S)-3-[6-(4-acetylpiperazin-1-yl)-3- |
| | pyridyl]-2-amino-propanoic acid |
| 3AzaTyrEtNAc | (2S)-3-[6-(2-acetamidoethoxy)-3- |
| | pyridyl]-2-amino-propanoic acid |
| 3Pal | (2S)-2-amino-3-(3-pyridyl)propanoic acid |
| 3Pal4CO2H | 5-[(2S)-2-amino-2-carboxy- |
| | ethyl]pyridine-2-carboxylic acid |
| 3Pal4Ph4CO2H | 4-[5-[(2S)-2-amino-2-carboxy-ethyl]-2- |
| | pyridyl]benzoic acid |
| 3PyridaAla | (2S)-2-amino-3-pyridazin-3-yl-propanoic |
| | acid |
| 4Pal | (2S)-2-amino-3-(4-pyridyl)propanoic acid |
| 4PyridaAla | (2S)-2-amino-3-pyridazin-4-yl-propanoic |
| | acid |
| 4PyrimAla | (2S)-2-amino-3-pyrimidin-4-yl-propanoic |
| | acid |
| A | _{L}-Alanine |
| AA | Amino acid |
| Ac | Acetyl |
| Ac4cN | 3-aminoazetidine-3-carboxylic acid |
| ACN or MeCN | Acetonitrile |
| AcOH | Acetic acid |
| AEF2PyrimCO | (S)-2-amino-3-(4-(2-(pyrimidine-2- |
| | carboxamido)ethoxy)phenyl)propanoic |
| | acid |
| AEF4AcPip4c | (S)-3-(4-(2-(1-acetylpiperidine-4- |
| | carboxamido)ethoxy)phenyl)-2- |
| | aminopropanoic acid |
| AEF4PyrCO | (S)-2-amino-3-(4-(2-(isonicotinamido) |
| | ethoxy)phenyl)propanoic acid |
| AEF4PyrimCO | (S)-2-amino-3-(4-(2-(pyrimidine-4- |
| | carboxamido)ethoxy)phenyl)propanoic |
| | acid |
| AEFBz | (S)-2-amino-3-(4-(2- |
| | benzamidoethoxy)phenyl)propanoic acid |
| AEFBz4Piperaz | (S)-2-amino-3-(4-(2-(4-(piperazin-1- |
| | yl)benzamido)ethoxy)phenyl)propanoic |
| | acid |
| AEFcHex | (S)-2-amino-3-(4-(2- |
| | (cyclohexanecarboxamido)ethoxy)phenyl) |
| | propanoic acid |
| AEFPiv | (S)-2-amino-3-(4-(2- |
| | pivalamidoethoxy)phenyl)propanoic acid |
| AEFPyrzCO | (S)-2-amino-3-(4-(2-(pyrazine-2- |
| | carboxamido)ethoxy)phenyl)propanoic |
| | acid |
| Aib | 2-amino-2-methyl-propanoic acid |
| Ala2Oxa | (S)-2-amino-3-(oxazol-2-yl)propanoic |
| | acid |
| Ala3Isoxa5Me | (S)-2-amino-3-(5-methylisoxazol-3- |
| | yl)propanoic acid |
| AlaMph | (2S)-2-amino-3-morpholino-propanoic |
| | acid |
| AlaPent | (2S)-2-amino-3-(1-bicyclo[1.1.1] |
| | pentanyl)propanoic acid |
| AlaPip4 | (2S)-2-amino-3-(4-piperidyl)propanoic |
| | acid |
| AlaPiperaz | (2S)-2-amino-3-piperazin-1-yl-propanoic |
| | acid |
| AlaPiperazAc | (S)-3-(4-acetylpiperazin-1-yl)-2- |
| | aminopropanoic acid |
| AlaPyrim4CONH2 | (2S)-2-amino-3-(2-carbamoylpyrimidin- |
| | 5-yl)propanoic acid |
| AlaPyrim4COOH | 5-[(2S)-2-amino-2-carboxy- |
| | ethyl]pyrimidine-2-carboxylic acid |
| AlaPyrz | (2S)-2-amino-3-pyrazin-2-yl-propanoic |
| | acid |
| AlaTHP4 | (2S)-2-amino-3-tetrahydropyran-4-yl- |
| | propanoic acid |
| alI | _{L}-alloisoleucine |
| alT | _{L}-allothreonine |
| alThrOEt | O-ethyl-L-allothreonine |
| alTOMe | (2S,3S)-2-amino-3-methoxy-butanoic |
| | acid |
| aMeD | (2S)-2-amino-2-methyl-butanedioic acid |
| aMeDab | (2S)-2,4-diamino-2-methyl-butanoic acid |
| aMeK | (2S)-2,6-diamino-2-methyl-hexanoic acid |
| aMeS | (2S)-2-amino-3-hydroxy-2-methyl- |
| | propanoic acid |
| ASCVD | Atherosclerotic cardiovascular disease |
| Bip | (2S)-2-amino-3-(4- |
| | phenylphenyl)propanoic acid |
| Bip4Cl | (2S)-2-amino-3-[4-(4- |
| | chlorophenyl)phenyl]propanoic acid |
| Bip4CO2H | 4-[4-[(2S)-2-amino-2-carboxy- |
| | ethyl]phenyl]benzoic acid |
| Bip4CONH2 | (2S)-2-amino-3-[4-(4- |
| | carbamoylphenyl)phenyl]propanoic acid |
| Bip4CONH2 | (S)-2-amino-3-(4′-carbamoy]-[1,1′- |
| | biphenyl]-4-yl)propanoic acid |
| Bip4CONMe2 | (2S)-2-amino-3-[4-[4-(dimethylcarbamoyl) |
| | phenyl]phenyl]propanoic acid |
| Boc | tert-Butoxy-carbonyl |
| Boc₂O | Di-tert-butyl dicarbonate |
| CAN | Ammonium Cerium(IV) Nitrate |
| CANTOS | Canakinumab Anti-inflammatory |
| | Thrombosis Outcomes Study |
| cBuA | (S)-2-amino-3-cyclobutylpropanoic acid |
| CDCl₃ | Deuterated chloroform |
| Cha | (S)-2-amino-3-cyclohexylpropanoic acid |
| Cit | (2S)-2-amino-5-ureido-pentanoic acid |
| CRP | C-reactive protein |
| D | _{L}-Aspartic acid |
| d3Pal | (2R)-2-amino-3-(3-pyridyl)propanoic |
| | acid |
| Dab | (2S)-2,4-diaminobutanoic acid |
| DabMe3 | [(3S)-3-amino-4-oxo-butyl]-trimethyl- |
| | ammonium |
| daMeD | (2R)-2-amino-2-methyl-butanedioic acid |
| daMeDab | (2R)-2,4-diamino-2-methyl-butanoic acid |
| daMeF | (2R)-2-amino-2-methyl-3-phenyl- |
| | propanoic acid |
| daMeK | (2R)-2,6-diamino-2-methyl-hexanoic acid |
| daMeN | (2R)-2,4-diamino-2-methyl-4-oxo- |
| | butanoic acid |
| daMeS | (2R)-2-amino-3-hydroxy-2-methyl- |
| | propanoic acid |
| daMeSMe | (2R)-2-amino-3-methoxy-2-methyl- |
| | propanoic acid |
| Dap | (2S)-2,3-diaminopropanoic acid |
| DapUrea | (2S)-2-amino-3-ureido-propanoic acid |
| dba | Dibenzylideneacetone |
| dcBuA | (2R)-2-amino-3-cyclobutyl-propanoic |
| | acid |
| DCC | N,N′-dicyclohexylcarbodiimide |
| DCE | Dichloroethane |
| dCha | (2R)-2-amino-3-cyclohexyl-propanoic |
| | acid |
| DCM | Dichloromethane |
| dD | D-aspartic acid |
| dDab | (2R)-2,4-diaminobutanoic acid |
| dDap | (2R)-2,3-diaminopropanoic acid |
| dE | D-glutamic acid |
| dF | D-phenylalanine |
| DIC | N,N′-Diisopropylcarbodiimide |
| DIEA or DIPEA | N,N-Diisopropylethylamine or Hunig's |
| | base |
| dK | D-lysine |
| dL | D-leucine |
| DMA | N,N-dimethylacetamide |
| DMAP | 4-dimethylaminopyridine |
| DMF | N,N-dimethylformamide |
| DMSO | Dimethylsulfoxide |
| dOrn | D-ornithine |
| dPhe4F | (2R)-2-amino-3-(4- |
| | fluorophenyl)propanoic acid |
| dProc3Bn | (2R,3S)-3-benzylpyrrolidine-2-carboxylic |
| | acid |
| dProc3Bn4Br | (2R,3S)-3-[(4- |
| | bromophenyl)methyl]pyrrolidine-2- |
| | carboxylic acid |
| dProc3Bn4Cl | (2R,3S)-3-[(4- |
| | chlorophenyl)methyl]pyrrolidine-2- |
| | carboxylic acid |
| dProc3Bn4F | (2R,3S)-3-[(4- |
| | fluorophenyl)methyl]pyrrolidine-2- |
| | carboxylic acid |
| dProc3Bn4Im2NMe | (2R,3S)-3-(4-(1-methyl-1H-imidazol-2- |
| | yl)benzyl)pyrrolidine-2-carboxylic acid |
| dProc3Bn4Im4NMe | (2R,3S)-3-(4-(1-methyl-1H-imidazol-4- |
| | yl)benzyl)pyrrolidine-2-carboxylic acid |
| dProc3Bn4Iso3Me | (2R,3S)-3-(4-(3-methylisoxazol-4- |
| | yl)benzyl)pyrrolidine-2-carboxylic acid |
| dProc3Bn4Pyr2 | (2R,3S)-3-[[4-(2- |
| | pyridyl)phenyl┘methyl┘pyrrolidine-2- |
| | carboxylic acid |
| dProc3Bn4Pyr3 | (2R,3S)-3-[[4-(3- |
| | pyridyl)phenyl]methyl]pyrrolidine-2- |
| | carboxylic acid |
| dProc3Bn4Pyr4 | (2R,3S)-3-[[4-(4- |
| | pyridyl)phenyl]methyl]pyrrolidine-2- |
| | carboxylic acid |
| dProc3Bn4Pyraz5NH2 | (2R,3S)-3-[[4-(5-aminopyrazin-2- |
| | yl)phenyl]methyl]pyrrolidine-2- |
| | carboxylic acid |
| dProc3Bn4Pyrim | (2R,3S)-3-[(4-pyrimidin-5- |
| | ylphenyl)methyl]pyrrolidine-2-carboxylic |
| | acid |
| dProc3Bn4Pyrim2NH2 | (2R,3S)-3-[[4-(2-aminopyrimidin-5- |
| | yl)phenyl]methyl]pyrrolidine-2- |
| | carboxylic acid |
| dProc3Bn4Pyrim2NMe | (2R,3S)-3-[[4-[2- |
| | (methylamino)pyrimidin-5- |
| | yl]phenyl]methyl]pyrrolidine-2- |
| | carboxylic acid |
| dProc3Bn4Pyrim2OMe | (2R,3S)-3-[[4-(2-methoxypyrimidin-5- |
| | yl)phenyl]methyl]pyrrolidine-2- |
| | carboxylic acid |
| dProc3Bn4Pyrz | (2R,3S)-3-[(4-pyridazin-4- |
| | ylphenyl)methyl]pyrrolidine-2-carboxylic |
| | acid |
| dProc3CH24Pal | (2R,3S)-3-(4-pyridylmethyl)pyrrolidine- |
| | 2-carboxylic acid |
| dProc3CH2Pyrim | (2R,3S)-3-(pyrimidin-5- |
| | ylmethyl)pyrrolidine-2-carboxylic acid |
| dProc4Bn | (2R,4R)-4-benzylpyrrolidine-2-carboxylic |
| | acid |
| dProc4Bn4Br | (2R,4R)-4-└(4- |
| | bromophenyl)methyl]pyrrolidine-2- |
| | carboxylic acid |
| dProc4Bn4Cl | (2R,4R)-4-[(4- |
| | chlorophenyl)methyl]pyrrolidine-2- |
| | carboxylic acid |
| dProc4Bn4F | (2R,4R)-4-[(4- |
| | fluorophenyl)methyl]pyrrolidine-2- |
| | carboxylic acid |
| dProc4CH21Naph4F | (2R,4R)-4-[(4-fluoro-1- |
| | naphthyl)methyl]pyrrolidine-2-carboxylic |
| | acid |
| dProc4CH24Pal | (2R,4R)-4-(4-pyridylmethyl)pyrrolidine- |
| | 2-carboxylic acid |
| dPyrimA | (2R)-2-amino-3-pyrimidin-5-yl- |
| | propanoic acid |
| dS | D-serine |
| dtbbpy | 4,4′-di-tert-butyl-2,2′-dipyridyl |
| dtbpf | (di-tert-butylphosphino)ferrocene |
| E | _{L}-Glutamic acid |
| EA | ethyl acetate |
| ESI | electrospray ionization |
| EtOAc | Ethyl acetate |
| F | _{L}-Phenylalanine |
| F4bcpA | 3-[4-[(2S)-2-amino-2-carboxy- |
| | ethyl]phenyl]bicyclo[1.1.1]pentane-1- |
| | carboxylic acid |
| F4pcCCA | cis-4-[4-[(2S)-2-amino-2-carboxy- |
| | ethyl┘phenyl┘cyclohexanecarboxylic acid |
| F4ptCCA | trans-4-[4-[(2S)-2-amino-2-carboxy- |
| | ethyl]phenyl]cyclohexanecarboxylic acid |
| Fmoc | 9H-fluoren-9-ylmethoxycarbonyl |
| G | Glycine |
| h | hour |
| H | L-histidine |
| h4Pal | (2S)-2-amino-4-(4-pyridyl)butanoic acid |
| HATU | 1-[Bis(dimethylamino)methylene]-1H- |
| | 1,2,3-triazolo[4,5-b]pyridinium 3-oxid |
| | hexafluorophosphate or N- |
| | [(Dimethylamino)-1H-1,2,3-triazolo-[4,5- |
| | b]pyridin-1-ylmethylene]-N- |
| | methylmethanaminium |
| | hexafluorophosphate N-oxide |
| hCha | (S)-2-amino-4-cyclohexylbutanoic acid |
| hF | L-homophenylalanine |
| HFIP | Hexafluoroisopropanol |
| HFpEF | Heart failure with preserved ejection |
| | fraction |
| HPLC | High-performance liquid chromatography |
| hS | _{L}-homoserine |
| hsCRP | High sensitivity C-reactive protein |
| hY | L-homotyrosine |
| IPA | Isopropanol |
| K | _{L}-Lysine |
| L | L-leucine |
| LCMS | Liquid chromatography mass |
| | spectrometry |
| LDA | Lithium diisopropylamide |
| LDL | Low-density lipoprotein |
| LysMe3 | [(5S)-5-amino-5-carboxy-pentyl]- |
| | trimethyl-ammonium |
| MACE | Major adverse cardiac events |
| MeOH | Methanol |
| min | Minute |
| MO2 | (2S)-2-amino-4-methylsulfonyl-butanoic |
| | acid |
| MS | Mass spectrometry |
| NIS | N-iodosuccinimide |
| NMeDab | (2S)-4-amino-2-(methylamino)butanoic |
| | acid |
| NMO | N-Methylmorpholine N-oxide |
| NMP | N-Methyl-2-pyrrolidone |
| NMR | Nuclear magnetic resonance |
| NsCl | 4-Nitrobenzenesulfonyl chloride |
| Nva | (2S)-2-aminopentanoic acid |
| NvaF3 | (2S)-2-amino-5,5,5-trifluoro-pentanoic |
| | acid |
| o.n. | Over night |
| OMpe | β-methylpentyl ester |
| orbOHorPhe4COOH | 4-[(1R,2S)-2-amino-2-carboxy-1- |
| | hydroxy-ethyl]benzoic acid |
| Orn | _{L}-Ornithine |
| OrnMe3 | [(4S)-4-amino-5-oxo-pentyl]-trimethyl- |
| | ammonium |
| OSu | O-succinimide |
| Oxyma | Ethyl cyano(hydroxyimino)acetate |
| Pbf | 2,2,4,6,7-pentamethyldihydrobenzofuran- |
| | 5-sulfonyl |
| PDC | Pyridinium dichromate |
| PE | Petroleum ether |
| PfTrpB-7E6 | Engineered tryptophan synthase from |
| | Pyrococcus furiosus |
| Phe2Cl4COOH | 4-[(2S)-2-amino-2-carboxy-ethyl]-3- |
| | chloro-benzoic acid |
| Phe3Cl | (2S)-2-amino-3-(3- |
| | chlorophenyl)propanoic acid |
| Phe3Cl4COOH | (S)-4-(2-amino-2-carboxyethyl)-2- |
| | chlorobenzoic acid |
| Phe42ImMe | (S)-2-amino-3-(4-(1-methyl-1H- |
| | imidazol-2-yl)phenyl)propanoic acid |
| Phe4pcCCA | (1R,4s)-4-(4-((S)-2-amino-2- |
| | carboxyethyl)phenyl)cyclohexane-1- |
| | carboxylic acid |
| Phe44PyNH3 | (S)-2-amino-3-(4-(2-aminopyridin-4- |
| | yl)phenyl)propanoic acid |
| Phe4AcPip | (2S)-3-[4-(4-acetylpiperazin-1- |
| | yl)phenyl]-2-amino-propanoic acid |
| Phe4Br | (2S)-2-amino-3-(4- |
| | bromophenyl)propanoic acid |
| Phe4CONH2 | (2S)-2-amino-3-(4- |
| | carbamoylphenyl)propanoic acid |
| Phe4COOH | 4-[(2S)-2-amino-2-carboxy-ethyl]benzoic |
| | acid |
| Phe4F | (2S)-2-amino-3-(4- |
| | fluorophenyl)propanoic acid |
| Phe4ImMe | (S)-2-amino-3-(4-(1-methyl-1H- |
| | imidazol-4-yl)phenyl)propanoic acid |
| Phe4Pyr4 | (S)-2-amino-3-(4-(pyridin-4- |
| | yl)phenyl)propanoic acid |
| Phe4Pyrim | (S)-2-amino-3-(4-(pyrimidin-5- |
| | yl)phenyl)propanoic acid |
| Phe4Pyrim5CO2H | 2-[4-[(2S)-2-amino-2-carboxy- |
| | ethyl]phenyl]pyrimidine-5-carboxylic |
| | acid |
| Phe4PyrimOMe | (S)-2-amino-3-(4-(2-methoxypyrimidin- |
| | 5-yl)phenyl)propanoic acid |
| Phe4tButyl | (2S)-2-amino-3-(4-tert- |
| | butylphenyl)propanoic acid |
| PipH | 4-aminopiperidine-4-carboxylic acid |
| PLP | 3-hydroxy-2-methyl-5- |
| | ([phosphonooxy]methyl)-4- |
| | pyridinecarboxaldehyde or pyridoxal 5- |
| | phosphate |
| PPh₃ | Triphenylphosphine |
| Proc4F | (2S,4S)-4-fluoropyrrolidine-2-carboxylic |
| | acid |
| Proc4OH | (2S,4S)-4-hydroxypyrrolidine-2- |
| | carboxylic acid |
| psi | Pound-force per square inch |
| PyAOP | (7-Azabenzotriazol-1- |
| | yloxy)tripyrrolidinophosphonium |
| | hexafluorophosphate |
| PyrimAla | (2S)-2-amino-3-pyrimidin-5-yl-propanoic |
| | acid |
| PyrimAla4AcPip | (2S)-3-[2-(4-acetylpiperazin-1- |
| | yl)pyrimidin-5-yl]-2-amino-propanoic |
| | acid |
| PyrimAla4Ph4CO2H | 4-[5-[(2S)-2-amino-2-carboxy- |
| | ethyl]pyrimidin-2-yl]benzoic acid |
| Q | _{L}-Glutamine |
| R | _{L}-Arginine |
| RbOH4Pal | (2S,3R)-2-amino-3-hydroxy-3-(4- |
| | pyridyl)propanoic acid |
| RP | Reverse phase |
| rpm | Revolutions per minute |
| RT | Retention time |
| rt | Room temperature |
| S | _{L}-Serine |
| Sar | 2-(methylamino)acetic acid |
| sbMe1Nal | (2S,3S)-2-amino-3-(1-naphthyl)butanoic |
| | acid |
| SbMeW4Cl | (2S,3S)-2-amino-3-(4-chloro-1H-indol-3- |
| | yl)butanoic acid |
| SbMeW4F | (2S,3S)-2-amino-3-(4-fluoro-1H-indol-3- |
| | yl)butanoic acid |
| sbOH4Pal | (2S,3S)-2-amino-3-hydroxy-3-(4- |
| | pyridyl)propanoic acid |
| SbOHPhe4CONH2 | (2S,3S)-2-amino-3-(4-carbamoylphenyl)- |
| | 3-hydroxypropanoic acid |
| SFC | Supercritical fluid chromatography |
| SPPS | Solid-Phase Peptide Synthesis |
| Su | Succinimide |
| T | _{L}-threonine |
| TBAB | Tetrabutylammonium bromide |
| TBAF | Tetrabutylammonium fluoride |
| TBAI | Tetrabutylammonium iodide |
| tBu | tert-Butyl |
| t-BuOH | tert-Butanol |
| TEA | Triethylamine |
| TFA | Trifluoroacetic acid |
| THF | Tetrahydrofuran |
| ThrOBn | (2S,3R)-2-amino-3-benzyloxy-butanoic |
| | acid |
| ThrOEt | O-ethyl-L-threonine |
| ThrOMe | (2S,3R)-2-amino-3-methoxy-butanoic |
| | acid |
| TIS | Triisopropylsilane |
| Trp4Az | (2S)-2-amino-3-(1H-pyrrolo┌3,2- |
| | b]pyridin-3-yl)propanoic acid |
| Trp4Cl | (2S)-2-amino-3-(4-chloro-1H-indol-3- |
| | yl)propanoic acid |
| Trp4F | (2S)-2-amino-3-(4-fluoro-1H-indol-3- |
| | yl)propanoic acid |
| TyrEtNAc | (2S)-3-[4-(2-acetamidoethoxy)phenyl]-2- |
| | amino-propanoic acid |
| UPLC | Ultra Performance Liquid |
| | Chromatography or Ultrahigh Pressure |
| | Liquid Chromatography |
| UV | ultraviolet |
| V | _{L}-valine |
| W | _{L}-Tryptophan |
| wt | weight |
| Y | L-tyrosine |

### Intermediate Syntheses

The following schemes describe suitable syntheses for certain protected amino acid precursors used to prepare the compounds of the present disclosure.

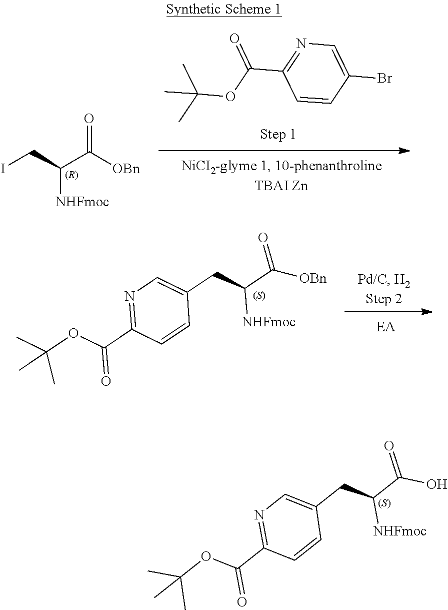

### Precursor to 3Pal4CO2H

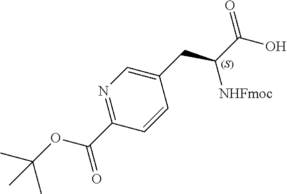

### (S)-2-((((9H-Fluoren-9-yl)methoxy)carbonyl)amino)-3-(6-(tert-butoxycarbonyl)pyridin-3-yl)propanoic acid

Step 1: To a stirred solution of NiCl₂-glyme (710 mg, 3.23 mmol) in DMA (160 mL) was added 1,10-phenanthroline (700 mg, 3.23 mmol) at 25° C. under nitrogen atmosphere. The resulted mixture was stirred at 50° C. for 1 h then tert-butyl 5-bromopicolinate (4.17 g, 16.15 mmol), benzyl (R)-2-((((9H-fluoren-9-yl)methoxy)carbonyl)amino)-3-iodopropanoate (8.51 g, 16.15 mmol), TBAI (6.19 g, 16.15 mmol) and Zn (2.11 g, 32.3 mmol) were added to at room temperature. After the resulted mixture was stirred at 25° C. for 2 h, it was quenched with H₂O (200 mL), extracted with EA (2×500 mL). The combined organic layer was washed with brine (3×200 mL), dried over anhydrous Na₂SO₄ and filtered. The filtrate was concentrated under reduced pressure and the residue was purified by silica gel column chromatography, eluted with 0-20% EA in PE to give tert-butyl (S)-5-(2-((((9H-fluoren-9-yl)methoxy)carbonyl)amino)-3-(benzyloxy)-3-oxopropyl)picolinate (7.5 g, 12.96 mmol, 80% yield) as an off-white solid. MS ESI calculated for C₃₅H₃₅N₂O₆ [M+H]⁺ 579.24, found 579.40.

Step 2: To a stirred solution of tert-butyl (S)-5-(2-((((9H-fluoren-9-yl)methoxy)carbonyl)amino)-3-(benzyloxy)-3-oxopropyl)picolinate (7.5 g, 12.96 mmol) in EA (75 mL) was added Pd—C(1.379 g, 12.96 mmol, dry, 10% wt) at room temperature under nitrogen atmosphere. The resulting mixture was degassed with hydrogen for 3 times and stirred at 25° C. for 4 h. The solid was filtered out and the filtrate was concentrated under reduced pressure. The residue was purified by RP-flash with the following conditions: Column: Flash C¹⁸ (330 g); Mobile Phase A: water (0.1% TFA), Mobile Phase B: ACN; (Gradient: 5% B hold 5 min, up to 30% B within 15 min, 30% B hold 5 min; up to 95% B within 20 min. 95% B hold 10 min); Flow rate: 90 m/min; Detector: UV 210 nm; RT=40 min. The product-containing fractions were collected and evaporated in vacuo to give (S)-2-((((9H-fluoren-9-yl)methoxy)carbonyl)amino)-3-(6-(tert-butoxycarbonyl)pyridin-3-yl)propanoic acid (5.4625 g, 11.18 mmol, 86% yield) as a yellow solid. MS ESI calculated for C₂₈H₂₉N₂O₆ [M+H]⁺ 489.19, found 489.20; ¹H NMR (300 MHz, CD₃OD) δ 8.57 (s, 1H), 8.03-7.91 (m, 1H), 7.86-7.74 (m, 3H) 7.54-7.53 (m, 2H), 7.36-7.28 (m, 4H), 4.51-4.40 (m, 1H), 4.32-4.13 (m, 3H), 3.39-3.33 (m, 1H), 3.06-3.05 (m, 1H), 1.65-1.59 (m, 9H).

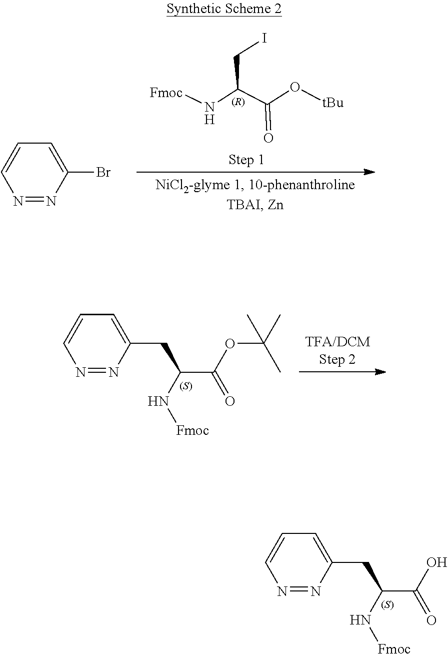

### Precursor to 3PyridaAla

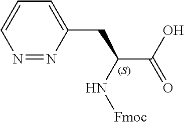

### (S)-2-((((9H-Fluoren-9-yl)methoxy)carbonyl)amino)-3-(pyridazin-3-yl)propanoic acid

Step 1: To a mixture of NiCl₂-glyme (0.713 g, 3.24 mmol) in DMA (20 mL) was added 1,10-phenanthroline (0.703 g, 3.24 mmol) at room temperature. The resulted mixture was stirred at 50° C. for 1 h then cooled down to room temperature and injected the mixture of 3-bromopyridazine (5.16 g, 32.4 mmol), tert-butyl (R)-2-((((9H-fluoren-9-yl)methoxy)carbonyl)amino)-3-iodopropanoate (8.0 g, 16.22 mmol), TBAI (5.99 g, 16.22 mmol) in 120 mL DMA and then Zinc (2.120 g, 32.4 mmol) was added at room temperature. The resulting mixture was stirred for 5 h at room temperature. The reaction was diluted with water (200 mL) and extracted with EA (3×200 mL). The combined organic layer was washed with brine (3×100 mL), dried over Na₂SO₄ and filtered. The filtrate was concentrated under vacuum. The residue was purified by silica gel column chromatography, eluting with EA/PE (4/1) to afford tert-butyl (S)-2-((((9H-fluoren-9-yl)methoxy)carbonyl)amino)-3-(pyridazin-3-yl)propanoate (3.6 g, 8.08 mmol, 50% yield) as a yellow solid. MS ESI calculated for C₂₆H₂₈N₃O₄ [M+H]⁺ 446.20, found 446.30.

Step 2: To a solution of tert-butyl (S)-2-((((9H-fluoren-9-yl)methoxy)carbonyl)amino)-3-(pyridazin-3-yl)propanoate (3.6 g, 8.08 mmol) in DCM (10 mL) was added TFA (20 mL) at room temperature. After the resulting mixture was stirred room temperature for 2 h, it was concentrated under vacuum. The residue was purified by RP-flash, eluting with 0-50% MeCN in water (0.05% TFA) to afford (S)-2-((((9H-fluoren-9-yl)methoxy)carbonyl)amino)-3-(pyridazin-3-yl)propanoic acid (2.5 g, 6.42 mmol, 79% yield) as a light-yellow solid. MS ESI calculated for C₂₂H₂₀N₃O₄ [M+H]⁺ 390.14, found 390.10. ¹H NMR (300 MHz, CD₃OD) δ 9.17 (s, 1H), 7.90-7.77 (m, 4H), 7.61-7.59 (m, 2H), 7.41-7.36 (m, 2H), 7.31-7.26 (m, 2H), 4.74-4.70 (m, 1H), 4.29 (d, J=6.0 Hz, 2H), 4.18-4.14 (m, 1H), 3.67-3.61 (m, 1H), 3.42-3.37 (m, 1H).

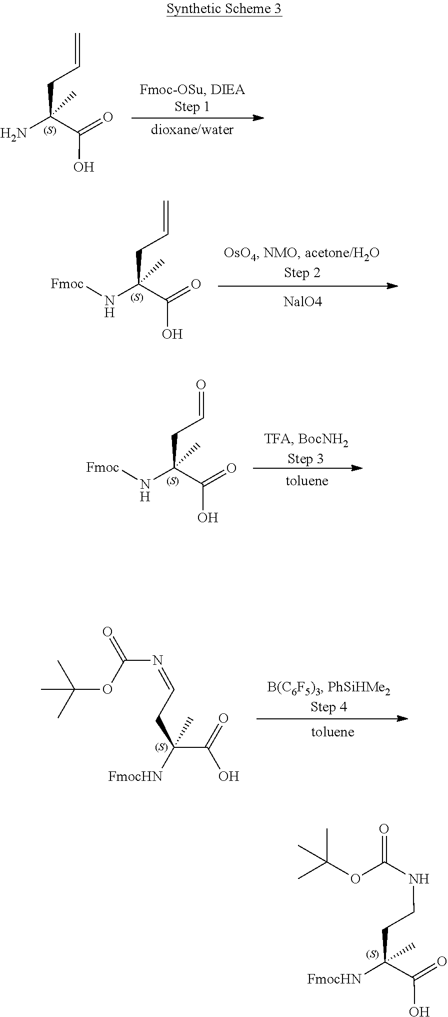

### Precursor to aMeDab

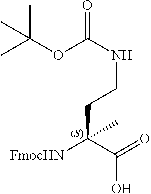

### (S)-2-((((9H-fluoren-9-yl)methoxy)carbonyl)amino)-4-((tert-butoxycarbonyl)amino)-2-methylbutanoic acid

Step 1: To a stirred solution of (S)-2-amino-2-methylpent-4-enoic acid (2 g, 15.48 mmol) in dioxane (20 mL) and water (20 mL) were added DIEA (8.11 mL, 46.5 mmol) and Fmoc-OSu (5.22 g, 15.48 mmol) at room temperature. The resulting solution was stirred at 25° C. for 16 h. The pH of the solution was adjusted to 3 with 6 N HCl and purified by RP-flash with the following conditions: 5%-5% in 5 min. 5%-60% in 30 min, 98%-98% in 5 min, MeCN in water (0.05% TFA), RT=35 min to give (S)-2-((((9H-fluoren-9-yl)methoxy)carbonyl)amino)-2-methylpent-4-enoic acid (4.6 g, 12.44 mmol, 80% yield) as an off-white solid. MS ESI calculated for C₂₁H₂₀NO₄ [M−H]⁺ 350.15, found 350.00. ¹H NMR (300 MHz, CDCl₃) δ 9.67 (s, 1H), 7.82-7.68 (m, 2H), 7.58 (d, J=7.4 Hz, 2H), 7.45-7.19 (m, 4H), 5.83-5.53 (m, 1H), 5.52-5.33 (m, 1H), 5.16-5.12 (m, 2H), 4.64-4.27 (m, 2H), 4.21 (t, J=6.6 Hz, 1H), 2.94-2.48 (m, 2H).

Step 2: To a stirred solution of (S)-2-((((9H-fluoren-9-yl)methoxy)carbonyl)amino)-2-methylpent-4-enoic acid (4.6 g, 13.09 mmol) in acetone (80 mL) were added 4-methylmorpholine (2.91 g, 14.40 mmol, 50% in water) and OSO₄ (3.33 g, 1.309 mmol, 10% in water) at 25° C. The resulting solution was stirred at 25° C. for 4 h. Then sodium periodate (10.27 g, 14.40 mmol, 30% in water) was added and the resulting mixture was stirred at 25° C. for 16 h. The mixture was diluted with water (100 mL), extracted with EA (3×200 mL). The combined organic layer was washed with brine (2×100 mL), dried over anhydrous Na₂SO₄. After filtration, the filtrate was concentrated under reduced pressure to give (S)-2-((((9H-fluoren-9-yl)methoxy)carbonyl)amino)-2-methyl-4-oxobutanoic acid (4.9 g, 11.79 mmol, 90% yield) as a black solid. MS ESI calculated for C₂₀H₁₈NO₅ [M−H]⁻ 352.13, found 352.10.

Step 3: To a stirred solution of (S)-2-((((9H-fluoren-9-yl)methoxy)carbonyl)amino)-2-methyl-4-oxobutanoic acid (1 g, 2.83 mmol) in toluene (50 mL) were added TFA (0.968 g, 8.49 mmol) and tert-butyl carbamate (1.989 g, 16.98 mmol) at room temperature. The resulting solution was stirred at 25° C. for 2 h. The solvent was concentrated under reduced pressure to give (S,Z)-2-((((9H-fluoren-9-yl)methoxy)carbonyl)amino)-4-((tert-butoxycarbonyl)imino)-2-methylbutanoic acid (1.3 g, 2.011 mmol, 71% yield) as a black solid. MS ESI calculated for C₂₅H₂₇N₂O₆ [M−H]⁻ 451.19, found 451.00.

Step 4: To a stirred solution of (S,Z)-2-((((9H-fluoren-9-yl)methoxy)carbonyl)amino)-4-((tert-butoxycarbonyl)imino)-2-methylbutanoic acid (5 g, 7.73 mmol) and dimethyl(phenyl)silane (5.27 g, 38.7 mmol) in toluene (50 mL) was added tris(pentafluorophenyl)borane (0.396 g, 0.773 mmol) at room temperature. The resulting solution was stirred at 25° C. for 16 h. The solvent was concentrated under reduced pressure and the residue was purified by RP-flash with the following conditions: C¹⁸ column, 330 g, 5%-5% in 5 min, 5%-50% in 30 min, 98%-98% in 5 min, MeCN in water (0.025% TFA), RT=35 min to give (S)-2-((((9H-fluoren-9-yl)methoxy)carbonyl)amino)-4-((tert-butoxycarbonyl)amino)-2-methylbutanoic acid (2.5183 g, 5.26 mmol, 68% yield) as an off-white solid. MS ESI calculated for C₂₅H₂₉N₂O₆ [M−H]⁻ 453.21. found 453.00.¹H NMR (400 MHz, DMSO-d₆) δ 12.49 (s, 1H), 7.90 (d, J=6.8 Hz, 2H), 7.73 (d, J=7.6 Hz, 2H), 7.54 (s, 1H), 7.44-7.40 (m, 2H), 7.36-7.32 (m, 2H), 6.77 (s, 1H), 4.25-4.22 (m, 3H), 2.93-2.91 (m, 2H), 1.97-1.81 (m, 2H), 1.37-1.34 (m, 9H).

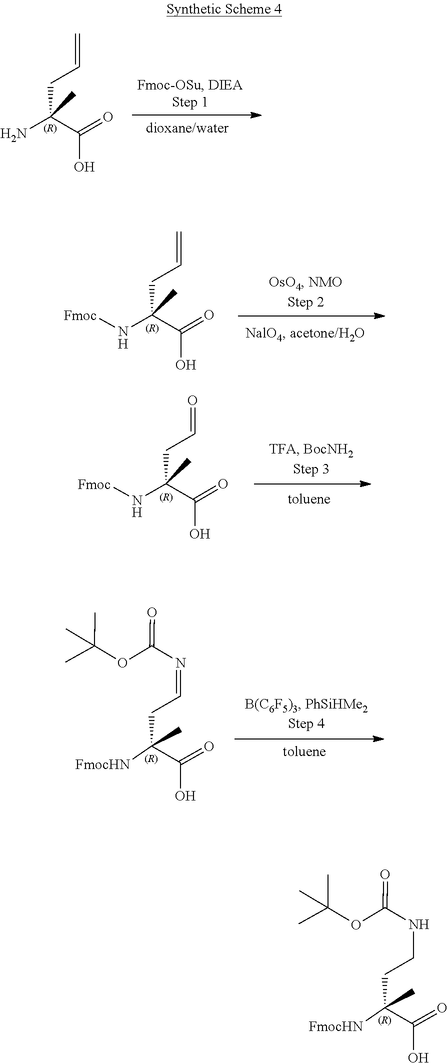

### Precursor to daMeDab

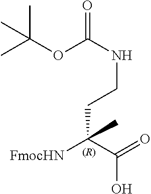

### (R)-2-((((9H-Fluoren-9-yl)methoxy)carbonyl)amino)-4-((tert-butoxycarbonyl)amino)-2-methylbutanoic acid

Step 1: To a stirred solution of (R)-2-amino-2-methylpent-4-enoic acid (1.3 g, 10.07 mmol) and DIEA (5.27 mL, 30.2 mmol) in dioxane (20 mL) and water (20 mL) was added Fmoc-OSu (3.73 g, 11.07 mmol) at room temperature. The resulting solution was stirred at 25° C. for 16 h. The pH was adjusted to 3 with 1 N HCl and the solution was purified by RP-flash with the following conditions: C¹⁸ column, 330 g, 5%-5% in 5 min. 5%-50% in 30 min, 98%-98% in 5 min. MeCN in water (0.05% TFA), RT=30 min to give (R)-2-((((9H-fluoren-9-yl)methoxy)carbonyl)amino)-2-methylpent-4-enoic acid (3 g, 8.11 mmol, 81% yield) as an off-white solid. MS ESI calculated for C₂₁H₂₁NO₄Na [M+Na]⁺ 374.15, found 374.05. ¹H NMR (400 MHz, CDCl₃) δ 7.79 (d, J=7.5 Hz, 2H), 7.61 (d, J=7.5 Hz, 2H), 7.48-7.29 (m, 4H), 5.71 (s, 1H), 5.48 (s, 1H), 5.20-5.16 (m, 2H), 4.44-4.40 (m, 2H), 4.25 (t, J=6.7 Hz, 1H), 2.82-2.71 (m, 2H).

Step 2: To a stirred solution of (R)-2-((((9H-fluoren-9-yl)methoxy)carbonyl)amino)-2-methylpent-4-enoic acid (3 g, 8.54 mmol) and NMO (2.200 g, 9.39 mmol, 50% in water) in acetone (60 mL) was added OsO₄ (2.170 g, 0.854 mmol, 10% in water) at room temperature. The resulting solution was stirred at 25° C. for 4 h. Sodium periodate (2.009 g, 9.39 mmol) in water (20 mL) was added to the solution and the resulting mixture was stirred at 25° C. for 16 h. The reaction mixture was diluted with water (50 mL), extracted with EA (3×200 mL). The combined organic layer was washed with brine (2×100 mL), dried over anhydrous Na₂SO₄. After filtration, the filtrate was concentrated under reduced pressure to give (R)-2-((((9H-fluoren-9-yl)methoxy)carbonyl)amino)-2-methyl-4-oxobutanoic acid (3.4 g, 7.70 mmol, 90% yield) as a black solid. MS ESI calculated for C₂₀H₂₀NO₅ [M+H]⁻ 354.13, found 354.00.

Step 3: To a stirred solution of (R)-2-((((9H-fluoren-9-yl)methoxy)carbonyl)amino)-2-methyl-4-oxobutanoic acid (3 g, 6.79 mmol) in toluene (120 mL) were added tert-butyl carbamate (4.77 g, 40.8 mmol) and TFA (2.323 g, 20.38 mmol) at room temperature. The resulting solution was stirred at 25° C. for 2 h. The solution was concentrated under reduced pressure to give (R,Z)-2-((((9H-fluoren-9-yl)methoxy)carbonyl)amino)-4-((tert-butoxycarbonyl)imino)-2-methylbutanoic acid (3.1 g, 5.48 mmol, 81% yield) as a black solid. MS ESI calculated for C₂₅H₂₇N₂O₆ [M−H]⁺ 451.19, found 450.90.

Step 4: To a stirred solution of (R,Z)-2-((((9H-fluoren-9-yl)methoxy)carbonyl)amino)-4-((tert-butoxycarbonyl)imino)-2-methylbutanoic acid (3.1 g, 5.48 mmol) and dimethyl(phenyl)silane (1.867 g, 13.70 mmol) in toluene (120 mL) was added tris(pentafluorophenyl)borane (0.281 g, 0.548 mmol) at room temperature. The solution was stirred at 25° C. for 16 h. The solvent was concentrated under reduced pressure and the residue was purified by RP-flash with the following conditions: 330 g C¹⁸ column, 5%-5% in 5 min, 5%-55% in 30 min, 98%-98% in 5 min, MeCN in water (0.05% TFA), RT=35 min to give (R)-2-((((9H-fluoren-9-yl)methoxy)carbonyl)amino)-4-((tert-butoxycarbonyl)amino)-2-methylbutanoic acid (2.2528 g, 4.81 mmol, 88% yield) as an off-white solid. MS ESI calculated for C₂₅H₃₁N₂O₆ [M+H]⁺ 455.21, found 455.10; ¹H NMR (400 MHz, DMSO-d₆) δ 12.48 (s, 1H), 7.90 (d, J=7.2 Hz, 2H), 7.73 (d, J=7.2 Hz, 2H), 7.54 (s, 1H), 7.44-7.41 (m, 2H), 7.36-7.32 (m, 2H), 6.78 (s, 1H), 4.25-4.23 (m, 3H), 2.92-2.91 (m, 2H), 1.99-1.93 (m, 1H), 1.87-1.81 (m, 1H), 1.37-1.34 (m, 9H).

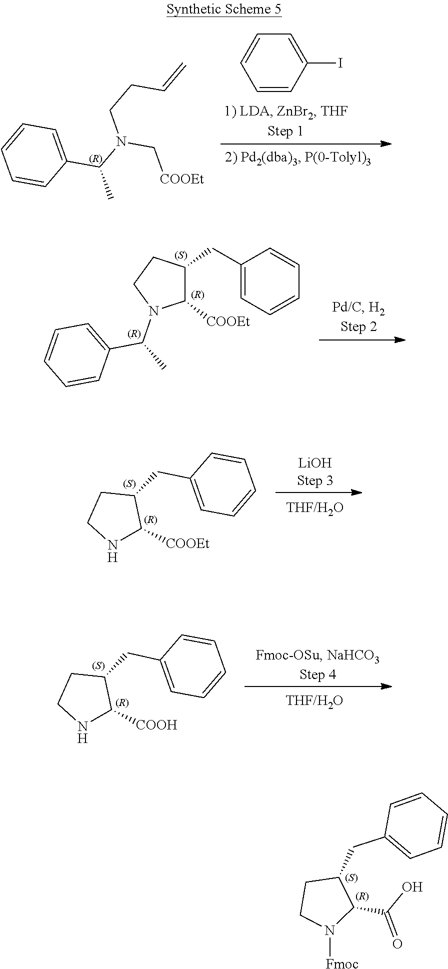

### Precursor to dProc3Bn

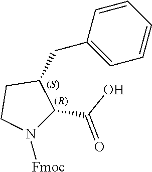

### (2R,3S)-1-(((9H-Fluoren-9-yl)methoxy)carbonyl)-3-benzylpyrrolidine-2-carboxylic acid

Step 1: To a mixture of ethyl (R)—N-(but-3-en-1-yl)-N-(1-phenylethyl)glycinate (10.48 g, 40.1 mmol) in THF (100 mL) was added LDA (20.05 mL, 40.1 mmol, 2 M in THF) under argon at −78° C. The mixture was stirred at −78° C. for 30 min. then to the mixture was added dried ZnBr₂ (120 mL, 120 mmol, 1 N in THF) at −78° C. The resulting mixture was slowly warmed to ambient temperature and stirred at ambient temperature for 4 h. Iodobenzene (10.63 g, 52.1 mmol), Pd₂(dba)₃ (1.102 g, 1.203 mmol) and tri-o-tolylphosphine (1.587 g, 5.21 mmol) were successively added and the reaction was stirred at ambient temperature for 16 h. The resulting mixture was quenched with saturated NH₄Cl (100 mL) and extracted with EA (3×200 mL). The organic layers were combined, washed with brine (2×130 mL), dried over anhydrous Na₂SO₄ and filtered. The filtrate was concentrated under reduced pressure and the residue was purified by silica gel chromatography, eluting with a gradient of 0-15% EA in PE to afford ethyl (2R,3S)-3-benzyl-1-((R)-1-phenylethyl)pyrrolidine-2-carboxylate (8.6 g, 25.5 mmol, 64% yield) as an orange semi-solid. MS ESI calculated for C₂₂H₂₈NO₂ [M+H]⁺ 338.20, found 338.30.

Step 2: To a stirred solution of ethyl (2R,3S)-3-benzyl-1-((R)-1-phenylethyl)pyrrolidine-2-carboxylate (10 g, 29.6 mmol) in EtOH (100 mL) was added Pd/C (4 g, 37.6 mmol, dry, 10% wt) at 25° C. under nitrogen. The resulting mixture was stirred at 25° C. for 10 min, then degassed under vacuum and purged with H₂ several times. The resulting mixture was stirred for 6 h at 60° C. under 2 atm H₂. After filtration, the filtrate was concentrated in vacuo to afford ethyl (2R,3S)-3-benzylpyrrolidine-2-carboxylate (6 g, 25.7 mmol, 87% yield) as a colorless oil. MS ESI calculated for C₁₄H₂₀NO₂ [M+H]⁺ 234.14, found 234.20.

Step 3: To a stirred solution of ethyl (2R,3S)-3-benzylpyrrolidine-2-carboxylate (6 g, 25.7 mmol) in THF (60 mL) was added LiOH (51.4 mL, 51.4 mmol, 1 N in water) at room temperature. The solution was stirred at 25° C. for 12 h, the pH value of the solution was adjusted to 7 with 1 N HCl. The solution was used to the next step directly without any further purification. MS ESI calculated for C₁₂H₁₆NO₂ [M+H]⁺ 206.11, found 206.15.

Step 4: To a stirred solution of ((2R,3S)-3-benzylpyrrolidin-2-yl)(l1-oxidaneyl)methanone (5 g, 24.48 mmol) in THF (50 mL) and water (50 mL) was added NaHCO₃ (10.28 g, 122 mmol) at 25° C. under nitrogen atmosphere. The resulted mixture was stirred at 25° C. for 10 min. Fmoc-OSu (7.43 g, 22.03 mmol) was added to the mixture and stirred at 25° C. for 2 h. The pH value of the solution was adjusted to 3 with 1 N HCl. The aqueous phase was extracted with EA (2×500 mL). The combined organic layer was washed with brine (3×50 mL), dried over anhydrous Na₂SO₄ and filtered. The filtrate was concentrated under reduced pressure and the residue was purified by Rp-flash with the following conditions: Column: Flash C¹⁸ (330 g); Mobile Phase A: water (0.1% TFA). Mobile Phase B: ACN; (Gradient: 5% B hold 5 min, up to 30% B within 15 min, 30% B hold 5 min; up to 95% B within 20 min, 95% B hold 10 min); Flow rate: 90 mL/min; Detector: UV 210 nm; RT=40 min. The product-containing fractions were collected and roto-evaporated in vacuo to give (2R,3S)-1-(((9H-fluoren-9-yl)methoxy)carbonyl)-3-benzylpyrrolidine-2-carboxylic acid (8.2727 g, 19.35 mmol, 79% yield) as an off-white solid. MS ESI calculated for C₂₇H₂₆NO₄ [M+H]⁺ 428.18, found 428.10.¹H NMR (400 MHz, CD₃OD) δ 7.79-7.77 (m, 2H), 7.65-7.60 (m, 2H), 7.40-7.36 (m, 2H), 7.33-7.18 (m, 7H), 4.42-4.18 (m, 4H), 3.68-3.64 (m, 1H), 3.31-3.30 (m, 1H), 3.05-3.02 (m, 1H), 2.78-2.65 (m, 1H), 2.37-2.34 (m, 1H), 1.80-1.77 (m, 2H).

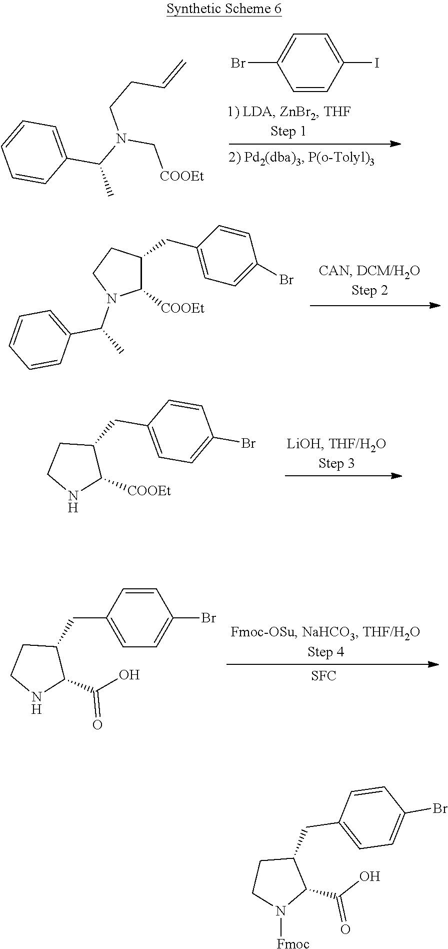

### Precursor to dProc3Bn4Br

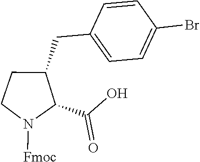

### (2R,3S)-1-(((9H-Fluoren-9-yl)methoxy)carbonyl)-3-(4-bromobenzyl)pyrrolidine-2-carboxylic acid

Step 1: To a solution of ethyl (R)—N-(but-3-en-1-yl)-N-(1-phenylethyl)glycinate (5 g, 19.13 mmol) in THF (30 mL) was added LDA (9.57 mL, 19.13 mmol, 2 M in THF) under argon at −78° C. The solution was stirred at −78° C. for 30 min. And then to the solution was added dried ZnBr₂ (57.4 mL, 57.4 mmol, 1 N in THF) at −78° C. The reaction was slowly warmed to ambient temperature and stirred at ambient temperature for 4 h, 1-Bromo-4-iodobenzene (7.04 g, 24.87 mmol), Pd₂(dba)₃ (0.526 g, 0.574 mmol) and tri-o-tolylphosphine (0.757 g, 2.487 mmol) were then successively added and the reaction was stirred at ambient temperature for 16 h. The resulting solution was quenched with saturated NH₄Cl (50 mL) and extracted with EA (3×150 mL). The organic layers were combined, washed with brine (2×80 mL), dried over anhydrous Na₂SO₄ and filtered. After concentration, the residue was purified by silica gel chromatography, eluted with a gradient of EA in PE from 0% to 11% to afford ethyl (2R,3S)-3-(4-bromobenzyl)-1-((R)-1-phenylethyl)pyrrolidine-2-carboxylate (3.6 g, 8.65 mmol, 45% yield) as an orange semi-solid. MS ESI calculated for C₂₂H₂₇BrNO₂ [M+H]⁺ 416.11, 418.11, found 416.05, 418.05.

Step 2: To a mixture of ethyl (2R,3S)-3-(4-bromobenzyl)-1-((R)-1-phenylethyl)pyrrolidine-2-carboxylate (3.7 g, 8.97 mmol) in DCM (220 mL) was added CAN (14.76 g, 26.9 mmol, dissolved in 44 mL water) at 0° C. for 3 min. The resulting mixture was stirred at ambient temperature for 6 h. The reaction was quenched with 60 mL saturated NaHCO₃ and extracted with EA (3×250 mL). The organic layers were combined, washed with brine (2×150 mL), dried over anhydrous Na₂SO₄ and filtered. The filtrate was concentrated in vacuo and purified by Rp-flash with the following conditions: Column: Flash C¹⁸ (330 g); Mobile Phase A: water (0.05% TFA), Mobile Phase B: ACN; (Gradient: 2% B hold 5 min, up to 32% B within 15 min, 32% B hold 4 min; up to 98% B within 10 min, 98% B hold 3 min); Flow rate: 35 mL/min; Detector: UV 220 nm; RT=38 min. The product-containing fractions were collected and concentrated in vacuo to give ethyl (2R,3S)-3-(4-bromobenzyl)pyrrolidine-2-carboxylate (1.1 g, 3.52 mmol, 39% yield) as a brown semi-solid. MS ESI calculated for C₁₄H₁₉BrNO₂ [M+H]⁺ 312.05, 314.05, found 312.10, 314.10.

Step 3: To a mixture of ethyl (2R,3S)-3-(4-bromobenzyl)pyrrolidine-2-carboxylate (3.3 g, 10.57 mmol) in THF (22 mL) was added LiOH (21.14 mL, 21.14 mmol, 1 M in water) at 0° C. After the reaction was stirred at ambient temperature for 4 h, it was acidified with aqueous HCl to pH 3~4 and concentrated in vacuo to afford crude product (2R,3S)-3-(4-bromobenzyl)pyrrolidine-2-carboxylic acid (3.5 g, 9.85 mmol, 93% yield) as a white solid. MS ESI calculated for C₁₂H₁₅BrNO₂ [M+H]⁺ 284.02, 286.02, found 284.05, 286.05.

Step 4: To a mixture of (2R,3S)-3-(4-bromobenzyl)pyrrolidine-2-carboxylic acid (3.5 g, 9.85 mmol) in THF (30 mL) and water (30 mL) were added NaHCO₃ (4.14 g, 49.3 mmol) and Fmoc-OSu (2.99 g, 8.87 mmol) at ambient temperature. The reaction was stirred at ambient temperature for 4 h. The resulting solution was acidified with aqueous HCl to pH 3~4 and extracted with EA (3×100 mL). The organic layers were combined, washed with brine (2×100 mL), dried over anhydrous Na₂SO₄ and filtered. The filtrate was concentrated in vacuo. The residue was purified by Rp-flash with the following conditions: Column: C¹⁸ (330 g); Mobile Phase A: water (0.05% TFA), Mobile Phase B: ACN; (Gradient: 5% B hold 5 min, up to 78% B within 28 min, 78% B hold 8.5 min; up to 95% B within 2 min, 95% B hold 10 min); Flow rate: 90 mL/min; Detector: UV 210 nm; RT=53.5 min. The product-containing fractions were collected and concentrated in vacuo to give crude product. The crude product was separated by Prep-SFC with the following conditions: Column: CHIRALPAK IH, 3×25 cm, 5 μm; Mobile Phase A: CO₂, Mobile Phase B: IPA:ACN=1:1 (0.1% 2 M NH₃-MeOH); Flow rate: 70 mL/min; Gradient: isocratic 35% B; Column Temperature (° C.): 35; Back Pressure(bar): 100; wavelength: 220 nm; The slower peak was obtained at 8.39 min. The collected fractions were combined and concentrated under vacuum. The residue was lyophilized to afford (2R,3S)-1-(((9H-fluoren-9-yl)methoxy)carbonyl)-3-(4-bromobenzyl)pyrrolidine-2-carboxylic acid (3.0099 g, 5.74 mmol, 58% yield) as an off-white solid. MS ESI calculated for C₂₇H₂₅BrNO₄ [M+H]⁺ 506.09, 508.09, found 506.05, 508.05. ¹H NMR (400 MHz, CD₃OD) δ 7.78-7.76 (m, 2H), 7.64-7.60 (m, 2H), 7.45-7.36 (m, 4H), 7.32-7.28 (m, 2H), 7.18-7.14 (m, 2H), 4.39-4.17 (m, 4H), 3.66-3.63 (m, 1H), 3.32-3.28 (m, 1H), 3.00-2.95 (m, 1H), 2.76-2.62 (m, 1H), 2.38-2.35 (m, 1H), 1.82-1.77 (m, 2H).

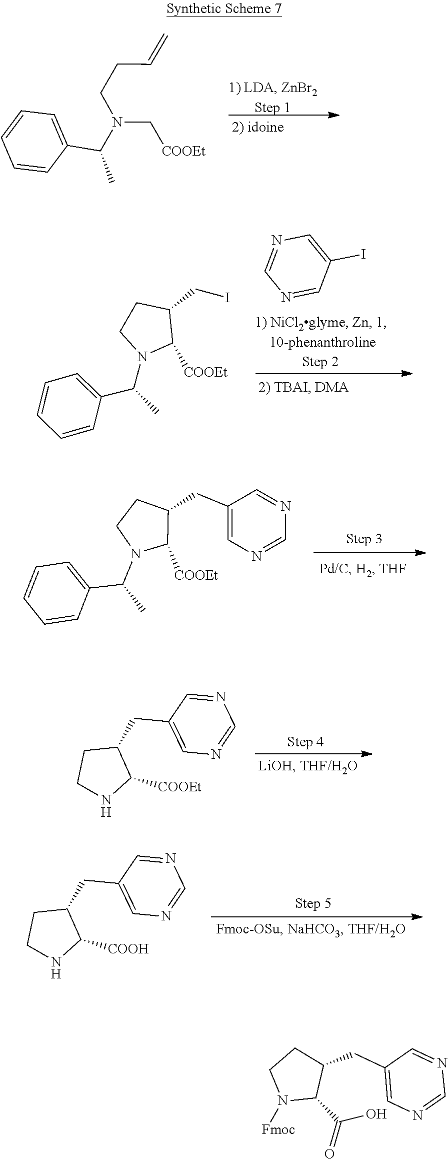

### Precursor to dProc3CH2Pyrim

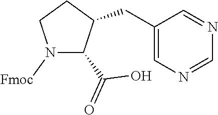

### (2R,3S)-1-(((9H-Fluoren-9-yl)methoxy)carbonyl)-3-(pyrimidin-5-ylmethyl)pyrrolidine-2-carboxylic acid

Step 1: To a solution of ethyl (R)—N-(but-3-en-1-yl)-N-(1-phenylethyl)glycinate (2.61 g, 10 mmol) in THF (10 mL) was added LDA (6 mL, 12.00 mmol, 2 M in THF) under argon at −78° C. After the reaction solution was stirred at −20° C. for 30 min, a solution of ZnBr₂ in THF (15 mL, 30.0 mmol) was added into the reaction at −78° C. After the resulting mixture was warmed naturally and stirred at ambient temperature for 4 h, I₂ (2.66 g, 10.50 mmol) in THF (10 mL) was added. The reaction mixture was stirred at ambient temperature for 16 h then quenched with saturated NH₄Cl (100 mL), extracted with EtOAc (2×100 mL). The combined organic layer was washed with brine (2×100 mL), dried over Na₂SO₄, filtered. The filtrate was concentrated under reduced pressure and the residue was purified by Rp-flash with the following conditions: Column: C¹⁸ gel column (330 g); Mobile Phase A: water (5 mM NH₄HCO₃); Mobile Phase B: MeCN; (Gradient: 0% B hold 5 min, up to 36% B within 20 min, 36% B hold 10 min; up to 95% B within 5 min, 95% B hold 10 min); Flow rate: 80 mL/min; Detector: UV 254 & 210 nm; RT: 35.32 min. The product-containing fractions were collected and roto-evaporated in vacuo to give ethyl (2R,3S)-3-(iodomethyl)-1-((R)-1-phenylethyl)pyrrolidine-2-carboxylate (3.5 g, 9.04 mmol, 90% yield) as a colorless oil. MS ESI calculated for C₁₆H₂₃INO₂ [M+H]⁺ 388.08, found 388.15.

Step 2: Argon gas was bubbled through a mixture of ethyl (2R,3S)-3-(iodomethyl)-1-((R)-1-phenylethyl)pyrrolidine-2-carboxylate (2 g, 5.16 mmol), 5-iodopyrimidine (1.383 g, 6.71 mmol) and TBAI (1.908 g, 5.16 mmol) in DMA (10 mL), which was marked as solution A. Argon gas was bubbled through a mixture of NiCl-glyme (0.227 g, 1.033 mmol) and 1,10-phenanthroline (0.186 g, 1.033 mmol) in DMA (10 mL), which was stirred at 50° C. for 0.5 h and marked as solution B. The solution A was syringed into solution B, followed by adding Zn (0.675 g, 10.33 mmol). The reaction solution was degassed by syringe with argon while stirring for 10 minutes before stirring at 30° C. The reaction was stirred at 30° C. for 16 h. The reaction mixture was purified by Rp-flash with the following conditions: Column: Flash C¹⁸ column (120 g); Mobile Phase A: water (0.1% TFA), Mobile Phase B: ACN; (Gradient: 5% B hold 5 min, up to 76% B within 30 min, 76% B hold 8.6 min; up to 95% B within 2 min, 95% B hold 10 min); Flow rate: 60 mL/min; Detector: UV 210 nm; RT=34 min. The product-containing fractions were collected and roto-evaporated in vacuo to give ethyl (2R,3S)-1-((R)-1-phenylethyl)-3-(pyrimidin-5-ylmethyl)pyrrolidine-2-carboxylate (760 mg, 2.239 mmol, 43% yield) as a yellow solid. MS ESI calculated for C₂₀H₂₆N₃O₂ [M+H]⁺ 340.20, found 340.25.

Step 3: Ethyl (2R,3S)-1-((R)-1-phenylethyl)-3-(pyrimidin-5-ylmethyl)pyrrolidine-2-carboxylate (710 mg, 2.092 mmol) was dissolved in THF (20 mL) then evacuated and an atmosphere of argon was applied at ambient temperature. Then Pd/C (100 mg, 0.094 mmol, dry, 10% wt) was added under argon atmosphere. The suspension was degassed under vacuum and purged with H₂ for several times. After the reaction solution was stirred for 1.5 h at 50° C. under 2 atm H₂. The suspension was filtered and the filtrate was concentrated under reduced pressure to give ethyl (2R,3S)-3-(pyrimidin-5-ylmethyl)pyrrolidine-2-carboxylate (492 mg, 2.092 mmol, 100% yield) as a light yellow solid. MS ESI calculated for C₁₂H₁₈N₃O₂ [M+H]⁺ 236.14, found 236.15.

Step 4: To a stirred mixture of ethyl (2R,3S)-3-(pyrimidin-5-ylmethyl)pyrrolidine-2-carboxylate (0.492 g, 2.092 mmol) in THF (10 mL) was added a solution of LiOH (4.18 mL, 4.18 mmol, 1 N in water) at ambient temperature. The resulting mixture was stirred for 2 h at ambient temperature then concentrated under reduced pressure to give crude (11-oxidaneyl)((2R,3S)-3-(pyrimidin-5-ylmethyl)pyrrolidin-2-yl)methanone (0.431 g, 2.092 mmol, 100% yield) as a yellow oil. MS ESI calculated for C₁₀H₁₄N₃O₂ [M+H]⁺ 208.11, found 208.15.

Step 5: To a solution of (11-oxidaneyl)((2R,3S)-3-(pyrimidin-5-ylmethyl)pyrrolidin-2-yl)methanone (431 mg, 2.092 mmol) in THF (5 mL) and water (5 mL) were added NaHCO₃ (879 mg, 10.46 mmol) and Fmoc-OSu (635 mg, 1.883 mmol) at ambient temperature. The reaction mixture was stirred at room temperature for 16 h then 1 M HCl was added to adjust pH to 5. The resulting solution was diluted with EA (100 mL) and washed with brine (3×30 mL). The organic layer was dried over anhydrous Na₂SO₄ and filtered. The filtrate was concentrated and the residue was purified by Column: Flash C¹⁸ (330 g); Mobile Phase A: water (0.1% TFA), Mobile Phase B: ACN; (Gradient: 5% B hold 5 min, up to 60% B within 30 min, 60% B hold 2.6 min; up to 95% B within 2 min, 95% B hold 5 min); Flow rate: 80 mL/min; Detector: UV 210 nm; RT=31 min. The product-containing fractions were collected and roto-evaporated in vacuo to give crude product. The crude product was separated by Prep-SFC with the following conditions: Column: CHIRALPAK IH, 3×25 cm, 5 μm; Mobile Phase A: CO₂, Mobile Phase B: MeOH (0.1% 2 M NH₃-MeOH); Flow rate: 60 mL/min; Gradient: isocratic 30% B; Column Temperature (° C.): 35; Back Pressure (bar): 100; Wavelength: 220 nm; RT1 (min): 5.72; RT2 (min): 7.18; Sample Solvent: MeOH (0.1% 2 M NH₃-MeOH); Injection Volume: 1.5 mL; Number of Runs: 20. The fractions of second peak (RT2: 7.18 min) were collected, concentrated in vacuo and then lyophilized overnight to give (2R,3S)-1-(((9H-fluoren-9-yl)methoxy)carbonyl)-3-(pyrimidin-5-ylmethyl)pyrrolidine-2-carboxylic acid (450 mg, 1.048 mmol, 50% yield) as a white solid. MS ESI calculated for C₂₅H₂₄N₃O₄ [M+H]⁺ 430.18, found 430.20.1H NMR (400 MHz, CD₃OD) δ 9.03 (s, 1H), 8.76-8.75 (m, 2H), 7.80-7.79 (m, 2H), 7.77-7.63 (m, 2H), 7.41-7.35 (m, 2H), 7.32-7.29 (m, 2H), 4.40-4.33 (m, 3H), 4.32-4.17 (m, 1H), 3.67-3.66 (m, 1H), 3.37-3.35 (m, 1H), 3.06-2.96 (m, 1H), 2.86-2.72 (m, 1H), 2.67-2.64 (m, 1H), 1.94-1.90 (m, 2H).

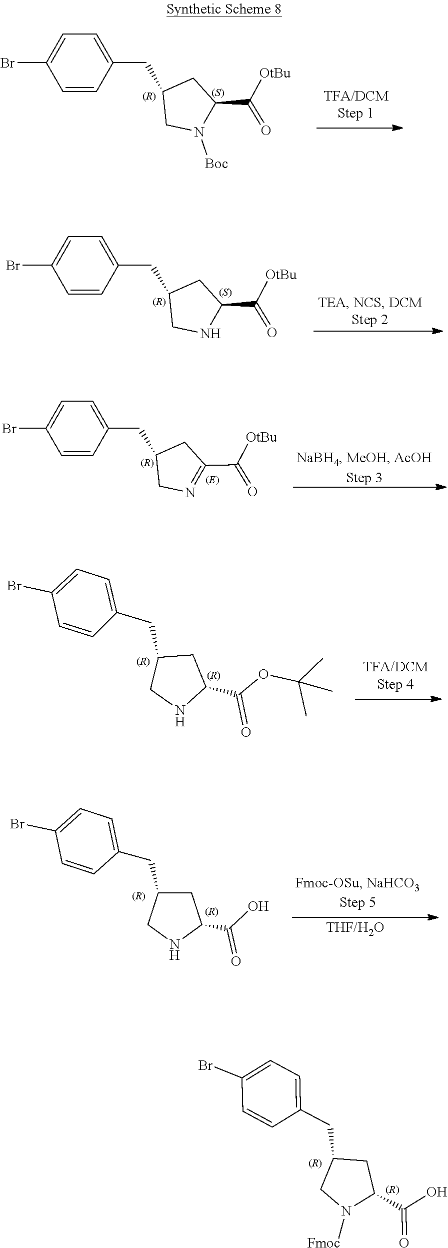

### Precursor to dProc4Bn4Br

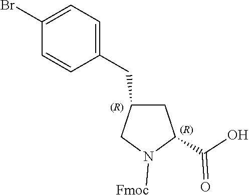

### (2R,4R)-1-(((9H-fluoren-9-yl)methoxy)carbonyl)-4-(4-bromobenzyl)pyrrolidine-2-carboxylic acid

Step 1: To a stirred solution of di-tert-butyl (2S,4R)-4-(4-bromobenzyl)pyrrolidine-1,2-dicarboxylate (4 g, 9.08 mmol) in DCM (40 mL) was added TFA (8 mL) at 0° C. The solution was stirred at 25° C. for 40 min. The solution was concentrated under reduced pressure and the residue was purified by RP-flash with the following conditions: 330 g, C¹⁸ column, 5%-5% in 5 min, 5%-35% in 20 min, 98%-98% in 5 min, MeCN in water (0.05% TFA), RT=25 min. The collections contained desired product were combined. The pH of the solution was adjusted to 10 with sat'd NaHCO₃. The aqueous was extracted with EA (3×100 mL). The combined organic layer was washed with brine (100 mL), dried over anhydrous Na₂SO₄. After filtration, the filtrate was concentrated under reduced pressure to give tert-butyl (2S,4R)-4-(4-bromobenzyl)pyrrolidine-2-carboxylate (2.4 g, 6.70 mmol, 73.8% yield) as a yellow solid. MS ESI calculated for C₁₆H₂₃NO₂ [M+H]⁺ 340.08, 342.08, found 340.10, 342.10.

Step 2: To a stirred solution of tert-butyl (2S,4R)-4-(4-bromobenzyl)pyrrolidine-2-carboxylate (2.4 g, 7.05 mmol) and TEA (1.071 g, 10.58 mmol) in DCM (40 mL) was added NCS (1.036 g, 7.76 mmol) at 0° C. under nitrogen atmosphere. The solution was stirred at 25° C. for 2 h. The solvent was concentrated under reduced pressure and the residue was purified by a silica gel column chromatography, eluted with 0-20% EA in PE to afford tert-butyl (R)-3-(4-bromobenzyl)-3,4-dihydro-2H-pyrrole-5-carboxylate (2.2 g, 6.18 mmol, 88% yield) as a light-yellow oil. MS ESI calculated for C₁₆H₂₁BrNO₂ [M+H]⁺ 338.07, 340.07 found 338.00, 340.00. ¹H NMR (300 MHz, CDCl₃) δ 7.39-7.31 (m, 2H), 7.00-6.92 (m, 2H), 4.06 (dd, J=17.7, 6.9 Hz, 1H), 3.74 (dd, J=16.2, 3.6 Hz, 1H), 2.84-2.81 (m, 1H), 2.68-2.41 (m, 4H), 1.48 (s, 9H).

Step 3: To a stirred solution of tert-butyl (R)-3-(4-bromobenzyl)-3,4-dihydro-2H-pyrrole-5-carboxylate (2.2 g, 6.50 mmol) in MeOH (40 mL) and AcOH (10 mL) was added NaBH₄ (0.492 g, 13.01 mmol) at −45° C. under nitrogen atmosphere. The solution was stirred at −45° C. for 2 h. The solution was quenched with water (2 mL). The solution was purified by RP-flash with the following conditions: 330 g, C¹⁸ column, 5%-5% in 5 min, 5%-35% in 20 min, 98%-98% in 5 min, MeCN in water (0.05% TFA), RT=25 min. The collections contained desired product were combined. The pH of the solution was adjusted to 10 with sat'd NaHCO₃. The aqueous was extracted with EA (3×100 mL). The combined organic layer was washed with brine (100 mL), dried over anhydrous Na₂SO₄. After filtration, the filtrate was concentrated under reduced pressure to give the crude product (1.6 g, 75:25 in SFC). The crude product was separated by SFC with the following conditions: Column: Chiral Art Amylose-SA, 3*25 cm, 5 μm; Mobile Phase A: CO₂, Mobile Phase B: MeOH (0.1% 2 M NH₃-MeOH); Flow rate: 100 mL/min; Gradient: isocratic 15% B; Column Temperature (° C.): 35; Back Pressure (bar): 100; Wavelength: 220 nm; RT1 (min): 3.13; RT2 (min): 3.8; Sample Solvent: MeOH (0.1% 2 M NH₃-MeOH); Injection Volume: 1 mL; Number of Runs: 20. The fractions at 3.8 min were collected and concentrated under reduced pressure to give tert-butyl (2R,4R)-4-(4-bromobenzyl)pyrrolidine-2-carboxylate (0.95 g, 2.65 mmol, 40.8% yield) as a yellow oil. MS ESI calculated for C₁₆H₂₃BrNO₃ [M+H]⁺ 340.08, 342.08, found 340.00, 342.00.

Step 4: To a stirred solution of tert-butyl (2R,4R)-4-(4-bromobenzyl)pyrrolidine-2-carboxylate (950 mg, 2.79 mmol) in DCM (10 mL) was added TFA (20 mL) at room temperature. The solution was stirred at 25° C. for 2 h. The solvent was concentrated under reduced pressure to give (2R,4R)-4-(4-bromobenzyl)pyrrolidine-2-carboxylic acid (0.8 g, 2.53 mmol, 91% yield) as a light-yellow oil. MS ESI calculated for C₁₂H₁₅BrNO₂ [M+H]⁺ 284.02, 286.02 found 284.00, 286.00.

Step 5: To a stirred solution of (2R,4R)-4-(4-bromobenzyl)pyrrolidine-2-carboxylic acid (0.8 g, 2.53 mmol) in THF (20 mL) and water (20.00 mL) were added NaHCO₃ (0.639 g, 7.60 mmol) and Fmoc-OSu (0.855 g, 2.53 mmol) at room temperature. The mixture was stirred at 25° C. for 16 h. The pH was adjusted to 3 with 1 N HCl. The aqueous phase was extracted with EA (3×200 mL). The combined organic layer was washed with brine (100 mL), dried over anhydrous Na₂SO₄. After filtration, the filtrate was concentrated under reduced pressure and the residue was purified by RP-flash with the following conditions: 120 g C¹⁸ column, 5%-5% in 5 min, 5%-60% in 30 min, 98%-98% in 5 min, MeCN in water (0.05% TFA), RT=35 min to give (2R,4R)-1-(((9H-fluoren-9-yl)methoxy)carbonyl)-4-(4-bromobenzyl)pyrrolidine-2-carboxylic acid (1.1603 g, 2.268 mmol, 90% yield) as an off-white solid. MS ESI calculated for C₂₇H₂₅BrNO₄ [M+H]⁺ 506.09, 508.09, found 506.00, 508.00. ¹H NMR (400 MHz, CD₃OD) δ 7.77-7.26 (m, 10H), 7.14-7.09 (m, 2H), 4.46-4.14 (m, 4H), 3.69-3.31 (m, 1H), 3.15-2.97 (m, 1H), 2.72-2.46 (m, 4H), 1.74-1.58 (m, 1H).

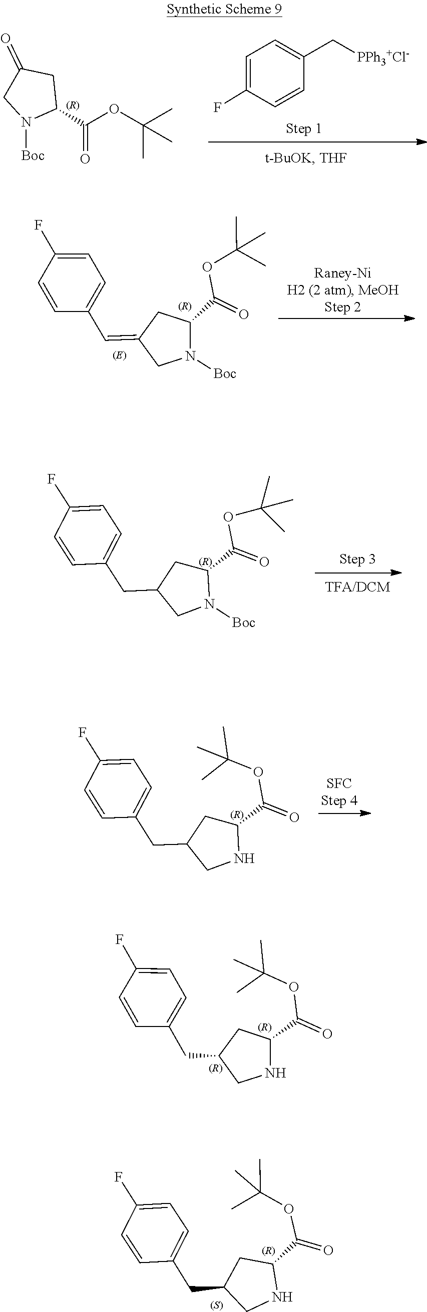

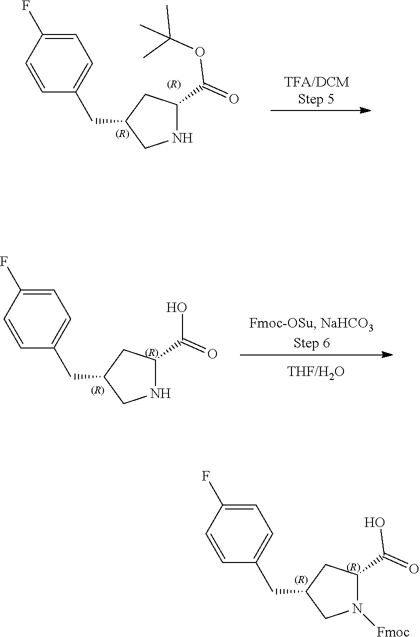

### Precursor to dProc4Bn4F

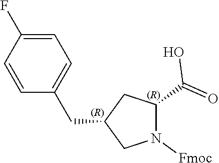

### (2R,4R)-1-(((9H-Fluoren-9-yl)methoxy)carbonyl)-4-(4-fluorobenzyl)pyrrolidine-2-carboxylic acid

Step 1: To a mixture of (4-fluorobenzyl)triphenylphosphonium chloride (17.82 g, 43.8 mmol) in THF (30 mL) was added potassium 2-methylpropan-2-olate (4.92 g, 43.8 mmol) under argon. The reaction was stirred at room temperature for 1 h. To the mixture was added a solution of di-tert-butyl (R)-4-oxopyrrolidine-1,2-dicarboxylate (5 g, 17.52 mmol) in THF (20 mL). The reaction was stirred at room temperature for 2 h. The resulting solution was quenched with water (50 mL) and extracted with ethyl acetate (3×300 mL). The organic layers were combined, washed with brine (2×50 mL), dried over anhydrous sodium sulfate and filtrated. The filtrate was concentrated in vacuo and the residue was purified by silica gel chromatography, eluted with a gradient of ethyl acetate:petroleum ether—0:1 to 1:5 to afford di-tert-butyl (R,E)-4-(4-fluorobenzylidene)pyrrolidine-1,2-dicarboxylate (4.8 g, 12.72 mmol, 73% yield) as a colorless oil. MS ESI calculated for C₂₁H₂₉FNO₄ [M+H]⁺ 378.21, found 378.20.

Step 2: To a mixture of di-tert-butyl (R,E)-4-(4-fluorobenzylidene)pyrrolidine-1,2-dicarboxylate (5 g, 13.25 mmol) in MeOH (50 mL) was added Raney Ni (1.2 g, 20.45 mmol) at room temperature under argon. The suspension was degassed under vacuum and purged with H₂ several times, the reaction solution was stirred for 6 h at room temperature under 2 atm H₂. LCMS showed major was product. The resulting solution was filtrated. The filtrate was concentrated in vacuo to afford di-tert-butyl (2R)-4-(4-fluorobenzyl)pyrrolidine-1,2-dicarboxylate (4.5 g, 11.86 mmol, 90% yield) as a colorless oil. MS ESI calculated for C₂₁H₃₁FNO₄ [M+H]⁺ 380.22, found 380.20.

Step 3: To a stirred solution of di-tert-butyl (2R)-4-(4-fluorobenzyl)pyrrolidine-1,2-dicarboxylate (4.5 g, 11.86 mmol) in TFA (10 mL) and DCM (25 mL) at room temperature. The solution was stirred at room temperature for 1 h. The solvent was concentrated under reduced pressure and the residue was purified by RP flash with the following conditions: Column: Flash C 18 (330 g); Mobile Phase A: water (0.1% TFA), Mobile Phase B: ACN; (Gradient: 5% B hold 5 min, up to 42% B within 15 min, 42% B hold 5 min; up to 95% B within 5 min, 95% B hold 5 min); Flow rate: 90 mL/min; Detector: UV 210 nm; RT=26 min. The product-containing fractions were collected and roto-evaporated in vacuo to give tert-butyl (2R)-4-(4-fluorobenzyl)pyrrolidine-2-carboxylate (2.8 g, 10.02 mmol, 85% yield) as a light yellow oil. MS ESI calculated for C₁₆H₂₃FNO₂ [M+H]⁺ 280.17, found 280.25.

Step 4: tert-butyl (2R)-4-(4-fluorobenzyl)pyrrolidine-2-carboxylate (2.8 g, 10.02 mmol) was separated by Prep-SFC with the following conditions: Column: Lux Cellulose-4, 4.6*50 mm, 3 μm; Mobile Phase A: Hex(0.1% NH₃·H₂O), Mobile Phase B: MeOH Preparative; Flow rate: 1.0 mL/min; Gradient: 30% B; Column Temperature: 25° C.; Back Pressure: 100 bar; 190 nm; RT1:3.42 min; RT2: 4.16 min. The fractions of first peak (RT1: 3.42 min) were collected and roto-evaporated in vacuo to give tert-butyl (2R,4S)-4-(4-fluorobenzyl)pyrrolidine-2-carboxylate (300 mg, 1.074 mmol, 11% yield) as a yellow oil. MS ESI calculated for C₁₆H₂₃FNO₂ [M+H]⁺ 280.17, found 280.25. The fractions of second peak (RT2: 4.16 min) were collected and roto-evaporated in vacuo to give tert-butyl (2R,4R)-4-(4-fluorobenzyl)pyrrolidine-2-carboxylate (2.1 g, 7.52 mmol, 75% yield) as a yellow oil. MS ESI calculated for C₁₆H₂₃FNO₂ [M+H]⁺ 280.17, found 280.25. ¹H NMR (400 MHz, Methanol-d₄) δ 7.25-7.21 (m, 2H), 7.05-7.01 (m, 2H), 4.35-4.31 (m, 1H), 3.43-3.39 (m, 1H), 3.08-3.03 (m, 1H), 2.78-2.77 (m, 2H), 2.76-2.72 (m, 1H), 2.48-2.47 (m, 1H), 1.77-1.74 (m, 1H), 1.52 (s, 9H).

Step 5: The solution of tert-butyl (2R,4R)-4-(4-fluorobenzyl)pyrrolidine-2-carboxylate (2.1 g, 7.52 mmol) in CH₂Cl₂ (10 mL) and TFA (10.00 mL) was stirred at 25° C. for 3 hours. The reaction progress was monitored by LCMS. The reaction mixture was concentrated in vacuo to give crude (2R,4R)-4-(4-fluorobenzyl)pyrrolidine-2-carboxylic acid (1.678 g, ~7.52 mmol, 100% yield) as a yellow solid. MS ESI calculated for C₁₂H₁₅FNO₂ [M+H]⁺ 224.11, found 224.15.

Step 6: To a solution of (2R,4R)-4-(4-fluorobenzyl)pyrrolidine-2-carboxylic acid (1.678 g, 7.52 mmol) in THF (10 mL) and Water (10.00 mL) were added Sodium bicarbonate (3.95 g, 47.0 mmol) and N-(9-fluorenylmethoxycarbonyloxy)succinimide (2.86 g, 8.47 mmol) at room temperature. The reaction mixture was stirred at room temperature for 16 h then extracted with Ethyl acetate (3×200 mL). The combined organic layer was washed with brine (3×100 mL), dried over anhydrous Na₂SO₄ and filtrated. The filtrate was concentrated and was purified by Column: Flash C 18 (120 g); Mobile Phase A: water (0.1% TFA), Mobile Phase B: ACN; (Gradient: 5% B hold 5 min, up to 65% B within 25 min, 65% B hold 2.6 min; up to 95% B within 2 min, 95% B hold 5 min); Flow rate: 70 mL/min; Detector: UV 210 nm; RT=26 min. The product-containing fractions were collected and roto-evaporated in vacuo to give (2R,4R)-1-(((9H-fluoren-9-yl)methoxy)carbonyl)-4-(4-fluorobenzyl)pyrrolidine-2-carboxylic acid (2.6866 g, 6.03 mmol, 80% yield) as a white solid. MS ESI calculated for C₂₇H₂₅FNO₄ [M+H]⁺ 446.18. found 446.25. ¹H NMR (300 MHz, Methanol-d₄) δ 7.78-7.71 (m, 2H), 7.63-7.53 (m, 2H), 7.39-7.26 (m, 4H), 7.21-7.14 (m, 2H), 7.07-6.97 (m, 2H), 4.39-4.37 (m, 1H), 4.32-4.14 (m, 3H), 3.68-3.32 (m, 1H), 3.22-2.96 (m, 1H), 2.71-2.37 (m, 2H), 2.43-2.36 (m, 2H), 1.81-1.62 (m, 1H).

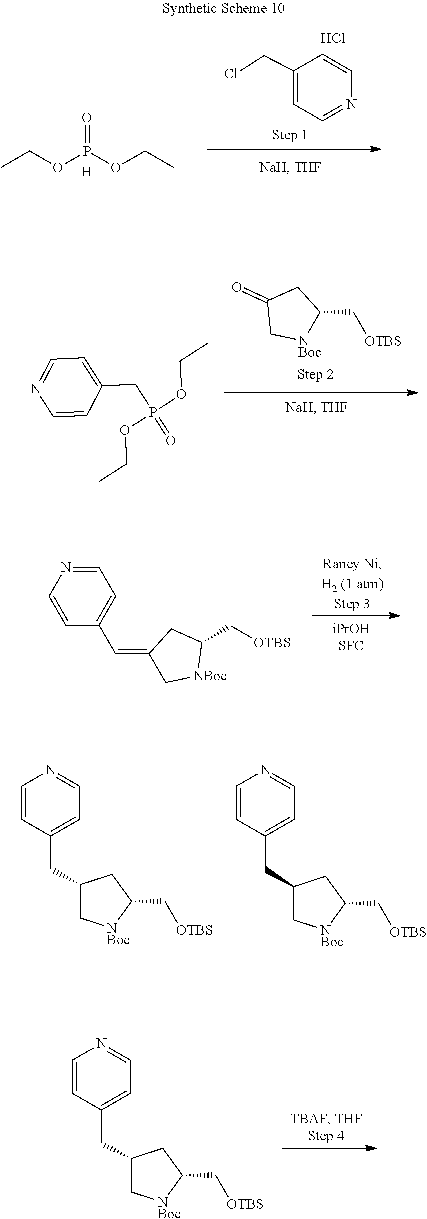

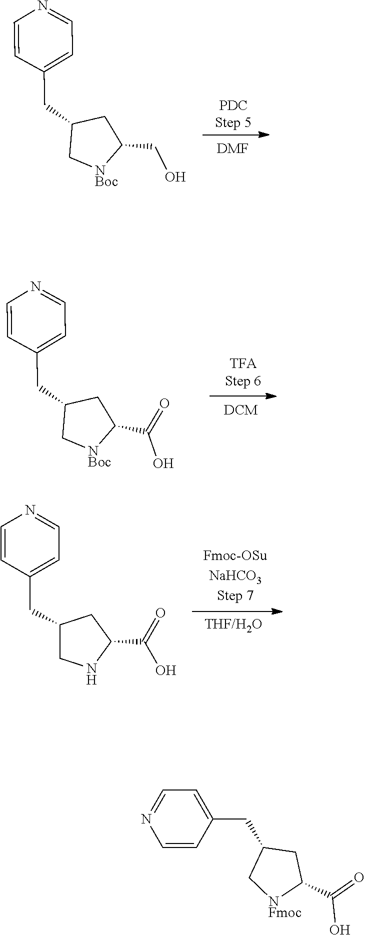

### Precursor to dProc4CH24Pal

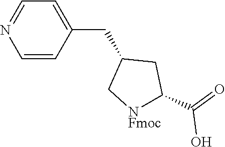

### (2R,4R)-1-(((9H-Fluoren-9-yl)methoxy)carbonyl)-4-(pyridin-4-ylmethyl)pyrrolidine-2-carboxylic acid

Step 1: To a mixture of diethyl phosphonate (8.29 g, 60.0 mmol) in THF (100 mL) was added sodium hydride (3.00 g, 125 mmol) at 0° C. under argon. The reaction was stirred at 0° C. for 30 min. then to the mixture was added 4-(chloromethyl)pyridine hydrochloride (8.20 g, 50 mmol) at 0° C. After the resulting mixture was stirred at ambient temperature for 2 h, it was quenched with water (150 mL) and extracted with ethyl acetate (3×300 mL). The organic layers were combined, washed with brine (2×180 mL), dried over anhydrous sodium sulfateand filtered. The filtrate was concentrated under reduced pressure. The residue was purified by silica gel chromatography, eluted with a gradient of dichloromethane:methanol—1:0 to 15:1 to afford diethyl (pyridin-4-ylmethyl)phosphonate (5.5 g, 23.99 mmol, 48% yield) as an orange oil. MS ESI calculated for C₁₀H₁₇NO₃P [M+H]⁺ 230.09, found 230.15.

Step 2: To a mixture of diethyl (pyridin-4-ylmethyl)phosphonate (2.4 g, 10.47 mmol) in THF (20 mL) was added NaH (60% in mineral oil) (0.523 g, 13.09 mmol) at 0° C. under argon. The mixture was stirred at 0° C. for 30 min then was added tert-butyl (R)-2-(((tert-butyldimethylsilyl)oxy)methyl)-4-oxopyrrolidine-1-carboxylate (3.11 g, 9.42 mmol) at 0° C. under argon. After the resulting mixture was stirred at ambient temperature for 1 h under argon, it was quenched with water (40 mL) and extracted with ethyl acetate (3×100 mL). The organic layers were combined, washed with brine (2×50 mL), dried over anhydrous sodium sulfateand filtered. The filtrate was concentrated under reduced pressure. The residue was purified by silica gel chromatography, eluting with a gradient of ethyl acetate:petroleum ether—0:1 to 1:3 to afford tert-butyl (R,E)-2-(((tert-butyldimethylsilyl)oxy)methyl)-4-(pyridin-4-ylmethylene)pyrrolidine-1-carboxylate (2.8 g, 6.92 mmol, 66% yield) as an orange semi-solid. MS ESI calculated for C₂₂H₃₇N₂O₃Si [M+H]⁺ 405.25, found 405.30.

Step 3: The tert-butyl (R,E)-2-(((tert-butyldimethylsilyl)oxy)methyl)-4-(pyridin-4-ylmethylene)pyrrolidine-1-carboxylate (7.58 g, 18.73 mmol)(combined with other batches) was dissolved in 2-Propanol (80 mL) and the resulting mixture was evacuated and an atmosphere of nitrogen was applied at ambient temperature. Then Raney Ni (washed with 2-Propanol several times, 30 g, 511 mmol) was added under Nitrogen atmosphere. The suspension was degassed under vacuum and purged with H₂ several times. After the resulting mixture was stirred for 12 h at 65° C. under 1 atm H₂, it was filtered by diatomite and washed by 2-propanol (2×150 mL). The filter was concentrated in vacuo to afford racemic product. The racemic product was separated by prep-SFC-HPLC with the following conditions: Column: CHIRAL ART Cellulose-SC, 5*25 cm, 5 m; Mobile Phase A: CO2, Mobile Phase B: IPA (0.5% 2M NH3-MeOH); Flow rate: 250 mL/min; Gradient: isocratic 44% B; Column Temperature(° C.): 35; Back Pressure(bar): 100; Wavelength: 220 nm; Sample Solvent: MeOH:DCM=2:1; Injection Volume: 3 mL. The fast peak was obtained at 7.3 min. The collected fractions were combined and concentrated under vacuum. The residue was lyophilized to afford tert-butyl (2R,4S)-2-(((tert-butyldimethylsilyl)oxy)methyl)-4-(pyridin-4-ylmethyl)pyrrolidine-1-carboxylate (420 mg, 1.03 mmol, 6% yield) as a light yellow semi-solid. MS ESI calculated for C₂₂H₃₉NO₃Si [M+H]⁺ 407.27. found 407.30.

The slower peak was obtained at 8.6 min. The collected fractions were combined and concentrated under vacuum. The residue was lyophilized to afford tert-butyl (2R,4R)-2-(((tert-butyldimethylsilyl)oxy)methyl)-4-(pyridin-4-ylmethyl)pyrrolidine-1-carboxylate (3.4 g, 8.36 mmol, 45% yield) as a light yellow semi-solid. MS ESI calculated for C₂₂H₃₉NO₃Si [M+H]⁺ 407.27. found 407.70.

Step 4: To a mixture of tert-butyl (2R,4R)-2-(((tert-butyldimethylsilyl)oxy)methyl)-4-(pyridin-4-ylmethyl)pyrrolidine-1-carboxylate (3.4 g, 8.36 mmol) in THF (16.72 mL) was added TBAF (1M in THF, 16.72 mL, 16.72 mmol). The reaction was stirred at ambient temperature for 2 h then concentrated in vacuo to afford crude product. The residue was purified by RP flash with the following conditions: Column: Flash C 18 (330 g); Mobile Phase A: water (0.05% TFA), Mobile Phase B: ACN; (Gradient: 2% B hold 3 min, up to 44% B within 15 min, 44% B hold 4.5 min; up to 95% B within 5 min, 95% B hold 5 min); Flow rate: 80 mL/min; Detector: UV 210 nm; RT=32.5 min. The product-containing fractions were collected and concentrated in vacuo to give tert-butyl (2R,4R)-2-(hydroxymethyl)-4-(pyridin-4-ylmethyl)pyrrolidine-1-carboxylate (2.2 g, 7.52 mmol, 90% yield) as a light orange oil. MS ESI calculated for C₁₆H₂₅N₂O₃ [M+H]⁺ 293.18, found 293.20.

Step 5: To a mixture of tert-butyl (2R,4R)-2-(hydroxymethyl)-4-(pyridin-4-ylmethyl)pyrrolidine-1-carboxylate (2.2 g, 7.52 mmol) in DMF (20 mL) was added PDC (14.15 g, 37.6 mmol). The reaction was stirred at ambient temperature for 12 h. After completion, the pH value of the solution was adjusted to 3 with 1 N HCl. The aqueous phase was extracted with EA (2×250 mL). The combined organic layer was washed with brine (2×25 mL), dried over anhydrous Na₂SO₄ and filtered. The filtrate was concentrated under reduced pressure and the residue was purified by RP flash with the following conditions: Column: Flash C 18 (330 g); Mobile Phase A: water (0.05% TFA), Mobile Phase B: ACN; (Gradient: 2% B hold 5 min, up to 33% B within 12 min, 33% B hold 8 min; up to 95% B within 5 min, 95% B hold 5 min); Flow rate: 80 m/min; Detector: UV 210 nm; RT=35 min. The product-containing fractions were collected and concentrated in vacuo to give (2R,4R)-1-(tert-butoxycarbonyl)-4-(pyridin-4-ylmethyl)pyrrolidine-2-carboxylic acid (2 g, 6.53 mmol, 87% yield) as a black semi-solid. MS ESI calculated for C₁₆H₂₃N₂O₄ [M+H]⁺ 307.16, found 307.15.

Step 6: To a mixture of (2R,4R)-1-(tert-butoxycarbonyl)-4-(pyridin-4-ylmethyl)pyrrolidine-2-carboxylic acid (2.0 g, 6.53 mmol) in CH₂Cl₂ (10 mL) was added TFA (10 mL, 130 mmol). The reaction was stirred at ambient temperature for 1 h. The reaction was concentrated in vacuo to afford crude product (2R,4R)-4-(pyridin-4-ylmethyl)pyrrolidine-2-carboxylic acid (2.3 g, 5.58 mmol, 85% yield) as a black semi-solid. MS ESI calculated for C₁₁H₁₅N₂O₂ [M+H]⁺ 207.11. found 207.20.

Step 7: To a mixture of (2R,4R)-4-(pyridin-4-ylmethyl)pyrrolidine-2-carboxylic acid (2.3 g, 5.58 mmol) in THF (10 mL) and Water (10.00 mL) was added sodium bicarbonate (2.342 g, 27.9 mmol) and N-(9-fluorenylmethoxycarbonyloxy)succinimide (1.693 g, 5.02 mmol). The reaction was stirred at ambient temperature for 4 h then concentrated in vacuo. The residue was purified by RP flash with the following conditions: Column: Flash C 18 (330 g); Mobile Phase A: water (0.05% TFA), Mobile Phase B: ACN; (Gradient: 5% B hold 5 min, up to 35% B within 20 min, 35% B hold 5.8 min; up to 95% B within 5 min, 95% B hold 5 min); Flow rate: 80 mL/min; Detector: UV 210 nm; RT=40.8 min. The product-containing fractions were collected and concentrated in vacuo to give (2R,4R)-1-(((9H-fluoren-9-yl)methoxy)carbonyl)-4-(pyridin-4-ylmethyl)pyrrolidine-2-carboxylic acid (2.02 g, 4.71 mmol, 85% yield) as an off-white solid. MS ESI calculated for C₂₆H₂₅N₂O₄ [M+H]⁺ 429.17, found 429.20.¹H NMR (400 MHz, Methanol-d₄) δ 8.78-8.74 (m, 2H), 7.98-7.93 (m, 2H), 7.79-7.74 (m, 2H), 7.64-7.57 (m, 2H), 7.40-7.27 (m, 4H), 4.45-4.43 (m, 1H), 4.35-4.16 (m, 3H), 3.75-3.73 (m, 1H), 3.51-3.48 (m, 1H), 3.23-3.05 (m, 3H), 2.60-2.55 (m, 2H), 1.82-1.65 (m, 1H).

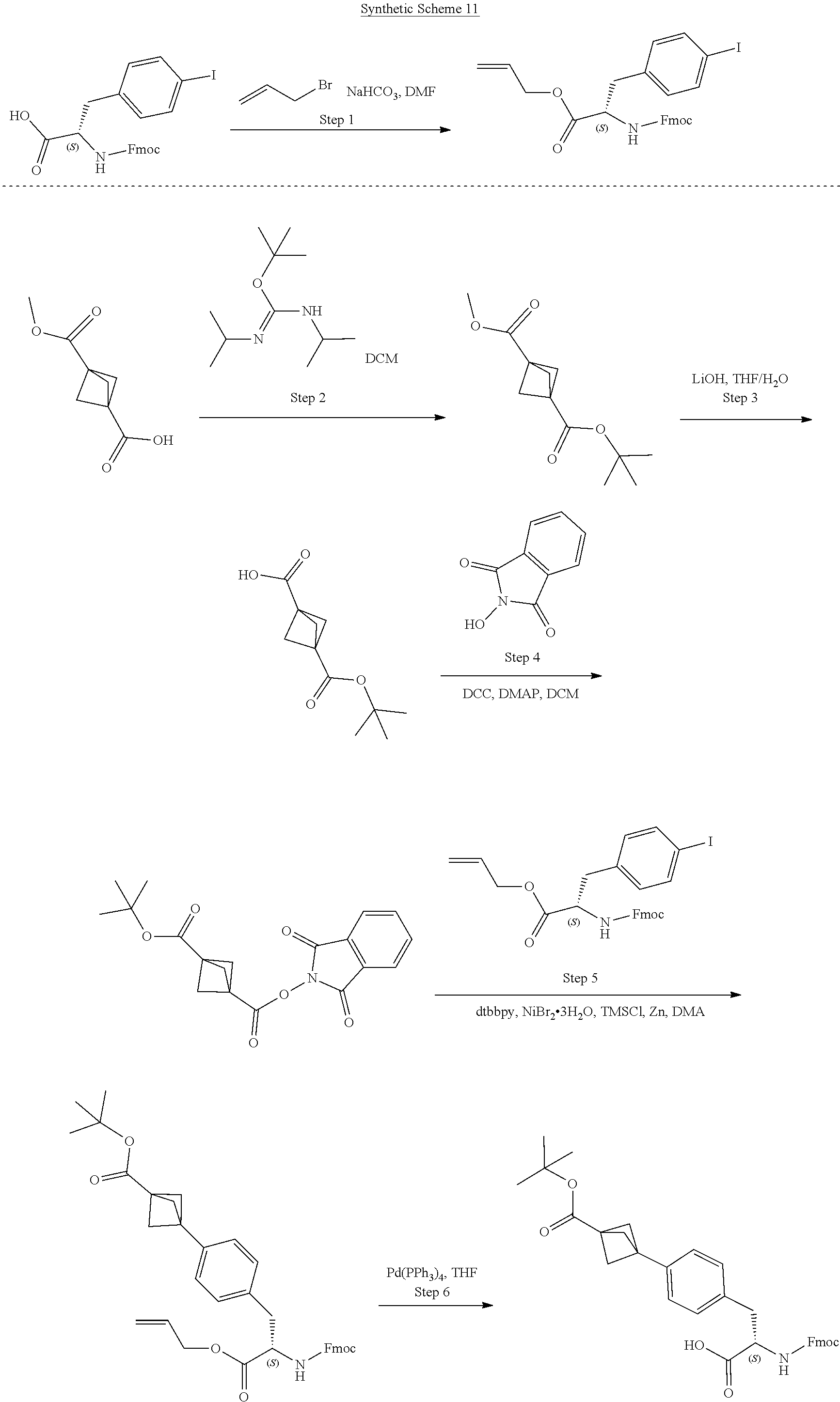

### Precursor to F4bcpA

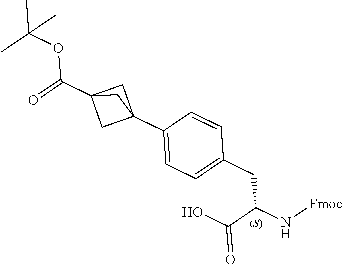

### (S)-2-((((9H-Fluoren-9-yl)methoxy)carbonyl)amino)-3-(4-(3-(tert-butoxycarbonyl)bicyclo[1.1.1]pentan-1-yl)phenyl)propanoic acid

Step 1: To a stirred mixture of (S)-2-((tert-butoxycarbonyl)amino)-3-(4-iodophenyl)propanoic acid (3.91 g, 10 mmol) in DMF (40 mL) were added 3-bromoprop-1-ene (3.63 g, 30.0 mmol) and NaHCO₃ (0.840 g, 10.00 mmol) at 0° C. under argon atmosphere. The resulting mixture was stirred at 40° C. for 16 h. The reaction mixture was cooled to room temperature and quenched with water (200 mL), extracted with EA (2×200 mL). The combined organic layer was washed with brine (3×100 mL), dried over Na₂SO₄ and filtered. The filtrate was concentrated under reduced pressure and the residue was purified by silica gel column chromatography (eluted with 0~40% EA in PE) and the product-containing fractions were collected and roto-evaporated in vacuo to afford allyl (S)-2-((tert-butoxycarbonyl)amino)-3-(4-iodophenyl)propanoate (4 g, 9.27 mmol, 93% yield) as an off-white solid. MS ESI calculated for C₂₇H₂₅INO₄ [M+H]⁺ 554.08, found 554.20.

Step 2: To a stirred solution of 3-(methoxycarbonyl)bicyclo[1.1.1]pentane-1-carboxylic acid (8 g, 47.0 mmol) in DCM (100 mL) was added tert-butyl (Z)—N,N′-diisopropylcarbamimidate (37.7 g, 188 mmol) at room temperature. The resulting solution was stirred at 40° C. for 2 h. The mixture was cooled to room temperature. The solid was filtered out and the filtrate was concentrated under reduced pressure to give 1-(tert-butyl) 3-methyl bicyclo[1.1.1]pentane-1,3-dicarboxylate (15 g, 39.8 mmol, 85% yield) as a light-yellow oil. ¹H NMR (400 MHz, DMSO-d₆) δ 3.61 (s, 3H), 2.17 (s, 6H), 1.39 (s, 9H).

Step 3: To a stirred solution of 1-(tert-butyl) 3-methyl bicyclo[1.1.1]pentane-1,3-dicarboxylate (15 g, 39.8 mmol) in THF (150 mL) was added LiOH (119 mL, 119 mmol, 1 N in water) at room temperature. The resulting solution was stirred at 25° C. for 5 h. The pH of the solution was adjusted to 3 with 1 N HCl and then was extracted with EA (3×100 mL). The combined organic layer was washed with brine (2×50 mL), dried over anhydrous Na₂SO₄. After filtration, the filtrate was concentrated under reduced pressure to give 3-(tert-butoxycarbonyl)bicyclo[1.1.1]pentane-1-carboxylic acid (10 g, 28.3 mmol, 71% yield) as a light-yellow solid. MS ESI calculated for C₁₁H₅O₄ [M−H]⁻ 211.10, found 211.10.

Step 4: To a stirred solution of 3-(tert-butoxycarbonyl)bicyclo[1.1.1]pentane-1-carboxylic acid (10 g, 28.3 mmol), 2-hydroxyisoindoline-1,3-dione (6.00 g, 36.7 mmol) and DMAP (0.345 g, 2.83 mmol) in DCM (100 mL) was added DCC (6.42 g, 31.1 mmol) at room temperature. The resulting mixture was stirred at 25° C. for 16 h. The solid was filtered out and the filtrate was concentrated under reduced pressure. The residue was purified by a silica gel column chromatography, eluted with 0-40% EA in PE to afford 1-(tert-butyl) 3-(1,3-dioxoisoindolin-2-yl) bicyclo[1.1.1]pentane-1,3-dicarboxylate (4.8 g, 12.09 mmol, 43% yield) as an off-white solid. ¹H NMR (400 MHz, CDCl₃) δ 7.89 (dd, J=5.5, 3.1 Hz, 2H), 7.79 (dd, J=5.5, 3.1 Hz, 2H), 2.50 (s, 6H), 1.47 (s, 9H).

Step 5: To a stirred solution of NiBr_{2·}3H₂O (0.791 g, 2.90 mmol) in DMA (80 mL) was added dtbbpy (0.973 g, 3.63 mmol) at room temperature under nitrogen atmosphere. After the resulting mixture was stirred at 50° C. for 30 min., it was cooled to room temperature, 1-(Tert-butyl) 3-(1,3-dioxoisoindolin-2-yl) bicyclo[1.1.1]pentane-1,3-dicarboxylate (4.8 g, 12.09 mmol), allyl (S)-2-((((9H-fluoren-9-yl)methoxy)carbonvl)amino)-3-(4-iodophenyl)propanoate (6.69 g, 12.09 mmol), TMSCl (0.131 g, 1.209 mmol) and Zinc (3.95 g, 60.4 mmol) were added to the above mixture at room temperature. The resulting mixture was stirred at 25° C. for 2 h. The reaction was quenched by brine (150 mL), extracted with EA (3×100 mL). The combined organic layer was washed with brine (3×100 mL), dried over anhydrous Na₂SO₄. After filtration, the filtrate was concentrated under reduced pressure and the residue was purified by a silica gel column chromatography, eluted with 0-40% EA in PE to afford tert-butyl (S)-3-(4-(2-((((9H-fluoren-9-yl)methoxy)carbonyl)amino)-3-(allyloxy)-3-oxopropyl)phenyl)bicyclo[1.1.1]pentane-1-carboxylate (2.3 g, 1.550 mmol, 13% yield) as an off-white solid. MS ESI calculated for C₃₇H₄₀NO₆ [M+H]⁺ 594.28, found 594.30.

Step 6: To a stirred solution of tert-butyl (S)-3-(4-(2-((((9H-fluoren-9-yl)methoxy)carbonyl)amino)-3-(allyloxy)-3-oxopropyl)phenyl)bicyclo[1.1.1]pentane-1-carboxylate (2.3 g, 1.550 mmol) and phenylsilane (0.335 g, 3.10 mmol) in THF (30 mL) was added Pd(Ph₃P)₄ (0.090 g, 0.077 mmol) at room temperature under nitrogen atmosphere. The resulting mixture was stirred at room temperature for 1 h. then concentrated under reduced pressure and the residue was purified by RP-flash with the following conditions: C¹⁸ column, 330 g, 5%-5% in 5 min, 5%-70% in 40 min. MeCN in water (0.05% TFA) to give (S)-2-((((9H-fluoren-9-yl)methoxy)carbonyl)amino)-3-(4-(3-(tert-butoxycarbonyl)bicyclo[1.1.1]pentan-1-yl)phenyl)propanoic acid (522.8 mg, 0.897 mmol, 58% yield) as a light-yellow solid. MS ESI calculated for C₃₄H₃₄NO₆ [M−H]⁻ 552.25, found 552.30.¹H NMR (400 MHz, CD₃OD) δ 7.80-7.77 (m, 2H), 7.67-7.65 (m, 2H), 7.48-7.23 (m, 4H), 7.19-7.17 (m, 2H), 7.10-7.07 (m, 2H), 4.45-4.44 (m, 1H), 4.42-4.41 (m, 1H), 4.34-4.29 (m, 2H), 3.24-3.18 (m, 1H), 2.94-2.86 (m, 1H), 2.12 (s, 6H), 1.45 (s, 9H).

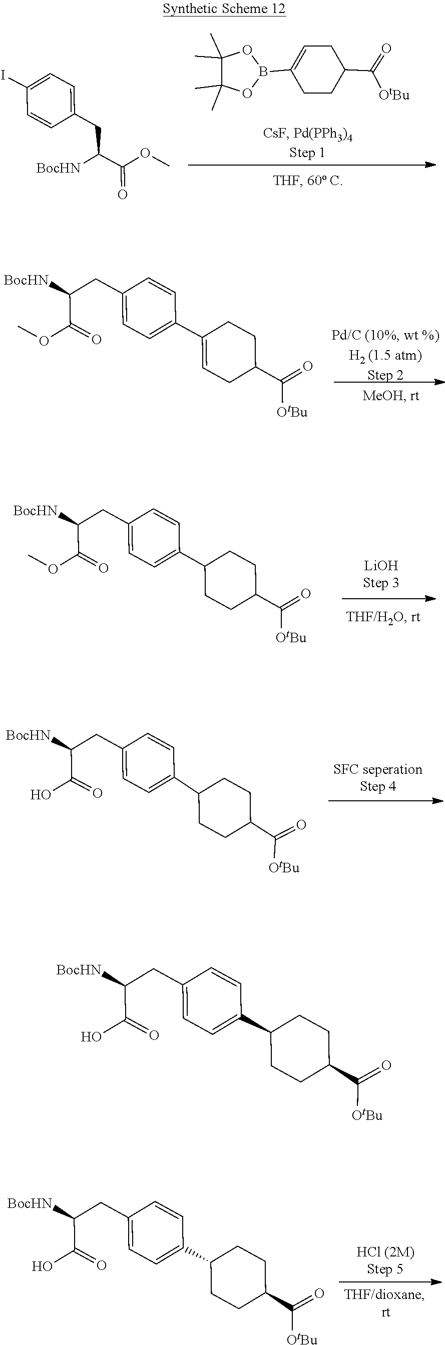

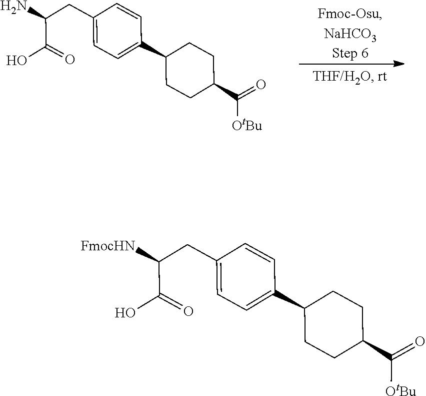

### Precursor to F4pcCCA

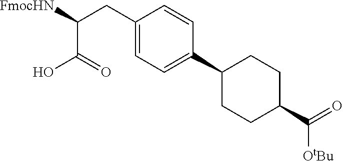

### (S)-2-((((9H-Fluoren-9-yl)methoxy)carbonyl)amino)-3-(4-((1s,4R)-4-(tert-butoxycarbonyl)cyclohexyl)phenyl)propanoic acid

Step 1: To a mixture of tert-butyl 4-(4,4,5,5-tetramethyl-1,3,2-dioxaborolan-2-yl) cyclohex-3-ene-1-carboxylate (29.5 g, 96 mmol) in THF (30 mL) was added methyl (S)-2-((tert-butoxycarbonyl) amino)-3-(4-iodophenyl) propanoate (15.5 g, 38.2 mmol), and Pd(Ph₃P)₄ (2.210 g, 1.912 mmol) at room temperature. The reaction was warmed to 60° C. for 4 h. The resulting solution was quenched with water (100 mL) and extracted with ethyl acetate (3×300 mL). The organic layers were combined, washed with brine (2×200 mL), dried over anhydrous sodium sulfate and filtered. The filtrate was concentrated in vacuo to afford crude product. The residue was purified by silica gel chromatography, eluting with a gradient of ethyl acetate:petroleum ether—0:1 to 1:4 to afford tert-butyl 4′-((S)-2-((tert-butoxycarbonyl)amino)-3-methoxy-3-oxopropyl)-2,3,4,5-tetrahydro-[1,1′-biphenyl]-4-carboxylate (16 g, 34.8 mmol, 91% yield) as a colorless solid. MS ESI calculated for C₂₆H₃₈NO₆Na [M+Na]⁺, 482.26 found 482.10.

Step 2: To a mixture of tert-butyl 4′-((S)-2-((tert-butoxycarbonyl) mino)-3-methoxy-3-oxopropyl)-2,3,4,5-tetrahydro-[1,1′-biphenyl]-4-carboxylate (16 g, 34.8 mmol) in methanol (160 mL) was added Pd—C (10% on carbon, wetted with ca.55% water, 5.3 g, 4.98 mmol) at room temperature was degassed with H₂ three times and stirred for 1 h at room temperature under the atmosphere of H₂ (1.5 atm). The resulting solution was filtered. The filtrate was concentrated in vacuo to afford tert-butyl(S)-4-(4-(2-((tert-butoxycarbonyl) amino)-3-methoxy-3-oxopropyl)phenyl)cyclohexane-1-carboxylate (15 g, 29.2 mmol, 84% yield) as a colorless semi-solid. MS ESI calculated for C₂₆H₄₁NO₆ [M+H]⁺, 462.28, found 462.30.

Step 3: To a stirred solution of tert-butyl(S)-4-(4-(2-((tert-butoxycarbonyl) amino)-3-methoxy-3-oxopropyl)phenyl)cyclohexane-1-carboxylate (15 g, 32.5 mmol) in THF (300 mL) was added LiOH (65.0 mL, 65.0 mmol) at room temperature. The solution was stirred at 20° C. for 1 h. The pH value of the solution was adjusted to 3 with 1 N HCl. The reaction was concentrated under reduced pressure to give (S)-2-((tert-butoxycarbonyl)amino)-3-(4-(4-(tert-butoxycarbonyl)cyclohexyl)phenyl)propanoic acid (14 g, 31.3 mmol, 96% yield) as a white solid. MS ESI calculated for C₂₅H₃₈NO₆Na [M+Na]⁺, 470.26, found 470.30.

Step 4: (S)-2-((tert-butoxycarbonyl)amino)-3-(4-(4-(tert-butoxycarbonyl) cyclohexyl) phenyl) propanoic acid (14 g, 31.3 mmol) was separated with Prep-SFC with following conditions: Column: CHIRAL ART Cellulose-SB, 3×25 cm, 5 μm; Mobile Phase A: CO₂, Mobile Phase B: MeOH (0.1% 2M NH₃-MEOH); Flow rate: 80 mL/min; Gradient: 10% B; 220 nm; RT1: 7.45; RT2: 8.38; Injection Volume: 1.3 ml; Number Of Runs: 131; to afford (S)-2-((tert-butoxycarbonyl) amino)-3-(4-((1s,4R)-4-(tert-butoxycarbonyl) cyclohexyl) phenyl) propanoic acid (8.3 g, 18.54 mmol, 59.3% yield) fast peak as a white solid; MS ESI calculated for C₂₅H₃₈NO₆Na [M+Na]⁺, 470.26 found 470.30. 1H NMR (300 MHz, Chloroform-d) δ 7.11 (d, J=3.0 Hz, 4H), 4.27 (s, 1H), 3.18-3.12 (m, 1H), 2.91 (s, 1H), 2.60 (s, 1H), 2.49 (s, 1H), 2.20 (d, J=10.9 Hz, 2H), 1.78-1.52 (m, 6H), 1.49 (d, J=0.7 Hz, 9H), 1.36 (s, 9H) and (S)-2-((tert-butoxycarbonyl)amino)-3-(4-((1r,4S)-4-(tert-butoxycarbonyl)cyclohexyl)phenyl)propanoic acid (3 g, 6.70 mmol, 21.43% yield) slow peak as a white solid; MS ESI calculated for C₂₅H₃₈NO₆Na [M+Na]⁺, 470.26 found 470.30. 1H NMR (300 MHz, Chloroform-d) δ 7.14-7.06 (m, 4H), 4.25 (s, 1H), 3.14 (d, J=12.3 Hz, 1H), 2.88 (s, 1H), 2.49-2.41 (m, 1H), 2.29-2.15 (m, 1H), 2.05 (d, J=12.2 Hz, 2H), 1.90 (d, J=12.1 Hz, 2H), 1.69-1.47 (m, 2H), 1.47 (s, 11H), 1.34 (s, 9H).

Step 5: A mixture of (S)-2-((tert-butoxycarbonyl) amino)-3-(4-((1s,4R)-4-(tert-butoxycarbonyl) cyclohexyl) phenyl) propanoic acid (8.3 g, 18.54 mmol) in THF (80 mL) was added hydrogen chloride (9.27 mL, 18.54 mmol) in portions at room temperature. The reaction was concentrated under reduced pressure to afford (S)-2-amino-3-(4-((1s,4R)-4-(tert-butoxycarbonyl)cyclohexyl) phenyl)propanoic acid (6 g, 17.27 mmol, 93% yield) as a white solid. MS ESI calculated for C₂₀H₃₀NO₄ [M+H]⁺, 348.21 found 348.25.

Step 6: To a stirred solution of (S)-2-amino-3-(4-((1s,4R)-4-(tert-butoxycarbonyl) cyclohexyl) phenyl) propanoic acid (6 g, 17.27 mmol) and NaHCO₃ (7.25 g, 86 mmol) in THF (60 mL) and water (60.0 mL) was added n-(9-fluorenylmethoxycarbonyloxy)succinimide (5.24 g, 15.54 mmol) at room temperature. The mixture was stirred at room temperature for 1 h. The pH value of the solution was adjusted to 3 with 1 N HCl. The aqueous phase was extracted with ether acetate (2×200 mL). The combined organic layer was washed with brine (150 mL), dried over anhydrous Na₂SO₄ and filtered. The filtrate was concentrated under reduced pressure, and the residue was purified by RP flash to give (S)-2-((((9H-fluoren-9-yl)methoxy)carbonyl)amino)-3-(4-((1s,4R)-4-(tert-butoxycarbonyl)cyclohexyl)phenyl)propanoic acid (7.7 g, 13.52 mmol, 78% yield) as an off-white solid. MS ESI calculated for C₃₅H₄₁NO₆ [M+H]⁺, 570.28 found 570.15. ¹H NMR (300 MHz, Methanol-d₄) δ 7.81 (d, J=7.5 Hz, 2H), 7.61 (d, J=7.5 Hz, 2H), 7.43-7.27 (m, 4H), 7.16 (d, J=7.8 Hz, 2H), 7.07 (d, J=7.9 Hz, 2H), 4.45-4.33 (m, 2H), 4.22-4.06 (m, 2H), 3.26-3.14 (m, 1H), 2.96-2.88 (m, 1H), 2.59 (s, 1H), 2.48 (s, 1H), 2.15 (s, 2H), 1.61 (d, J=5.5 Hz, 6H), 1.49 (s, 9H).

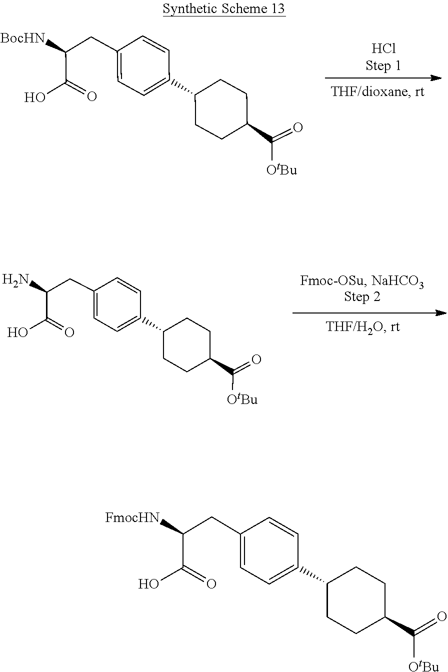

### Precursor to F4ptCCA

### (S)-2-((((9H-fluoren-9-yl) methoxy) carbonyl) amino)-3-(4-((1r,4S)-4-(tert-butoxycarbonyl) cyclohexyl) phenyl) propanoic acid

Step 1: A mixture of (S)-2-((tert-butoxycarbonyl) amino)-3-(4-((1r,4S)-4-(tert-butoxycarbonyl) cyclohexyl) phenyl) propanoic acid (3 g, 6.70 mmol) in THF (30 mL) was added hydrogen chloride (30 mL, 60.0 mmol) in portions at room temperature. The reaction was concentrated under reduced pressure to afford (S)-2-amino-3-(4-((1r,4S)-4-(tert-butoxycarbonyl)cyclohexyl)phenyl) propanoic acid (2 g, 5.76 mmol, 86% yield) as a white solid. MS ESI calculated for C₂₀H₃₁NO₄ [M+H]⁺, 348.21, found 348.25.

Step 2: To a stirred solution of (S)-2-amino-3-(4-((1r,4S)-4-(tert-butoxycarbonyl) cyclohexyl)phenyl) propanoic acid (2 g, 5.76 mmol) (1.748 g, 5.18 mmol) and NaHCO₃ (2.418 g, 28.8 mmol) in THF (20 mL) and water (20 mL) was added N-(9-fluorenylmethoxycarbonyloxy)succinimide (1.748 g, 5.18 mmol) at room temperature. The mixture was stirred at 20° C. for 1 h. The pH value of the solution was adjusted to 3 with 1 N HCl. The aqueous phase was extracted with ether acetate (2×200 mL). The combined organic layer was washed with brine (3×50 mL), dried over anhydrous Na₂SO₄ and filtered. The filtrate was concentrated under reduced pressure and the residue was purified by RP flash to give (S)-2-((((9H-fluoren-9-yl) methoxy) carbonyl) amino)-3-(4-((1r,4S)-4-(tert-butoxycarbonyl) cyclohexyl) phenyl) propanoic acid (1.89 g, 3.32 mmol, 57.6% yield) as an off-white solid. MS ESI calculated for C₃₅H₄₁NO₆ [M+H]⁺, 570.28, found 570.35. ¹H NMR (300 MHz, Methanol-d₄) δ 7.81 (d, J=7.6 Hz, 2H), 7.61 (d, J=7.5 Hz, 2H), 7.43-7.38 (m, 2H), 7.34-7.27 (m, 2H), 7.17 (d, J=8.0 Hz, 2H), 7.08 (d, J=7.8 Hz, 2H), 4.47-4.43 (m, 1H), 4.35-4.30 (m, 1H), 4.21-4.02 (m, 2H), 3.24-3.18 (m, 1H), 2.94-2.87 (m, 1H), 2.40 (s, 1H), 2.17 (d, J=11.9 Hz, 1H), 1.96 (s, 3H), 1.82-1.55 (m, 2H), 1.47 (s, 13H).

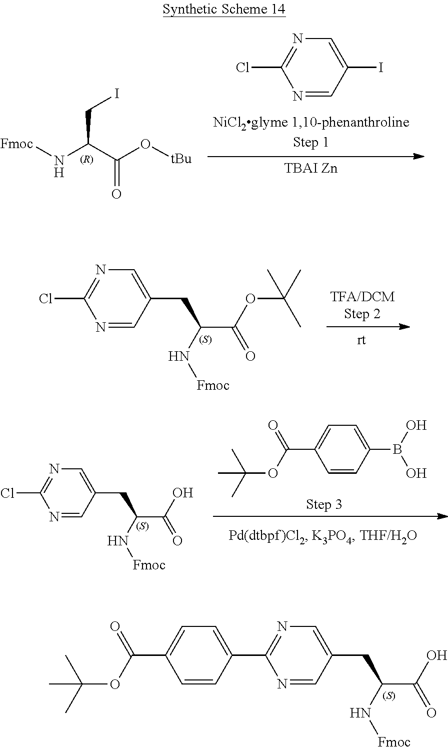

### Precursor to PyrimAla4Ph4CO2H

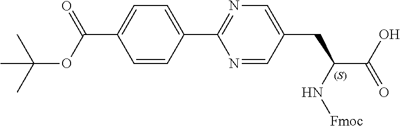

### (S)-2-((((9H-Fluoren-9-yl)methoxy)carbonyl)amino)-3-(2-(4-(tert-butoxycarbonyl)phenyl)pyrimidin-5-yl)propanoic acid

Step 1: To a stirred solution of NiBr₂-glyme (0.951 g, 2.432 mmol) in DMA (100 mL) was added 1,10-phenanthroline (0.527 g, 2.432 mmol) at 25° C. under nitrogen atmosphere. The resulted solution was stirred at 50° C. for 1 h then cooled to room temperature, to which 2-Chloro-5-iodopyrimidine (5.85 g, 24.32 mmol), tert-butyl (R)-2-((((9H-fluoren-9-yl)methoxy)carbonyl)amino)-3-iodopropanoate (6 g, 12.16 mmol), TBAI (4.66 g, 12.16 mmol) and zinc (1.590 g, 24.32 mmol) were added at room temperature. The resulting mixture was stirred at 25° C. for 2 h, then quenched with H₂O (200 mL), extracted with EA (2×500 mL). The combined organic layer was washed with brine (3×200 mL), dried over anhydrous Na₂SO₄ and filtered. The filtrate was concentrated under reduced pressure. The residue was purified by silica gel column chromatography, eluted with 0-20% EA in PE to give tert-butyl (S)-2-((((9H-fluoren-9-yl)methoxy)carbonyl)amino)-3-(2-chloropyrimidin-5-yl)propanoate (4 g, 8.35 mmol, 70% yield) as an off-white solid. MS ESI calculated for C₂₆H₂₇ClN₃O₄ [M+H]⁺ 480.16, found 480.25.

Step 2: To a stirred solution of tert-butyl (S)-2-((((9H-fluoren-9-yl)methoxy)carbonyl)amino)-3-(2-chloropyrimidin-5-yl)propanoate (6 g, 12.50 mmol) in DCM (15 mL) was added TFA (30 mL) at room temperature. The solution was stirred at 25° C. for 3 h then concentrated under reduced pressure. The residue was purified by RP-flash with the following conditions: Column: Flash C¹⁸ (330 g); Mobile Phase A: water (0.1% TFA), Mobile Phase B: ACN; (Gradient: 5% B hold 5 min, up to 30% B within 15 min, 30% B hold 5 min; up to 95% B within 20 min. 95% B hold 10 min); Flow rate: 90 mL/min; Detector: UV 210 nm; RT=40 min. The product-containing fractions were collected and evaporated in vacuo to give (S)-2-((((9H-fluoren-9-yl)methoxy)carbonyl)amino)-3-(2-chloropyrimidin-5-yl)propanoic acid (2.56 g, 6.04 mmol, 51% yield) as a yellow oil. MS ESI calculated for C₂₂H₁₉ClN₃O₄ [M+H]⁺ 424.10, found 424.15.

Step 3: To a stirred mixture of (S)-2-((((9H-fluoren-9-yl)methoxy)carbonyl)amino)-3-(2-chloropyrimidin-5-yl)propanoic acid (2.56 g, 6.04 mmol), (4-(tert-butoxycarbonyl)phenyl)boronic acid (1.609 g, 7.25 mmol) and K₃PO₄ (6.41 g, 30.2 mmol) in water (20 mL) and dioxane (20 mL) was added Pd(dtbpf)Cl₂(0.590 g, 0.906 mmol) at room temperature. The resulting mixture was stirred at 80° C. for 2 h. then cooled to room temperature and concentrated under reduced pressure. The residue was purified by RP-flash with the following conditions: Column: Flash C¹⁸ (330 g); Mobile Phase A: water (0.1% TFA), Mobile Phase B: ACN; (Gradient: 5% B hold 5 min, up to 30% B within 15 min, 30% B hold 5 min; up to 95% B within 20 min. 95% B hold 10 min); Flow rate: 90 mL/min; Detector: UV 210 nm; RT=40 min. The product-containing fractions were collected and evaporated in vacuo to give (S)-2-((((9H-fluoren-9-yl)methoxy)carbonyl)amino)-3-(2-(4-(tert-butoxycarbonyl)phenyl)pyrimidin-5-yl)propanoic acid (2.5629 g, 4.53 mmol, 75% yield) as a yellow oil. MS ESI calculated for C₃₃H₃₂N₃O₆ [M+H]⁺ 566.22, found 566.40. ¹H NMR (300 MHz, CD₃OD) δ 8.75 (s, 2H), 8.42 (d, J=8.3 Hz, 2H), 8.02 (d, J=8.2 Hz, 2H), 7.75 (d, J=7.5 Hz, 2H), 7.66-7.45 (m, 2H), 7.42-7.15 (m, 4H), 4.62-4.44 (m, 1H), 4.38-4.20 (m, 2H), 4.12 (t, J=7.0 Hz, 1H), 3.35-3.33 (m, 1H), 3.11-2.97 (m, 1H), 1.62 (s, 9H).

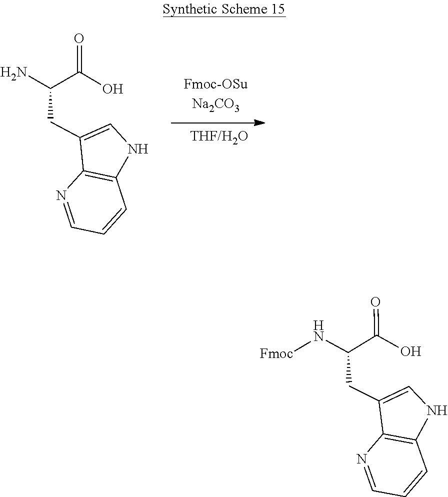

### Precursor to Trp4Az

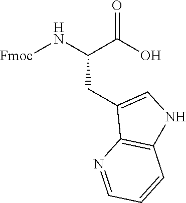

### (S)-2-((((9H-Fluoren-9-yl)methoxy)carbonyl)amino)-3-(1H-pyrrolo[3,2-b]pyridin-3-yl)propanoic acid

To a solution of (S)-2-amino-3-(1H-pyrrolo[3,2-b]pyridin-3-yl)propanoic acid (250 mg, 1.22 mmol) and Na₂CO₃ (232 mg, 2.2 mmol) in THF (4 mL) and water (2 mL) was added Fmoc-OSu (431 mg, 1.28 mmol) at room temperature. The mixture was stirred for 16 h, then adjusted to pH 3 with 1 N HCl and concentrated in vacuo. The resulting aqueous suspension was washed with EtOAc (3×100 mL), then extracted with 20% i-PrOH/DCM (3×100 mL). The combined organic extracts were dried over Na₂SO₄, filtered, and concentrated in vacuo to afford (S)-2-((((9H-fluoren-9-yl)methoxy)carbonyl)amino)-3-(1H-pyrrolo[3,2-b]pyridin-3-yl)propanoic acid (470 mg, 1.10 mmol, 90% yield) as a white solid. MS-ESI [M+H]⁺=428.4. 1H NMR (500 MHz, DMSO-d₆) δ 11.14 (br s, 1H), 8.30 (dd, J=4.6, 1.3 Hz, 1H), 7.88 (d, J=7.6 Hz, 2H), 7.75 (dd, J=8.1, 1.3 Hz, 1H), 7.63 (t, J=7.9 Hz, 2H), 7.47 (d, J=2.3 Hz, 1H), 7.40 (td, J=7.3, 3.4 Hz, 2H), 7.32-7.24 (m, 2H), 7.12 (dd, J=8.1, 4.6 Hz, 1H), 4.23 (td, J=8.1, 4.3 Hz, 1H), 4.19-4.15 (m, 3H), 3.30 (dd, J=14.5, 4.0 Hz, 1H), 3.09 (dd, J=14.6, 8.6 Hz, 1H).

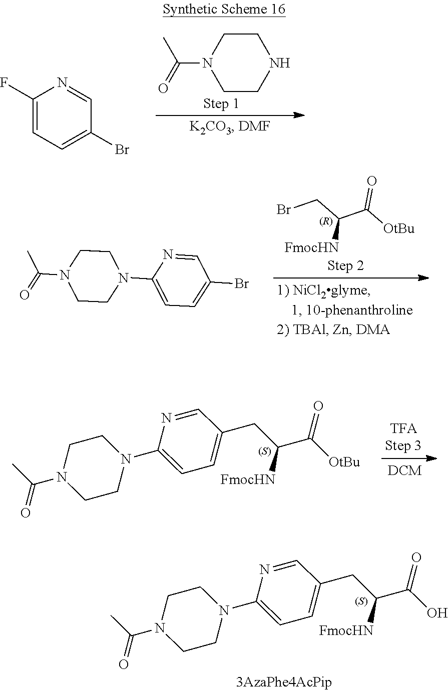

### Precursor to 3AzaPhe4AcPip

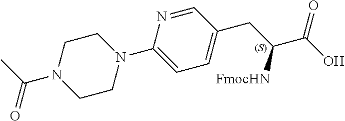

### (S)-2-((((9H-fluoren-9-yl)methoxy)carbonyl)amino)-3-(6-(4-acetylpiperazin-1-yl)pyridin-3-yl)propanoic acid

Step 1: To a mixture of 1-(piperazin-1-yl)ethan-1-one (21.85 g, 170 mmol) in DMF (150 mL) was added 5-bromo-2-fluoropyridine (15 g, 85 mmol) under argon at rt. The reaction was stirred at 100° C. for 2 h, then diluted with 300 mL EtOAc and washed with H₂O (3×80 mL), aqueous saturated NaCl (80 mL), dried over Na₂SO₄ and filtered. The filtrate was concentrated under reduced pressure. The residue was purified by silica gel column chromatography, eluting with a gradient of 0%-100% EtOAc in PE. The fractions containing desired product were combined and concentrated under reduced pressure to afford 1-(4-(5-bromopyridin-2-yl)piperazin-1-yl)ethan-1-one. MS ESI calculated for C11H15BrN3O [M+H]⁺ 284.03 and 286.03. found 283.90 and 285.90.

Step 2: The mixture of nickel (II) chloride ethylene glycol dimethyl ether complex (0.696 g, 3.17 mmol) and 1,10-phenanthroline (0.571 g, 3.17 mmol) in DMA (2 mL) was heated at 50° C. for 0.5 hours. The mixture of 1-(4-(5-bromopyridin-2-yl)piperazin-1-yl)ethan-1-one (4.5 g, 15.84 mmol), tert-butyl (R)-2-((((9H-fluoren-9-yl)methoxy)carbonyl)amino)-3-bromopropanoate (7.78 g, 17.42 mmol) and tetrabutylammonium iodide (5.85 g, 15.84 mmol) in DMA (2 mL) were added at 25° C. Then zinc powder (2.071 g, 31.7 mmol) was added. The resulting mixture was stirred for 1 h at 50° C. The resulting mixture was poured into water (300 mL) and extracted with EtOAc (3×300 mL). The organic layer was washed with water (100 mL) and brine (2×80 mL), dried over anhydrous sodium sulfate and concentrated under reduced pressure. The residue was purified by column chromatography on silica gel eluting with DCM-MeOH (10:1) to afford tert-butyl (S)-2-((((9H-fluoren-9-yl)methoxy)carbonyl)amino)-3-(6-(4-acetylpiperazin-1-yl)pyridin-3-yl)propanoate. MS ESI calculated for C₃₃H₃₉N₄O₅ [M+H]⁺ 571.28, found 571.40.

Step 3: To a mixture of tert-butyl (S)-2-((((9H-fluoren-9-yl)methoxy)carbonyl)amino)-3-(6-(4-acetylpiperazin-1-yl)pyridin-3-yl)propanoate (7.3 g, 12.79 mmol) in DCM (70 mL) was added TFA (70 mL, 909 mmol) under argon at rt. The reaction was stirred at rt for 1 h then concentrated under reduced pressure. The residue was dissolved in THF (20 mL) and the resulting mixture was purified by reverse phase Combi-Flash with the following conditions: Column C18 silica gel column (330 g), 20-35 μm; Mobile Phase A: 5 mM aq. TFA; Mobile Phase B: MeCN; (Gradient: 0% B hold 10 min, up to 42.3% B within 35 min, 42.3% B hold 3.2 min; up to 95% B within 2 min, 95% B hold 10 min); Flow rate: 60 mL/min; Detector: UV 254 & 210 nm; RT: 35.32 min. The product-containing fractions were collected and concentrated under vacuum to afford (S)-2-((((9H-fluoren-9-yl)methoxy)carbonyl)amino)-3-(6-(4-acetylpiperazin-1-yl)pyridin-3-yl)propanoic acid. MS ESI calculated for C29H31N4O5 [M+H]⁺ 515.22, found 515.15. ¹H NMR (300 MHz, Methanol-d₄) δ 7.87-7.79 (m, 4H), 7.62-7.55 (m, 2H), 7.42-7.27 (m, 4H), 7.11-7.09 (m, 1H), 4.51-4.46 (m, 1H), 4.32-4.09 (m, 3H), 3.65-3.54 (m, 8H), 3.29-3.20 (m, 1H), 2.94-2.89 (m, 1H), 2.12 (s, 3H).

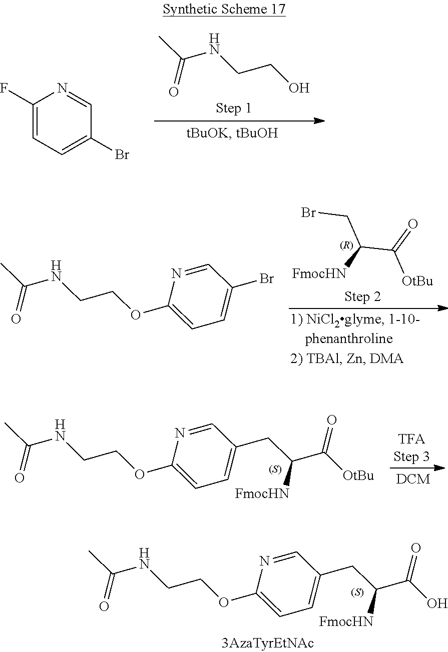

### Precursor to 3AzaTyrEtNAc

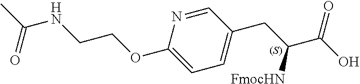

### (S)-2-((((9H-fluoren-9-yl)methoxy)carbonyl)amino)-3-(6-(2-acetamidoethoxy)pyridin-3-yl)propanoic acid

Step 1: To a mixture of N-(2-hydroxyethyl)acetamide (17.58 g, 170 mmol) in t-BuOH (150 mL) was added under argon at rt 5-bromo-2-fluoropyridine (15 g, 85 mmol) followed by potassium tert-butoxide (19.13 g, 170 mmol). The reaction was stirred at rt for 1 h then concentrated under reduced pressure. The residue was diluted with 500 mL EtOAc and washed with aqueous saturated NaHCO₃ (3×250 mL), aqueous saturated NaCl (250 mL), dried over Na₂SO₄ and filtered. The filtrate was concentrate under reduced pressure to afford crude N-(2-((5-bromopyridin-2-yl)oxy)ethyl)acetamide. MS ESI calculated for C9H12BrN2O2 [M+H]⁺ 259.00 and 261.00, found 258.90 and 261.90.

Step 2: The mixture of nickel (II) chloride ethylene glycol dimethyl ether complex (1.187 g, 5.40 mmol) and 1,10-phenanthroline (0.974 g, 5.40 mmol) in DMA (70 mL) was heated at 50° C. for 0.5 hours. The mixture of N-(2-((5-bromopyridin-2-yl)oxy)ethyl)acetamide (7 g, 27.0 mmol), tert-butyl (R)-2-((((9H-fluoren-9-yl)methoxy)carbonyl)amino)-3-bromopropanoate (13.26 g, 29.7 mmol) and tetrabutylammonium iodide (9.98 g, 27.0 mmol) in DMA (70 mL) were added at 25° C. Then zinc powder (3.53 g, 54.0 mmol) was added and the resulting mixture was stirred for 1 h at 50° C. The reaction mixture was diluted with 300 mL EtOAc and washed with aqueous saturated NaHCO₃ (3×80 mL), brine (80 mL), dried over Na₂SO₄ and filtered. The filtrate was concentrated under reduced pressure. The residue was purified by silica gel column chromatography, eluting with gradient 0%-20% DCM in MeOH. The fractions containing the desired product were combined and concentrated under reduced pressure to afford tert-butyl (S)-2-((((9H-fluoren-9-yl) methoxy)carbonyl)amino)-3-(6-(2-acetamidoethoxy)pyridin-3-yl)propanoate. MS ESI calculated for C31H36N3O6 [M+H]⁺ 546.25, found 546.40.

Step 3: To a mixture of tert-butyl (S)-2-((((9H-fluoren-9-yl)methoxy)carbonyl)amino)-3-(6-(2-acetamidoethoxy)pyridin-3-yl)propanoate (8 g, 14.66 mmol) in DCM (80 mL) was added TFA (80 mL, 1038 mmol) under argon at rt. The reaction was stirred at rt for 1 h then concentrated under reduced pressure. The residue was purified by RP flash column chromatography with the following conditions: Column: C18 silica gel column (330 g), 20-35 μm; Mobile Phase A: 5 mM aq. NH₄HCO₃; Mobile Phase B: MeCN; (Gradient: 0% B hold 5 min, up to 45% B within 20 min, 45% B hold 10 min; up to 95% B within 15 min, 95% B hold 10 min); Flow rate: 60 mL/min; Detector: UV 254 & 210 nm; RT: 35.32 min. The product-containing fractions were collected and concentrated under reduced pressure to give (S)-2-((((9H-fluoren-9-yl)methoxy)carbonyl)amino)-3-(6-(2-acetamidoethoxy)pyridin-3-yl)propanoic acid. MS ESI calculated for C27H28N3O6 [M+H]⁺ 490.19, found 490.10. ¹H NMR (400 MHz, Methanol-d₄) δ 8.02-8.00 (m, 1H), 7.79-7.77 (m, 2H), 7.67-7.64 (m, 1H), 7.61-7.57 (m, 2H), 7.40-7.36 (m, 2H), 7.31-7.27 (m, 2H), 6.82-6.80 (m, 1H), 4.42-4.39 (m, 1H), 4.33-4.22 (m, 4H), 4.16-4.13 (m, 1H), 3.53-3.51 (m, 2H), 3.20-3.15 (m, 1H), 2.93-2.87 (m, 1H), 1.92 (s, 3H).

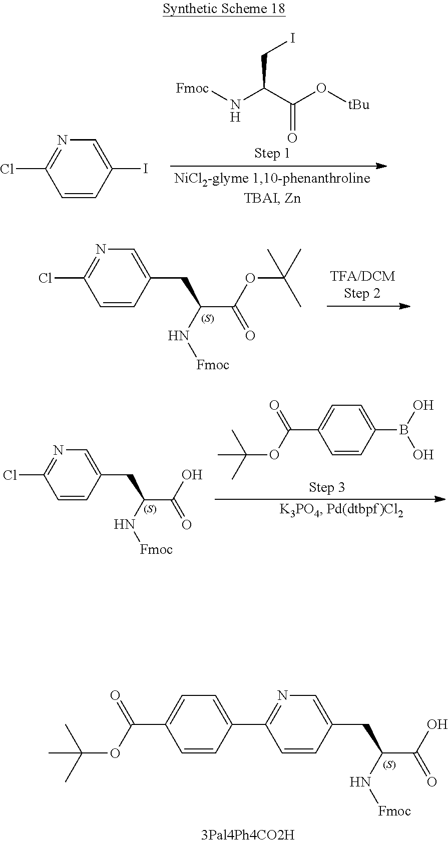

### Precursor to 3Pal4Ph4CO2H

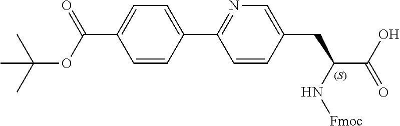

### (S)-2-((((9H-fluoren-9-yl)methoxy)carbonyl)amino)-3-(6-(4-(tert-butoxycarbonyl)phenyl)pyridin-3-yl)propanoic acid

Step 1: To a stirred solution of NiCl₂-glyme (0.918 g, 4.18 mmol) in DMA (100 mL) was added 1,10-phenanthroline (0.905 g, 4.18 mmol) at rt under nitrogen atmosphere. The resulted solution was stirred at 50° C. for 1 h, 2-Chloro-5-iodopyridine (5 g, 20.88 mmol), tert-butyl (R)-2-((((9H-fluoren-9-yl)methoxy)carbonyl)amino)-3-iodopropanoate (12.36 g, 25.06 mmol), TBAI (8.01 g, 20.88 mmol) and Zn (2.73 g, 41.8 mmol) were added to the mixture above at rt and the resulted mixture was stirred at 25° C. for 2 h. The reaction was quenched with H₂O (200 mL), extracted with EtOAc (2×500 mL). The combined organic layer was washed with brine (3×200 mL), dried over anhdrous Na₂SO₄ and filtered. The filtrate was concentrated under reduced pressure and the residue was purified by silica gel column chromatography, eluted with 0-30% EtOAc in PE to give tert-butyl (S)-2-((((9H-fluoren-9-yl)methoxy)carbonyl)amino)-3-(6-chloropyridin-3-yl)propanoate. MS ESI calculated for C₂₇H₂₈ClN₂O₄ [M+H]⁺ 479.17, found 479.20.

Step 2: To a stirred solution of tert-butyl (S)-2-((((9H-fluoren-9-yl)methoxy)carbonyl)amino)-3-(6-chloropyridin-3-yl)propanoate (5 g, 10.48 mmol) in DCM (5 mL) was added TFA (10 mL) at rt. The solution was stirred at 25° C. for 1 h. The solvent was concentrated under reduced pressure and the residue was purified by RP-flash with the following conditions: Column: Flash C¹⁸ (330 g); Mobile Phase A: water (0.1% TFA), Mobile Phase B: ACN; (Gradient: 5% B hold 5 min, up to 30% B within 15 min, 30% B hold 5 min; up to 95% B within 20 min, 95% B hold 10 min); Flow rate: 90 mL/min; Detector: UV 210 nm; RT=40 min. The product-containing fractions were collected and evaporated in vacuo to give (S)-2-((((9H-fluoren-9-yl)methoxy)carbonyl)amino)-3-(6-chloropyridin-3-yl)propanoic acid. MS ESI calculated for C₂₃H₂₀ClN₂O₄ [M+H]⁺ 423.10, found 423.10.

Step 3: To a stirred solution of (S)-2-((((9H-fluoren-9-yl)methoxy)carbonyl)amino)-3-(6-chloropyridin-3-yl)propanoic acid (3 g, 7.09 mmol) in THF (25 mL) and water (5 mL) were added (4-(tert-butoxycarbonyl)phenyl)boronic acid (1.890 g, 8.51 mmol) and K₃PO₄ (7.53 g, 35.5 mmol) at 25° C. under nitrogen. The resulting solution was stirred at 25° C. for 10 min. Pd(dtbpf)Cl₂ (0.694 g, 1.064 mmol) was added to the solution and the mixture was then stirred at 60° C. for 16 h. The reaction was cooled to rt and quenched with H₂O (200 mL) and extracted with EtOAc (2×500 mL). The combined organic layer was washed with brine (3×200 mL), dried over anhydrous Na₂SO₄, and filtered. The filtrate was concentrated under reduced pressure and the residue was purified by RP-flash with the following conditions: Column: Flash C¹⁸ (330 g); Mobile Phase A: water (0.1% TFA), Mobile Phase B: ACN; (Gradient: 5% B hold 5 min, up to 60% B within 15 min. 60% B hold 15 min; up to 95% B within 10 min, 95% B hold 10 min); Flow rate: 90 mL/min; Detector: UV 210 nm; RT=55 min. The product-containing fractions were collected and evaporated in vacuo to give (S)-2-((((9H-fluoren-9-yl)methoxy)carbonyl)amino)-3-(6-(4-(tert-butoxycarbonyl)phenyl)pyridin-3-yl)propanoic acid. MS ESI calculated for C₃₄H₃₃N₂O₆ [M+H]⁺ 565.23, found 565.15; ¹H NMR (400 MHz. Methanol-d₄) δ 8.66 (d, J=1.9 Hz, 1H), 8.12-8.07 (m, 3H), 7.99-7.92 (m, 3H), 7.77 (d, J=7.5 Hz, 2H), 7.59-7.56 (m, 2H), 7.37-7.33 (m, 2H), 7.30-7.22 (m, 2H), 4.60-4.56 (m, 1H), 4.29-4.27 (m, 2H), 4.14-4.10 (m, 1H), 3.45-3.41 (m, 1H), 3.14-3.10 (m, 1H), 1.62 (s, 9H).

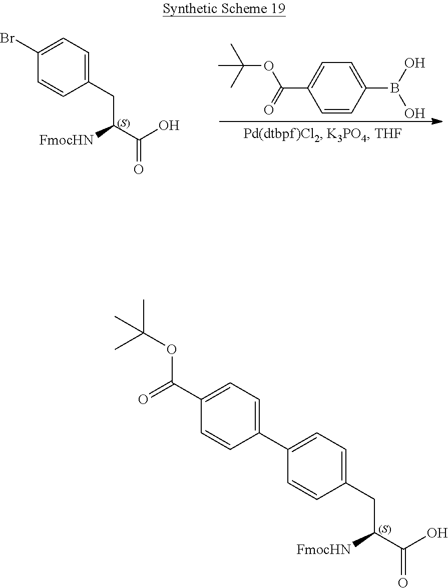

### Precursor to Bip4CO2H

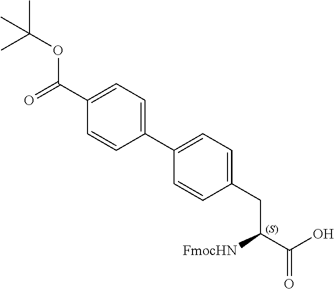

### (S)-2-((((9H-fluoren-9-yl)methoxy)carbonyl)amino)-3-(4′-(tert-butoxycarbonyl)-[1,1′-biphenyl]-4-yl)propanoic acid

Argon gas was bubbled through a mixture of (S)-2-((((9H-fluoren-9-yl)methoxy)carbonyl)amino)-3-(4-bromophenyl)propanoic acid (6 g, 12.87 mmol), (4-(tert-butoxycarbonyl)phenyl)boronic acid (4.29 g, 19.30 mmol) and K₃PO₄ (8.19 g, 38.6 mmol) in THF (40 mL) for 10 min, then [1,1′-bis(di-tert-butylphosphino)ferrocene]dichloropalladium (II) (0.839 g, 1.287 mmol) was added. After the resulting mixture was stirred at 50° C. for 16 h, it was diluted with EtOAc (300 mL) and washed with aqueous saturated NaHCO₃ (3×80 mL), brine (2×40 mL), dried over Na₂SO₄ and filtered. The filtrate was concentrated under reduced pressure. The residue was purified by silica gel column chromatography, eluted with gradient 0%-50% EtOAc in PE. The product-containing fractions were collected and roto-evaporated in vacuo. The residue was re-purified by combi-Flash with the following conditions: Column: Column: C18 gel column (330 g), 20-35 μm; Mobile Phase A: 0.5% aq. TFA; Mobile Phase B: MeCN; (Gradient: 0% B hold 10 min, up to 62.3% B within 25 min, 62.3% B hold 6.2 min; up to 95% B within 2 min, 95% B hold 10 min); Flow rate: 90 mL/min; Detector: UV 254 & 210 nm; RT: 32.32 min. The product-containing fractions were collected and concentrated under reduced pressure to afford (S)-2-((((9H-fluoren-9-yl)methoxy)carbonyl)amino)-3-(4′-(tert-butoxycarbonyl)-[1,1′-biphenyl]-4-yl)propanoic acid. MS ESI calculated for C₃₅H₃₄NO₆ [M+1]⁺ 564.23, found 564.15. ¹H NMR (300 MHz, Methanol-d₄) δ 7.97-7.95 (m, 2H), 7.78-7.76 (m, 2H), 7.61-7.53 (m, 6H), 7.38-7.21 (m, 2H), 4.51-4.11 (m, 4H), 3.32-3.25 (m, 1H), 3.03-2.95 (m, 1H), 1.61 (s, 9H).

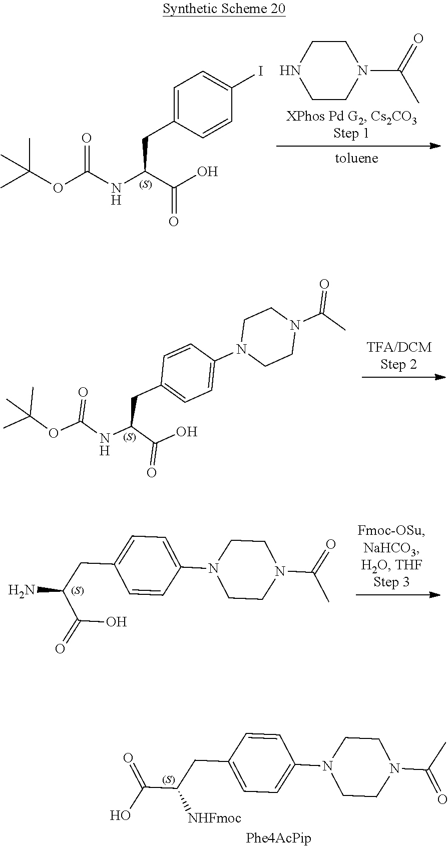

### Precursor to Phe4AcPip

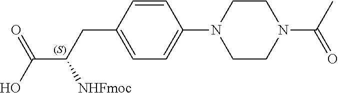

### (S)-2-((((9H-fluoren-9-yl)methoxy)carbonyl)amino)-3-(4-(4-acetylpiperazin-1-yl)phenyl)propanoic acid

Step 1: To a stirred solution of (S)-3-(4-bromophenyl)-2-((tert-butoxycarbonyl)amino)propanoic acid (6 g, 17.43 mmol) in toluene (180 mL) was added XPhos Pd G₂ (2.057 g, 2.61 mmol) at 25° C. under nitrogen. The resulting solution was stirred at 100° C. for 10 min. 1-(Piperazin-1-yl)ethan-1-one (2.234 g, 17.43 mmol) and Cs₂CO₃ (5.04 g, 26.1 mmol) were added and the resulting solution was stirred at 110° C. for 2 h. The reaction was cooled to rt and quenched with H₂O (500 mL), extracted with EtOAc (2×500 mL). The combined organic layer was washed with brine (3×200 mL), dried over anhydrous Na₂SO₄ and filtered. The filtrate was concentrated under reduced pressure and the residue was purified by silica gel column chromatography, eluted with 0-60% EtOAc in PE to give (S)-3-(4-(4-acetylpiperazin-1-yl)phenyl)-2-((tert-butoxycarbonyl)amino)propanoic acid. MS ESI calculated for C₂₀H₃₀N₃O₅[M+H]⁺ 392.21, found 392.25. ¹H NMR (400 MHz, Methanol-d₄) δ 7.12 (d, J=8 Hz, 2H), 6.89 (d, J=8 Hz, 2H), 4.16-4.13 (m, 1H), 3.72-3.65 (m, 4H), 3.14-3.04 (m, 4H), 2.93-2.81 (m, 2H), 2.13 (s, 3H), 1.38-1.29 (m, 9H).

Step 2: To a stirred solution of (S)-3-(4-(4-acetylpiperazin-1-yl)phenyl)-2-((tert-butoxycarbonyl)amino)propanoic acid (10 g, 25.5 mmol) in DCM (30 mL) was added TFA (30 mL) at rt. The solution was stirred at 25° C. for 1 h then concentrated under reduced pressure. The crude (S)-3-(4-(4-acetylpiperazin-1-yl)phenyl)-2-aminopropanoic acid was used to the next step directly without any further purification. MS ESI calculated for C₁₅H₂₂N₃O₃[M+H]⁺ 292.16, found 292.20.

Step 3: To a stirred solution of (S)-3-(4-(4-acetylpiperazin-1-yl)phenyl)-2-(carboxyamino)propanoic acid (7 g, 20.87 mmol) in THF (25 mL) and water (25 mL) was added Fmoc-OSu (6.34 g, 18.79 mmol) at 25° C. under nitrogen. And then NaHCO₃ (8.77 g, 104 mmol) was added. The resulting mixture was stirred at 25° C. for 2 h. The pH was adjusted to 5 with 1 N HCl and extracted with EtOAc (2×200 mL). The combined organic layer was washed with brine (3×100 mL), dried over anhydrous Na₂SO₄ and filtered. The filtrate was concentrated under reduced pressure and the residue was purified by reverse phase flash chromatography with the following conditions: Column: C18 silica gel (330 g); Mobile Phase A: water (0.1% TFA), Mobile Phase B: ACN; (Gradient: 5% B hold 5 min, up to 55% B within 15 min, 55% B hold 5 min; up to 95% B within 20 min, 95% B hold 5 min); Flow rate: 90 mL/min; Detector: UV 210 nm; RT=45 min. The product-containing fractions were collected and concentrated in vacuo to give (S)-2-((((9H-fluoren-9-yl)methoxy)carbonyl)amino)-3-(4-(4-acetylpiperazin-1-yl)phenyl)propanoic acid. MS ESI calculated for C₃₀H₃₂N₃O₅ [M+H]⁺ 514.23, found 514.30. ¹H NMR (400 MHz, Methanol-d₄) δ 7.78-7.76 (m, 2H), 7.61-7.52 (m, 2H), 7.40-7.37 (m, 2H), 7.32-7.22 (m, 4H), 7.08-6.98 (m, 2H), 4.47-4.43 (m, 1H), 4.33-4.31 (m, 1H), 4.14-4.02 (m, 2H), 3.68-3.63 (m, 4H), 3.23-3.08 (m, 5H), 2.91-2.85 (m, 1H), 2.11 (s, 3H).

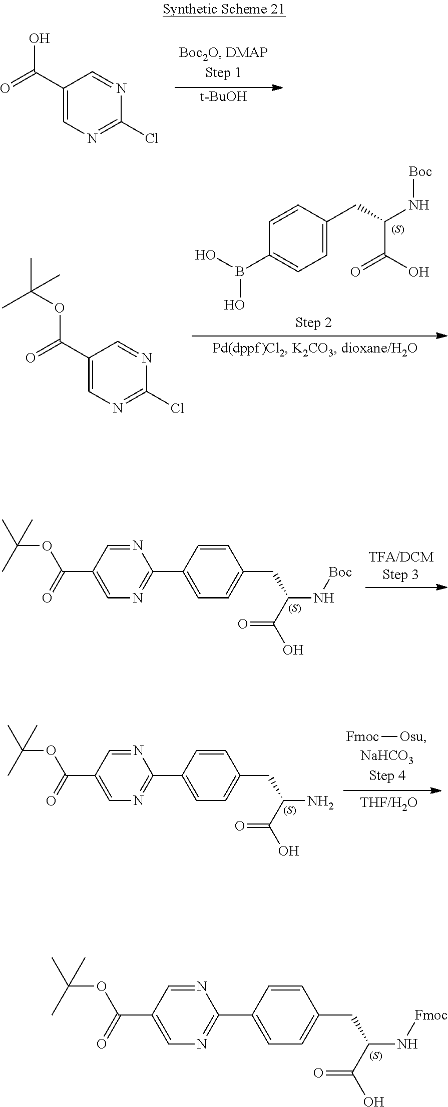

### Precursor to Phe4Pyrim5CO2H

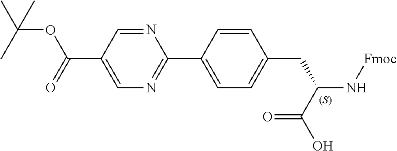

### (S)-2-((((9H-fluoren-9-yl)methoxy)carbonyl)amino-3-(4-(5-(tert-butoxycarbonyl)pyrimidin-2-yl)phenyl)propanoic acid

Step 1: To a mixture of 2-chloropyrimidine-5-carboxylic acid (10 g, 63.1 mmol) in t-BuOH (100 mL) were added DMAP (0.771 g, 6.31 mmol) and Boc₂O (16.52 g, 76 mmol) under argon at rt. The resulting mixture was stirred at 50° C. for 16 h, then it was cooled to rt and concentrated under vacuum. The residue was purified by silica gel column chromatography, eluted with 0-60% EtOAc in PE to give tert-butyl 2-chloropyrimidine-5-carboxylate. MS ESI calculated for C₉H₁₂ClN₂O₂ [M+H]⁺ 215.05, found 214.95.

Step 2: To a mixture of tert-butyl 2-chloropyrimidine-5-carboxylate (4 g, 18.64 mmol) in 1,4-Dioxane (40 mL) and water (8 mL) were added (S)-3-(4-boronophenyl)-2-((tert-butoxycarbonyl)amino) propanoic acid (8.64 g, 28.0 mmol), PdCl₂(dppf) (1.364 g, 1.864 mmol) and K₂CO₃ (7.73 g, 55.9 mmol) under argon at rt. The resulting mixture was stirred at 80° C. for 3 h, then the pH was adjusted to 4 with 1 N HCl and extracted with EtOAc (2×300 mL). The combined organic layer was washed with brine (3×50 mL), dried over anhydrous Na₂SO₄ and filtered. The filtrate was concentrated under reduced pressure. The residue was purified by silica gel column chromatography, eluted with 0-100% EtOAc in PE to give (S)-2-((tert-butoxycarbonyl) amino)-3-(4-(5-(tert-butoxycarbonyl) pyrimidin-2-yl) phenyl)propanoic acid. MS ESI calculated for C₂₃H₃₀N₃O₆ [M+H]⁺ 444.21, found 444.35. ¹H NMR (400 MHz, CDCl₃) δ 9.27 (s, 2H), 8.42 (d, J=7.8 Hz, 2H), 7.36 (d, J=7.9 Hz, 2H), 4.69-4.68 (m, 1H), 3.29-3.24 (m, 2H), 1.63 (s, 9H), 1.44 (s, 9H).

Step 3: To a mixture of (S)-2-((tert-butoxycarbonyl)amino)-3-(4-(5-(tert-butoxycarbonyl)pyrimidin-2-yl)phenyl)propanoic acid (7 g, 15.78 mmol) in DCM (70 mL) was added TFA (14 mL, 182 mmol) under argon at rt. After stirring at rt for 1 h, the reaction was concentrated to obtain (S)-2-amino-3-(4-(5-(tert-butoxycarbonyl)pyrimidin-2-yl)phenyl)propanoic acid. MS ESI calculated for C₁₈H₂₂N₃O₄ [M+H]⁺ 344.15, found 344.20.

Step 4: To a mixture of (S)-2-amino-3-(4-(5-(tert-butoxycarbonyl) pyrimidin-2-yl) phenyl) propanoic acid (4 g, 11.65 mmol) in THF (40 mL) and H₂O (40 mL) were added NaHCO₃ (4.89 g, 58.2 mmol) and Fmoc-OSu (3.54 g, 10.48 mmol) under argon at rt. After stirring at rt for 1 h, the pH was adjusted to 4 with 1 N HCl and the solution was extracted with EtOAc (2×300 mL). The combined organic layer was washed with brine (150 mL), dried over anhydrous Na₂SO₄, and filtered. The filtrate was concentrated under reduced pressure and the residue was purified by RP-flash with the following conditions: Column: C¹⁸ column (330 g); Mobile Phase A: water (0.05% TFA); Mobile Phase B: MeCN; (Gradient: 0% B hold 5 min. up to 73% B within 30 min, 73% B hold 10 min; up to 95% B within 4 min, 95% B hold 10 min); Flow rate: 60 mL/min; Detector: UV 254 & 210 nm; RT: 35.32 min to give (S)-2-((((9H-fluoren-9-yl)methoxy)carbonyl)amino)-3-(4-(5-(tert-butoxycarbonyl) pyrimidin-2-yl) phenyl) propanoic acid. MS ESI calculated for C₃₃H₃₂N₃O₆ [M+H]⁺ 566.22, found 566.35. ¹H NMR (300 MHz, DMSO-d₆) δ 12.85 (s, 1H), 9.24 (s, 2H), 8.37 (d, J=8.0 Hz, 2H), 7.88-7.79 (m, 3H), 7.66-7.14 (m, 8H), 4.30-4.14 (m, 4H), 3.22-3.16 (m, 1H), 3.05-2.82 (m, 1H), 1.59 (s, 9H).

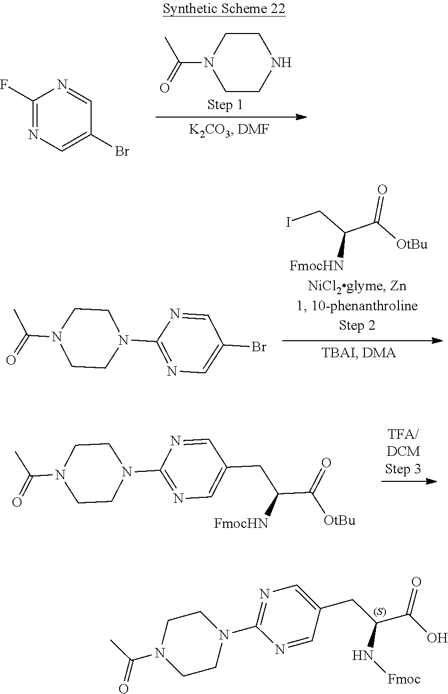

### Precursor to PyrimAla4AcPip

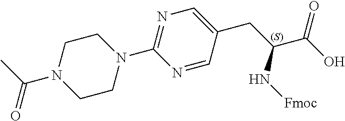

### (S)-2-((((9H-fluoren-9-yl)methoxy)carbonyl)amino)-3-(2-(4-acetylpiperazin-1-yl)pyrimidin-5-yl)propanoic acid

Step 1: To a mixture of 5-bromo-2-fluoropyrimidine (7 g, 39.6 mmol) in DMF (70 ml) were added 1-(piperazin-1-yl)ethan-1-one (10.14 g, 79 mmol) and K₂CO₃ (10.93 g, 79 mmol) at rt. The reaction mixture was stirred for 2 h at 100° C. then cooled back down to rt. The reaction mixture was extracted with 500 mL EtOAc, washed with H₂O (3×100 mL) and brine (80 mL), dried over Na₂SO₄, filtered and the filtrate was concentrated under reduced pressure. The residue was purified by silica gel column chromatography, eluting with gradient 0%-80% EtOAc in PE. The fractions containing desired product were combined and concentrated under reduced pressure to afford 4-(5-bromopyrimidin-2-yl)piperazine-1-carbaldehyde. MS ESI calculated for C₁₀H₁₄BrN₄O [M+H]⁺ 285.03, 287.02, found 284.95, 286.95. ¹H NMR (300 MHz, CDCl₃) δ 8.32 (s, 2H), 3.90-3.74 (m, 4H), 3.74-3.64 (m, 2H), 3.58-3.48 (m, 2H), 2.15 (s, 3H).

Step 2: A mixture of Nickel (II) chloride ethylene glycol dimethyl ether complex (0.693 g, 3.16 mmol) and 1,10-phenanthroline (0.569 g, 3.16 mmol) in DMA (50 mL) was heated at 50° C. for 0.5 hours. The solution of 1-(4-(5-bromopyrimidin-2-yl)piperazin-1-yl)ethan-1-one (4.5 g, 15.78 mmol), tert-butyl (R)-2-((((9H-fluoren-9-yl)methoxy)carbonyl)amino)-3-iodopropanoate (11.68 g, 23.67 mmol) and TBAI (5.83 g, 15.78 mmol) in DMA (50 mL) were added at 25° C. Then Zn (2.064 g, 31.6 mmol) was added. After the resulting mixture was stirred for 24 h at 30° C., the reaction mixture was filtrated and washed with DCM. The organic phase was concentrated under reduced pressure. The residue was purified by RP-flash with the following conditions: Column: C¹⁸ column (330 g); Mobile Phase A: water (0.05% TFA); Mobile Phase B: MeCN; (Gradient: 0% B hold 5 min, up to 82% B within 30 min, 82% B hold 6 min; up to 95% B within 2 min, 95% B hold 3 min); Flow rate: 60 mL/min; Detector: UV 254 & 210 nm; RT: 35 min to give tert-butyl (S)-2-((((9H-fluoren-9-yl)methoxy)carbonyl)amino)-3-(2-(4-acetylpiperazin-1-yl)pyrimidin-5-yl)propanoate. MS ESI calculated for C₃₂H₃₈N₅O₅ [M+H]⁺ 572.28, found 572.30. ¹H NMR (300 MHz, CDCl₃) δ 8.19 (s, 2H), 7.77 (d, J=7.6 Hz, 2H), 7.63-7.52 (m, 2H), 7.46-7.26 (m, 4H), 4.48-4.42 (m, 2H), 4.35-4.30 (m, 1H), 4.22-4.17 (m, 1H), 3.89-3.81 (m, 4H), 3.71-3.68 (m, 2H), 3.56-3.52 (m, 2H), 3.08-3.02 (m, 1H), 2.92-2.87 (m, 1H), 2.17 (s, 3H), 1.48 (s, 9H).

Step 3: To a mixture of tert-butyl (S)-2-((((9H-fluoren-9-yl)methoxy)carbonyl)amino)-3-(2-(4-acetylpiperazin-1-yl)pyrimidin-5-yl)propanoate (4.6 g, 8.05 mmol) in DCM (40 mL) was added TFA (80 mL, 1038 mmol) under argon at rt. The reaction was stirred at rt for 3 h, then concentrated under reduced pressure. The residue was purified by RP-flash with the following conditions: Column: C¹⁸ gel column (330 g); Mobile Phase A: water (0.05% TFA); Mobile Phase B: MeCN; (Gradient: 0% B hold 5 min, up to 72% B within 30 min, 72% B hold 6 min; up to 95% B within 2 min, 95% B hold 10 min); Flow rate: 60 mL/min; Detector: UV 254 & 210 nm; RT: 35 min to give (S)-2-((((9H-fluoren-9-yl)methoxy)carbonyl)amino)-3-(2-(4-acetylpiperazin-1-yl)pyrimidin-5-yl)propanoic acid. MS ESI calculated for C₂₈H₃₀N₅O₅ [M+H]⁺ 516.22, found 516.35. ¹H NMR (300 MHz, DMSO-d₆) δ 8.31 (s, 2H), 7.89 (d, J=7.5 Hz, 2H), 7.81 (d, J=8.6 Hz, 1H), 7.74-7.62 (m, 2H), 7.59-7.22 (m, 4H), 4.29-4.09 (m, 4H), 3.74-3.60 (m, 4H), 3.47-3.46 (m, 4H), 2.98-2.92 (m, 1H), 2.76-2.68 (m, 1H), 2.03 (s, 3H).

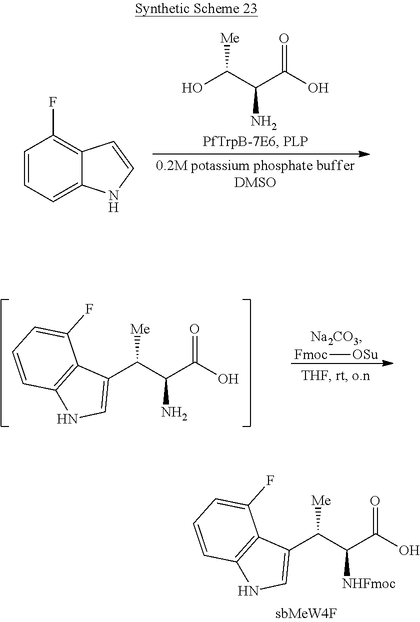

### Precursor to sbMeW4F

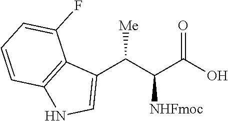

### (2S,3S)-2-amino-3-(4-fluoro-1H-indol-3-yl)butanoic acid

Into a 1-L 3-necked round-bottom flask purged and maintained with an inert atmosphere of nitrogen, was placed 4-fluoro-1H-indole (10 g, 1.00 eq), L-threonine (10.6 g, 1.20 eq). DMSO (100 mL), potassium phosphate buffer (0.2 M, 300 mL, pH=7.4). The reaction mixture was heated to 65° C., then PfTrpB-7E6 (2.5 g, 25 wt %) and 3-hydroxy-2-methyl-5-([phosphonooxy]methyl)-4-pyridinecarboxaldehyde (0.078 g, 0.004 eq) were added. The resulting solution was stirred overnight at 65° C. The mixture was then cooled to rt and used directly in the next step.

Into the above reaction mixture, THF (100 mL), sodium carbonate (23.56 g, 3.0 eq.) and 2,5-dioxopyrrolidin-1-yl 9H-fluoren-9-ylmethyl carbonate (29.96 g, 1.20 eq.) were added at 0° C. The resulting solution was stirred overnight at rt. The pH was adjusted to 4 by 3 M HCl and the solid precipitate was filtered away. The resulting solution was extracted with EtOAc (3×500 mL). The organic fractions were combined, and washed with brine (1 L), dried over anhydrous sodium sulfate and concentrated under vacuum. The mixture was applied onto a silica gel column with MeOH:DCM=1:5. HPLC-MS: (ES, m/z): [M+1]: 459. ¹H NMR (300 MHz, DMSO-d₆) δ 12.60 (s, 1H), 11.15 (s, 1H), 7.87 (d, J=7.6 Hz, 2H), 7.76-7.49 (m, 3H), 7.47-7.34 (m, 2H), 7.34-7.16 (m, 4H), 7.03 (td, J=7.9, 5.0 Hz, 1H), 6.73 (dd, J=11.8, 7.7 Hz, 1H), 4.36 (t, J=8.5 Hz, 1H), 4.31-4.02 (m, 3H), 3.51 (q, J=7.4 Hz, 1H), 1.31 (d, J=7.0 Hz, 4H), 0.78 (s, 1H).

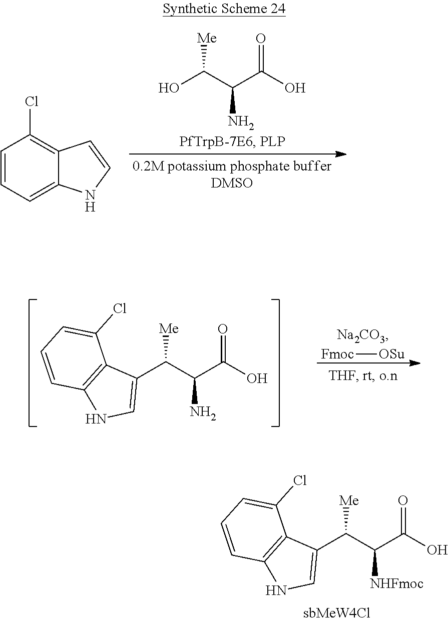

### Precursor to sbMeW4CI

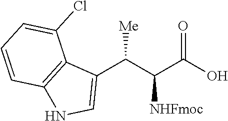

### (2S,3S)-2-amino-3-(4-chloro-1H-indol-3-yl)butanoic acid

Into a 1-L 3-necked round-bottom flask purged and maintained with an inert atmosphere of nitrogen, was placed 4-chloro-1H-indole (10 g, 1.00 eq.), L-threonine (14.09 g, 1.8 eq.), DMSO (100 mL), potassium phosphate buffer (0.2 M, 300 mL, PH=7.4), the reaction mixture was heated to 65° C., then PfTrpB-7E6 (7.5 g, 25 wt %) and 3-hydroxy-2-methyl-5-([phosphonooxy]methyl)-4-pyridinecarboxaldehyde (174 mg, 0.01 eq.) were added. The resulting solution was stirred for 36 h at 65° C. The mixture was then cooled to rt and used directly in the next step.

Into the above reaction mixture, THF (100 mL), sodium carbonate (20.9 g, 3.0 eq.) and 2,5-dioxopyrrolidin-1-yl 9H-fluoren-9-ylmethyl carbonate (31.0 g, 1.40 eq.) were added at 0° C. The resulting solution was stirred overnight at rt. The pH was adjusted to 4 by 3 M HCl and the solid precipitate was filtered away. The resulting solution was extracted with EtOAc (3×500 mL). The organic fractions were combined, and washed with brine (1 L), dried over anhydrous sodium sulfate and concentrated under vacuum. HPLC-MS: (ES, m/z): [M+1]: 475

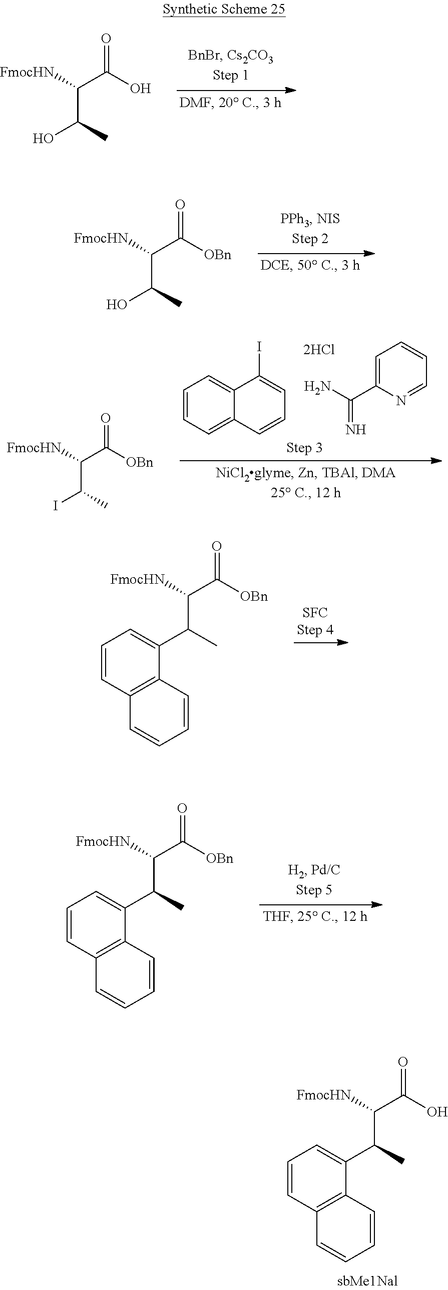

### Precursor to sbMe1Nal

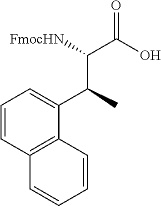

### (2S,3S)-2-amino-3-(naphthalen-1-yl)butanoic acid

Step 1: To a solution of (2S,3R)-2-((((9H-fluoren-9-yl)methoxy)carbonyl)amino)-3-hydroxybutanoic acid (250 g, 1.00 eq) in DMF (1.5 L) was added benzyl bromide (250 g, 2.00 eq) dropwise at 20° C. Then, cesium carbonate (477 g, 2.00 eq) was added and the solution was stirred at 20° C. for 3 h. The reaction was poured into ice H₂O (3 L) and extracted with EtOAc (500 mL×3). The organic phase was washed with 3% LiCl solution (500 mL×2) and brine (500 mL), dried over sodium sulfate and concentrated under vacuum at 40° C. The crude product was triturated with methyl tert-butyl ether:PE=6:1. ¹H NMR (400 MHz, CDCl₃): δ 7.77 (d, J=7.6 Hz, 1H), 7.40 (d, J=8.0 Hz, 1H), 7.31-7.36 (m, 10H), 5.65-5.71 m, 1H), 5.13-5.31 (m, 3H), 4.39-4.43 (m, 3H), 4.22-4.25 (m, 1H), 1.25 (d, J=6.4 Hz, 3H)

Step 2: To a 3-neck round-bottom flask was added (2S,3R)-benzyl 2-((((9H-fluoren-9-yl)methoxy)carbonyl)amino)-3-hydroxybutanoate (125 g, 1.00 eq) and DCE (750 mL) with an inert atmosphere of nitrogen. The reaction was cooled to 0° C. followed by the addition of NIS (195 g, 3.00 eq) and PPh₃ (228 g, 3.00 eq). The temperature was raised to 50° C. and the reaction mixture was stirred for 3 h. The reaction was poured into ice H₂O (500 mL) and extracted with DCM (500 mL×2). The organic phase was dried over sodium sulfate and concentrated under vacuum at 40° C. The residue was purified by silica gel column chromatography (PE/EtOAc=1/0 to 0/1). ¹H NMR (400 MHz, CDCl₃): δ 7.78 (d, J=7.6 Hz, 2H), 7.68 (d, J=7.2 Hz, 2H), 7.33-7.43 (m, 9H), 5.27-5.68 (m, 1H), 5.21-5.23 (m, 2H), 4.39-4.52 (m, 3H), 4.25-4.38 (m, 1H), 1.91-1.95 (m, 3H).

Step 3: To a 3-neck round-bottom flask was placed 2-((((9H-fluoren-9-yl) methoxy) carbonyl)amino)-3-iodobutanoate, 1-iodonaphthalene (42.2 g, 1.20 eq), TBAI (76.7 g, 1.50 eq), Zn (19.0 g, 2.10 eq) and DMA (750 mL). To a second 3-neck round-bottom flask was placed picolinimidamide.2HCl (42.2 g, 1.20 eq), NiCl₂.glyme (7.61 g, 0.25 eq) and DMA (750 mL) at 25° C. Under argon, the contents of the second flask were added to the first flask. The resulting mixture was then stirred for 12 h at 25° C. The reaction was poured into ice H₂O (3 L) and extracted with EtOAc (1 L×2). The organic phase was dried over sodium sulfate and concentrated under vacuum at 40° C. The crude product was purified by reversed-phase HPLC (MeCN:H₂O). HPLC-MS: [M+23]: 564. ¹H NMR (400 MHz, CDCl₃) δ: 8.17-8.24 (m, 1H), 8.15-8.17 (m, 1H), 7.77-7.87 (m, 2H), 7.76-7.77 (m, 4H), 7.30-7.41 (m, 10H), 5.30-5.38 (m, 1H), 4.96-5.04 (m, 3H), 4.85-4.87 (m, 1H), 4.30-4.34 (m, 1H), 4.18-4.26 (m, 4H), 1.43-1.45 (m, 3H).

Step 4: 143 g of (2S)-benzyl 2-((((9H-fluoren-9-yl) methoxy) carbonyl) amino)-3-(naphthalen-1-yl)butanoate was separated by SFC. The organic phase was concentrated under vacuum at 35° C.

Peak 1: (2S,3R)-benzyl 2-((((9H-fluoren-9-yl)methoxy)carbonyl) amino)-3-(naphthalene-1-yl)butanoate. ¹H NMR (400 MHz, DMSO-d₆): δ 8.11-8.12 (m, 2H), 8.10-8.11 (m, 1H), 7.88-7.90 (m, 2H), 7.54-7.88 (m, 1H), 7.44-7.53 (m, 2H), 7.42-7.44 (m, 4H), 7.33-7.42 (m, 3H), 7.27-7.33 (m, 6H), 7.08-7.09 (m, 2H), 4.91-4.94 (m, 1H), 4.79-4.82 (m, 1H), 4.58 (t, J=8.0 Hz), 4.17-4.25 (m, 4H), 1.39 (d, J=6.8 Hz, 3H). Peak 2: (2S,3S)-benzyl 2-((((9H-fluoren-9-yl)methoxy) carbonyl) amino)-3-(naphthalen-1-yl)butanoate. ¹H NMR (400 MHz, DMSO-d₆): δ 7.92-8.15 (m, 1H), 7.86-7.92 (m, 1H), 7.84-7.86 (m, 1H), 7.57-7.84 (m, 2H), 7.56-7.57 (m, 1H), 7.41-7.54 (m, 4H), 7.30-7.38 (m, 4H), 7.27-7.30 (m, 7H), 5.08-5.14 (m, 2H), 4.65 (t, J=8.0 Hz), 4.23-4.26 (m, 1H), 4.05-4.18 (m, 3H), 1.30 (d, J=6.8 Hz, 3H).

Step 5: To a 3-neck round-bottom flask was added (2S,3S)-benzyl 2-((((9H-fluoren-9-yl) methoxy) carbonyl)amino)-3-(naphthalen-1-yl)butanoate (40.0 g, 1.00 eq) and THF (200 mL), 10% wet Pd/C (7.00 g) was added and the reaction was purged 3 times with H₂ and stirred at 25° C. for 12 h under H₂(15 psi). The reaction was filtered through a celite pad and concentrated under vacuum at 35° C. The crude product was triturated with PE at 25° C. for 1 h. After filtration, the filter cake was dissolved in MeCN (100 mL) and concentrated under vacuum at 35° C. to remove residual solvent. HPLC-MS: [M+23]: 474. ¹H NMR (400 MHz, DMSO-d₆) δ 12.78 (s, 1H), 8.23 (d, J=7.6 Hz, 1H), 7.86-7.88 (m, 1H), 7.80-7.86 (m, 2H), 7.61-7.80 (m, 2H), 7.55-7.59 (m, 4H), 7.481-7.55 (m, 1H), 7.40-7.48 (m, 3H), 7.27-7.29 (m, 2H), 4.28-4.60 (m, 1H), 4.24-4.28 (m, 1H), 4.17-4.24 (m, 2H), 4.04-4.15 (m, 1H), 1.36 (d, J=6.8 Hz, 3H).

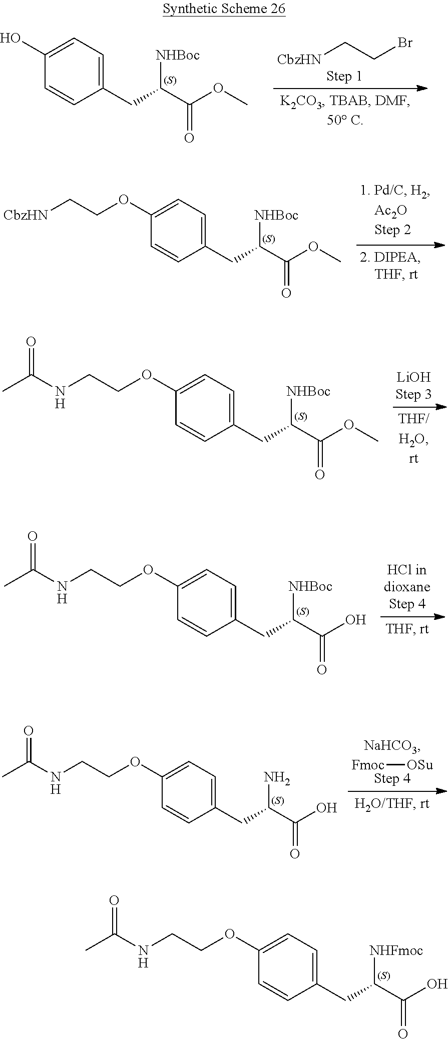

### Precursor to TyrEtNAc

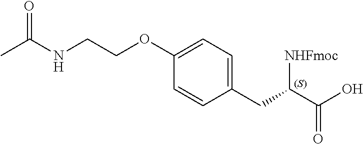

### L-Tyrosine O-ethyl acetamide or (S)-3-(4-(2-acetamidoethoxy)phenyl)-2-aminopropanoic acid

Step 1: To a stirred solution of methyl (tert-butoxycarbonyl)-L-tyrosinate (10.0 g, 33.9 mmol), benzyl (2-bromoethyl)carbamate (26.2 g, 102 mmol) and TBAB (5.46 g, 16.93 mmol) in DMF (150 mL) was added potassium carbonate (14.04 g, 102 mmol) at rt. The mixture was then stirred at 50° C. for 24 h. The mixture was cooled to rt, quenched with water (250 mL) and extracted with EtOAc (2×500 mL). The combined organic layers were washed with brine (3×150 mL), dried over anhydrous sodium sulfate and filtered. The filtrate was concentrated under reduced pressure, and the residue was purified by silica gel column chromatography, eluted with 0-30% EtOAc in PE. MS ESI calculated for C₂₅H₃₂N₂O₇ [M+Na]⁺ 495.22, found 495.10; ¹H NMR (300 MHz, CDCl₃) δ 7.38-7.32 (m, 5H), 7.04 (d, J=8.4 Hz, 2H), 6.81 (d, J=8.4 Hz, 2H), 5.31 (br, 1H), 5.21 (s, 2H), 4.97 (br, 1H), 4.56-4.53 (m, 1H), 4.03 (t, J=5.0 Hz, 2H), 3.72 (s, 3H), 3.64-3.58 (m, 2H), 3.06-3.01 (m, 2H), 1.43 (s, 9H).

Step 2: To a stirred solution of methyl (S)-3-(4-(2-(((benzyloxy)carbonyl)amino)ethoxy)phenyl)-2-((tert-butoxycarbonyl)amino)propanoate (16.0 g, 33.9 mmol) and acetic anhydride (6.39 mL, 67.7 mmol) in THF (200 mL) was added Pd/C (3.60 g, 33.9 mmol, dry, 10% wt) at rt under nitrogen atmosphere. The mixture was degassed with hydrogen 3 times and stirred at 20° C. for 4 h. DIPEA (17.74 mL, 102 mmol) was added to the mixture and stirred at 20° C. for 1 h. The mixture was filtered, and the filtrate was concentrated under reduced pressure. The residue was purified by silica gel column chromatography, eluted with 0-3% MeOH in DCM. MS ESI calculated for C₁₉H₂₈N₂O₆ [M+Na]⁺ 403.19, found 403.10; ¹H NMR (300 MHz, CDCl₃) δ 7.05 (d, J=8.4 Hz, 2H), 6.85-6.80 (m, 2H), 5.99 (br, 1H), 5.32 (br, 1H), 4.99-4.97 (m, 1H), 4.02 (t, J=5.0 Hz, 2H), 3.72 (s, 3H), 3.69-3.63 (m, 2H), 3.10-2.89 (m, 2H), 2.02 (s, 3H), 1.42 (s, 9H).

Step 3: To a stirred solution of methyl (S)-3-(4-(2-acetamidoethoxy)phenyl)-2-((tert-butoxycarbonyl)amino)propanoate (12.5 g, 32.9 mmol) THF (100 mL) was added lithium hydroxide (65.7 mL, 65.7 mmol, 1 N in water) at rt. The solution was stirred at 20° C. for 2 h. The pH of the solution was adjusted to 3 with 1 N HCl. The aqueous layer was extracted with EtOAc (2×250 mL). The combined organic layers were washed with brine (150 mL), dried over anhydrous sodium sulfate, filtered, and concentrated under reduced pressure.

MS ESI [M+H]⁺: 367.10.

Step 4: To a stirred solution of (S)-3-(4-(2-acetamidoethoxy)phenyl)-2-((tert-butoxycarbonyl)amino)propanoic acid (12.5 g, 30.7 mmol) in THF (20 mL) was added 4 N HCl in dioxane (200 mL) at rt. The solution was stirred at 20° C. for 1 h. The solvent was concentrated under reduced pressure. MS ESI [M+H]⁺: 267.05.

Step 5: To a stirred mixture of (S)-3-(4-(2-acetamidoethoxy)phenyl)-2-aminopropanoic acid hydrochloride (9.50 g, 25.1 mmol) and NaHCO₃ (10.54 g, 126 mmol) in THF (100 mL) and water (100 mL) was added Fmoc-OSu (7.62 g, 22.59 mmol) at rt. The mixture was stirred at 20° C. for 1 h. The pH value of the solution was adjusted to 3 with 1 N HCl. The aqueous phase was extracted with EtOAc (2×500 mL). The combined organic layers were washed with brine (150 mL), dried over anhydrous sodium bicarbonate and filtered. The filtrate was concentrated under reduced pressure, and the residue was recrystallized from EtOAc (200 mL). The solid was collected by filtration and dried under vacuum. MS ESI [M+H]⁺: 489.05; ¹H NMR (300 MHz, Methanol-d₄) δ 7.79 (d, J=7.6 Hz, 2H), 7.62-7.57 (m, 2H), 7.42-7.26 (m, 4H), 7.17-7.14 (m, 2H), 6.83 (d, J=8.4 Hz, 2H), 4.41-4.31 (m, 2H), 4.29-4.10 (m, 2H), 3.96 (t, J=4.8 Hz, 2H), 3.51 (t, J=5.4 Hz, 2H), 3.19-3.13 (m, 1H), 2.92-2.84 (m, 1H), 1.94 (s, 3H).

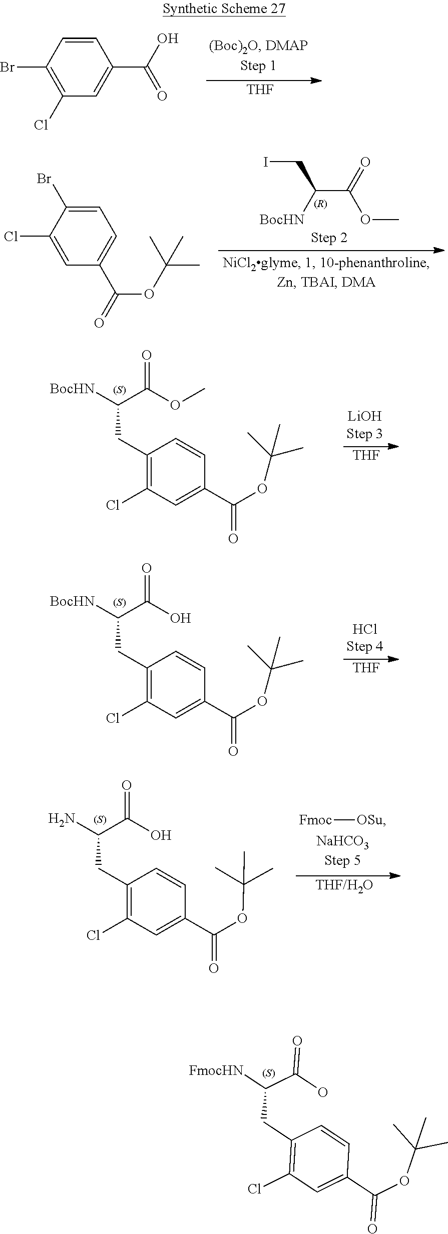

### Precursor to Phe2Cl4COOH

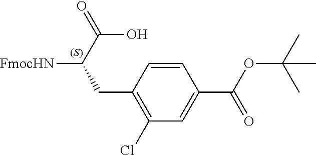

### (S)-2-((((9H-fluoren-9-yl)methoxy)carbonyl)amino)-3-(4-(tert-butoxycarbonyl)-2-chlorophenyl)propanoic acid

Step 1: To a stirred mixture of 4-bromo-3-chlorobenzoic acid (25 g, 106 mmol) in THF (100 mL) was added Boc₂O (27.8 g, 127 mmol) and DMAP (1.297 g, 10.62 mmol) at 25° C. under argon nitrogen atmosphere. The resulting mixture was stirred for 16 h at 25° C. The reaction progress was monitored by LCMS. After completion, the reaction mixture was concentrated under reduced pressure and the residue was purified by silica gel column chromatography(eluted with 0~10% EA in PE) and the product-containing fractions were collected and roto-evaporated in vacuum to afford tert-butyl 4-bromo-3-chlorobenzoate (25 g, 86 mmol, 81% yield) as a colorless oil. ¹H NMR (400 MHz, Chloroform-d) δ 8.04-8.03 (m, 1H), 7.73-7.65 (m, 2H), 1.59 (s, 9H).

Step 2: To a stirred solution of nickel (II) chloride ethylene glycil dimethyl ether complex (3.01 g, 13.72 mmol) in DMA (10 mL) was added 1,10-phenanthroline (2.97 g, 13.72 mmol) at room temperature under argon. The resulted solution was stirred at 50° C. for 30 min. tert-butyl 4-bromo-3-chlorobenzoate (20 g, 68.6 mmol), methyl (R)-2-((tert-butoxycarbonyl)amino)-3-iodopropanoate (22.58 g, 68.6 mmol) and Zn (8.97 g, 137 mmol) were added to the solution and the resulted mixture was stirred at 30° C. for 2 h. After completion, the reaction was quenched with H₂O (500 mL), extracted with EA (2×500 mL). The combined organic layer was washed with brine (3×200 mL), dried over anhydrous Na₂SO₄ and filtered. The filtrate was concentrated under reduced pressure and the residue was purified by silica gel column chromatography, eluted with 0-60% EA in PE to give tert-butyl (S)-4-(2-((tert-butoxycarbonyl)amino)-3-methoxy-3-oxopropyl)-3-chlorobenzoate (10.1 g, 24.40 mmol, 35.6% yield) as an off-white solid.MS ESI calculated for C₂₀H₂₇ClNO₆ [M−H]⁻ 412.16 found 412.10.

Step 3: To a stirred solution of tert-butyl (S)-4-(2-((tert-butoxycarbonyl)amino)-3-methoxy-3-oxopropyl)-3-chlorobenzoate (10.1 g, 24.40 mmol) in THF (70 mL) was added LiOH (1M in water) (70 mL, 24.40 mmol) at 25° C. under argon. The solution was stirred at 25° C. for 1 h. After completion, the solvent was concentrated under reduced pressure to give (S)-3-(4-(tert-butoxycarbonyl)-2-chlorophenyl)-2-((tert-butoxycarbonyl)amino)propanoic acid (8.5 g, 21.26 mmol, 87% yield) as a light yellow solid. The crude product was used to the next step directly without any further purification. MS ESI calculated for C₂₁H₂₉ClNO₆Na [M+ACN+Na]⁺ 463.14. found 463.20.

Step 4: To a stirred solution of (S)-3-(4-(tert-butoxycarbonyl)-2-chlorophenyl)-2-((tert-butoxycarbonyl)amino)propanoic acid (8.5 g, 21.26 mmol) in THF (70 mL) and HCl (4M in dioxane) (70 mL, 852 mmol) at 25° C. The solution was stirred at 25° C. for 2 h then the solvent was concentrated under reduced pressure to give (S)-2-amino-3-(4-(tert-butoxycarbonyl)-2-chlorophenyl)propanoic acid (5.5 g, 18.35 mmol, 86% yield) as a yellow solid. The crude product was used to the next step directly without any further purification.MS ESI calculated for C₁₄H₁₉ClNO₄ [M+H]⁺ 300.09, found 300.15.

Step 5: To a stirred mixture of (S)-2-amino-3-(4-(tert-butoxycarbonyl)-2-chlorophenyl)propanoic acid (5.5 g, 18.35 mmol)and NaHCO₃ (7.71 g, 92 mmol) in THF (40 mL) and water (40 mL) was added Fmoc-Su (5.57 g, 16.51 mmol) at room temperature. The mixture was stirred at 25° C. for 2 h. After completion, the pH value of the solution was adjusted to 3 with 1 N HCl. The aqueous phase was extracted with EA (2×500 mL). The combined organic layer was washed with brine (150 mL), dried over anhydrous Na₂SO₄ and filtrated. The filtrate was concentrated under reduced pressure and the solvent was concentrated under reduced pressure and the residue was purified by RP flash with the following conditions: Column: Flash C 18 (330 g); Mobile Phase A: water (0.1% TFA), Mobile Phase B: ACN; (Gradient: 5% B hold 5 min, up to 60% B within 25 min, 60% B hold 15 min; up to 95% B within 10 min, 95% B hold 10 min); Flow rate: 90 mL/min; Detector: UV 210 nm; RT=30 min. The product-containing fractions were collected and roto-evaporated in vacuo to give (S)-2-((((9H-fluoren-9-yl)methoxy)carbonyl)amino)-3-(4-(tert-butoxycarbonyl)-2-chlorophenyl)propanoic acid (6.2303 g, 11.94 mmol, 65.0% yield) as an off-white solid. MS ESI calculated for C₂₉H₂₈ClNO₆Na [M+Na]⁺ 544.16, found 544.20. ¹H NMR (300 MHz, Methanol-d₄) δ 7.89 (s, 1H), 7.78-7.70 (m, 3H), 7.58-7.23 (m, 7H), 4.62-4.57 (m, 1H), 4.32-4.26 (m, 1H), 4.16-4.03 (m, 2H), 3.54-3.48 (m, 1H), 3.09-3.01 (m, 1H), 1.52 (s, 9H).

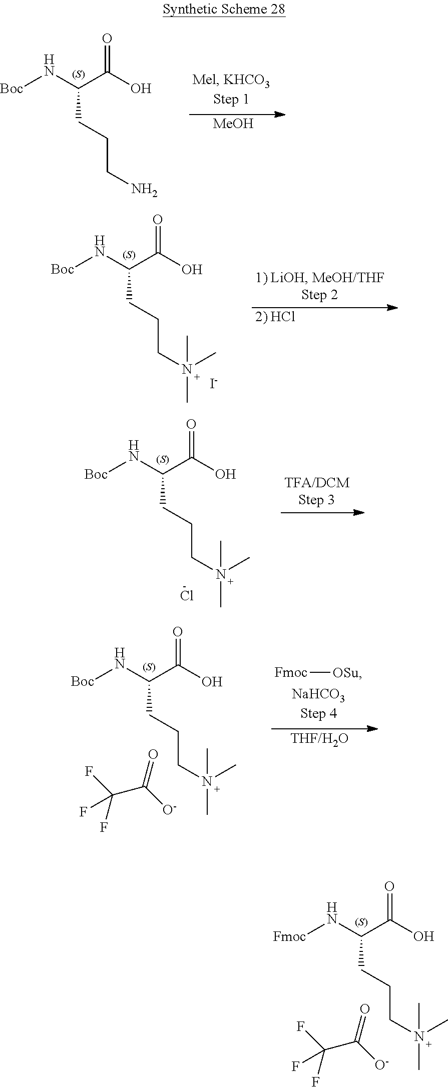

### Precursor to OrnMe3

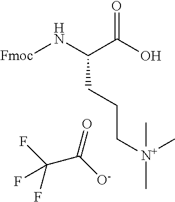

### (S)-4-((((9H-fluoren-9-yl)methoxy)carbonyl)amino)-4-carboxy-N,N,N-trimethylbutan-1-aminium 2,2,2-trifluoroacetate

Step 1: To a mixture of (S)-5-amino-2-((tert-butoxycarbonyl)amino)pentanoic acid (4.6 g, 19.80 mmol) in MeOH (50 mL) were added MeI (33.7 g, 238 mmol) and KHCO₃ (1.983 g, 19.80 mmol) at ambient temperature. The resulting mixture was warmed to 50° C. and stirred for 12 h. The reaction was cooled to room temperature and filtered. The filtrate was concentrated in vacuum and the residue was dissolved in DCM (80 mL) and then filtered again. The filtrate was concentrated in vacuum to afford (S)-4-((tert-butoxycarbonyl)amino)-5-methoxy-N,N,N-trimethyl-5-oxopentan-1-aminium iodide (7.4 g, 16.00 mmol, 81% yield) as an off-pink solid. MS ESI calculated for C₁₄H₂₉N₂O₄ [M−I]⁺ 289.21, found 289.35.

Step 2: To a mixture of (S)-4-((tert-butoxycarbonyl)amino)-5-methoxy-N,N,N-trimethyl-5-oxopentan-1-aminium iodide (7.4 g, 16.00 mmol) in MeOH (48 mL) and THF (24 mL) was added LiOH (48.0 mL, 48.0 mmol, 1 M in water) at ambient temperature. The reaction was stirred at ambient temperature for 2 h then concentrated in vacuum. And then 48 mL 1 N HCl was added. The solvent was concentrated in vacuum to afford (S)-4-((tert-butoxycarbonyl)amino)-4-carboxy-N,N,N-trimethylbutan-1-aminium chloride (7.5 g, 14.48 mmol, 90% yield) as a white solid. MS ESI calculated for C₁₃H₂₇N₂O₄ [M−Cl]⁺ 275.20. found 275.20.

Step 3: To a mixture of (S)-4-((tert-butoxycarbonyl)amino)-4-carboxy-N,N,N-trimethylbutan-1-aminium chloride (7.5 g, 14.48 mmol) in DCM (50 mL) was added TFA (25 mL, 324 mmol) at ambient temperature. The reaction was stirred at ambient temperature for 1 h then concentrated in vacuum to afford (S)-4-amino-4-carboxy-N,N,N-trimethylbutan-1-aminium 2,2,2-trifluoroacetate (10 g, 13.88 mmol, 96% yield) as an orange oil. MS ESI calculated for C₈H₁₉N₂O₂ [M−CF₃COO]⁺175.14, found 175.20.

Step 4: To a mixture of (S)-4-amino-4-carboxy-N,N,N-trimethylbutan-1-aminium 2,2,2-trifluoroacetate (10 g, 13.88 mmol) in THF (30 mL) and water (30 mL) were added NaHCO₃ (9.33 g, 111 mmol) and Fmoc-OSu (4.21 g, 12.49 mmol) at ambient temperature. The reaction was stirred at ambient temperature for 2 h. The resulting solution was acidified with aqueous HCl to pH 3~4 and then filtered. The filtrate was purified by Rp-flash with the following conditions: Column: Flash C¹⁸ (330 g); Mobile Phase A: water (0.05% TFA), Mobile Phase B: ACN; (Gradient: 0% B hold 5 min, up to 33% B within 18 min, 33% B hold 7 min; up to 95% B within 5 min, 95% B hold 5 min); Flow rate: 90 mE/min Detector: UV 210 nm; RT=40 min. The product-containing fractions were collected and lyophilized to afford (S)-4-((((9H-fluoren-9-yl)methoxy)carbonyl)amino)-4-carboxy-N,N,N-trimethylbutan-1-aminium 2,2,2-trifluoroacetate (6.2043 g, 12.15 mmol, 88% yield) as an off-white solid. MS ESI calculated for C₂₃H₂₉N₂O₄ [M−CF₃COO]⁺ 397.21, found 397.15. ¹H NMR (400 MHz, DMSO-d₆) δ 12.85 (br, 1H), 7.92-7.89 (m, 2H), 7.74-7.63 (m, 3H), 7.44-7.40 (m, 2H), 7.33-7.31 (m, 2H), 4.38-4.22 (m, 3H), 4.02-3.98 (m, 1H), 3.34-3.20 (m, 2H), 3.09 (s, 9H), 1.79-1.51 (m, 4H), ¹⁹F-NMR (376 MHz, DMSO-d₆) −73.64.

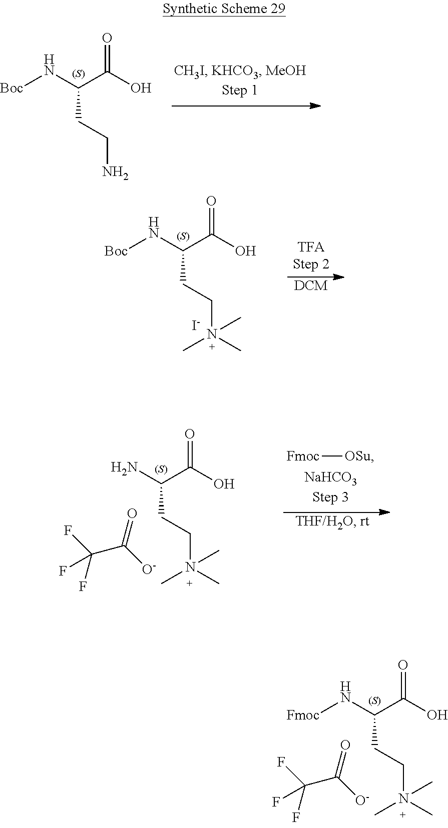

### Precursor to DabMe3

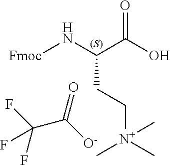

### (S)-3-((((9H-fluoren-9-yl)methoxy)carbonvl)amino)-3-carboxy-N,N,N-trimethylpropan-1-aminium 2,2,2-trifluoroacetate

Step 1: To a mixture of (S)-4-amino-2-((tert-butoxycarbonyl)amino)butanoic acid (5.46 g, 25 mmol) in MeOH (20 mL) were added CH₃I (7.82 mL, 125 mmol) and KHCO₃ (12.51 g, 125 mmol) at ambient temperature. The reaction was warmed to 35° C. for 12 h. The resulting mixture was filtered. The filtrate was concentrated in vacuum and the residue was dissolved in ethanol and then filtered again. The filtrate was concentrated in vacuum to afford (S)-3-((tert-butoxycarbonyl)amino)-3-carboxy-N,N,N-trimethylpropan-1-aminium iodide (13.3 g, 22.27 mmol, 89% yield) as an off-white solid. MS ESI calculated for C₁₂H₂₅IN₂O₄[M−I]⁺ 261.18, found 261.25.

Step 2: To a mixture of (S)-3-((tert-butoxycarbonyl)amino)-3-carboxy-N,N,N-trimethylpropan-1-aminium iodide (13.3 g, 20.55 mmol) in DCM (60 mL) was added TFA (30 mL, 389 mmol) at ambient temperature. The resulting mixture was stirred at ambient temperature for 1 h then concentrated in vacuum to afford (S)-3-amino-3-carboxy-N,N,N-trimethylpropan-1-aminium 2,2,2-trifluoroacetate (10.5 g, 19.14 mmol, 93% yield) as an orange oil. MS ESI calculated for C₉H₁₇F₃N₂O₄ [M−CF₃COO]⁺ 161.13, found 161.25.

Step 3: To a mixture of (S)-3-amino-3-carboxy-N,N,N-trimethylpropan-1-aminium 2,2,2-trifluoroacetate (10.5 g, 19.14 mmol) in THF (40 mL) and water (40 mL) were added NaHCO₃ (8.04 g, 96 mmol) and Fmoc-OSu (5.81 g, 17.23 mmol). The resulting mixture was stirred at ambient temperature for 2 h then acidified with aqueous HCl to pH 3~4 and then filtered. The filtrate was purified by RP-flash with the following conditions: Column: Flash C¹⁸ (330 g); Mobile Phase A: water (0.05% TFA), Mobile Phase B: ACN; (Gradient: 0% B hold 5 min, up to 33% B within 19 min, 38% B hold 7 min; up to 95% B within 5 min, 95% B hold 5 min); Flow rate: 80 m/min; Detector: UV 210 nm; RT=41 min. The product-containing fractions were collected and lyophilized to afford (S)-3-((((9H-fluoren-9-yl)methoxy)carbonyl)amino)-3-carboxy-N,N,N-trimethylpropan-1-aminium 2,2,2-trifluoroacetate (5.2253 g, 10.52 mmol, 55% yield) as an light yellow solid. MS ESI calculated for C₂₄H₂₇F₃N₂O₆ [M−CF₃COO]⁻ 383.20, found 383.30. ¹H NMR (300 MHz, DMSO-d₆) δ 7.91 (d, J=7.5 Hz, 2H), 7.81-7.78 (m, 1H), 7.74-7.71 (m, 2H), 7.46-7.41 (m, 2H), 7.37-7.32 (m, 2H), 4.42-4.22 (m, 3H), 4.07-4.00 (m, 1H), 3.49-3.41 (m, 1H), 3.34-3.28 (m, 1H), 3.07 (s, 9H), 2.22-2.04 (m, 2H). ¹⁹F-NMR (282 MHz, DMSO-d₆) δ 73.849.

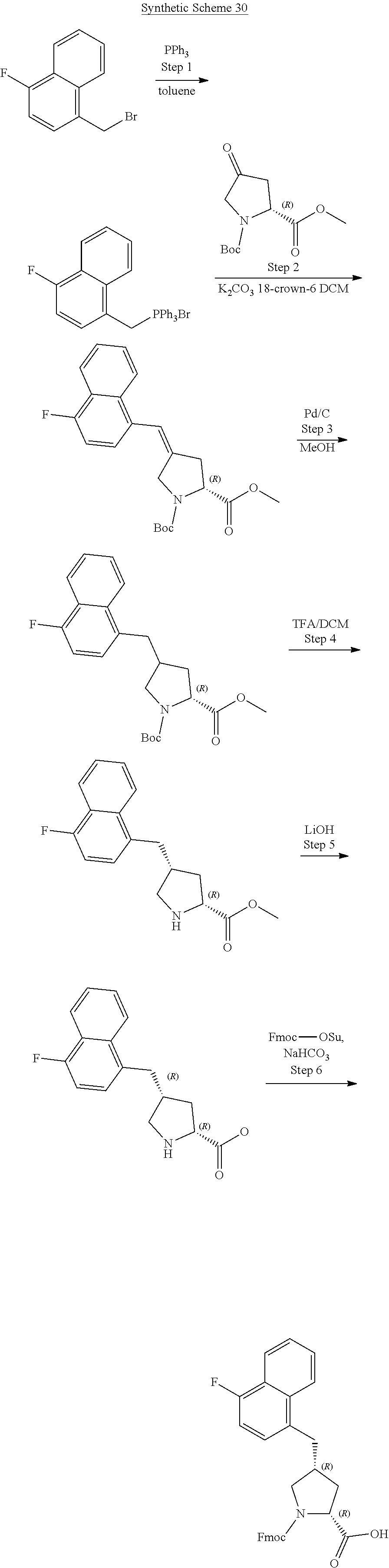

### Precursor to dProc4CH21Naph4F

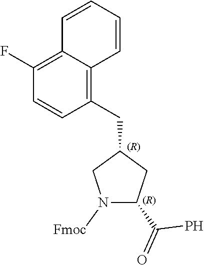

### (2R,4R)-1-(((9H-fluoren-9-yl)methoxy)carbonyl)-4-((4-fluoronaphthalen-1-yl)methyl)pyrrolidine-2-carboxylic acid

Step 1: To a stirred solution of 1-(bromomethyl)-4-fluoronaphthalene (30 g, 125 mmol) in toluene (300 mL) was added PPh₃ (36.2 g, 138 mmol) at 25° C. under nitrogen atmosphere. The resulted solution was stirred at 110° C. for 16 h. The mixture was cooled to room temperature and the solid was collected by filtration to give bromo((4-fluoronaphthalen-1-yl)methyl)triphenyl-15-phosphane (60 g, 120 mmol, 95% yield) as an off-white solid. MS ESI calculated for C₂₉H₂₃FP [M−Br+H]⁺ 421.14, found 421.20.

Step 2: To a stirred solution of bromo((4-fluoronaphthalen-1-yl)methyl)triphenyl-15-phosphane (46.4 g, 92 mmol) in DCM (1160 mL) was added K₂CO₃ (7.67 g, 55.5 mmol) at 25° C. under nitrogen atmosphere. The resulted mixture was stirred at 25° C. for 50 min. 18-Crown-6 (0.978 g, 3.70 mmol) and 1-(tert-butyl) 2-methyl (R)-4-oxopyrrolidine-1,2-dicarboxylate (9 g, 37.0 mmol) were added to the mixture and the resulted mixture was stirred at 45° C. for 16 h. The reaction was cooled to room temperature and quenched with H₂O (500 mL), extracted With EA (2×250 mL). The combined organic layer was washed with brine (3×200 mL), dried over anhydrous Na₂SO₄ and filtered. The filtrate was concentrated under reduced pressure and the residue was purified by silica gel column chromatography, eluted with 0-60% EA in PE to give 1-(tert-butyl) 2-methyl (R,Z)-4-((4-fluoronaphthalen-1-yl)methylene)pyrrolidine-1,2-dicarboxylate (8 g, 20.76 mmol, 56% yield) as an off-white solid. MS ESI calculated for C₂₂H₂₅FNO₄ [M−Boc+H]⁺ 286.17, found 286.10.

Step 3: To a stirred solution of 1-(tert-butyl) 2-methyl (R,Z)-4-((4-fluoronaphthalen-1-yl)methylene)pyrrolidine-1,2-dicarboxylate (8 g, 20.76 mmol) in MeOH (160 mL) was added Pd—C (2.21 g, 2.07 mmol, dry, 10% wt) at room temperature under nitrogen atmosphere. The mixture was degassed with hydrogen for 3 times and stirred at room temperature for 2 h. The solid was filtered out and the filtrate was concentrated under reduced pressure to give 1-(tert-butyl) 2-methyl (2R,4R)-4-((4-fluoronaphthalen-1-yl)methyl)pyrrolidine-1,2-dicarboxylate (7.9 g, 20.39 mmol, 98% yield) as a light-yellow oil. MS ESI calculated for C₂₂H₂₇FNO₄ [M−Boc+H]⁺ 288.18. found 288.20.

Step 4: To a stirred solution of 1-(tert-butyl) 2-methyl (2R)-4-((4-fluoronaphthalen-1-yl)methyl)pyrrolidine-1,2-dicarboxylate (8.8 g, 22.71 mmol) in DCM (80 mL) was added TFA (80 mL) at room temperature. The solution was stirred at 25° C. for 1 h. The solvent was concentrated under reduced pressure to give a crude product. The crude product was separated by Prep-SFC with the following conditions: Column: Chiral Art Amylose-C NEO, 7*25 cm, 10 μm; Mobile Phase A: CO₂, Mobile Phase B: MeOH (0.1% 2 M NH₃-MeOH); Flow rate: 220 mL/min; Gradient: isocratic 30% B; Column Temperature (° C.): 35; Back Pressure (bar): 100; Wavelength: 220 nm; RT1 (min): 8.23; RT2 (min): 9.4; Sample Solvent: MeOH; Injection Volume: 2 mL; Number of Runs: 10. The fractions at 9.4 min were collected and concentrated under reduced pressure to give methyl (2R)-4-((4-fluoronaphthalen-1-yl)methyl)pyrrolidine-2-carboxylate (3.0 g, 10.44 mmol, 46% yield) as a light yellow solid. MS ESI calculated for C₁₇H₁₉FNO₂ [M+H]⁺ 288.13, found 288.20.

Step 5: To a stirred solution of methyl (2R,4R)-4-((4-fluoronaphthalen-1-yl)methyl)pyrrolidine-2-carboxylate (3 g, 10.44 mmol) in THF (30 mL) was added LiOH (20.88 mL, 20.88 mmol, 1 N in water) at room temperature. The solution was stirred at 25° C. for 2 h. The pH of the solution was adjusted to 7 with 1 N HCl. The solution was concentrated under reduced pressure to give (2R,4R)-4-((4-fluoronaphthalen-1-yl)methyl)pyrrolidine-2-carboxylic acid (2.85 g, 9.39 mmol, 90% yield) as a yellow oil. MS ESI calculated for C₁₆H₁₇FNO₂ [M+H]⁺ 274.12, found 274.10.

Step 6: To a stirred solution of (2R,4R)-4-((4-fluoronaphthalen-1-yl)methyl)pyrrolidine-2-carboxylic acid (2.85 g, 9.39 mmol) and NaHCO₃ (2.365 g, 28.2 mmol) in THF (30 mL) and water (30 mL) was added Fmoc-OSu (3.17 g, 9.39 mmol) at room temperature. The mixture was stirred at 25° C. for 16 h. The pH of the mixture was adjusted to 3 with 1 N HCl. The solution was extracted with EA (3×100 mL). The combined organic layer was washed with brine (100 mL), dried over anhydrous Na₂SO₄. After filtration, the filtrate was concentrated under reduced pressure and the residue was purified by RP-flash with the following conditions: 330 g C¹⁸ column, 5%-5% in 5 min, 5%-77% in 30 min, 98%-98% in 5 min, MeCN in water (0.05 TFA), RT=35 min to give (2R,4R)-1-(((9H-fluoren-9-yl)methoxy)carbonyl)-4-((4-fluoronaphthalen-1-yl)methyl)pyrrolidine-2-carboxylic acid (2.9229 g, 5.84 mmol, 62.2% yield) as an off-white solid. MS ESI calculated for C₃₁H₂₇FNO₄ [M+H]⁺ 496.18, found 496.05. ¹H NMR (300 MHz, CD₃OD) δ 8.11-8.09 (m, 2H), 7.77-7.45 (m, 5H), 7.37-7.10 (m, 7H), 4.52-4.11 (m, 4H), 3.31-3.00 (m, 4H), 2.44-2.42 (m, 2H), 1.88-1.85 (m, 1H).

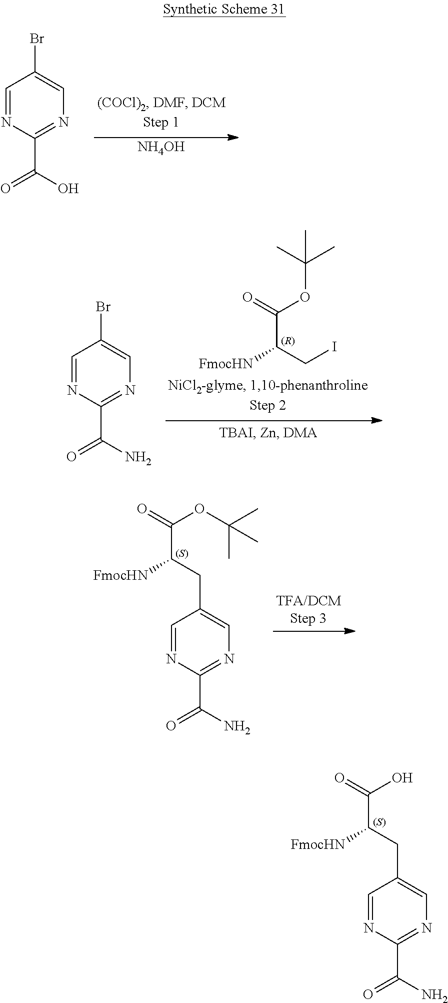

### Precursor to AlaPyrim4CONH2

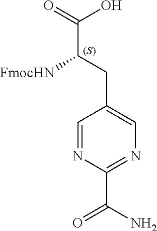

### (S)-2-((((9H-fluoren-9-yl)methoxy)carbonyl)amino)-3-(2-carbamoylpyrimidin-5-yl)propanoic acid

Step 1: To a stirred solution of 5-bromopyrimidine-2-carboxylic acid (4 g, 19.70 mmol) in DCM (80 mL) were added oxalyl chloride (5.00 g, 39.4 mmol) and DMF (0.153 mL, 1.970 mmol) at 0° C. The mixture was stirred at 25° C. for 3 h. Then ammonia aqueous (3.45 g, 99 mmol, 28%) was added to the solution and stirred at 25° C. for 1 h. The reaction was concentrated under reduced pressure and the residue was purified by RP-flash chromatography with the following conditions: Column: C¹⁸ gel column (330 g); Mobile Phase A: water (0.05% TFA); Mobile Phase B: MeCN; (Gradient: 0% B hold 5 min, up to 21% B within 15 min, 21% B hold 10 min; up to 95% B within 20 min, 95% B hold 10 min); Flow rate: 60 mL/min; Detector: UV 254 & 210 nm; RT: 20 min to afford 5-bromopyrimidine-2-carboxamide (2.2 g, 10.89 mmol, 55.3% yield) as a white solid. MS ESI calculated for C₅H₅BrN₃O [M+H]⁺ 201.95, 203.95 found 202.00, 204.00. ¹H NMR (300 MHz, DMSO-d₆) δ 9.13 (s, 2H), 8.21 (s, 1H), 7.86 (s, 1H).

Step 2: The mixture of nickel(II) chloride ethylene glycol dimethyl ether complex (0.457 g, 2.079 mmol) and 1,10-phenanthroline (0.375 g, 2.079 mmol) in DMA (20 mL) was heated at 50° C. for 0.5 hours. The solution of 5-bromopyrimidine-2-carboxamide (2.1 g, 10.40 mmol), tert-butyl (R)-2-((((9H-fluoren-9-yl)methoxy)carbonyl)amino)-3-iodopropanoate (5.13 g, 10.40 mmol) and TBAI (3.84 g, 10.40 mmol) in DMA (20 mL) was added at 25° C. Then zinc (1.359 g, 20.79 mmol) was added and stirred for 1 h at room temperature. The reaction was quenched with water (300 mL) at room temperature. The mixture was extracted with EA (3×300 mL). The organic layers were washed with brine (200 mL), dried over anhydrous Na₂SO₄ and filtered. The filtrate was concentrated under reduced pressure. The residue was purified by RP flash with the following conditions: Column: C¹⁸ gel column (330 g); Mobile Phase A: water (0.05% TFA); Mobile Phase B: MeCN; (Gradient: 0% B hold 5 min, up to 41% B within 20 min, 41% B hold 10 min; up to 95% B within 10 min, 95% B hold 5 min); Flow rate: 60 mL/min; Detector: UV 254 & 210 nm; RT: 25 min to give tert-butyl (S)-2-((((9H-fluoren-9-yl)methoxy)carbonyl)amino)-3-(2-carbamoylpyrimidin-5-yl)propanoate (1.6 g, 3.28 mmol, 31.5% yield) as a yellow solid. MS ESI calculated for C₂₇H₂₉N₄O₅ [M+H]⁺ 489.21, found 489.15.

Step 3: To a stirred solution of tert-butyl (S)-2-((((9H-fluoren-9-yl)methoxy)carbonyl)amino)-3-(2-carbamoylpyrimidin-5-yl)propanoate (2.1 g, 4.30 mmol) in DCM (20 mL) was added TFA (20 mL, 260 mmol) at room temperature. The solution was stirred at 25° C. for 4 h. The solvent was concentrated under reduced pressure and the residue was purified by RP-flash with the following conditions: Column: C¹⁸ gel column (330 g); Mobile Phase A: water (0.05% TFA); Mobile Phase B: MeCN; (Gradient: 0% B hold 5 min, up to 23% B within 15 min, 23% B hold 10 min; up to 95% B within 15 min, 95% B hold 10 min); Flow rate: 60 mL/min; Detector: UV 254 & 210 nm; RT: 30 min to give (S)-2-((((9H-fluoren-9-yl)methoxy)carbonyl)amino)-3-(2-carbamoylpyrimidin-5-yl)propanoic acid (1.13 g, 2.61 mmol, 60.8% yield) as an off-white solid. MS ESI calculated for C₂₃H₂₁N₄O₅ [M+H]⁺ 433.14, found 433.00. ¹H NMR (400 MHz, CD₃OD) δ 8.83 (s, 2H), 7.80 (d, J=7.6 Hz, 2H), 7.62-7.59 (m, 2H), 7.41-7.25 (m, 4H), 4.57-4.53 (m, 1H), 4.40-4.08 (m, 3H), 3.41-3.34 (m, 1H), 3.13-3.07 (m, 1H).

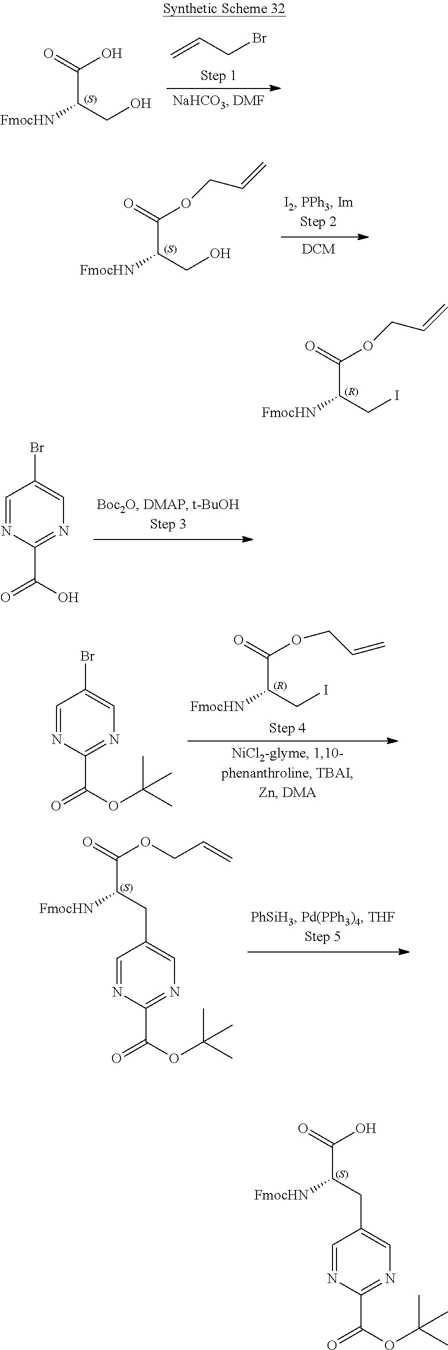

### Precursor to AlaPyrim4COOH

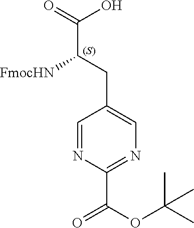

### (S)-2-((((9H-fluoren-9-yl)methoxy)carbonyl)amino)-3-(2-(tert-butoxycarbonyl)pyrimidin-5-yl)propanoic acid

Step 1: To a solution of (((9H-fluoren-9-yl)methoxy)carbonyl)-L-serine (10 g, 30.5 mmol) in DMF (150 mL) were added NaHCO₃ (12.83 g, 153 mmol), 3-bromoprop-1-ene (11.09 g, 92 mmol) at 0° C. under Ar atmosphere. The resulted mixture was stirred for 12 h at room temperature. The mixture was diluted with water (500 mL), extracted with EA (1000 mL). The combined organic layer was washed with brine (3×500 mL), dried over anhydrous Na₂SO₄ and filtered. The filtrate was concentrated under reduced pressure and the residue was purified by silica gel column chromatography, eluted with 0-50% EA in PE to give allyl (((9H-fluoren-9-yl)methoxy)carbonyl)-L-serinate (9.6 g, 26.1 mmol, 85% yield) as a colorless oil. MS ESI calculated for C₂₁H₂₁NO₅Na [M+Na]⁺390.14, found 390.20. ¹H NMR (300 MHz, CDCl₃) δ 7.80-7.21 (m, 8H), 5.99-5.67 (m, 2H), 5.40-5.19 (m, 2H), 4.69-4.67 (m, 2H), 4.43-4.41 (m, 3H), 4.22-4.20 (m, 1H), 4.04-3.79 (m, 2H), 2.17 (s, 1H).

Step 2: To a mixture of allyl (((9H-fluoren-9-yl)methoxy)carbonyl)-L-serinate (9.6 g, 26.1 mmol) in DCM (960 mL) were added imidazole (3.56 g, 52.3 mmol), Ph₃P (11.65 g, 44.4 mmol) and iodine (9.95 g, 39.2 mmol) sequentially at room temperature. The reaction was stirred at room temperature for 4 h. The resulting solution was quenched with saturated Na₂S₂O₃ (300 mL) and extracted with DCM (500 mL). The organic layers were combined, washed with brine (2×400 mL), dried over anhydrous Na₂SO₄ and filtered. The filtrate was concentrated under reduced pressure. The residue was purified by silica gel chromatography, eluting with a gradient of EA in PE from 0% to 25% to afford allyl (R)-2-((((9H-fluoren-9-yl)methoxy)carbonyl)amino)-3-iodopropanoate (6.3 g, 13.15 mmol, 50.3% yield) as a white solid. MS ESI calculated for C₂₁H₂₀INO₄Na [M+Na]⁺ 500.04, found 500.15. ¹H NMR (400 MHz, CDCl₃) δ 7.78-7.76 (m, 2H), 7.63-7.61 (m, 2H), 7.41-7.39 (m, 2H), 7.33-7.31 (m, 2H), 5.94-5.92 (m, 1H), 5.68-5.66 (m, 1H), 5.49-5.22 (m, 2H), 4.74-4.73 (m, 2H), 4.62-4.60 (m, 1H), 4.41-4.40 (m, 2H), 4.25-4.24 (m, 1H), 3.72-3.52 (m, 2H).

Step 3: To a mixture of 5-bromopyrimidine-2-carboxylic acid (5 g, 24.63 mmol) in t-BuOH (75 mL) were added DMAP (0.301 g, 2.463 mmol) and Boc₂O (6.45 g, 29.6 mmol) under argon at room temperature. The reaction was stirred at 50° C. for 16 h. The reaction was cooled to room temperature and concentrated in vacuum. The residue was purified by silica gel column chromatography, eluted with 0-20% EA in PE to give tert-butyl 5-bromopyrimidine-2-carboxylate (3.55 g, 13.58 mmol, 55.1% yield) as a white solid. MS ESI calculated for C₉H₁₁BrN₂O₂ [M−tBu+H]⁺ 203.00, 205.00, found 202.95, 204.95.

Step 4: The mixture of Nickel (II) chloride ethylene glycol dimethyl ether complex (63.6 mg, 0.289 mmol) and pyridine-2-carboximidamide hydrochloride (91 mg, 0.579 mmol) in DMA (20 mL) was heated at 50° C. for 1 hours. The solution of allyl (R)-2-((((9H-fluoren-9-yl)methoxy)carbonyl)amino)-3-iodopropanoate (1.38 g, 2.89 mmol), tert-butyl 5-bromopyrimidine-2-carboxylate (750 mg, 2.89 mmol) and TBAI (2.14 g, 5.79 mmol) in DMA (25 mL) was added at 25° C. Then zinc (378 mg, 5.79 mmol) was added and stirred for 2 h at 25° C. The reaction was quenched with H₂O (200 mL) and extracted with EA (2×400 mL). The combined organic layer was washed with brine (3×200 mL), dried over anhydrous Na₂SO₄ and filtered. The filtrate was concentrated under reduced pressure. The residue was purified by silica gel column chromatography, eluted with 0%-45% EA in PE to afford tert-butyl (S)-5-(2-((((9H-fluoren-9-yl)methoxy)carbonyl)amino)-3-(allyloxy)-3-oxopropyl)pyrimidine-2-carboxylate (570 mg, 1.022 mmol, 35.3% yield) as a colorless semi-solid. MS ESI calculated for C₃₀H₃₂N₃O₆ [M+H]⁺ 530.22, found 530.25.

Step 5: To a stirred solution of tert-butyl (S)-5-(2-((((9H-fluoren-9-yl)methoxy)carbonyl)amino)-3-(allyloxy)-3-oxopropyl)pyrimidine-2-carboxylate (1.44 g, 2.72 mmol) in THF (25 mL) were added Pd(PPh₃)₄ (0.157 g, 0.136 mmol) and phenylsilane (0.588 g, 5.44 mmol) at room temperature. The resulting mixture was stirred at 25° C. for 1 h. The solvent was concentrated under reduced pressure and the residue was purified by RP-flash with the following conditions: 330 g C¹⁸ column, 2%-2% in 5 min, 2%-50% in 30 min, MeCN in water (0.05% TFA) to give (S)-2-((((9H-fluoren-9-yl)methoxy)carbonyl)amino)-3-(2-(tert-butoxycarbonyl)pyrimidin-5-yl)propanoic acid (1.01 g, 2.051 mmol, 75% yield) as a white soild. MS ESI calculated for C₂₇H₂₈N₃O₆ [M+H]⁺ 490.19, found 490.15. ¹H NMR (300 MHz, DMSO-d₆) δ 8.86 (s, 2H), 7.86-7.85 (m, 3H), 7.69-7.52 (m, 2H), 7.46-7.24 (m, 4H), 4.33-4.32 (m, 1H), 4.20-4.18 (m, 3H), 3.23-3.21 (m, 1H), 2.98-2.97 (m, 1H), 1.55 (s, 9H).

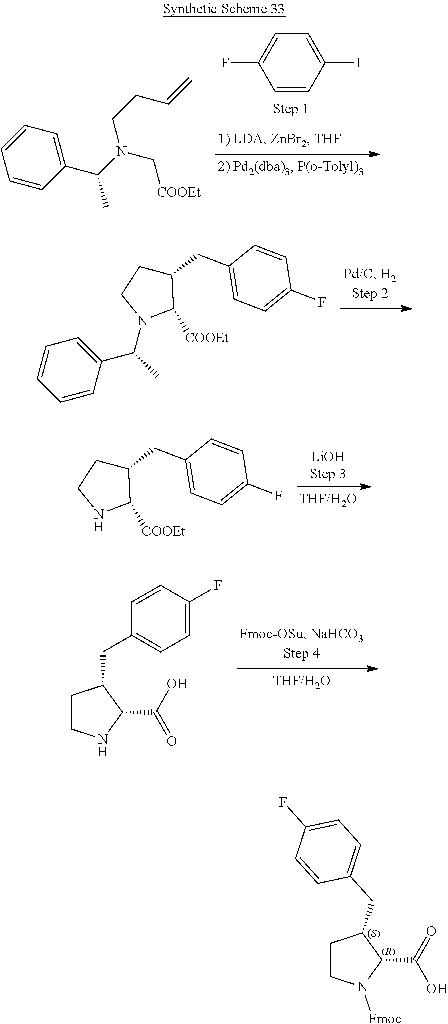

### Precursor to dProc3Bn4F

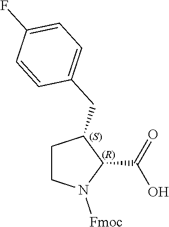

### (2R,3S)-1-(((9H-fluoren-9-yl)methoxy)carbonyl)-3-(4-fluorobenzyl)pyrrolidine-2-carboxylic acid

Step 1: To a solution of ethyl (R)—N-(but-3-en-1-yl)-N-(1-phenylethyl)glycinate (5 g, 19.13 mmol) in THF (50 mL) was added LDA (9.57 mL, 19.13 mmol, 2 N in THF) at −78° C. The resulted solution was stirred at −78° C. for 1 h. A solution of zinc (II) bromide (12.92 g, 57.4 mmol) in THF (50 mL) was added at −78° C. The resulted mixture was stirred for 1 h. at room temperature. Tris(dibenzylideneacetone)dipalladium (0) (0.526 g, 0.574 mmol), tri-o-tolylphosphane (0.757 g, 2.487 mmol) and 1-fluoro-4-iodobenzene (5.52 g, 24.87 mmol) were added at room temperature. The resulted mixture was stirred for 16 h. at room temperature. The reaction was quenched with aqueous NH₄Cl (2 M, 50 mL), and extracted with EA (3×200 mL). The combined organic layer was washed with brine (3×60 mL), dried over Na₂SO₄ and filtered. The filtrate was concentrated under vacuum to afford a yellow oil. The residue was purified by silica gel column chromatography, eluting with EA/PE (1/10) to afford ethyl (2R,3S)-3-(4-fluorobenzyl)-1-((R)-1-phenylethyl)pyrrolidine-2-carboxylate (2.5 g, 4.57 mmol, 23.9% yield) as a yellow oil. MS ESI calculated for C₂₂H₂₇FNO₂ [M+H]⁺ 356.19, found 356.25.

Step 2: To a solution of ethyl (2R,3S)-3-(4-fluorobenzyl)-1-((R)-1-phenylethyl)pyrrolidine-2-carboxylate (2 g, 5.63 mmol) in t-BuOH (40 mL) was added Pd/C (0.599 g, 5.63 mmol, dry, 10% wt) at room temperature. The reaction mixture was degassed with nitrogen for 3 times and stirred under hydrogen for 3 h at room temperature. The mixture was filtered. The filter cake was washed with MeOH (3×100 mL). The filtrate was concentrated under reduced pressure. The residue was purified by RP-flash with the following conditions: 330 g C¹⁸ column, 2%-2% in 5 mins, 2%-30% in 25 mins, 98%-98% in 5 mins, MeCN in water (0.05% TFA), RT=30 min to afford ethyl (2R,3S)-3-(4-fluorobenzyl)pyrrolidine-2-carboxylate (0.6 g, 2.388 mmol, 42.4% yield) as a colorless oil. MS ESI calculated for C₁₄H₁₉FNO₂ [M+H]⁺ 252.13, found 252.25. ¹H NMR (400 MHz, DMSO-d₆) 7.28-7.25 (m, 2H), 7.17-7.12 (m, 2H), 4.45 (d, J=7.6 Hz, 1H), 4.25-4.15 (m, 2H), 3.46-3.39 (m, 1H), 3.21-3.18 (m, 1H), 2.84-2.72 (m, 2H), 2.44-2.38 (m, 1H), 1.94-1.93 (m, 1H), 1.68-1.64 (m, 1H), 1.26-1.22 (m, 3H).

Step 3: To a mixture of ethyl (2R,3S)-3-(4-fluorobenzyl)pyrrolidine-2-carboxylate (2 g, 7.96 mmol) in THF (20 mL) was added LiOH (23.88 mL, 23.88 mmol, 1 N in water) at room temperature. The resulted mixture was stirred for 18 h. at room temperature. The pH value of the reaction solution was adjusted to 7 with HCl (1 M). The mixture was used directly to next step. MS ESI calculated for C₁₂H₁₅FNO₂ [M+H]⁺ 224.10, found 224.05.

Step 4: To a mixture of (2R,3S)-3-(4-fluorobenzyl)pyrrolidine-2-carboxylic acid (5 g, 22.40 mmol), NaHCO₃ (5.64 g, 67.2 mmol) in THF (40 mL) and water (40 mL) was added Fmoc-OSu (7.56 g, 22.40 mmol) at room temperature. The resulted mixture was stirred for 12 h. at room temperature. The pH value of the reaction solution was adjusted to 5 with HCl (0.1 M). The reaction was extracted with EA (3×100 mL). The combined organic layer was washed with brine (3×150 mL), dried over Na₂SO₄ and filtered. The filtrate was concentrated under vacuum to afford a pink oil. The residue was purified by RP-flash with the following conditions: 330 g C¹⁸ column, 2%-2% in 5 mins, 2%-500% in 30 mins, 98%-98% in 5 mins, MeCN in water (0.05% TFA), RT=35 min to afford 10.7 g of crude product. The crude product was separated by SFC with the following conditions: Column: Chiralpak IH, 3*25 cm, 5 μm; Mobile Phase A: CO₂, Mobile Phase B: MeOH; Flow rate: 120 mL/min; Gradient: isocratic 30% B; Column Temperature (° C.): 35; Back Pressure (bar): 100; Wavelength: 220 nm; RT1 (min): 3.13; RT2 (min): 3.72; Sample Solvent: MeOH:ACN:DCM=1:1:1 (0.1% DEA); Injection Volume: 3 mL; Number of Runs: 82. The fractions at 3.13 min were collected and concentrated under reduced pressure to afford (2R,3S)-1-(((9H-fluoren-9-yl)methoxy)carbonyl)-3-(4-fluorobenzyl)pyrrolidine-2-carboxylic acid (5.1912 g, 11.65 mmol, 52% yield) as an off-white solid. MS ESI calculated for C₂₇H₂₅FNO₄ [M+H]⁺ 446.17, found 446.10. ¹H NMR (400 MHz, DMSO-d₆) δ 7.93-7.88 (m, 2H), 7.70-7.64 (m, 2H), 7.44-7.40 (m, 2H), 7.35-7.26 (m, 4H), 7.13-7.08 (m, 2H), 4.42-4.15 (m, 4H), 3.56-3.53 (m, 1H), 3.29-3.24 (m, 1H), 3.02-2.91 (m, 1H), 2.51-2.50 (m, 1H), 2.34-2.29 (m, 1H), 1.74-1.70 (m, 2H).

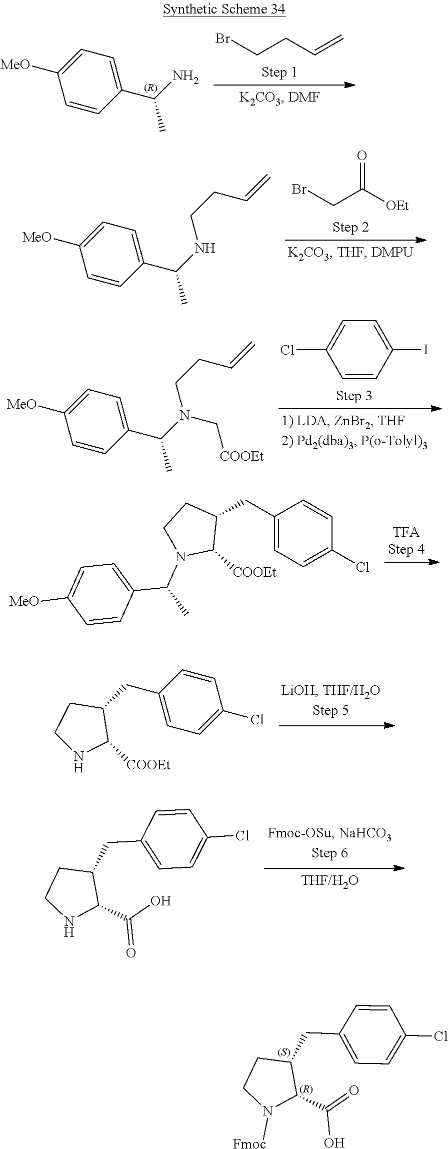

### Precursor to dProc3Bn4C₁

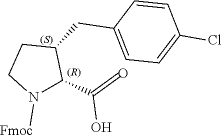

### (2R,3S)-1-(((9H-fluoren-9-yl)methoxy)carbonyl)-3-(4-chlorobenzyl)pyrrolidine-2-carboxylic acid

Step 1: To a solution of (R)-1-(4-methoxyphenyl)ethan-1-amine (10 g, 66.1 mmol) in DMF (170 mL) was added K₂CO₃ (18.28 g, 132 mmol) at room temperature and the resulting mixture was stirred for 10 min at room temperature. Then 4-bromobut-1-ene (8.04 g, 59.5 mmol) was added. After the resulting mixture was stirred for 18 h at room temperature, it was diluted with water (300 mL) and extracted with EA (3×300 mL). The combined organic layer was washed with brine (3×150 mL), dried over Na₂SO₄ and filtered. The filtrate was concentrated under vacuum. The residue was purified by silica gel column chromatography, eluting with EA/PE (2/3) to afford (R)—N-(1-(4-methoxyphenyl)ethyl)but-3-en-1-amine (8.00 g, 39.0 mmol, 58.9% yield) as a colorless oil. MS ESI calculated for C₁₃H₂₀NO [M+H]⁺ 206.15, found 206.20.

Step 2: To a mixture of (R)—N-(1-(4-methoxyphenyl)ethyl)but-3-en-1-amine (10 g, 48.7 mmol) in THF (40 mL) and DMPU (40 mL) were added ethyl 2-bromoacetate (8.13 g, 48.7 mmol) and K₂CO₃ (7.40 g, 53.6 mmol) at room temperature. The resulting mixture was stirred for 18 h at room temperature. The reaction was diluted with water (100 mL) and extracted with EA (3×100 mL). The combined organic layer was washed with brine (3×150 mL), dried over Na₂SO₄ and filtered. The filtrate was concentrated under vacuum. The residue was purified by silica gel column chromatography, eluting with EA/PE (1/5) to afford ethyl (R)—N-(but-3-en-1-yl)-N-(1-(4-methoxyphenyl)ethyl)glycinate (10 g, 34.3 mmol, 70.5% yield) as a colorless oil. MS ESI calculated for C₁₇H₂₆NO₃ [M+H]⁺ 292.18, found 292.25. ¹H NMR (400 MHz, CDCl₃) δ 7.28 (d, J=8.4 Hz, 2H), 6.84 (d, J=8.4 Hz, 2H), 5.81-5.70 (m, 1H), 5.04-5.03 (m, 1H), 4.99-4.94 (m, 1H), 4.16-4.10 (m, 2H), 4.00-3.97 (m, 1H), 3.80 (s, 3H), 3.40 (d, J=17.2 Hz, 1H), 3.25 (d, J=17.2 Hz, 1H), 2.68-2.64 (m, 2H), 2.22-2.16 (m, 2H), 1.33-1.32 (m, 3H), 1.27-1.23 (m, 3H).

Step 3: To a solution of ethyl (R)—N-(but-3-en-1-yl)-N-(1-(4-methoxyphenyl)ethyl)glycinate (2.6 g, 8.92 mmol) in THF (20 mL) was added LDA (6.69 mL, 13.38 mmol, 2 N in THF) at −78° C. The resulting mixture was stirred for 1 h at −78° C. The solution of Zinc (II) bromide (6.03 g, 26.8 mmol) in THF (20 mL) was added at −78° C. After the resulting mixture was stirred for 1 h at room temperature, Pd₂(dba)₃ (0.245 g, 0.268 mmol), P(o-Tolyl)₃ (0.353 g, 1.160 mmol) and 1-chloro-4-iodobenzene (2.77 g, 11.60 mmol) were added at room temperature. The resulting mixture was stirred for 16 h at room temperature. The reaction was quenched with aqueous NH₄Cl (2 M, 50 mL), and extracted with EA (3×200 mL). The combined organic layer was washed with brine (3×160 mL), dried over Na₂SO₄ and filtered. The filtrate was concentrated under vacuum. The residue was purified by a silica gel column chromatography, eluting with EA/PE (1/10) to afford ethyl (2R,3S)-3-(4-chlorobenzyl)-1-((R)-1-(4-methoxyphenyl)ethyl)pyrrolidine-2-carboxylate (1.20 g, 2.99 mmol, 33.5% yield) as a yellow oil. MS ESI calculated for C₂₃H₂₉ClNO₃ [M+H]⁺ 402.18, found 402.25.

Step 4: A solution of ethyl (2R,3S)-3-(4-chlorobenzyl)-1-((R)-1-(4-methoxyphenyl)ethyl)pyrrolidine-2-carboxylate (1 g, 2.488 mmol) in TFA (30 mL) was stirred for 18 h at 80° C. The reaction was cooled to room temperature and concentrated under vacuum. The residue was purified by RP-flash with the following conditions: C¹⁸ column, 330 g, 2%-2% in 5 min, 2%-30% in 20 min, 98%-98% in 5 min, MeCN in water (0.05% TFA) to afford ethyl (2R,3S)-3-(4-chlorobenzyl)pyrrolidine-2-carboxylate (0.3 g, 1.120 mmol, 45% yield) as a yellow oil. MS ESI calculated for C₁₄H₁₉ClNO₂ [M+H]⁺ 268.10, found 268.15.

Step 5: To a solution of ethyl (2R,3S)-3-(4-chlorobenzyl)pyrrolidine-2-carboxylate (4.3 g, 16.06 mmol) in THF (50 mL) was added a solution of LiOH (1.154 g, 48.2 mmol) in water (50 mL) at room temperature. The resulting mixture was stirred for 18 h at room temperature. The pH value of the reaction solution was adjusted to 7.0 with HCl (1 M). The resulting mixture was used directly in next step. MS ESI calculated for C₁₂H₁₅ClNO₂ [M+H]⁺ 240.07, found 240.10.

Step 6: To a stirred solution of (2R,3S)-3-(4-chlorobenzyl)pyrrolidine-2-carboxylic acid (5.5 g, 22.95 mmol) in THF (100 mL) and water (100 mL) were added NaHCO₃ (5.78 g, 68.8 mmol) and Fmoc-OSu (7.74 g, 22.95 mmol) at 25° C. The resulting mixture was stirred at 25° C. for 2 h. The pH of the mixture was adjusted to 3 with 1 N HCl then extracted with EA (3×200 mL). The organic fractions were washed with brine (2×100 mL), dried over Na₂SO₄ and filtered. The filtrate was concentrated under vacuum. The residue was purified by RP flash with the following conditions: C¹⁸ column, 330 g, 2%-2% in 5 min. 2%-60% in 40 min. 98%-98% in 5 min. MeCN in water (0.05% TFA) to afford crude product. The crude product was separated by SFC with the following conditions: Column: Chiralpak IG 3*25 cm, 5 μm; Mobile Phase A: CO₂, Mobile Phase B: MeOH; Flow rate: 110 mL/min; Gradient: isocratic 50% B; Back Pressure (bar): 100; Wavelength: 254 nm; RT1 (min): 10.1; RT2 (min): 16.8; Sample Solvent: DCM/MeOH=1:1 (0.1% AcOH); Injection Volume: 5 mL; Number of Runs: 30. The fractions at 10.1 min were collected and concentrated under reduced pressure to give (2R,3S)-1-(((9H-fluoren-9-yl)methoxy)carbonyl)-3-(4-chlorobenzyl)pyrrolidine-2-carboxylic acid (7.8731 g, 16.87 mmol, 73.5% yield) as an off-white solid. MS ESI calculated for C₂₇H₂₃ClNO₄ [M−H]⁻ 460.14, found 460.05. ¹H NMR (400 MHz, DMSO-d₆) δ 12.86 (s, 1H), 7.90 (d, J=7.5 Hz, 2H), 7.68-7.65 (m, 2H), 7.44-7.27 (m, 8H), 4.42-4.17 (m, 4H), 3.56-3.30 (m, 1H), 3.28-3.26 (m, 1H), 2.95-2.88 (m, 1H), 2.51 (s, 1H), 2.35-2.31 (m, 1H), 1.75-1.69 (m, 2H).

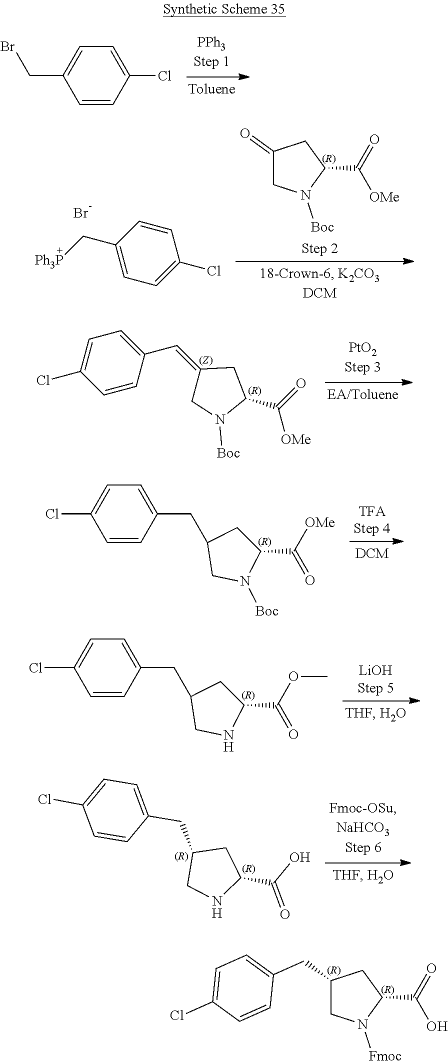

### Precursor to dProc4Bn4Cl

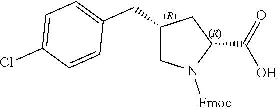

### (2R,4R)-1-(((9H-fluoren-9-yl)methoxy)carbonyl)-4-(4-chlorobenzyl)pyrrolidine-2-carboxylic acid

Step 1: To a mixture of 1-(bromomethyl)-4-chlorobenzene (10 g, 48.7 mmol) in toluene (100 mL) was added triphenylphosphane (14.04 g, 53.5 mmol) at ambient temperature. The resulted mixture was stirred for 6 h at 110° C. then cooled down to ambient temperature. The solid was collected by filtration to afford (4-chlorobenzyl)triphenylphosphonium bromide (18 g, 38.5 mmol, 79% yield) as a white solid. MS ESI calculated for C₂₅H₂₁ClP [M−Br]⁺ 387.11, found 387.15. ¹H NMR (300 MHz, CDCl₃) δ 7.82-7.73 (m, 9H), 7.64-7.58 (m, 6H), 7.16-7.12 (m, 2H), 7.04 (d, J=8.1 Hz, 2H), 5.61 (d, J=14.7 Hz, 2H).

Step 2: To a mixture of (4-chlorobenzyl)triphenylphosphonium bromide (40.9 g, 87 mmol), K₂CO₃ (12.07 g, 87 mmol) in DCM (400 mL) was added 18-crown-6 (0.462 g, 1.747 mmol) at room temperature. The resulted mixture was stirred for 1 h at room temperature. 1-(Tert-butyl) 2-methyl (R)-4-oxopyrrolidine-1,2-dicarboxylate (8.5 g, 34.9 mmol) was added and the resulted mixture was stirred for 48 h at 48° C. The reaction was cooled down to ambient temperature, then diluted with water (200 mL) and extracted with EA (3×200 mL). The combined organic layer was washed with brine (3×150 mL), dried over Na₂SO₄ and filtered. The filtrate was concentrated under vacuum. The residue was purified by silica gel column chromatography, eluting with EA/PE (1/6) to afford 1-(tert-butyl) 2-methyl (R,Z)-4-(4-chlorobenzylidene)pyrrolidine-1,2-dicarboxylate (7.0 g, 19.90 mmol, 56.9% yield) as a yellow solid. MS ESI calculated for C₁₈H₂₃ClNO₄ [M+H]⁺ 352.12, found 352.15. ¹H NMR (400 MHz, CDCl₃) δ 7.34-7.26 (m, 2H), 7.16-7.09 (m, 2H), 6.42-6.33 (m, 1H), 4.63-4.26 (m, 3H), 3.71 (s, 3H), 3.19-3.12 (m, 1H), 2.91-2.77 (m, 1H), 1.47-1.43 (m, 9H).

Step 3: To a mixture of 1-(tert-butyl) 2-methyl (R,Z)-4-(4-chlorobenzylidene)pyrrolidine-1,2-dicarboxylate (1 g, 2.84 mmol) in EA (10 mL) and toluene (10 mL) was added PtO₂ (0.065 g, 0.284 mmol) at room temperature. The reaction mixture was degassed with hydrogen for 3 times and stirred under hydrogen for 16 h at ambient temperature. The reaction was filtered and the filtrate was concentrated under vacuum. The residue was purified by silica gel column chromatography, eluting with EA/PE (1/5) to afford 1-(tert-butyl) 2-methyl (2R,4R)-4-(4-chlorobenzyl)pyrrolidine-1,2-dicarboxylate (0.7 g, 1.978 mmol, 69.6% yield) as a colorless oil. MS ESI calculated for C₁₈H₂₅ClNO₄ [M+H]⁺ 354.14, found 354.15. ¹H NMR (400 MHz, CDCl₃) δ 7.30-7.26 (m, 2H), 7.11-7.08 (m, 2H), 4.28-4.15 (m, 1H), 3.76-3.60 (m, 4H), 3.17-3.12 (m, 1H), 2.75-2.60 (m, 2H), 2.47-2.33 (m, 2H), 2.08-2.05 (m, 1H), 1.48-1.41 (m, 9H).

Step 4: To a solution of 1-(tert-butyl) 2-methyl (2R,4R)-4-(4-chlorobenzyl)pyrrolidine-1,2-dicarboxylate (1 g, 2.83 mmol) in DCM (10 mL) was added TFA (2 mL) at room temperature. The resulted mixture was stirred for 2 h at room temperature then concentrated under vacuum to afford colorless oil which was used directly in next step. MS ESI calculated for C₁₃H₁₇ClNO₂ [M+H]⁺ 254.09, found 254.10.

Step 5: To a mixture of methyl (2R,4R)-4-(4-chlorobenzyl)pyrrolidine-2-carboxylate (1 g, 3.94 mmol) in THF (10 mL) was added a mixture of LiOH (0.283 g, 11.82 mmol) in water (10 mL) at room temperature. The resulted mixture was stirred for 18 h at room temperature. The pH value of the reaction solution was adjusted to 7.0 with HCl (1 M) and then used directly in next step. MS ESI calculated for C₁₂H₁₅ClNO₂ [M+H]⁺ 240.07, found 240.05.

Step 6: To a mixture of (2R,4R)-4-(4-chlorobenzyl)pyrrolidine-2-carboxylic acid (9 g, 37.5 mmol) in water (140 mL) and THF (140 mL) were added Fmoc-OSu (12.67 g, 37.5 mmol) and NaHCO₃ (15.77 g, 188 mmol) at room temperature. The resulted mixture was stirred for 2 h at room temperature. The pH value of the reaction solution was adjusted to 3 with HCl (1M), extracted with EA (3×300 mL). The combined organic layer was washed with brine (3×100 mL), dried over Na₂SO₄ and filtered. The filtrate was concentrated under vacuum to afford yellow oil. The residue was purified by RP-flash with the following conditions: C18 column, 330 g, 2-2% in 5 min, 2%-50% in 30 min, 98%-98% in 5 min, MeCN in water (0.05% TFA) to afford 14 g crude product. The crude product was separated by SFC with the following conditions: Column: Chiralpak IG 3*25 cm, 5 μm; Mobile Phase A: CO₂. Mobile Phase B: MeOH:MeCN=1:1; Flow rate: 100 mL/min; Gradient: isocratic 45% B; Column Temperature (° C.): 35; Back Pressure (bar): 100; RT1 (min): 4; RT2 (min): 5.43; Sample Solvent: MeOH; Injection Volume: 2 mL; Number of Runs: 15. The fractions at 5.43 min were collected and concentrated under reduced pressure to afford (2R,4R)-1-(((9H-fluoren-9-yl)methoxy)carbonyl)-4-(4-chlorobenzyl)pyrrolidine-2-carboxylic acid (7.6433 g, 16.55 mmol, 44.1% yield) as an off-white solid. MS ESI calculated for C₂₇H₂₃ClNO₄ [M−H]⁻ 460.14, found 460.05. ¹H NMR (300 MHz, DMSO-d₆) δ 7.88-7.70 (m, 2H), 7.68-7.57 (m, 2H), 7.43-7.19 (m, 8H), 4.27-4.24 (m, 2H), 4.15-4.01 (m, 2H), 3.52-3.36 (m, 1H), 3.05-3.00 (m, 1H), 2.68-2.50 (m, 2H), 2.37-2.25 (m, 2H), 1.72-1.42 (m, 1H).

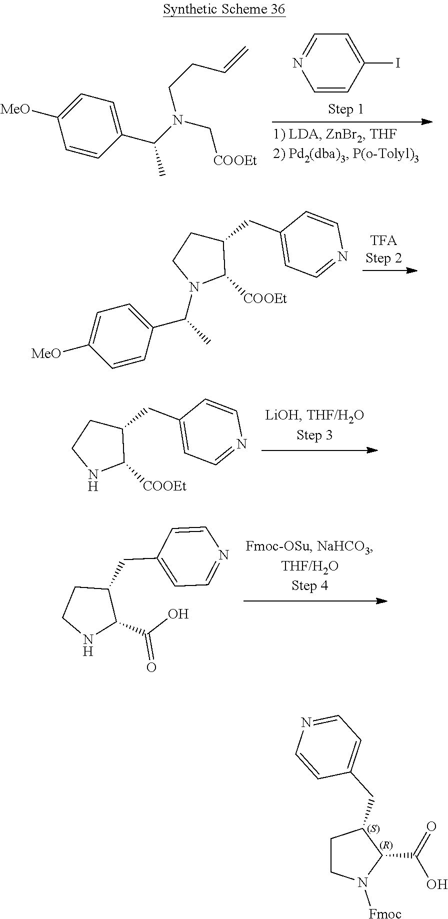

### Precursor to dProc3CH24Pal

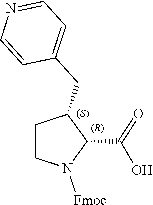

### (2R,3S)-1-(((9H-fluoren-9-yl)methoxy)carbonyl)-3-(pyridin-4-ylmethyl)pyrrolidine-2-carboxylic acid

Step 1: To a solution of ethyl (R)—N-(but-3-en-1-yl)-N-(1-(4-methoxyphenyl)ethyl)glycinate (5 g, 17.16 mmol) in THF (50 mL) was added LDA (8.58 mL, 17.16 mmol, 2 N in THF) at −78° C. The resulting mixture was stirred for 1 h at −78° C. The solution of Zinc(II) bromide (11.59 g, 51.5 mmol) in THF (50 mL) was added at −78° C. After the resulting mixture was stirred for 1 h at room temperature, Pd₂(dba)₃ (0.471 g, 0.515 mmol), P(o-Tolyl)₃ (0.679 g, 2.231 mmol) and 4-iodopyridine (4.57 g, 22.31 mmol) were added at room temperature. After the resulting mixture was stirred for 18 h at room temperature, it was quenched with aqueous NH₄Cl (2 M, 150 mL) and extracted with EA (3×200 mL). The combined organic layer was washed with brine (3×150 mL), dried over Na₂SO₄ and filtered. The filtrate was concentrated under vacuum. The residue was purified by silica gel column chromatography, eluting with MeOH/DCM (1/4) to afford ethyl (2R,3S)-1-((R)-1-(4-methoxyphenyl)ethyl)-3-(pyridin-4-ylmethyl)pyrrolidine-2-carboxylate (2.0 g, 5.43 mmol, 31.6% yield) as a yellow oil. MS ESI calculated for C₂₂H₂₉N₂O₃ [M+H]⁺ 369.21, found 369.15.

Step 2: A mixture of ethyl (2R,3S)-1-((R)-1-(4-methoxyphenyl)ethyl)-3-(pyridin-4-ylmethyl)pyrrolidine-2-carboxylate (4 g, 10.86 mmol) in TFA (80 mL) was stirred for 12 h at 80° C. The reaction was cooled to room temperature and concentrated under vacuum. The residue was purified by RP-flash with the following conditions: C¹⁸ column, 330 g, 2%-2% in 5 min, 2%-30% in 20 min, 98%-98% in 5 min, MeCN in water (0.05% TFA) to afford ethyl (2R,3S)-3-(pyridin-4-ylmethyl)pyrrolidine-2-carboxylate (1.2 g, 5.12 mmol, 47.2% yield) as a yellow oil. MS ESI calculated for C₁₃H₁₉N₂O₂ [M+H]⁺ 235.14, found 235.10.

Step 3: To a solution of ethyl (2R,3S)-3-(pyridin-4-ylmethyl)pyrrolidine-2-carboxylate (2 g, 8.54 mmol) in THF (30 mL) was added a solution of LiOH (0.613 g, 25.6 mmol) in water (30 mL) at room temperature. The resulting mixture was stirred for 18 h at room temperature. The pH value of the reaction solution was adjusted to 7.0 with HCl (1 M) and used directly in next step. MS ESI calculated for C₁₁H₁₅N₂O₂ [M+H]⁺ 207.11, found 207.00.

Step 4: To a mixture of (2R,3S)-3-(pyridin-4-ylmethyl)pyrrolidine-2-carboxylic acid (6 g, 29.1 mmol) in THF (60 mL) and water (60 mL) were added Fmoc-OSu (9.81 g, 29.1 mmol) and NaHCO₃ (12.22 g, 145 mmol) at room temperature. The resulting mixture was stirred for 2 h at room temperature. The pH value of the reaction solution was adjusted to 5 with HCl (1 M) then extracted with EA (3×200 mL). The combined organic layer was washed with brine (3×50 mL), dried over Na₂SO₄ and filtered. The filtrate was concentrated under vacuum. The residue was purified by RP flash with the following conditions: C¹⁸ column, 330 g, 2%-2% in 5 min, 2%-50% in 40 min, 98%-98% in 5 min. MeCN in water (0.05% TFA) to afford a crude product. The crude product was separated by SFC with the following conditions: Column: Chiral art Cellulose-SZ 3*25 cm, 5 μm; Mobile Phase A: CO₂, Mobile Phase B: EtOH; Flow rate: 100 mL/min; Gradient: isocratic 10% B; Column Temperature (° C.): 35; Back Pressure (bar): 100; Wavelength: 220 nm; RT1 (min): 4.88; RT2 (min): 6.55; Sample Solvent: MeOH; Injection Volume: 1.8 mL. The fractions at 6.55 min were collected and concentrated under reduced pressure to give (2R,3S)-1-(((9H-fluoren-9-yl)methoxy)carbonyl)-3-(pyridin-4-ylmethyl)pyrrolidine-2-carboxylic acid (7.5851 g, 17.52 mmol, 60.2% yield) as a yellow solid. MS ESI calculated for C₂₆H₂₅N₂O₄ [M+H]⁺ 429.17, found 429.10. ¹H NMR (400 MHz, CD₃OD) δ 8.76-8.73 (m, 2H), 8.00-7.98 (m, 2H), 7.82-7.78 (m, 2H), 7.66-7.61 (m, 2H), 7.43-7.39 (m, 2H), 7.34-7.30 (m, 2H), 4.44-4.19 (m, 4H), 3.70-3.68 (m, 1H), 3.42-3.34 (m, 1H), 3.33-3.32 (m, 1H), 3.32-3.21 (m, 2H), 2.93-1.94 (m, 2H).

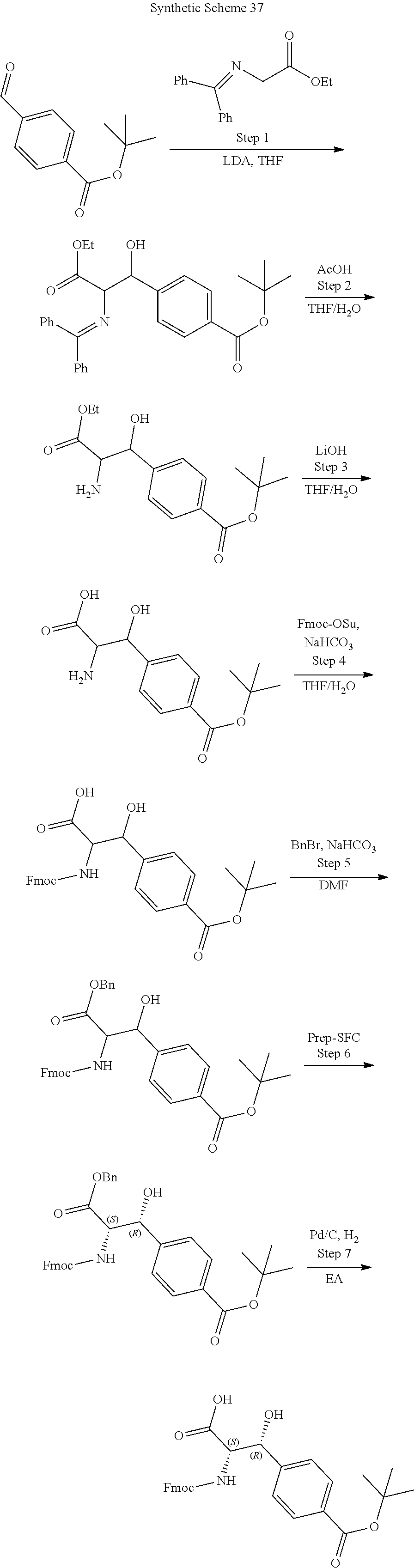

### Precursor to orbOHorPhe4COOH

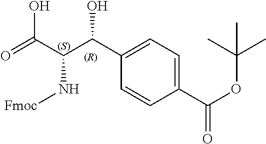

### (2S,33R)-2-((((9H-fluoren-9-yl)methoxy)carbonyl)amino)-3-(4-(tert-butoxycarbonyl)phenyl)-3-hydroxypropanoic acid

Step 1: The ethyl 2-((diphenylmethylene)amino)acetate (19.44 g, 72.7 mmol) was dissolved in THF (150 mL). The mixture was cooled to −70° C. and added LDA (36.4 mL, 72.7 mmol, 2 M in THF) under argon. The reaction was stirred at −70° C. for 1 h. And then to the mixture was added tert-butyl 4-formylbenzoate (10 g, 48.5 mmol) under argon at −70° C. The reaction was stirred at −70° C. for 2 h. The resulting solution was quenched with aqueous NH₄Cl (100 mL) and extracted with EA (3×500 mL). The organic layers were combined, washed with brine (2×300 mL), dried over anhydrous Na₂SO₄ and filtered. The residue was concentrated in vacuum to afford tert-butyl 4-(2-((diphenylmethylene)amino)-3-ethoxy-1-hydroxy-3-oxopropyl)benzoate (30 g, 41.2 mmol, 85% yield) as an orange semi-solid. MS ESI calculated for C₂₉H₃₂NO₅ [M+H]⁺ 474.22. found 474.30.

Step 2: To a mixture of tert-butyl 4-(2-((diphenylmethylene)amino)-3-ethoxy-1-hydroxy-3-oxopropyl)benzoate (30 g, 41.2 mmol) in THF (200 mL) and water (200 mL) was added AcOH (47.1 mL, 824 mmol) at ambient temperature. The reaction was stirred at ambient temperature for 16 h. The resulting solution was extracted with EtOAc (3×300 mL). The aqueous layers were combined and concentrated in vacuum to afford tert-butyl 4-(2-amino-3-ethoxy-1-hydroxy-3-oxopropyl)benzoate (16.8 g, 38.0 mmol, 92% yield) as a white solid. MS ESI calculated for C₁₆H₂₄NO₅ [M+H]⁺ 310.16, found 310.10.

Step 3: To a mixture of tert-butyl 4-(2-amino-3-ethoxy-1-hydroxy-3-oxopropyl)benzoate (16.8 g, 38.0 mmol) in THF (152 mL) and water (76 mL) was added LiOH (1 M, 76 mL, 76 mmol) at ambient temperature. The reaction was stirred at ambient temperature for 2 h. The resulting solution was acidified to pH 5 with diluted HCl (1 M) and concentrated in vacuum to afford 2-amino-3-(4-(tert-butoxycarbonyl)phenyl)-3-hydroxypropanoic acid (21.5 g, 37.5 mmol, 99% yield) as a white semi-solid. MS ESI calculated for C₁₄H₂₀NO₅ [M+H]⁺ 282.13, found 282.10.

Step 4: To a mixture of 2-amino-3-(4-(tert-butoxycarbonyl)phenyl)-3-hydroxypropanoic acid (21.5 g, 37.5 mmol) in THF (200 mL) and water (200 mL) were added NaHCO₃ (15.73 g, 187 mmol) and Fmoc-OSu (11.37 g, 33.7 mmol) at ambient temperature. The reaction was stirred at ambient temperature for 4 h. The resulting solution was acidified to pH 5 with diluted HCl (1 M) and extracted with EtOAc (3×500 mL). The organic layers were combined, washed with brine (2×200 mL), dried over anhydrous Na₂SO₄ and filtered. The residue was purified by RP flash with the following conditions: Column: Flash C¹⁸ (300 g); Mobile Phase A: water (0.03% TFA), Mobile Phase B: ACN; (Gradient: 2% B hold 5 min, up to 52% B within 25 min, 52% B hold 5 min; up to 95% B within 5 min, 95% B hold 5 min); Flow rate: 50 mL/min; Detector: UV 210 nm; RT=45 min. The product-containing fractions were collected and concentrated in vacuum to give 2-((((9H-fluoren-9-yl)methoxy)carbonyl)amino)-3-(4-(tert-butoxycarbonyl)phenyl)-3-hydroxypropanoic acid (17.3 g, 32.6 mmol, 87% yield) as an orange solid. MS ESI calculated for C₂₉H₃₀NO₇ [M+H−tBu]+ 448.19, found 448.05.

Step 5: To a mixture of 2-((((9H-fluoren-9-yl)methoxy)carbonyl)amino)-3-(4-(tert-butoxycarbonyl)phenyl)-3-hydroxypropanoic acid (17 g, 32.0 mmol) in DMF (150 mL) were added NaHCO₃ (8.07 g, 96 mmol) and (bromomethyl)benzene (8.21 g, 48.0 mmol) at ambient temperature. The reaction was stirred at ambient temperature for 16 h. The resulting solution was diluted with water (100 mL) and extracted with EtOAc (3×200 mL). The organic layers were combined, washed with brine (2×150 mL), dried over anhydrous Na₂SO₄ and filtered. The filtrate was concentrated in vacuum to afford crude product. The residue was purified by silica gel chromatography, eluting with a gradient of EtOAc in PE from 0% to 28% to afford tert-butyl 4-(2-((((9H-fluoren-9-yl)methoxy)carbonyl)amino)-3-(benzyloxy)-1-hydroxy-3-oxopropyl)benzoate (14.8 g, 24.93 mmol, 78% yield) as a white solid. MS ESI calculated for C₃₆H₃₆NO₇ [M+H−tBu]⁺ 538.24, found 538.10.

Step 6: The tert-butyl 4-(2-((((9H-fluoren-9-yl)methoxy)carbonyl)amino)-3-(benzyloxy)-1-hydroxy-3-oxopropyl)benzoate (14.8 g, 24.93 mmol) was separated by Prep-SFC-HPLC Column: Chiralpak IH, 7*25 cm, 10 m; Mobile Phase A: CO₂, Mobile Phase B: MeOH; Flow rate: 250 mL/min; Gradient: isocratic 30% B; Column Temperature (° C.): 35; Back Pressure (bar): 100; Wavelength: 220 nm; RT1 (min): 8.95. The collected fractions were combined and concentrated under vacuum. The residue was lyophilized to afford tert-butyl 4-((1R,2S)-2-((((9H-fluoren-9-yl)methoxy)carbonyl)amino)-3-(benzyloxy)-1-hydroxy-3-oxopropyl)benzoate (1.4 g, 2.358 mmol, 9.46% yield) as a white solid. MS ESI calculated for C₃₆H₃₆NO₇ [M+H]⁺ 594.24, found 594.15.

Step 7: To a solution of tert-butyl 4-((1R,2S)-2-((((9H-fluoren-9-yl)methoxy)carbonyl)amino)-3-(benzyloxy)-1-hydroxy-3-oxopropyl)benzoate (1.4 g, 2.358 mmol) in THF (15 mL) and EtOAc (15 mL) was added Pd/C (300 mg, 0.282 mmol, 10% wt, dry) under nitrogen atmosphere. The suspension was degassed under vacuum and purged with H₂ several times; the reaction mixture was stirred for 6 h at room temperature under 2 atm H₂. The resulting suspension was filtered by diatomite and washed by THF/EtOH (1:1, 2×40 mL). The filtrate was concentrated in vacuum. The residue was purified by RP flash with the following conditions: Column: Flash C¹⁸ (120 g); Mobile Phase A: water (0.05% TFA), Mobile Phase B: ACN; (Gradient: 2% B hold 3 min, up to 60% B within 27 min. 60% B hold 5 min; up to 95% B within 5 min, 95% B hold 5 min); Flow rate: 60 mL/min; Detector: UV 210 nm; RT=45 min. The product-containing fractions were collected and concentrated in vacuum to give (2S,3R)-2-((((9H-fluoren-9-yl)methoxy)carbonyl)amino)-3-(4-(tert-butoxycarbonyl)phenyl)-3-hydroxypropanoic acid (1.0498 g, 2.085 mmol, 88% yield) as a brown solid. MS ESI calculated for C₂₉H₂₉NO₇Na [M+Na]⁺ 526.19, found 526.25. ¹H NMR (300 MHz, CD₃OD) δ 7.87 (d, J=8.2 Hz, 2H), 7.77 (d, J=7.5 Hz, 2H), 7.57-7.19 (m, 8H), 5.42 (d, J=2.8 Hz, 1H), 4.55 (d, J=2.8 Hz, 1H), 4.35-4.20 (m, 1H), 4.08-3.97 (m, 2H), 1.50 (s, 9H).

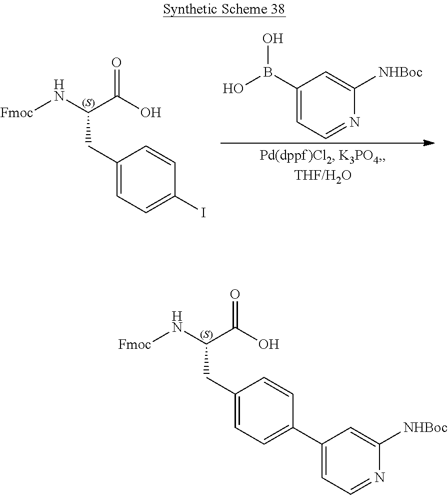

### Precursor to Phe44PyNH3

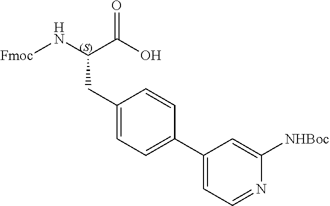

### (S)-2-((((9H-fluoren-9-yl)methoxy)carbonyl)amino)-3-(4-(2-((tert-butoxycarbonyl)amino)pyridin-4-yl)phenyl)propanoic acid

To a stirred solution of (S)-2-((((9H-fluoren-9-yl)methoxy)carbonyl)amino)-3-(4-iodophenyl)propanoic acid (15 g, 29.2 mmol) in THF (176 mL) and water (88 mL) were added tert-butyl (4-(4,4,5,5-tetramethyl-1,3,2-dioxaborolan-2-yl)pyridin-2-yl)carbamate (14.03 g, 43.8 mmol), potassium phosphate (18.61 g, 88 mmol) and Pd(dppf)Cl₂ (1.904 g, 2.92 mmol) at room temperature under nitrogen atmosphere. The resulting mixture was stirred at 50° C. for 2 h. The pH of the mixture was adjusted to 3 with 1 N HCl. The mixture was extracted with EA (3×100 mL). The combined organic layer was washed with brine (100 mL), dried over anhydrous Na₂SO₄. The residue was purified by silica gel column chromatography and eluted with DCM/MeOH=10:1 to afford (S)-2-((((9H-fluoren-9-yl)methoxy)carbonyl)amino)-3-(4-(2-((tert-butoxycarbonyl)amino)pyridin-4-yl)phenyl)propanoic acid (4.8015 g, 7.70 mmol, 26.4% yield) as a brown solid. MS ESI calculated for C₃₄H₃₄N₃O₆ [M+H]⁺ 580.24, found 580.45. ¹H NMR (400 MHz, DMSO-d₆) δ 9.95 (s, 1H), 8.28 (d, J=5.3 Hz, 1H), 8.05 (s, 1H), 7.88-7.75 (m, 3H), 7.68-7.60 (m, 4H), 7.46-7.25 (m, 7H), 4.29-4.10 (m, 4H), 3.17-3.15 (m, 1H), 3.00-2.89 (m, 1H), 1.50 (s, 9H).

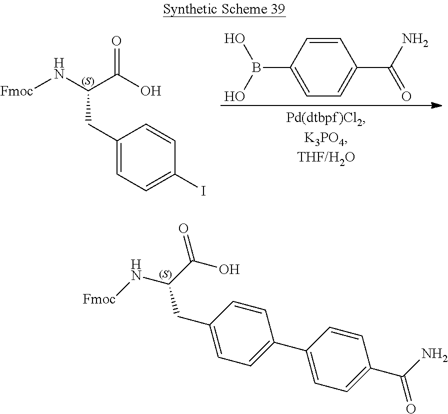

### Precursor to Bip4CONH2

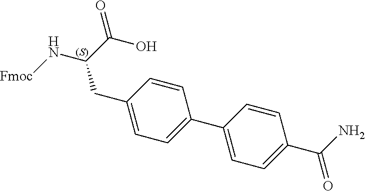

### (S)-2-((((9H-fluoren-9-yl)methoxy)carbonyl)amino)-3-(4′-carbamoyl-[1,1′-biphenyl]-4-yl)propanoic acid

To a stirred solution of (S)-2-((((9H-fluoren-9-yl)methoxy)carbonyl)amino)-3-(4-iodophenyl)propanoic acid (15 g, 29.2 mmol) in THF (246 mL) were added PdCl₂(dtbpf) (2.86 g, 4.38 mmol) and potassium phosphate tribasic (88 mL, 88 mmol, 1 N in water) at room temperature. The solution was stirred at 50° C. for 2 h. The resulting solution was cooled to room temperature. The pH was adjusted to 3 with 1H HCl and extracted with EA (3×250 mL). The organic layers were combined, washed with brine (4×200 mL), dried over anhydrous Na₂SO₄ and filtered. The filtrate was concentrated under reduced pressure and the residue was recrystallized from EtOH (100 mL) to give (S)-2-((((9H-fluoren-9-yl)methoxy)carbonyl)amino)-3-(4′-carbamoyl-[1,1′-biphenyl]-4-yl)propanoic acid (7.5221 g, 14.12 mmol, 48.3% yield) as a brown solid. MS ESI calculated for C₃₁H₂₇N₂O₅ [M+H]⁺ 507.18, found 507.45. ¹H NMR (400 MHz, DMSO-d₆) δ 12.87 (s, 1H), 8.02 (s, 1H), 8.07-7.96 (m, 2H), 7.94-7.82 (m, 2H), 7.78-7.60 (m, 7H), 7.39-7.29 (m, 5H), 7.29-7.19 (m, 2H), 4.20-4.13 (m, 4H), 3.17-3.13 (m, 1H), 2.93-2.90 (m, 1H).

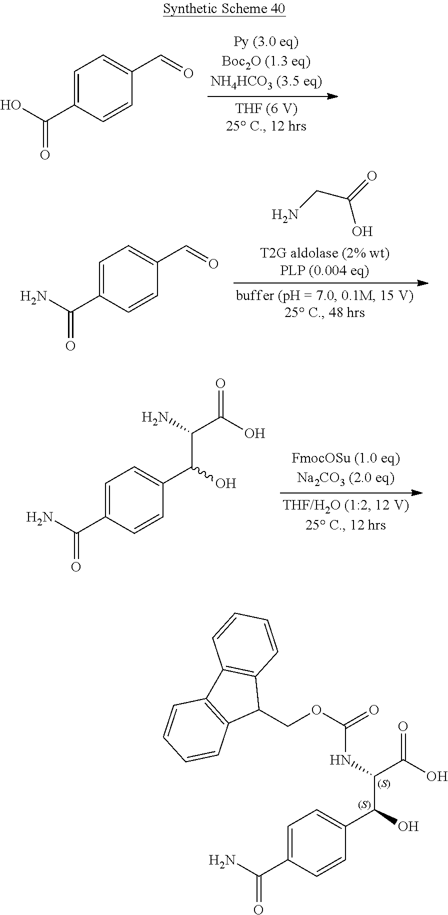

### Precursor to SbOHPhe4CONH2

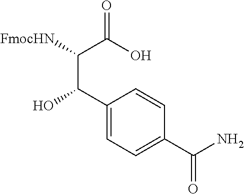

### (2S,3S)-2-((((9H-fluoren-9-yl)methoxy)carbonyl)amino)-3-(4-carbamoylphenyl)-3-hydroxypropanoic acid

4-Formylbenzoic acid (200 g, 1.33 mol), pyridine (322 mL, 4.00 mol), NH₄HCO₃ (368 g, 4.66 mol, 3.50 eq) were added to THF (1.20 L). Boc₂O ((377 g, 1.73 mol, 397 mL) was added to the reaction mixture at 0° C. The reaction mixture was stirred at 25° C. for 4 h. The pH of the reaction mixture was adjusted to 5 by HCl (12 M). After filtration and washing of the filter cakes with H₂O, the crude product was triturated with isopropanol and H₂O at 25° C. for 1 h to obtain the carboxamide intermediate.

To a solution of buffer (NaH₂PO₄ (0.10 M, 6.70 L), Na₂HPO₄ (0.10 M, 6.70 L) were added glycine (187 g, 2.50 mol), pyridoxal-5′-phosphate (PLP) (662 mg, 2.68 mmol), T2G aldolase (3.0 g) and the carboxamide intermediate (100 g, 670 mmol). The mixture was stirred at 25° C. for 2 hours. The amino acid intermediate was collected by filtration and washed with water.

The amino acid intermediate (120 g, 535 mmol) was added to a mixture of THF (480 mL) and H₂O (960 mL). Na₂CO₃ (113 g, 1.07 mol) was added to the mixture. FmocOSu (180 g, 535 mmol) was added to the mixture. The mixture was stirred at 25° C. for 12 h. The pH of the mixture was adjusted to 1 by HCl (12 M). After filtration and washing of the filter cakes with H₂O, the crude product was triturated with acetone:ethyl acetate (1:1) at 25° C. for 2 h to obtain the title compound.

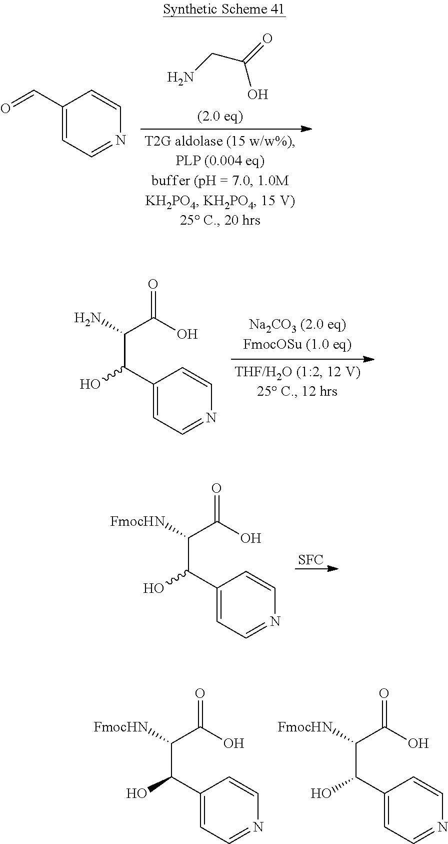

### Precursor to RbOH4Pal and sbOH4Pal

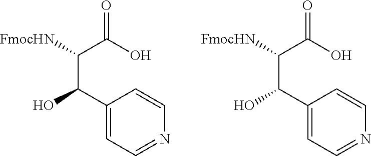

### (2S,3R)-2-((((9H-fluoren-9-yl)methoxy)carbonyl)amino)-3-hydroxy-3-(pyridin-4-yl)propanoic acid and (2S,3S)-2-((((9H-fluoren-9-yl)methoxy)carbonyl)amino)-3-hydroxy-3-(pyridin-4-yl)propanoic acid

To a solution of buffer (KH₂PO₄, 1.0 M, 15) at 25° C. were added glycine (140 g, 1.87 mol), pyridoxal-5′-phosphate (PLP) (922 mg, 3.73 mmol), T2G aldolase (15.0 g) and isonicotinaldehyde (100 g, 933 mmol). The mixture was stirred at 25° C. for 2 hours. The product was collected after filtration and water wash.

To a mixture of the hydroxy-substituted amino acid intermediate in THF (1.5 L) at 25° C. were added Na₂CO₃ (174 g, 1.65 mol) and FmocOSu (277 g, 823 mmol). The mixture was stirred at 25° C. for 12 hours. The pH of the mixture was adjusted to 4 by addition of HCl (12 M). The mixture was filtered, the filter cakes washed with H₂O, and the crude product was triturated with acetonitrile. The Fmoc-protected product was purified by SFC to provide the two title compounds.

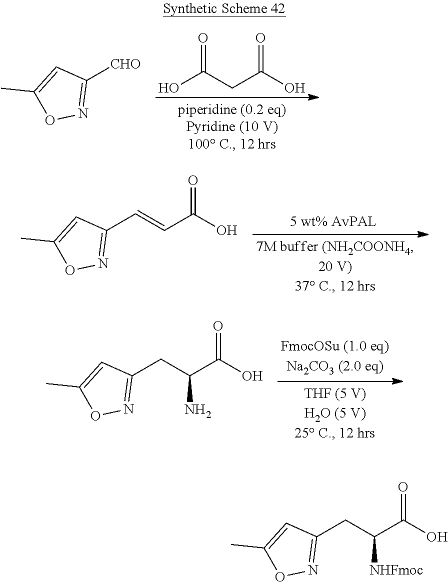

### Precursor to Ala3Isoxa5Me

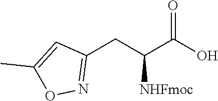

### (S)-2-((((9H-fluoren-9-yl)methoxy)carbonyl)amino)-3-(5-methylisoxazol-3-yl)propanoic acid

To a mixture of the aldehyde (30.0 g, 270 mmol) in pyridine (300 mL) at 25° C. were added malonic acid (56.2 g, 540 mmol) and piperidine (4.60 g, 54.0 mmol). The reaction mixture was stirred at 100° C. for 12 hours. The reaction mixture was concentrated under reduced pressure to give the crude product. The crude product was triturated with MTBE at 25° C. for 60 min to provide the carboxylic acid intermediate.

To a mixture of the carboxylic acid intermediate in 7 M ammonium carbonate buffer was added Anabaena variabilis phenylalanine ammonia-lyase (AvPAL, 1.45 g) at 25° C. The reaction mixture was stirred at 37° C. for 12 h. The reaction mixture was concentrated under reduced pressure to provide the amino acid intermediate.

To a mixture of the amino acid intermediate (32.0 g) in H₂O (160 mL) and THF (160 mL) at 25° C. were added Na2CO3 (39.8 g, 376 mmol) and FmocOSu (63.4 g, 188 mmol). The reaction mixture was stirred at 25° C. for 12 h and the concentrated under reduced pressure. The crude residue was acidified to pH=3 with 2 N HCl (aq.) (180 mL). The mixture was filtered and filter cake was washed with water. The filtrate was concentrated under reduced pressure to give crude product. The crude product was triturated with acetonitrile to provide the title compound.

### Precursor to Ala2Oxa

### (S)-2-((((9H-fluoren-9-yl)methoxy)carbonyl)amino)-3-(oxazol-2-yl)propanoic acid

The Fmoc-protected precursor to Ala2Oxa, (S)-2-((((9H-fluoren-9-yl)methoxy)carbonyl)amino)-3-(oxazol-2-yl)propanoic acid, was prepared using a similar method as above starting from oxazole-2-carbaldehyde.

### Preparation of Final Compounds:

### A. Generalized Procedure for Synthesizing Linear Peptide Precursors

Peptides in Table 1 were synthesized using standard solid-phase synthesis using Fmoc/tBu chemistry as exemplified in Chan, W. C.; White, P. D. “Fmoc Solid-Phase Synthesis: a Practical Approach”, Oxford University Press, Oxford, 2000; Steward, J.; Young, J. “Solid Phase Peptide Synthesis”, Pierce Chemical Company, Rockford, 1984.; Benoiton, N. L. “Chemistry of Peptide Synthesis”, CRC Press, New York, 2006; and Lloyd-Williams. P.; Albericio, F.; Giralt, E. “Chemical Approaches to the Synthesis of Peptides and Proteins”, CRC Press, New York, 1997.

During peptide chain elongation, the α-amino group of each amino acid was protected with a 9H-fluoren-9-ylmethoxycarbonyl group (Fmoc). To avoid any side reactions during the chain elongation steps, any reactive amino acid side chains also carry acid-labile protecting groups, effectively masking the reactive groups until removal upon treatment with strong acid. After completion of each coupling step, the Fmoc group of the N-terminal amino acid was removed with piperidine or 4-methylpiperidine and the resin was thoroughly washed to prepare for the coupling of the subsequent Fmoc-protected amino acid derivative.

The side chain protecting groups used were: tert-butyl (tBu) for 3Pal4CO₂H, 3Pal4Ph4CO2H, alT, aMeD, aMeS, Bip4CO2H, daMeD, daMeS, F4bcpA, F4pcCCA, F4ptCCA, hS, Phe4COOH, Phe4Pyrim5CO2H, Proc4OH, PyrimAla4Ph4CO2H, S, T; tert-butoxy-carbonyl (Boc) for Ac4cN, AlaPip4, AlaPiperaz, aMeDab, aMeK, Dab, Dap, daMeDab, daMeK, K, PipH, Orn, trityl (Trt) for Q; 2,2,4,6,7-pentamethyldihydrobenzofuran-5-sulfonyl (Pbf) for R; and, 3-methylpentyl ester (OMpe) for D.

Fmoc-protected amino acids were typically obtained from vendors such as Sigma-Aldrich, Novabiochem, Chem-Impex, and Combi-Block.

### B. Synthetic Procedures Used to Prepare Cyclic Peptides

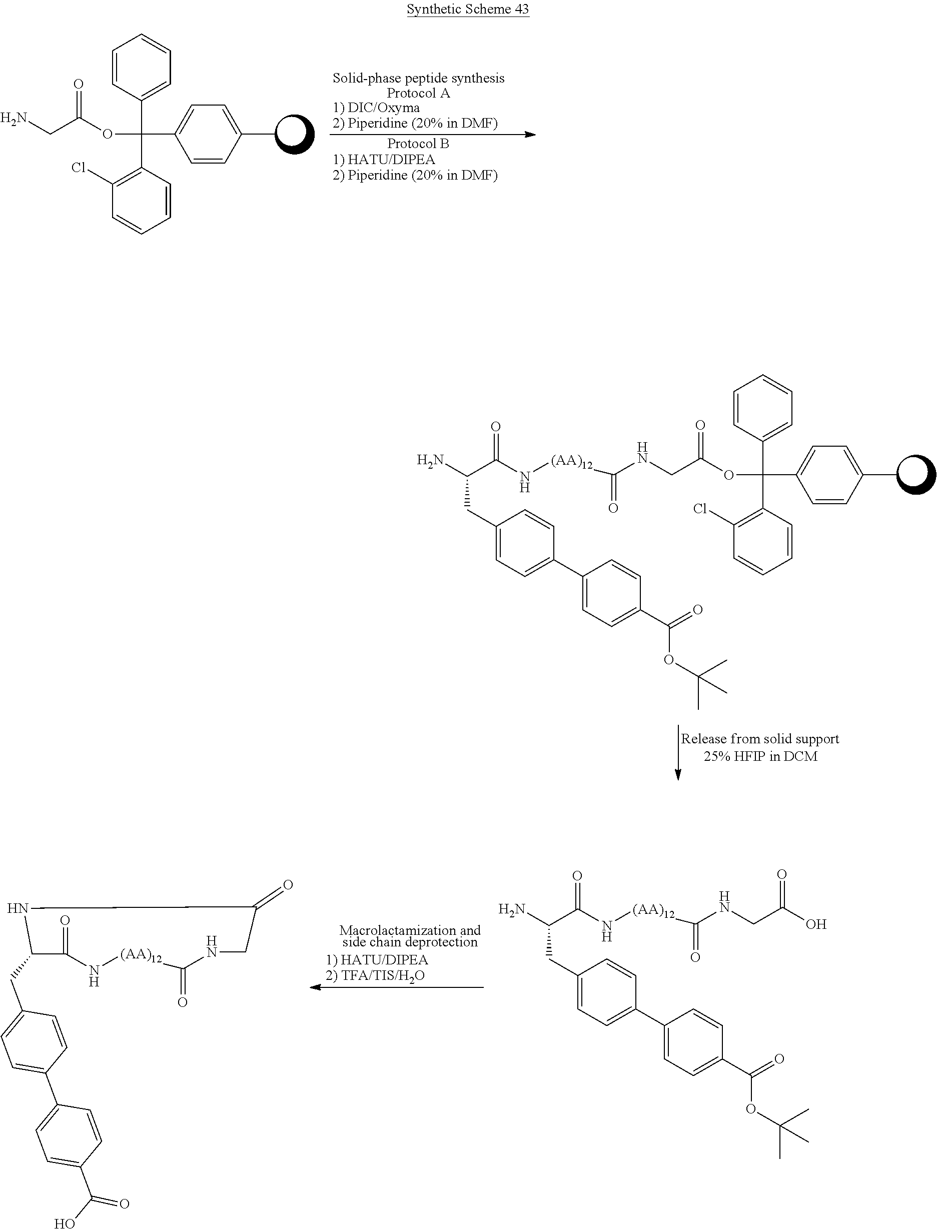

### Solid-Phase Synthesis of Peptides Protocol A

Peptides were synthesized on a Liberty Blue™ synthesizer from CEM Corporation, using standard solid-phase synthesis using Fmoc/tBu chemistry as summarized above in Scheme 44.

N,N′-Diisopropylcarbodiimide (DIC) with ethyl cyano(hydroxyimino)acetate (Oxyma) were used as coupling agents to form the amide bond between the free amino terminus of the resin-bound protected peptide and the carboxylic acid of the Fmoc-protected amino acid.

H-Gly-loaded 2-chlorotrityl resin (200-400 mesh, 0.79 mmol/g loading, 1% cross-linked polystyrene, Novabiochem) was used for synthesis. All the amino acids were dissolved at a 0.2 M concentration in DMF (N,N-dimethylformamide). The amino acids were activated with equimolar amounts of Oxyma solution (0.5 M in DMF), and a 2-fold molar excess of DIC solution (1.0 M in DMF). Alternatively, amino acids were dissolved at a 0.125 M concentration in DMF (N,N-dimethylformamide). The amino acids were activated with equimolar amounts of Oxyma Pure solution (0.125 M in DMF; with 0.05 M DIEA), and a 2-fold molar excess of DIC solution (0.25 M in DMF). Reactions were typically performed at the 25 μmol scale.

Every synthesis cycle included: Fmoc-amino acid deprotection by 20% piperidine in DMF (90° C. microwave assisted heating, 2 min) and coupling (potentially repeated twice for difficult couplings) with Fmoc-protected amino acid/DIC/Oxyma (5, 5, and 10 eq respectively; 90° C. microwave assisted heating, 2 min or 4 min). Cycles of Fmoc deprotection and Fmoc-protected amino acid coupling were repeated with the desired monomers until the full linear peptide was formed.

### Solid-Phase Synthesis of Peptides Protocol B

Alternatively, peptides were synthesized manually on a Biotage® Syro II peptide synthesizer using standard solid-phase synthesis using Fmoc/tBu chemistry as summarized above in Scheme 43. HATU with DIPEA were used as coupling agents to create the amide bond between the free amino terminus of the resin-bound protected peptide and the carboxylic acid of the Fmoc-protected amino acid. H-Gly-loaded 2-chlorotrityl resin (200-400 mesh, 0.79 mmol/g loading, 1% cross-linked polystyrene, Novabiochem) was used for synthesis. All the amino acids were dissolved at a 0.2 M concentration in 1:1 DMF:NMP. Reactions were typically performed at the 12 μmol scale.

Every synthesis cycle included: (1) Coupling (repeated twice) with Fmoc-protected amino acid/HATU/DIPEA (4, 4 and 8 eq, respectively; rt; 15 min). The mixture was filtered, and the peptidyl resin was washed with DMF (2×1 mL); (2) Fmoc deprotection (repeated three times): 20% 4-methyl piperidine in DMF (1 mL; rt; 3 min). The mixture was filtered, and the peptidyl resin was washed with DMF (4×1 mL). Cycles of Fmoc deprotection and Fmoc-protected amino acid coupling were repeated with the desired monomers until the full linear peptide was formed.

### Selective Cleavage of Protected Peptide and Macrolactamization

For the cleavage of the protected linear peptide from the solid support, the peptidyl resin (~16 mg) was treated with 25% hexafluoroisopropanol (HFIP) in DCM for 20 min at rt, filtered, and the solvent was removed under reduced pressure. The resulting residue was dissolved in DMF (5 mL). HATU (0.44 eq) and DIPEA (2.5 eq) were added. The mixture was stirred for 5 min at rt. Then an additional 0.66 eq of HATU was added. Upon completion of the macrolactamization, monitored by UPLC-MS, the solvent was removed under reduced pressure.

### Final Side Chain Deprotection

A solution of TFA/H₂O/TIS (90/8/2, v/v/v, 1 mL) was added to the crude protected cyclic peptide. The mixture was stirred for 10 min at rt. Cold diethyl ether (15 mL) was added to the solution. The peptide was precipitated by centrifugation (3200 rpm, −10° C.). The precipitate was washed with diethyl ether (2×10 mL) and dried under vacuum overnight to give the crude deprotected cyclic peptide as a solid.

### HPLC Purification

Purification was performed by preparative reversed-phase high performance liquid chromatography (RP-HPLC) on Waters X-Bridge Prep C18 OBD Prep column (130 Å, 5 pm, column size 19*100 mm) using a Waters MS-Directed AutoPurification HPLC/MS system. Mobile phase: (A) 0.16% TFA in HPLC water and (B) 0.16% TFA in HPLC acetonitrile; flow rate: 25 mL/min; UV wavelength λ=215 nm; gradient: 25-50% B over 5 min. Alternatively purification was performed on Waters CSH—C18 Column (19×250 mm, 5 uM) using an Agilent, with 1290 infinity II preparative LC system and LC-MSD XT mass spectrometer. Moblie phase: (A) 0.1% formic acid in HPLC water and (B) 0.1% formic acid in HPLC acetonitrile; flow rate: 25 mL/min; UV wavelength λ=215 nm; gradient: 20% B over 2.5 min, 55% B over 2.5-20 min. UV absorbing fractions containing the target m/z ions were collected and the fractions containing product were confirmed by LC/MS.

Purity of fractions were confirmed by UPLC, which was measured by a reverse phase Waters Acquity UPLC-MS system. Column: Waters XSelect CSH C18 Column (130 Å, 2.5 pm, column size 2.1*50 mm). Mobile phase: (A) 0.05% TFA in HPLC water and (B) 0.05% TFA in HPLC acetonitrile; injection volume: 1 μL; flow rate: 1 mL/min; UV wavelength λ=215 nm; gradient: 5-100% B in 5 min. Lyophilization of combined fractions containing pure peptide resulted in the final cyclized product as a powder.

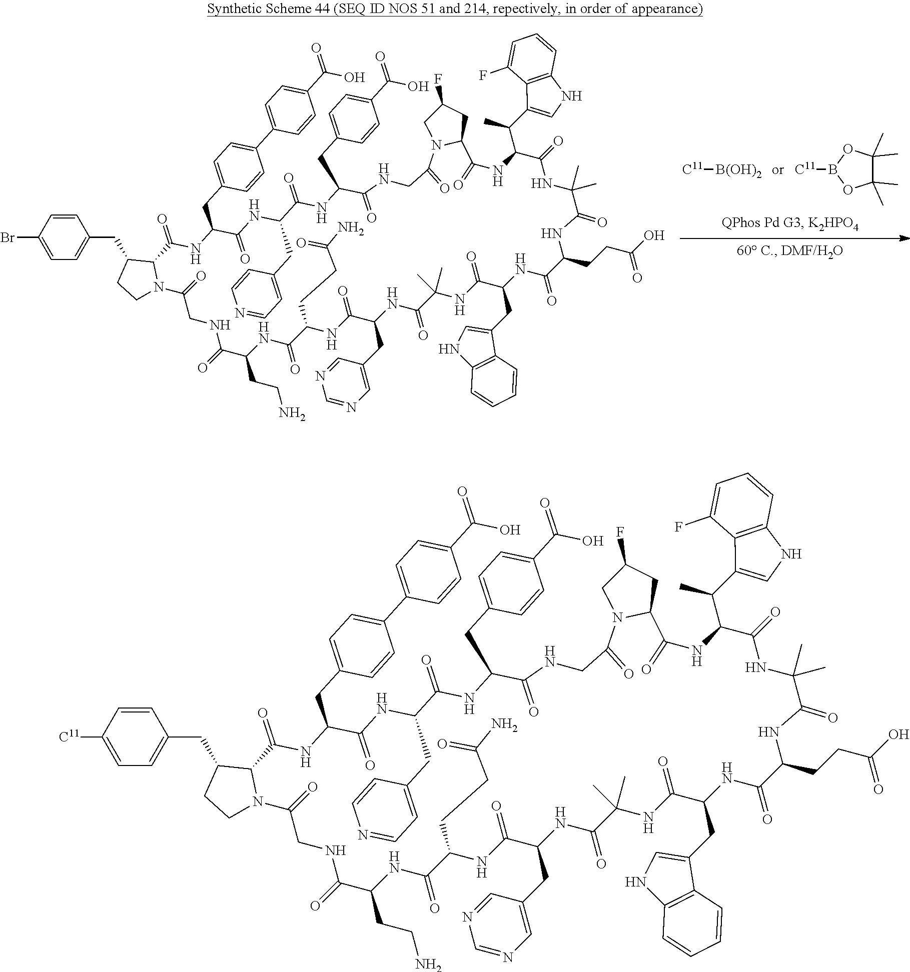

For certain compounds, wherein R¹¹ is —CH₂—C¹¹—C^{a} and C¹¹ is phenyl, a final aryl//heteroaryl coupling was used to complete their syntheses. An aryl/heteroaryl bromide precursor was synthesized using the general procedures described in Synthetic Scheme 6 and in the solid phase syntheses of peptide protocols.

In a N₂-filled glove box to an aryl boronic acid or pinacol ester (0.07 mmol) at room temperature was added a solution of aryl bromide (0.007 mmol) in 150 μL of DMF, followed by addition of an aqueous solution of K₂HPO₄ (1.25 M, 48 μL) and solution of QPhos Pd G₃ (1.15 mg) in DMF (15 μL). The resulting mixture was heated to 60° C. for 21 h. The reaction mixture was then filtered and purified by HPLC Purification procedure described above.

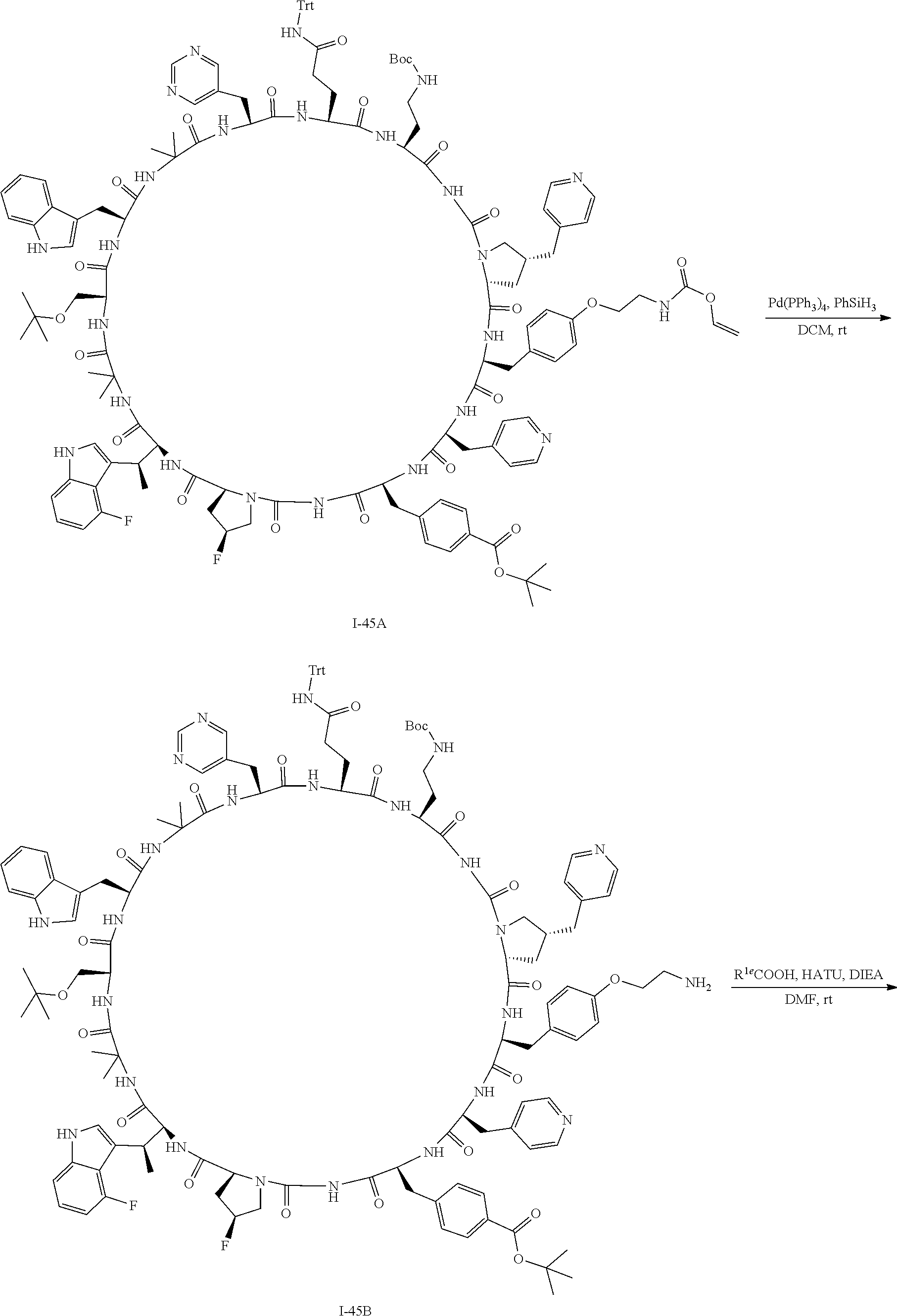

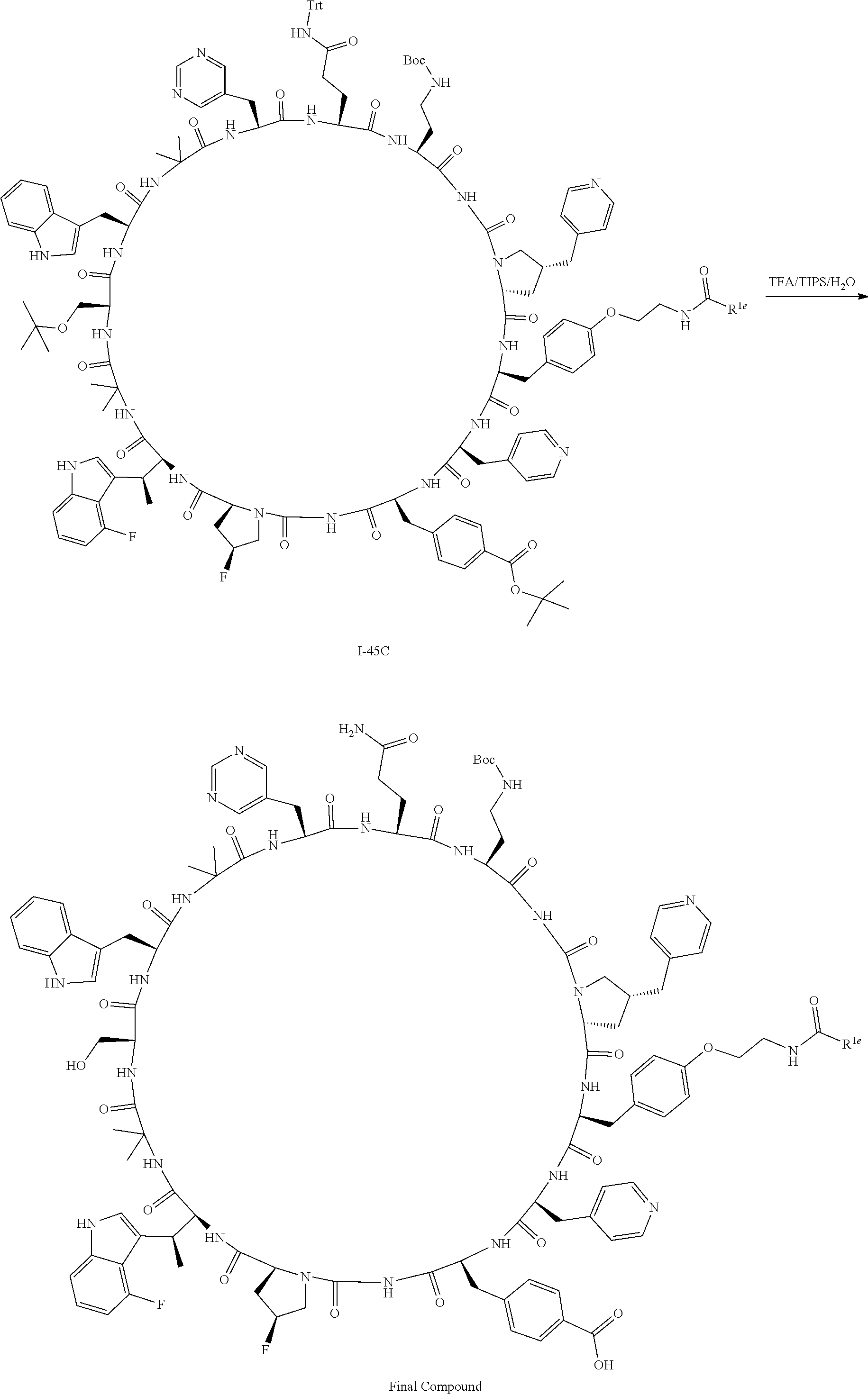

For certain compounds, wherein R¹ is R^{1e}—C(O)NH—CH₂CH₂—O—, additional transformations were performed to provide the final compounds. These transformations are exemplified in the scheme above and the procedures below.

Tert-butyl 4-(((2R,9S,12S,15S,21S,24S,30S,32aS,34S,41S,44S,47S,49aR)-21-((1H-indol-3-yl)methyl)-9-(2-((tert-butoxycarbonyl)amino)ethyl)-24-(tert-butoxymethyl)-34-fluoro-30-((S)-1-(4-fluoro-1H-indol-3-yl)ethyl)-18,18,27,27-tetramethyl-5,8,11,14,17,20,23,26,29,32,37,40,43,46,49-pentadecaoxo-12-(3-oxo-3-(tritylamino)propyl)-2,44-bis(pyridin-4-ylmethyl)-15-(pyrimidin-5-ylmethyl)-47-(4-(2-(((vinyloxy)carbonyl)amino)ethoxy)benzyl)octatetracontahydro-1H-dipyrrolo[1,2-a:1′,2′-e1][1,4,7,10,13,16,19,22,25,28,31,34,37,40,43]pentadecaazacyclopentatetracontin-41-yl)methyl)benzoate (I-45A, 0.606 g, 0.24 mmol) in CH₂Cl₂ (10 mL) was bubbled with N₂, Pd(PPh₃)₄ (0.021 g, 0.018 mmol) and phenylsilane (0.118 mL, 0.960 mmol) was added, bubbled with N₂ again, then stirred at rt for 2 h, solvent was removed, the crude mixture of I-45B was taken on to next step without purification.

To acid monomer R^{1e}COOH (0.055 mmol) at rt was added I-45B (0.011 mmol, in 0.82 mL DMF), HATU (0.4 M in DMF, 121 μL) and DIEA (2M in NMP, 48 μL), the resulting mixture was stirred at rt for 2 h, and then evaporated to dryness to give I-45C. I-45C was treated with 1.00 mL of TFA cleavage cocktail (90% TFA, 2% TIPS, 8% H₂O) at rt for 0.5 h, the reaction mixture was then transferred to 10 mL of chilled Et₂O, centrifuged, decanted, and dried under vacuum. DMSO (1 mL) was added and the crude mixture was purified by reverse phase HPLC to give the final compounds.

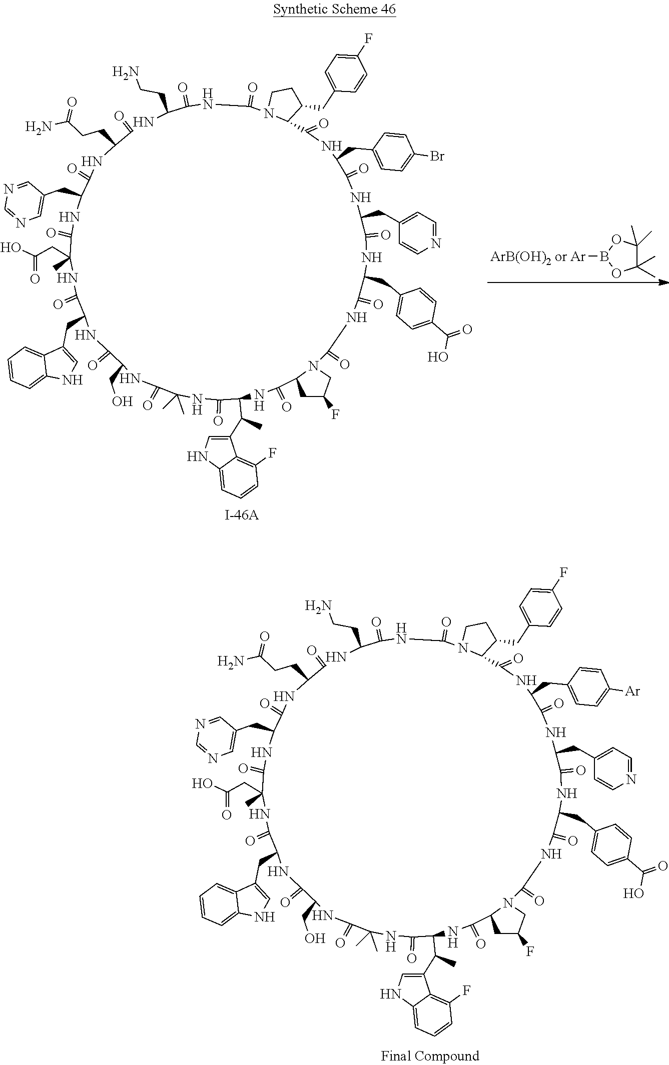

For certain compounds, wherein R¹ is C¹, wherein C¹ is aryl or heteroaryl, additional transformations were performed to provide the final compounds. These transformations are exemplified in the scheme above and the procedures below.

To ArB(OH)₂ or boronic ester (0.095 mmol, 10.7 eq) at room temperature in a glove box was added I-46A (0.009 mmol, 1 eq) in 190 ul DMF, followed by addition of K₂HPO₄ (1.25M aq, 58 μL, 8 eq) and QPhos Pd G3(0.19 mg in 19 μL DMF). The resulting mixture was heated at 60° C. for 21 h. The reaction mixture was then filtered, dissolved in DMSO (1 mL), and purified by reverse phase HPLC to give the final compounds.

### Biological Assays

### Procedure for IL-6 Assay in MRC5 Cells

The inhibition of IL-1β induced IL-6 secretion was evaluated in MRC5 cells. Recombinant human IL-1β (BioLegend 579404) at 2λ EC₈₀ concentration was prepared in seeding medium. EMEM (ATCC 30-2003) with 0.025% BSA (Sigma A9576), 1×penicillin/streptomycin (Gibco 15070-063), 1×NEAA (Gibco 11140-050), 1×GlutaMax (Gibco 35050-061), and 1×sodium pyruvate (Gibco 11360-070), 20 μL of IL-1β was added to a 384-well collagen-coated plate (Corning 354664) and pre-incubated at ambient temperature for 1 hour with 200 nL of compound dispensed using an ECHO 555 liquid handler. Human lung fibroblast MRC5 cells (ATCC CCL-171) were added at a density of 3000 cells/20 μL per well. The cells were prepared by passaging three times in growth medium, EMEM (ATCC 30-2003) with 10% fetal bovine serum (Gibco 16140-071), 1×penicillin/streptomycin (Gibco 15070-063), 1×NEAA (Gibco 11140-050), 1×GlutaMax (Gibco 35050-061), and 1×sodium pyruvate (Gibco 11360-070) in collagen-coated T175 flasks (Greiner 661950) and harvested in seeding medium after 5 minutes of 0.25% trypsin-EDTA (Gibco 25200-056) digestion. The 384-well collagen-coated plate, containing a final volume of 40 μL, was incubated at 37° C., 5% CO₂ overnight. 5 μL of the conditioned medium was transferred to a 384-well AlphaLISA plate (PerkinElmer 6005350) for detection of IL-6 using the human AlphaLISA IL-6 kit (PerkinElmer AL223F) as per the manufacturer's protocol. 20 μL of the acceptor bead/biotinylated antibody mix was added to the 384-well AlphaLISA plate and incubated for 1 hour at ambient temperature. The donor bead mix was protected from light and 25 μL was added to the plate and incubated for 30 minutes at ambient temperature. The AlphaLISA plate was read on an EnVision multimode plate reader (Perkin Elmer model 2104) using the AlphaScreen setting (laser exc at 680 nm and emis at 570 nm). Dose response curves and IC₅₀ values were analyzed using a 4-parameter logistic equation in Spotfire software (Tibco, Palo Alto, CA).

The amino acid sequences, biological activities (MRC IC₅₀s), calculated monoisotopic masses, molecular formulas, calculated molecular weights and mass spectral data (M+H), (M+2H/2) or (M+3H/3) of Example Nos. 1-213 and 215-285 (SEQ ID NOS: 1-213 and 215-285 are provided below in Table 1.

TABLE 1

| | | MRC5 | Exact | | Molecular | Measured | |
|---|---|---|---|---|---|---|---|
| SEQ ID | | IC₅₀ | Mass | Molecular | Weight | Mass | Observed |
| NO. | Sequence | (nM) | (Da) | Formula | (g/mol) | (Da) | Ion |
| 1 | Bip4CO2H-4Pal- | 0.093 | 2087.86943 | C108H119F2N19O23 | 2089.21 | 2088.67 | [M + H]+ |
| | Phe4COOH-G-Proc4F- | | | | | | |
| | SbMeW4F-Aib-E-W- | | | | | | |
| | Aib-F-Q-S-G- | | | | | | |
| | dProc3Bn | | | | | | |
| 2 | Bip4CO2H-4Pal- | 0.023 | 2119.88689 | C108H120F3N21O22 | 2121.23 | 2121.24 | [M + H]+ |
| | Phe4COOH-G-Proc4F- | | | | | | |
| | SbMeW4F-Aib-E-W- | | | | | | |
| | Aib-4Pal-Q-Dab-G- | | | | | | |
| | dProc4Bn4F | | | | | | |
| 3 | Bip4CO2H-4Pal- | 0.037 | 2107.8505 | C106H116F3N21O23 | 2109.18 | 1054.47 | [M + 2H]/2+ |
| | Phe4COOH-G-Proc4F- | | | | | | |
| | SbMeW4F-Aib-E-W- | | | | | | |
| | Aib-PyrimAla-Q-S-G- | | | | | | |
| | dProc4Bn4F | | | | | | |
| 4 | Bip4CO2H-4Pal- | 0.031 | 2120.88214 | C107H119F3N22O22 | 2122.22 | 1062.57 | [M + 2H]/2+ |
| | Phe4COOH-G-Proc4F- | | | | | | |
| | SbMeW4F-Aib-E-W- | | | | | | |
| | Aib-PyrimAla-Q-Dab- | | | | | | |
| | G-dProc4Bn4F | | | | | | |
| 5 | Bip4CO2H-4Pal- | 0.039 | 2016.84469 | C101H115F3N20O22 | 2018.11 | 1009.656 | [M + 2H]/2+ |
| | Phe4COOH-G-Proc4F- | | | | | | |
| | SbMeW4F-Aib-S-W- | | | | | | |
| | Aib-S-Q-Dab-G- | | | | | | |
| | dProc4Bn4F | | | | | | |
| 6 | Bip4CO2H-4Pal- | 0.057 | 2062.87666 | C105H117F3N22O20 | 2064.18 | 1033.282 | [M + 2H]/2+ |
| | Phe4COOH-G-Proc4F- | | | | | | |
| | SbMeW4F-Aib-A-W- | | | | | | |
| | Aib-PyrimAla-Q-Dab- | | | | | | |
| | G-dProc4Bn4F | | | | | | |
| 7 | Bip4CO2H-4Pal- | 0.026 | 2106.86649 | C106H117F3N22O22 | 2108.19 | 1054.944 | [M + 2H]/2+ |
| | Phe4COOH-G-Proc4F- | | | | | | |
| | SbMeW4F-Aib-E-W- | | | | | | |
| | Aib-PyrimAla-Q-Dap- | | | | | | |
| | G-dProc4Bn4F | | | | | | |
| 8 | Bip4CO2H-4Pal- | 0.026 | 2134.89779 | C108H121F3N22O22 | 2136.25 | 1068.962 | [M + 2H]/2+ |
| | Phe4COOH-G-Proc4F- | | | | | | |
| | SbMeW4F-Aib-E-W- | | | | | | |
| | Aib-PyrimAla-Q-Orn- | | | | | | |
| | G-dProc4Bn4F | | | | | | |
| 9 | Bip4CO2H-4Pal- | 0.028 | 2148.91344 | C109H123F3N22O22 | 2150.27 | 1075.968 | [M + 2H]/2+ |
| | Phe4COOH-G-Proc4F- | | | | | | |
| | SbMeW4F-Aib-E-W- | | | | | | |
| | Aib-PyrimAla-Q-K-G- | | | | | | |
| | dProc4Bn4F | | | | | | |
| 10 | TyrEtNAc-4Pal- | 0.027 | 2101.90869 | C104H122F3N23O22 | 2103.22 | 1052.467 | [M + 2H]/2+ |
| | Phe4COOH-G-Proc4F- | | | | | | |
| | SbMeW4F-Aib-E-W- | | | | | | |
| | Aib-PyrimAla-Q-Dab- | | | | | | |
| | G-dProc4Bn4F | | | | | | |
| 11 | TyrEtNAc-4Pal- | 0.045 | 2087.89304 | C103H120F3N23O22 | 2089.19 | 1045.458 | [M + 2H]/2+ |
| | Phe4COOH-G-Proc4F- | | | | | | |
| | SbMeW4F-Aib-E-W- | | | | | | |
| | Aib-PyrimAla-Q-Dap- | | | | | | |
| | G-dProc4Bn4F | | | | | | |
| 12 | 3AzaTyrEtNAc-4Pal- | 0.072 | 2102.90394 | C103H121F3N24O22 | 2104.21 | 1052.964 | [M + 2H]/2+ |
| | Phe4COOH-G-Proc4F- | | | | | | |
| | SbMeW4F-Aib-E-W- | | | | | | |
| | Aib-PyrimAla-Q-Dab- | | | | | | |
| | G-dProc4Bn4F | | | | | | |
| 13 | Bip4CO2H-AlaPent- | 0.022 | 2109.90254 | C107H122F3N21O22 | 2111.24 | 1055.963 | [M + 2H]/2+ |
| | Phe4COOH-G-Proc4F- | | | | | | |
| | SbMeW4F-Aib-E-W- | | | | | | |
| | Aib-PyrimAla-Q-Dab- | | | | | | |
| | G-dProc4Bn4F | | | | | | |
| 14 | Bip4CO2H-4Pal- | 0.035 | 2103.88681 | C106H119F2N23O22 | 2105.22 | 1054.443 | [M + 2H]/2+ |
| | Phe4COOH-G-Proc4F- | | | | | | |
| | SbMeW4F-Aib-E-W- | | | | | | |
| | Aib-PyrimAla-Q-Dab- | | | | | | |
| | G-dProc4CH24Pal | | | | | | |
| 15 | Bip4CO2H-4Pal- | 0.035 | 2102.89156 | C107H120F2N22O22 | 2104.23 | 1052.445 | [M + 2H]/2+ |
| | Phe4COOH-G-Proc4F- | | | | | | |
| | SbMeW4F-Aib-E-W- | | | | | | |
| | Aib-4Pal-Q-Dab-G- | | | | | | |
| | dProc4CH24Pal | | | | | | |
| 16 | TyrEtNAc-4Pal- | 0.051 | 2059.89812 | C102H120F3N23O21 | 2061.18 | 2061.586 | [M + H]+ |
| | Phe4COOH-G-Proc4F- | | | | | | |
| | SbMeW4F-Aib-S-W- | | | | | | |
| | Aib-PyrimAla-Q-Dab- | | | | | | |
| | G-dProc4Bn4F | | | | | | |
| 17 | Bip4CO2H-4Pal- | 0.029 | 2106.86649 | C106H117F3N22O22 | 2108.19 | 1055.03 | [M + 2H]/2+ |
| | Phe4COOH-G-Proc4F- | | | | | | |
| | SbMeW4F-Aib-D-W- | | | | | | |
| | Aib-PyrimAla-Q-Dab- | | | | | | |
| | G-dProc4Bn4F | | | | | | |
| 18 | Bip4CO2H-4Pal- | 0.027 | 2136.87705 | C107H119F3N22O23 | 2138.22 | 1069.131 | [M + 2H]/2+ |
| | Phe4COOH-G-Proc4F- | | | | | | |
| | SbMeW4F-Aib-E-W- | | | | | | |
| | aMeS-PyrimAla-Q- | | | | | | |
| | Dab-G-dProc4Bn4F | | | | | | |
| 19 | Bip4CO2H-4Pal- | 0.021 | 2136.87705 | C107H119F3N22O23 | 2138.22 | 1069.954 | [M + 2H]/2+ |
| | Phe4COOH-G-Proc4F- | | | | | | |
| | SbMeW4F-daMeS-E- | | | | | | |
| | W-Aib-PyrimAla-Q- | | | | | | |
| | Dab-G-dProc4Bn4F | | | | | | |
| 20 | Bip4CO2H-4Pal- | 0.062 | 2133.87739 | C107H118F3N23O22 | 2135.22 | 1068.543 | [M + 2H]/2+ |
| | Phe4COOH-G-Proc4F- | | | | | | |
| | SbMeW4F-Ac4cN-E- | | | | | | |
| | W-Aib-PyrimAla-Q- | | | | | | |
| | Dab-G-dProc4Bn4F | | | | | | |
| 21 | Bip4CO2H-4Pal- | 0.04 | 2133.87739 | C107H118F3N23O22 | 2135.22 | 1068.073 | [M + 2H]/2+ |
| | Phe4COOH-G-Proc4F- | | | | | | |
| | SbMeW4F-Aib-E-W- | | | | | | |
| | Ac4cN-PyrimAla-Q- | | | | | | |
| | Dab-G-dProc4Bn4F | | | | | | |
| 22 | Bip4CO2H-4Pal- | 0.027 | 2136.87705 | C107H119F3N22O23 | 2138.22 | 1069.954 | [M + 2H]/2+ |
| | Phe4COOH-G-Proc4F- | | | | | | |
| | SbMeW4F-aMeS-E- | | | | | | |
| | W-Aib-PyrimAla-Q- | | | | | | |
| | Dab-G-dProc4Bn4F | | | | | | |
| 23 | Bip4CO2H-4Pal- | 0.018 | 2136.87705 | C107H119F3N22O23 | 2138.22 | 1070.071 | [M + 2H]/2+ |
| | Phe4COOH-G-Proc4F- | | | | | | |
| | SbMeW4F-Aib-E-W- | | | | | | |
| | daMeS-PyrimAla-Q- | | | | | | |
| | Dab-G-dProc4Bn4F | | | | | | |
| 24 | Bip4CO2H-4Pal- | 0.049 | 2176.90835 | C110H123F3N22O23 | 2178.28 | 1089.459 | [M + 2H]/2+ |
| | Phe4COOH-G-Proc4F- | | | | | | |
| | SbMeW4F-Aib-E-W- | | | | | | |
| | Aib-PyrimAla-Q- | | | | | | |
| | AlaMph-G- | | | | | | |
| | dProc4Bn4F | | | | | | |
| 25 | TyrEtNAc-AlaPent- | 0.053 | 2090.92909 | C104H125F3N22O22 | 2092.23 | 1047.04 | [M + 2H]/2+ |
| | Phe4COOH-G-Proc4F- | | | | | | |
| | SbMeW4F-Aib-E-W- | | | | | | |
| | Aib-PyrimAla-Q-Dab- | | | | | | |
| | G-dProc4Bn4F | | | | | | |
| 26 | 3Pal4Ph4CO2H-4Pal- | 0.035 | 2121.87739 | C106H118F3N23O22 | 2123.21 | 1061.96 | [M + 2H]/2+ |
| | Phe4COOH-G-Proc4F- | | | | | | |
| | SbMeW4F-Aib-E-W- | | | | | | |
| | Aib-PyrimAla-Q-Dab- | | | | | | |
| | G-dProc4Bn4F | | | | | | |
| 27 | Bip4CO2H-4Pal- | 0.098 | 2089.85993 | C106H117F2N21O23 | 2091.19 | 1046.36 | [M + 2H]/2+ |
| | Phe4COOH-G-Proc4F- | | | | | | |
| | SbMeW4F-Aib-E-W- | | | | | | |
| | Aib-F-Q-S-G- | | | | | | |
| | dProc3CH2Pyrim | | | | | | |
| 28 | Bip4CO2H-4Pal- | 0.023 | 2089.85993 | C106H117F2N21O23 | 2091.19 | 2090.66 | [M + H]+ |
| | Phe4COOH-G-Proc4F- | | | | | | |
| | SbMeW4F-Aib-E-W- | | | | | | |
| | Aib-PyrimAla-Q-S-G- | | | | | | |
| | dProc3Bn | | | | | | |
| 29 | Bip4CO2H-4Pal- | 0.019 | 2102.89156 | C107H120F2N22O22 | 2104.23 | 2104.04 | [M + H]+ |
| | Phe4COOH-G-Proc4F- | | | | | | |
| | SbMeW4F-Aib-E-W- | | | | | | |
| | Aib-PyrimAla-Q-Dab- | | | | | | |
| | G-dProc3Bn | | | | | | |
| 30 | Bip4CO2H-4Pal- | 0.026 | 2089.85993 | C106H117F2N21O23 | 2091.19 | 2090.95 | [M + H]+ |
| | Phe4COOH-G-Proc4F- | | | | | | |
| | SbMeW4F-Aib-E-W- | | | | | | |
| | Aib-PyrimAla-Q-S-G- | | | | | | |
| | dProc4Bn | | | | | | |
| 31 | Bip4CO2H-4Pal- | 0.024 | 2102.89156 | C107H120F2N22O22 | 2104.23 | 2103.86 | [M + H]+ |
| | Phe4COOH-G-Proc4F- | | | | | | |
| | SbMeW4F-Aib-E-W- | | | | | | |
| | Aib-PyrimAla-Q-Dab- | | | | | | |
| | G-dProc4Bn | | | | | | |
| 32 | Bip4CO2H-4Pal- | 0.049 | 2114.8927 | C105H121F3N22O23 | 2116.21 | 2116 | [M + H]+ |
| | Phe4COOH-G-Proc4F- | | | | | | |
| | SbMeW4F-Aib-E-W- | | | | | | |
| | Aib-R-Q-S-G- | | | | | | |
| | dProc4Bn4F | | | | | | |
| 33 | Bip4CO2H-4Pal- | 0.033 | 2127.92434 | C106H124F3N23O22 | 2129.25 | 2129.11 | [M + H]+ |
| | Phe4COOH-G-Proc4F- | | | | | | |
| | SbMeW4F-Aib-E-W- | | | | | | |
| | Aib-R-Q-Dab-G- | | | | | | |
| | dProc4Bn4F | | | | | | |
| 34 | Bip4CO2H-4Pal- | 0.032 | 2106.85525 | C107H117F3N20O23 | 2108.19 | 1054.88 | [M + 2H]/2+ |
| | Phe4COOH-G-Proc4F- | | | | | | |
| | SbMeW4F-Aib-E-W- | | | | | | |
| | Aib-3Pal-Q-S-G- | | | | | | |
| | dProc4Bn4F | | | | | | |
| 35 | Bip4CO2H-4Pal- | 0.049 | 2174.92909 | C111H125F3N22O22 | 2176.31 | 2175.47 | [M + H]+ |
| | Phe4COOH-G-Proc4F- | | | | | | |
| | SbMeW4F-Aib-E-W- | | | | | | |
| | Aib-F-Q-R-G- | | | | | | |
| | dProc4Bn4F | | | | | | |
| 36 | Bip4CO2H-4Pal- | 0.054 | 2106.85525 | C107H117F3N20O23 | 2108.19 | 1054.51 | [M + 2H]/2+ |
| | Phe4COOH-G-Proc4F- | | | | | | |
| | SbMeW4F-Aib-E-W- | | | | | | |
| | Aib-2Pal-Q-S-G- | | | | | | |
| | dProc4Bn4F | | | | | | |
| 37 | Phe4AcPip-4Pal- | 0.033 | 2126.94032 | C106H125F3N24O21 | 2128.27 | 1064.991 | [M + 2H]/2+ |
| | Phe4COOH-G-Proc4F- | | | | | | |
| | SbMeW4F-Aib-E-W- | | | | | | |
| | Aib-PyrimAla-Q-Dab- | | | | | | |
| | G-dProc4Bn4F | | | | | | |
| 38 | Phe4Pyrim5CO2H- | 0.041 | 2122.87264 | C105H117F3N24O22 | 2124.2 | 1062.957 | [M + 2H]/2+ |
| | 4Pal-Phe4COOH-G- | | | | | | |
| | Proc4F-SbMeW4F- | | | | | | |
| | Aib-E-W-Aib- | | | | | | |
| | PyrimAla-Q-Dab-G- | | | | | | |
| | dProc4Bn4F | | | | | | |
| 39 | 3AzaPhe4AcPip-4Pal- | 0.063 | 2127.93557 | C105H124F3N25O21 | 2129.26 | 1064.901 | [M + 2H]/2+ |
| | Phe4COOH-G-Proc4F- | | | | | | |
| | SbMeW4F-Aib-E-W- | | | | | | |
| | Aib-PyrimAla-Q-Dab- | | | | | | |
| | G-dProc4Bn4F | | | | | | |
| 40 | PyrimAla4AcPip-4Pal- | 0.048 | 2128.93082 | C104H123F3N26O21 | 2130.25 | 1055.488 | [M + 2H]/2+ |
| | Phe4COOH-G-Proc4F- | | | | | | |
| | SbMeW4F-Aib-E-W- | | | | | | |
| | Aib-PyrimAla-Q-Dab- | | | | | | |
| | G-dProc4Bn4F | | | | | | |
| 41 | Bip4CO2H-4Pal- | 0.029 | 2045.88133 | C104H117F2N23O20 | 2047.18 | 1023.891 | [M + 2H]/2+ |
| | Phe4COOH-G-Proc4F- | | | | | | |
| | SbMeW4F-Aib-A-W- | | | | | | |
| | Aib-PyrimAla-Q-Dab- | | | | | | |
| | G-dProc4CH24Pal | | | | | | |
| 42 | Bip4CO2H-4Pal- | 0.021 | 2061.87624 | C104H117F2N23O21 | 2063.18 | 1032.352 | [M + 2H]/2+ |
| | Phe4COOH-G-Proc4F- | | | | | | |
| | SbMeW4F-Aib-S-W- | | | | | | |
| | Aib-PyrimAla-Q-Dab- | | | | | | |
| | G-dProc4CH24Pal | | | | | | |
| 43 | Bip4CO2H-AlaPent- | 0.026 | 2092.90721 | C106H122F2N22O22 | 2094.23 | 1047.392 | [M + 2H]/2+ |
| | Phe4COOH-G-Proc4F- | | | | | | |
| | SbMeW4F-Aib-E-W- | | | | | | |
| | Aib-PyrimAla-Q-Dab- | | | | | | |
| | G-dProc4CH24Pal | | | | | | |
| 44 | Bip4CO2H-AlaPent- | 0.021 | 2050.89665 | C104H120F2N22O21 | 2052.2 | 1026.359 | [M + 2H]/2+ |
| | Phe4COOH-G-Proc4F- | | | | | | |
| | SbMeW4F-Aib-S-W- | | | | | | |
| | Aib-PyrimAla-Q-Dab- | | | | | | |
| | G-dProc4CH24Pal | | | | | | |
| 45 | Bip4CO2H-AlaPent- | 0.027 | 2091.91196 | C107H123F2N21O22 | 2093.25 | 1047.392 | [M + 2H]/2+ |
| | Phe4COOH-G-Proc4F- | | | | | | |
| | SbMeW4F-Aib-E-W- | | | | | | |
| | Aib-4Pal-Q-Dab-G- | | | | | | |
| | dProc4CH24Pal | | | | | | |
| 46 | Bip4CO2H-AlaPent- | 0.034 | 2078.89156 | C105H120F2N22O22 | 2080.21 | 1040.4 | [M + 2H]/2+ |
| | Phe4COOH-G-Proc4F- | | | | | | |
| | SbMeW4F-Aib-E-W- | | | | | | |
| | Aib-PyrimAla-Q-Dap- | | | | | | |
| | G-dProc4CH24Pal | | | | | | |
| 47 | Bip4CO2H-4Pal- | 0.029 | 2088.87591 | C106H118F2N22O22 | 2090.2 | 1045.865 | [M + 2H]/2+ |
| | Phe4COOH-G-Proc4F- | | | | | | |
| | SbMeW4F-Aib-E-W- | | | | | | |
| | Aib-4Pal-Q-Dap-G- | | | | | | |
| | dProc4CH24Pal | | | | | | |
| 48 | Bip4CO2H-AlaPent- | 0.027 | 2077.89631 | C106H121F2N21O22 | 2079.22 | 1040.225 | [M + 2H]/2+ |
| | Phe4COOH-G-Proc4F- | | | | | | |
| | SbMeW4F-Aib-E-W- | | | | | | |
| | Aib-4Pal-Q-Dap-G- | | | | | | |
| | dProc4CH24Pal | | | | | | |
| 49 | TyrEtNAc-4Pal- | 0.047 | 2084.91336 | C103H122F2N24O22 | 2086.22 | 1043.867 | [M + 2H]/2+ |
| | Phe4COOH-G-Proc4F- | | | | | | |
| | SbMeW4F-Aib-E-W- | | | | | | |
| | Aib-PyrimAla-Q-Dab- | | | | | | |
| | G-dProc4CH24Pal | | | | | | |
| 50 | TyrEtNAc-AlaPent- | 0.044 | 2073.93376 | C103H125F2N23O22 | 2075.23 | 1037.875 | [M + 2H]/2+ |
| | Phe4COOH-G-Proc4F- | | | | | | |
| | SbMeW4F-Aib-E-W- | | | | | | |
| | Aib-PyrimAla-Q-Dab- | | | | | | |
| | G-dProc4CH24Pal | | | | | | |
| 51 | Bip4CO2H-4Pal- | 0.014 | 2180.80207 | C107H119BrF2N22O22 | 2183.12 | 1092.03 | [M + 2H]/2+ |
| | Phe4COOH-G-Proc4F- | | | | | | |
| | SbMeW4F-Aib-E-W- | | | | | | |
| | Aib-PyrimAla-Q-Dab- | | | | | | |
| | G-dProc3Bn4Br | | | | | | |
| 52 | Bip4CO2H-4Pal- | 0.021 | 2128.90835 | C106H123F3N22O23 | 2130.24 | 2129.67 | [M + H]+ |
| | Phe4COOH-G-Proc4F- | | | | | | |
| | SbMeW4F-Aib-E-W- | | | | | | |
| | Aib-Cit-Q-Dab-G- | | | | | | |
| | dProc4Bn4F | | | | | | |
| 53 | Bip4CO2H-4Pal- | 0.018 | 2180.91336 | C111H122F2N24O22 | 2182.3 | 727.765 | [M + 3H]/3+ |
| | Phe4COOH-G-Proc4F- | | | | | | |
| | SbMeW4F-Aib-E-W- | | | | | | |
| | Aib-PyrimAla-Q-Dab- | | | | | | |
| | G-dProc3Bn4Pyrim | | | | | | |
| 54 | Bip4CO2H-4Pal- | 0.021 | 2209.93991 | C112H125F2N25O22 | 2211.34 | 1106.18 | [M + 2H]/2+ |
| | Phe4COOH-G-Proc4F- | | | | | | |
| | SbMeW4F-Aib-E-W- | | | | | | |
| | Aib-PyrimAla-Q-Dab- | | | | | | |
| | G- | | | | | | |
| | dProc3Bn4Pyrim2NMe | | | | | | |
| 55 | Bip4CO2H-4Pal- | 0.024 | 2179.91811 | C112H123F2N23O22 | 2181.31 | 1090.62 | [M + 2H]/2+ |
| | Phe4COOH-G-Proc4F- | | | | | | |
| | SbMeW4F-Aib-E-W- | | | | | | |
| | Aib-PyrimAla-Q-Dab- | | | | | | |
| | G-dProc3Bn4Pyr4 | | | | | | |
| 56 | Bip4CO2H-4Pal- | 0.022 | 2179.91811 | C112H123F2N23O22 | 2181.31 | 1090.92 | [M + 2H]/2+ |
| | Phe4COOH-G-Proc4F- | | | | | | |
| | SbMeW4F-Aib-E-W- | | | | | | |
| | Aib-PyrimAla-Q-Dab- | | | | | | |
| | G-dProc3Bn4Pyr3 | | | | | | |
| 57 | Bip4CO2H-4Pal- | 0.019 | 2195.92426 | C111H123F2N25O22 | 2197.32 | 1098.77 | [M + 2H]/2+ |
| | Phe4COOH-G-Proc4F- | | | | | | |
| | SbMeW4F-Aib-E-W- | | | | | | |
| | Aib-PyrimAla-Q-Dab- | | | | | | |
| | G- | | | | | | |
| | dProc3Bn4Pyraz5NH2 | | | | | | |
| 58 | Bip4CO2H-4Pal- | 0.037 | 2180.91336 | C111H122F2N24O22 | 2182.3 | 1091.08 | [M + 2H]/2+ |
| | Phe4COOH-G-Proc4F- | | | | | | |
| | SbMeW4F-Aib-E-W- | | | | | | |
| | Aib-PyrimAla-Q-Dab- | | | | | | |
| | G-dProc3Bn4Pyrz | | | | | | |
| 59 | Bip4CO2H-4Pal- | 0.018 | 2195.92426 | C111H123F2N25O22 | 2197.32 | 732.822 | [M + 3H]/3+ |
| | Phe4COOH-G-Proc4F- | | | | | | |
| | SbMeW4F-Aib-E-W- | | | | | | |
| | Aib-PyrimAla-Q-Dab- | | | | | | |
| | G- | | | | | | |
| | dProc3Bn4Pyrim2NH2 | | | | | | |
| 60 | Bip4CO2H-4Pal- | 0.018 | 2210.92392 | C112H124F2N24O23 | 2212.33 | 1105.99 | [M + 2H]/2+ |
| | Phe4COOH-G-Proc4F- | | | | | | |
| | SbMeW4F-Aib-E-W- | | | | | | |
| | Aib-PyrimAla-Q-Dab- | | | | | | |
| | G- | | | | | | |
| | dProc3Bn4Pyrim2OMe | | | | | | |
| 61 | Bip4CO2H-4Pal- | 0.022 | 2179.91811 | C112H123F2N23O22 | 2181.31 | 1090.93 | [M + 2H]/2+ |
| | Phe4COOH-G-Proc4F- | | | | | | |
| | SbMeW4F-Aib-E-W- | | | | | | |
| | Aib-PyrimAla-Q-Dab- | | | | | | |
| | G-dProc3Bn4Pyr2 | | | | | | |
| 62 | Bip4CO2H-4Pal- | 0.021 | 2183.91302 | C111H123F2N23O23 | 2185.3 | 1093.04 | [M + 2H]/2+ |
| | Phe4COOH-G-Proc4F- | | | | | | |
| | SbMeW4F-Aib-E-W- | | | | | | |
| | Aib-PyrimAla-Q-Dab- | | | | | | |
| | G-dProc3Bn4Iso3Me | | | | | | |
| 63 | Bip4CO2H-4Pal- | 0.018 | 2182.92901 | C111H124F2N24O22 | 2184.32 | 2183.66 | [M + H]+ |
| | Phe4COOH-G-Proc4F- | | | | | | |
| | SbMeW4F-Aib-E-W- | | | | | | |
| | Aib-PyrimAla-Q-Dab- | | | | | | |
| | G-dProc3Bn4Im2NMe | | | | | | |
| 64 | Bip4CO2H-4Pal- | 0.014 | 2182.92901 | C111H124F2N24O22 | 2184.32 | 2184.27 | [M + H]+ |
| | Phe4COOH-G-Proc4F- | | | | | | |
| | SbMeW4F-Aib-E-W- | | | | | | |
| | Aib-PyrimAla-Q-Dab- | | | | | | |
| | G-dProc3Bn4Im4NMe | | | | | | |
| 65 | Bip4CO2H-4Pal- | 0.025 | 2161.90869 | C109H122F3N23O22 | 2163.27 | 1082.259 | [M + 2H]/2+ |
| | Phe4COOH-G-Proc4F- | | | | | | |
| | SbMeW4F-Aib-E-W- | | | | | | |
| | PipH-PyrimAla-Q- | | | | | | |
| | Dab-G-dProc4Bn4F | | | | | | |
| 66 | Bip4CO2H-4Pal- | 0.028 | 2177.93999 | C110H126F3N23O22 | 2179.31 | 1090.6 | [M + 2H]/2+ |
| | Phe4COOH-G-Proc4F- | | | | | | |
| | SbMeW4F-aMeK-E- | | | | | | |
| | W-Aib-PyrimAla-Q- | | | | | | |
| | Dab-G-dProc4Bn4F | | | | | | |
| 67 | Bip4CO2H-4Pal- | 0.021 | 2177.93999 | C110H126F3N23O22 | 2179.31 | 1090.866 | [M + 2H]/2+ |
| | Phe4COOH-G-Proc4F- | | | | | | |
| | SbMeW4F-Aib-E-W- | | | | | | |
| | aMeK-PyrimAla-Q- | | | | | | |
| | Dab-G-dProc4Bn4F | | | | | | |
| 68 | F4ptCCA-4Pal- | 0.029 | 2139.92434 | C107H124F3N23O22 | 2141.27 | 1071.564 | [M + 2H]/2+ |
| | Phe4COOH-G-Proc4F- | | | | | | |
| | SbMeW4F-Aib-E-W- | | | | | | |
| | Ac4cN-PyrimAla-Q- | | | | | | |
| | Dab-G-dProc4Bn4F | | | | | | |
| 69 | F4pcCCA-4Pal- | 0.03 | 2139.92434 | C107H124F3N23O22 | 2141.27 | 1071.212 | [M + 2H]/2+ |
| | Phe4COOH-G-Proc4F- | | | | | | |
| | SbMeW4F-Aib-E-W- | | | | | | |
| | Ac4cN-PyrimAla-Q- | | | | | | |
| | Dab-G-dProc4Bn4F | | | | | | |
| 70 | TyrEtNAc-4Pal- | 0.081 | 2085.90861 | C102H121F2N25O22 | 2087.2 | 2087.27 | [M + H]+ |
| | Phe4COOH-Proc4F- | | | | | | |
| | sbMeW4F-Aib-E-W- | | | | | | |
| | Aib-PyrimAla-Q-Dab- | | | | | | |
| | G-dProc3CH2Pyrim | | | | | | |
| 71 | Bip4CO2H-4Pal- | 0.058 | 2104.88206 | C105H118F2N24O22 | 2106.2 | 702.674 | [M + 3H]/3+ |
| | Phe4COOH-G-Proc4F- | | | | | | |
| | sbMeW4F-Aib-E-W- | | | | | | |
| | Aib-PyrimAla-Q-Dab- | | | | | | |
| | G-dProc3CH2Pyrim | | | | | | |
| 72 | Bip4CO2H-4Pal- | 0.027 | 2119.88689 | C108H120F3N21O22 | 2121.23 | 1060.9 | [M + 2H]/2+ |
| | Phe4COOH-G-Proc4F- | | | | | | |
| | sbMeW4F-Aib-E-W- | | | | | | |
| | Aib-3Pal-Q-Dab-G- | | | | | | |
| | dProc4Bn4F | | | | | | |
| 73 | Bip4CO2H-4Pal- | 0.031 | 2119.88689 | C108H120F3N21O22 | 2121.23 | 707.585 | [M + 3H]/3+ |
| | Phe4COOH-G-Proc4F- | | | | | | |
| | sbMeW4F-Aib-E-W- | | | | | | |
| | Aib-2Pal-Q-Dab-G- | | | | | | |
| | dProc4Bn4F | | | | | | |
| 74 | TyrEtNAc-4Pal- | 0.072 | 2108.95089 | C103H127F3N24O22 | 2110.25 | 1055.49 | [M + 2H]/2+ |
| | Phe4COOH- Proc4F- | | | | | | |
| | sbMeW4F-Aib-E-W- | | | | | | |
| | Aib-R-Q-Dab-G- | | | | | | |
| | dProc4Bn4F | | | | | | |
| 75 | Bip4CO2H-4Pal- | 0.056 | 2176.91959 | C109H123F3N24O22 | 2178.29 | 727.092 | [M + 3H]/3+ |
| | Phe4COOH-G-Proc4F- | | | | | | |
| | sbMeW4F-Aib-E-W- | | | | | | |
| | Aib-PyrimAla-Q-R-G- | | | | | | |
| | dProc4Bn4F | | | | | | |
| 76 | Bip4CO2H-4Pal- | 0.059 | 2175.92434 | C110H124F3N23O22 | 2177.3 | 2177.05 | [M + H]+ |
| | Phe4COOH-G-Proc4F- | | | | | | |
| | sbMeW4F-Aib-E-W- | | | | | | |
| | Aib-4Pal-Q-R-G- | | | | | | |
| | dProc4Bn4F | | | | | | |
| 77 | TyrEtNAc-4Pal- | 0.079 | 2070.88647 | C103H120F2N22O23 | 2072.19 | 1036.48 | [M + 2H]/2+ |
| | Phe4COOH-Proc4F- | | | | | | |
| | sbMeW4F-Aib-E-W- | | | | | | |
| | Aib-PyrimAla-Q-S-G- | | | | | | |
| | dProc3Bn | | | | | | |
| 78 | TyrEtNAc-4Pal- | 0.036 | 2083.91811 | C104H123F2N23O22 | 2085.23 | 1043.45 | [M + 2H]/2+ |
| | Phe4COOH-Proc4F- | | | | | | |
| | sbMeW4F-Aib-E-W- | | | | | | |
| | Aib-PyrimAla-Q-Dab- | | | | | | |
| | G-dProc3Bn | | | | | | |
| 79 | TyrEtNAc-4Pal- | 0.059 | 2070.88647 | C103H120F2N22O23 | 2072.19 | 1036.52 | [M + 2H]/2+ |
| | Phe4COOH-Proc4F- | | | | | | |
| | sbMeW4F-Aib-E-W- | | | | | | |
| | Aib-PyrimAla-Q-S-G- | | | | | | |
| | dProc4Bn | | | | | | |
| 80 | TyrEtNAc-4Pal- | 0.048 | 2083.91811 | C104H123F2N23O22 | 2085.23 | 1042.83 | [M + 2H]/2+ |
| | Phe4COOH-Proc4F- | | | | | | |
| | sbMeW4F-Aib-E-W- | | | | | | |
| | Aib-PyrimAla-Q-Dab- | | | | | | |
| | G-dProc4Bn | | | | | | |
| 81 | Phe4Pyrim5CO2H- | 0.049 | 2134.87264 | C106H117F3N24O22 | 2136.21 | 1068.156 | [M + 2H]/2+ |
| | 4Pal-Phe4COOH-G- | | | | | | |
| | Proc4F-SbMeW4F- | | | | | | |
| | Aib-E-W-Ac4cN-4Pal- | | | | | | |
| | Q-Dab-G-dProc4Bn4F | | | | | | |
| 82 | Phe4Pyrim5CO2H- | 0.054 | 2111.89304 | C105H120F3N23O22 | 2113.21 | 1056.756 | [M + 2H]/2+ |
| | AlaPent-Phe4COOH- | | | | | | |
| | G-Proc4F-SbMeW4F- | | | | | | |
| | Aib-E-W-Aib- | | | | | | |
| | PyrimAla-Q-Dab-G- | | | | | | |
| | dProc4Bn4F | | | | | | |
| 83 | Phe4Pyrim5CO2H- | 0.045 | 2069.88247 | C103H118F3N23O21 | 2071.18 | 1036.306 | [M + 2H]/2+ |
| | AlaPent-Phe4COOH- | | | | | | |
| | G-Proc4F-SbMeW4F- | | | | | | |
| | Aib-S-W-Aib- | | | | | | |
| | PyrimAla-Q-Dab-G- | | | | | | |
| | dProc4Bn4F | | | | | | |
| 84 | Phe4Pyrim5CO2H- | 0.047 | 2068.88722 | C104H119F3N22O21 | 2070.19 | 1035.601 | [M + 2H]/2+ |
| | AlaPent-Phe4COOH- | | | | | | |
| | G-Proc4F-SbMeW4F- | | | | | | |
| | Aib-S-W-Aib-4Pal-Q- | | | | | | |
| | Dab-G-dProc4Bn4F | | | | | | |
| 85 | Phe4Pyrim5CO2H- | 0.074 | 2046.87658 | C103H116F2N24O20 | 2048.17 | 1024.671 | [M + 2H]/2+ |
| | 4Pal-Phe4COOH-G- | | | | | | |
| | Proc4F-SbMeW4F- | | | | | | |
| | Aib-A-W-Aib-4Pal-Q- | | | | | | |
| | Dab-G- | | | | | | |
| | dProc4CH24Pal | | | | | | |
| 86 | Phe4Pyrim5CO2H- | 0.075 | 2093.90246 | C105H121F2N23O22 | 2095.22 | 1047.706 | [M + 2H]/2+ |
| | AlaPent-Phe4COOH- | | | | | | |
| | G-Proc4F-SbMeW4F- | | | | | | |
| | Aib-E-W-Aib-4Pal-Q- | | | | | | |
| | Dab-G- | | | | | | |
| | dProc4CH24Pal | | | | | | |
| 87 | Phe4Pyrim5CO2H- | 0.087 | 2036.89223 | C102H118F2N24O20 | 2038.17 | 1019.735 | [M + 2H]/2+ |
| | AlaPent-Phe4COOH- | | | | | | |
| | G-Proc4F-SbMeW4F- | | | | | | |
| | Aib-A-W-Aib- | | | | | | |
| | PyrimAla-Q-Dab-G- | | | | | | |
| | dProc4CH24Pal | | | | | | |
| 88 | Phe4Pyrim5CO2H- | 0.083 | 2035.89698 | C103H119F2N23O20 | 2037.19 | 1018.795 | [M + 2H]/2+ |
| | AlaPent-Phe4COOH- | | | | | | |
| | G-Proc4F-SbMeW4F- | | | | | | |
| | Aib-A-W-Aib-4Pal-Q- | | | | | | |
| | Dab-G- | | | | | | |
| | dProc4CH24Pal | | | | | | |
| 89 | Phe4Pyrim5CO2H- | 0.045 | 2051.89189 | C103H119F2N23O21 | 2053.19 | 1026.787 | [M + 2H]/2+ |
| | AlaPent-Phe4COOH- | | | | | | |
| | G-Proc4F-SbMeW4F- | | | | | | |
| | Aib-S-W-Aib-4Pal-Q- | | | | | | |
| | Dab-G- | | | | | | |
| | dProc4CH24Pal | | | | | | |
| 90 | Phe4AcPip-AlaPent- | 0.064 | 2114.96547 | C107H129F3N22O21 | 2116.3 | 1058.284 | [M + 2H]/2+ |
| | Phe4COOH-G-Proc4F- | | | | | | |
| | SbMeW4F-Aib-E-W- | | | | | | |
| | Aib-4Pal-Q-Dab-G- | | | | | | |
| | dProc4Bn4F | | | | | | |
| 91 | PyrimAla4Ph4CO2H- | 0.054 | 2122.87264 | C105H117F3N24O22 | 2124.2 | 1062.515 | [M + 2H]/2+ |
| | 4Pal-Phe4COOH-G- | | | | | | |
| | Proc4F-SbMeW4F- | | | | | | |
| | Aib-E-W-Aib- | | | | | | |
| | PyrimAla-Q-Dab-G- | | | | | | |
| | dProc4Bn4F | | | | | | |
| 92 | PyrimAla4Ph4CO2H- | 0.07 | 2111.89304 | C105H120F3N23O22 | 2113.21 | 1056.638 | [M + 2H]/2+ |
| | AlaPent-Phe4COOH- | | | | | | |
| | G-Proc4F-SbMeW4F- | | | | | | |
| | Aib-E-W-Aib- | | | | | | |
| | PyrimAla-Q-Dab-G- | | | | | | |
| | dProc4Bn4F | | | | | | |
| 93 | Bip4CO2H-AlaPent- | 0.059 | 2029.89631 | C102H121F2N21O22 | 2031.18 | 1016.092 | [M + 2H]/2+ |
| | Phe4COOH-G-Proc4F- | | | | | | |
| | SbMeW4F-Aib-E-W- | | | | | | |
| | Aib-Dap-Q-Dab-G- | | | | | | |
| | dProc4CH24Pal | | | | | | |
| 94 | Phe4Pyrim5CO2H- | 0.096 | 1991.86067 | C99H116F3N21O21 | 1993.1 | 996.85 | [M + 2H]/2+ |
| | AlaPent-Phe4COOH- | | | | | | |
| | G-Proc4F-SbMeW4F- | | | | | | |
| | Aib-A-W-Aib-S-Q- | | | | | | |
| | Dab-G-dProc4Bn4F | | | | | | |
| 95 | TyrEtNAc-4Pal- | 0.054 | 2100.91344 | C105H123F3N22O22 | 2102.23 | 1051.67 | [M + 2H]/2+ |
| | Phe4COOH-G-Proc4F- | | | | | | |
| | SbMeW4F-Aib-E-W- | | | | | | |
| | Aib-4Pal-Q-Dab-G- | | | | | | |
| | dProc4Bn4F | | | | | | |
| 96 | Bip4CO2H-AlaPent- | 0.067 | 2016.86468 | C101H118F2N20O23 | 2018.13 | 2018.081 | [M + H]+ |
| | Phe4COOH-G-Proc4F- | | | | | | |
| | SbMeW4F-Aib-E-W- | | | | | | |
| | Aib-Dap-Q-S-G- | | | | | | |
| | dProc4CH24Pal | | | | | | |
| 97 | Bip4CO2H-AlaPent- | 0.036 | 2021.86001 | C101H118F3N19O23 | 2023.13 | 2023.606 | [M + H]+ |
| | Phe4COOH-G-Proc4F- | | | | | | |
| | SbMeW4F-aMeS-S- | | | | | | |
| | W-Aib-S-Q-Dab-G- | | | | | | |
| | dProc4Bn4F | | | | | | |
| 98 | Bip4CO2H-AlaPent- | 0.058 | 2110.89779 | C106H121F3N22O22 | 2112.22 | 2113.292 | [M + H]+ |
| | 3Pal4CO2H-G-Proc4F- | | | | | | |
| | SbMeW4F-Aib-E-W- | | | | | | |
| | Aib-PyrimAla-Q-Dab- | | | | | | |
| | G-dProc4Bn4F | | | | | | |
| 99 | F4bcpA-AlaPent- | 0.031 | 2099.91819 | C106H124F3N21O22 | 2101.24 | 2102.008 | [M + H]+ |
| | Phe4COOH-G-Proc4F- | | | | | | |
| | SbMeW4F-Aib-E-W- | | | | | | |
| | Aib-PyrimAla-Q-Dab- | | | | | | |
| | G-dProc4Bn4F | | | | | | |
| 100 | 3Pal4Ph4CO2H- | 0.074 | 2047.88689 | C102H120F3N21O22 | 2049.17 | 683.909 | [M + 3H]/3+ |
| | AlaPent-Phe4COOH- | | | | | | |
| | G-Proc4F-SbMeW4F- | | | | | | |
| | Aib-E-W-Aib-Dap-Q- | | | | | | |
| | Dab-G-dProc4Bn4F | | | | | | |
| 101 | Bip4CO2H-4Pal- | 0.021 | 2120.88214 | C107H119F3N22O22 | 2122.22 | 1061.73 | [M + 2H]/2+ |
| | Phe4COOH-G-Proc4F- | | | | | | |
| | SbMeW4F-Aib-E-W- | | | | | | |
| | Aib-AlaPyrz-Q-Dab- | | | | | | |
| | G-dProc4Bn4F | | | | | | |
| 102 | Bip4CO2H-4Pal- | 0.021 | 2120.88214 | C107H119F3N22O22 | 2122.22 | 1061.55 | [M + 2H]/2+ |
| | Phe4COOH-G-Proc4F- | | | | | | |
| | SbMeW4F-Aib-E-W- | | | | | | |
| | Aib-3PyridaAla-Q- | | | | | | |
| | Dab-G-dProc4Bn4F | | | | | | |
| 103 | F4pcCCA-AlaPent- | 0.06 | 2131.9444 | C107H128F3N21O23 | 2133.28 | 2132.69 | [M + H]+ |
| | Phe4COOH-G-Proc4F- | | | | | | |
| | SbMeW4F-aMeS-E- | | | | | | |
| | W-Aib-PyrimAla-Q- | | | | | | |
| | Dab-G-dProc4Bn4F | | | | | | |
| 104 | F4pcCCA-AlaPent- | 0.085 | 2189.00225 | C110H135F3N22O23 | 2190.38 | 1095.71 | [M + 2H]/2+ |
| | Phe4COOH-G-Proc4F- | | | | | | |
| | SbMeW4F-aMeS-E- | | | | | | |
| | W-aMeK-PyrimAla-Q- | | | | | | |
| | Dab-G-dProc4Bn4F | | | | | | |
| 105 | F4pcCCA-AlaPent- | 0.083 | 2094.94916 | C105H129F3N20O23 | 2096.26 | 2095.64 | [M + H]+ |
| | Phe4COOH-G-Proc4F- | | | | | | |
| | SbMeW4F-Aib-E-W- | | | | | | |
| | PipH-S-Q-Dab-G- | | | | | | |
| | dProc4Bn4F | | | | | | |
| 106 | F4pcCCA-AlaPent- | 0.031 | 2011.91204 | C101H124F3N19O22 | 2013.17 | 2012.5 | [M + H]+ |
| | Phe4COOH-G-Proc4F- | | | | | | |
| | SbMeW4F-Aib-S-W- | | | | | | |
| | Aib-S-Q-Dab-G- | | | | | | |
| | dProc4Bn4F | | | | | | |
| 107 | F4pcCCA-AlaPent- | 0.063 | 2069.91752 | C103H126F3N19O24 | 2071.21 | 2071.16 | [M + H]+ |
| | Phe4COOH-G-Proc4F- | | | | | | |
| | SbMeW4F-aMeS-E- | | | | | | |
| | W-Aib-S-Q-Dab-G- | | | | | | |
| | dProc4Bn4F | | | | | | |
| 108 | Bip4CO2H-4Pal- | 0.024 | 2075.89189 | C105H119F2N23O21 | 2077.21 | 1039.597 | [M + 2H]/2+ |
| | Phe4COOH-G-Proc4F- | | | | | | |
| | SbMeW4F-Aib-S-W- | | | | | | |
| | Aib-PyrimAla-Q-Orn- | | | | | | |
| | G-dProc4CH24Pal | | | | | | |
| 109 | Bip4CO2H-AlaPent- | 0.036 | 2064.9123 | C105H122F2N22O21 | 2066.22 | 1034.073 | [M + 2H]/2+ |
| | Phe4COOH-G-Proc4F- | | | | | | |
| | SbMeW4F-Aib-S-W- | | | | | | |
| | Aib-PyrimAla-Q-Orn- | | | | | | |
| | G-dProc4CH24Pal | | | | | | |
| 110 | Bip4CO2H-AlaPent- | 0.038 | 2032.91238 | C103H123F3N20O21 | 2034.2 | 1018.09 | [M + 2H]/2+ |
| | Phe4COOH-G-Proc4F- | | | | | | |
| | SbMeW4F-Aib-S-W- | | | | | | |
| | Aib-Orn-Q-Dab-G- | | | | | | |
| | dProc4Bn4F | | | | | | |
| 111 | Bip4CO2H-4Pal- | 0.019 | 2077.87116 | C104H117F2N23O22 | 2079.18 | 1040.537 | [M + 2H]/2+ |
| | Phe4COOH-G-Proc4F- | | | | | | |
| | SbMeW4F-Aib-S-W- | | | | | | |
| | daMeS-PyrimAla-Q- | | | | | | |
| | Dab-G- | | | | | | |
| | dProc4CH24Pal | | | | | | |
| 112 | Bip4CO2H-AlaPent- | 0.018 | 2066.89156 | C104H120F2N22O22 | 2068.2 | 1034.661 | [M + 2H]/2+ |
| | Phe4COOH-G-Proc4F- | | | | | | |
| | SbMeW4F-Aib-S-W- | | | | | | |
| | daMeS-PyrimAla-Q- | | | | | | |
| | Dab-G- | | | | | | |
| | dProc4CH24Pal | | | | | | |
| 113 | Bip4CO2H-4Pal- | 0.018 | 2120.88214 | C107H119F3N22O22 | 2122.22 | 1061.19 | [M + 2H]/2+ |
| | Phe4COOH-G-Proc4F- | | | | | | |
| | SbMeW4F-Aib-E-W- | | | | | | |
| | Aib-2PyrimAla-Q- | | | | | | |
| | Dab-G-dProc4Bn4F | | | | | | |
| 114 | F4ptCCA-4Pal- | 0.035 | 2126.92909 | C107H125F3N22O22 | 2128.27 | 710.071 | [M + 3H]/3+ |
| | Phe4COOH-G-Proc4F- | | | | | | |
| | SbMeW4F-Aib-E-W- | | | | | | |
| | Aib-PyrimAla-Q-Dab- | | | | | | |
| | G-dProc4Bn4F | | | | | | |
| 115 | Bip4CO2H-4Pal- | 0.023 | 2120.88214 | C107H119F3N22O22 | 2122.22 | 2121.8 | [M + H]+ |
| | Phe4COOH-G-Proc4F- | | | | | | |
| | sbMeW4F-Aib-E-W- | | | | | | |
| | Aib-4PyridaAla-Q- | | | | | | |
| | Dab-G-dProc4Bn4F | | | | | | |
| 116 | Bip4CO2H- | 0.04 | 2121.87739 | C106H118F3N23O22 | 2123.21 | 2123.29 | [M + H]+ |
| | 4PyridaAla- | | | | | | |
| | Phe4COOH-G-Proc4F- | | | | | | |
| | sbMeW4F-Aib-E-W- | | | | | | |
| | Aib-PyrimAla-Q-Dab- | | | | | | |
| | G-dProc4Bn4F | | | | | | |
| 117 | F4pcCCA-4Pal- | 0.027 | 2126.92909 | C107H125F3N22O22 | 2128.27 | 2128.33 | [M + H]+ |
| | Phe4COOH-G-Proc4F- | | | | | | |
| | sbMeW4F-Aib-E-W- | | | | | | |
| | Aib-AlaPyrz-Q-Dab- | | | | | | |
| | G-dProc4Bn4F | | | | | | |
| 118 | Bip4CO2H-4Pal- | 0.016 | 2126.91786 | C108H125F3N20O23 | 2128.26 | 1064.14 | [M + 2H]/2+ |
| | Phe4COOH-G-Proc4F- | | | | | | |
| | sbMeW4F-Aib-E-W- | | | | | | |
| | Aib-AlaTHP4-Q-Dab- | | | | | | |
| | G-dProc4Bn4F | | | | | | |
| 119 | Bip4CO2H-4Pal- | 0.016 | 2125.93384 | C108H126F3N21O22 | 2127.28 | 1064.1 | [M + 2H]/2+ |
| | Phe4COOH-G-Proc4F- | | | | | | |
| | sbMeW4F-Aib-E-W- | | | | | | |
| | Aib-AlaPip4-Q-Dab- | | | | | | |
| | G-dProc4Bn4F | | | | | | |
| 120 | Bip4CO2H-4Pal- | 0.014 | 2126.92909 | C107H125F3N22O22 | 2128.27 | 1064.11 | [M + 2H]/2+ |
| | Phe4COOH-G-Proc4F- | | | | | | |
| | sbMeW4F-Aib-E-W- | | | | | | |
| | Aib-AlaPiperaz-Q- | | | | | | |
| | Dab-G-dProc4Bn4F | | | | | | |
| 121 | Bip4CO2H-4Pal- | 0.029 | 2112.9022 | C107H123F3N20O23 | 2114.24 | 1057.74 | [M + 2H]/2+ |
| | Phe4COOH-G-Proc4F- | | | | | | |
| | sbMeW4F-Aib-E-W- | | | | | | |
| | Aib-AlaPip4-Q-S-G- | | | | | | |
| | dProc4Bn4F | | | | | | |
| 122 | Bip4CO2H-4Pal- | 0.019 | 2113.89745 | C106H122F3N21O23 | 2115.22 | 2115.35 | [M + H]+ |
| | Phe4COOH-G-Proc4F- | | | | | | |
| | sbMeW4F-Aib-E-W- | | | | | | |
| | Aib-AlaPiperaz-Q-S- | | | | | | |
| | G-dProc4Bn4F | | | | | | |
| 123 | Bip4CO2H-4Pal- | 0.017 | 2127.9131 | C107H124F3N21O23 | 2129.25 | 1065.11 | [M + 2H]/2+ |
| | Phe4COOH-G-Proc4F- | | | | | | |
| | sbMeW4F-Aib-E-W- | | | | | | |
| | Aib-AlaMph-Q-Dab- | | | | | | |
| | G-dProc4Bn4F | | | | | | |
| 124 | Bip4CO2H-4Pal- | 0.018 | 2114.88147 | C106H121F3N20O24 | 2116.21 | 1058.53 | [M + 2H]/2+ |
| | Phe4COOH-G-Proc4F- | | | | | | |
| | sbMeW4F-Aib-E-W- | | | | | | |
| | Aib-AlaMph-Q-S-G- | | | | | | |
| | dProc4Bn4F | | | | | | |
| 125 | Bip4CO2H-4Pal- | 0.041 | 2114.88147 | C106H121F3N20O24 | 2116.21 | 1059.21 | [M + 2H]/2+ |
| | Phe4COOH-G-Proc4F- | | | | | | |
| | sbMeW4F-Aib-E-W- | | | | | | |
| | Aib-AlaMph-Q-S-G- | | | | | | |
| | dProc4Bn4F | | | | | | |
| 126 | Bip4CO2H-4Pal- | 0.022 | 2127.9131 | C107H124F3N21O23 | 2129.25 | 1065.27 | [M + 2H]/2+ |
| | Phe4COOH-G-Proc4F- | | | | | | |
| | sbMeW4F-Aib-E-W- | | | | | | |
| | Aib-AlaMph-Q-Dab- | | | | | | |
| | G-dProc4Bn4F | | | | | | |
| 127 | Bip4CO2H-4Pal- | 0.013 | 2076.87591 | C105H118F2N22O22 | 2078.19 | 1039.45 | [M + 2H]/2+ |
| | Phe4COOH-G- | | | | | | |
| | Proc4OH-SbMeW4F- | | | | | | |
| | Aib-S-W-Aib- | | | | | | |
| | PyrimAla-Q-Dab-G- | | | | | | |
| | dProc4Bn4F | | | | | | |
| 128 | Bip4CO2H-4Pal- | 0.009 | 2122.8614 | C106H117F3N22O23 | 2124.19 | 1062.45 | [M + 2H]/2+ |
| | Phe4COOH-G-Proc4F- | | | | | | |
| | SbMeW4F-Aib-S-W- | | | | | | |
| | daMeD-PyrimAla-Q- | | | | | | |
| | Dab-G-dProc4Bn4F | | | | | | |
| 129 | Bip4CO2H-4Pal- | 0.019 | 2107.89812 | C106H120F3N23O21 | 2109.22 | 703.65 | [M + 3H]/3+ |
| | Phe4COOH-G-Proc4F- | | | | | | |
| | SbMeW4F-aMeDab-S- | | | | | | |
| | W-Aib-PyrimAla-Q- | | | | | | |
| | Dab-G-dProc4Bn4F | | | | | | |
| 130 | Bip4CO2H-4Pal- | 0.039 | 2107.89812 | C106H120F3N23O21 | 2109.22 | 703.65 | [M + 3H]/3+ |
| | Phe4COOH-G-Proc4F- | | | | | | |
| | SbMeW4F-Aib-S-W- | | | | | | |
| | aMeDab-PyrimAla-Q- | | | | | | |
| | Dab-G-dProc4Bn4F | | | | | | |
| 131 | Bip4CO2H-4Pal- | 0.019 | 2107.89812 | C106H120F3N23O21 | 2109.22 | 703.65 | [M + 3H]/3+ |
| | Phe4COOH-G-Proc4F- | | | | | | |
| | SbMeW4F-daMeDab- | | | | | | |
| | S-W-Aib-PyrimAla-Q- | | | | | | |
| | Dab-G-dProc4Bn4F | | | | | | |
| 132 | Bip4CO2H-4Pal- | 0.015 | 2191.95564 | C111H128F3N23O22 | 2193.34 | 731.65 | [M + 3H]/3+ |
| | Phe4COOH-G-Proc4F- | | | | | | |
| | SbMeW4F-Aib-E-W- | | | | | | |
| | aMeK-PyrimAla-Q- | | | | | | |
| | Dab-Sar-dProc4Bn4F | | | | | | |
| 133 | Bip4CO2H-4Pal- | 0.021 | 2149.94507 | C109H126F3N23O21 | 2151.3 | 717.65 | [M + 3H]/3+ |
| | Phe4COOH-G-Proc4F- | | | | | | |
| | SbMeW4F-Aib-hS-W- | | | | | | |
| | aMeK-PyrimAla-Q- | | | | | | |
| | Dab-G-dProc4Bn4F | | | | | | |
| 134 | Bip4CO2H-4Pal- | 0.021 | 2119.93451 | C108H124F3N23O20 | 2121.28 | 707.65 | [M + 3H]/3+ |
| | Phe4COOH-G-Proc4F- | | | | | | |
| | SbMeW4F-Aib-A-W- | | | | | | |
| | aMeK-PyrimAla-Q- | | | | | | |
| | Dab-G-dProc4Bn4F | | | | | | |
| 135 | Bip4CO2H-4Pal- | 0.035 | 2147.96581 | C110H128F3N23O20 | 2149.33 | 717 | [M + 3H]/3+ |
| | Phe4COOH-G-Proc4F- | | | | | | |
| | SbMeW4F-Aib-V-W- | | | | | | |
| | aMeK-PyrimAla-Q- | | | | | | |
| | Dab-G-dProc4Bn4F | | | | | | |
| 136 | Bip4CO2H-4Pal- | 0.027 | 2161.98146 | C111H130F3N23O20 | 2163.36 | 721.65 | [M + 3H]/3+ |
| | Phe4COOH-G-Proc4F- | | | | | | |
| | SbMeW4F-Aib-alI-W- | | | | | | |
| | aMeK-PyrimAla-Q- | | | | | | |
| | Dab-G-dProc4Bn4F | | | | | | |
| 137 | Bip4CO2H-4Pal- | 0.022 | 2149.94507 | C109H126F3N23O21 | 2151.3 | 717.65 | [M + 3H]/3+ |
| | Phe4COOH-G-Proc4F- | | | | | | |
| | SbMeW4F-Aib-T-W- | | | | | | |
| | aMeK-PyrimAla-Q- | | | | | | |
| | Dab-G-dProc4Bn4F | | | | | | |
| 138 | Bip4CO2H-4Pal- | 0.021 | 2149.94507 | C109H126F3N23O21 | 2151.3 | 717.65 | [M + 3H]/3+ |
| | Phe4COOH-G-Proc4F- | | | | | | |
| | SbMeW4F-Aib-alT-W- | | | | | | |
| | aMeK-PyrimAla-Q- | | | | | | |
| | Dab-G-dProc4Bn4F | | | | | | |
| 139 | Bip4CO2H-4Pal- | 0.034 | 2094.84202 | C105H117ClF2N22O21 | 2096.64 | 699.3 | [M + 3H]/3+ |
| | Phe4COOH-G-Proc4F- | | | | | | |
| | SbMeW4Cl-Aib-S-W- | | | | | | |
| | Aib-PyrimAla-Q-Dab- | | | | | | |
| | G-dProc4Bn4F | | | | | | |
| 140 | Bip4CO2H-4Pal- | 0.033 | 2071.88575 | C107H119F2N21O21 | 2073.21 | 691.65 | [M + 3H]/3+ |
| | Phe4COOH-G-Proc4F- | | | | | | |
| | SbMe1Nal-Aib-S-W- | | | | | | |
| | Aib-PyrimAla-Q-Dab- | | | | | | |
| | G-dProc4Bn4F | | | | | | |
| 141 | Bip4CO2H-4Pal- | 0.011 | 2122.8614 | C106H117F3N22O23 | 2124.19 | 708.6 | [M + 3H]/3+ |
| | Phe4COOH-G-Proc4F- | | | | | | |
| | SbMeW4F-Aib-S-W- | | | | | | |
| | aMeD-PyrimAla-Q- | | | | | | |
| | Dab-G-dProc4Bn4F | | | | | | |
| 142 | Bip4CO2H-4Pal- | 0.019 | 2107.89812 | C106H120F3N23O21 | 2109.22 | 703.65 | [M + 3H]/3+ |
| | Phe4COOH-G-Proc4F- | | | | | | |
| | SbMeW4F-Aib-S-W- | | | | | | |
| | daMeDab-PyrimAla- | | | | | | |
| | Q-Dab-G-dProc4Bn4F | | | | | | |
| 143 | Bip4CO2H-4Pal- | 0.026 | 2180.80207 | C107H119BrF2N22O22 | 2183.12 | 1091.04 | [M + 2H]/2+ |
| | Phe4COOH-G-Proc4F- | | | | | | |
| | sbMeW4F-Aib-E-W- | | | | | | |
| | Aib-PyrimAla-Q-Dab- | | | | | | |
| | G-dProc4Bn4Br | | | | | | |
| 144 | Bip4CO2H-AlaPent- | 0.031 | 2124.94982 | C108H127F3N22O21 | 2126.29 | 709.3 | [M + 3H]/3+ |
| | Phe4COOH-G-Proc4F- | | | | | | |
| | SbMeW4F-Aib-S-W- | | | | | | |
| | aMeK-PyrimAla-Q- | | | | | | |
| | Dab-G-dProc4Bn4F | | | | | | |
| 145 | Bip4CO2H-AlaPent- | 0.041 | 2124.94982 | C108H127F3N22O21 | 2126.29 | 1063.45 | [M + 2H]/2+ |
| | Phe4COOH-G-Proc4F- | | | | | | |
| | SbMeW4F-Aib-S-W- | | | | | | |
| | daMeK-PyrimAla-Q- | | | | | | |
| | Dab-G-dProc4Bn4F | | | | | | |
| 146 | Bip4CO2H-AlaPent- | 0.034 | 2130.98554 | C109H133F3N20O22 | 2132.34 | 1066.5 | [M + 2H]/2+ |
| | Phe4COOH-G-Proc4F- | | | | | | |
| | SbMeW4F-Aib-S-W- | | | | | | |
| | aMeK-AlaTHP4-Q- | | | | | | |
| | Dab-G-dProc4Bn4F | | | | | | |
| 147 | Bip4CO2H-AlaPent- | 0.045 | 2138.96547 | C109H129F3N22O21 | 2140.32 | 1070.5 | [M + 2H]/2+ |
| | Phe4COOH-G-Proc4F- | | | | | | |
| | SbMeW4F-Aib-hS-W- | | | | | | |
| | aMeK-PyrimAla-Q- | | | | | | |
| | Dab-G-dProc4Bn4F | | | | | | |
| 148 | Bip4CO2H-4Pal- | 0.032 | 2136.85259 | C107H119ClF2N22O22 | 2138.67 | 2138.08 | [M + H]+ |
| | Phe4COOH-G-Proc4F- | | | | | | |
| | sbMeW4Cl-Aib-E-W- | | | | | | |
| | Aib-PyrimAla-Q-Dab- | | | | | | |
| | G-dProc4Bn4F | | | | | | |
| 149 | Bip4CO2H-4Pal- | 0.024 | 2113.89631 | C109H121F2N21O22 | 2115.25 | 1057.85 | [M + 2H]/2+ |
| | Phe4COOH-G-Proc4F- | | | | | | |
| | sbMe1Nal-Aib-E-W- | | | | | | |
| | Aib-PyrimAla-Q-Dab- | | | | | | |
| | G-dProc4Bn4F | | | | | | |
| 150 | Bip4CO2H-4Pal- | 0.019 | 2166.78642 | C106H117BrF2N22O22 | 2169.1 | 1084.34 | [M + 2H]/2+ |
| | Phe4COOH-G-Proc4F- | | | | | | |
| | sbMeW4F-Aib-E-W- | | | | | | |
| | Aib-PyrimAla-Q-Dap- | | | | | | |
| | G-dProc3Bn4Br | | | | | | |
| 151 | Bip4CO2H-4Pal- | 0.022 | 2194.81772 | C108H121BrF2N22O22 | 2197.15 | 1098.38 | [M + 2H]/2+ |
| | Phe4COOH-G-Proc4F- | | | | | | |
| | sbMeW4F-Aib-E-W- | | | | | | |
| | Aib-PyrimAla-Q-Orn- | | | | | | |
| | G-dProc3Bn4Br | | | | | | |
| 152 | Bip4CO2H-4Pal- | 0.031 | 2208.83337 | C109H123BrF2N22O22 | 2211.18 | 2209.66 | [M + H]+ |
| | Phe4COOH-G-Proc4F- | | | | | | |
| | sbMeW4F-Aib-E-W- | | | | | | |
| | Aib-PyrimAla-Q-K-G- | | | | | | |
| | dProc3Bn4Br | | | | | | |
| 153 | Bip4CO2H-4Pal- | 0.019 | 2173.81625 | C109H121BrFN21O22 | 2176.16 | 1088.34 | [M + 2H]/2+ |
| | Phe4COOH-G-Proc4F- | | | | | | |
| | sbMe1Nal-Aib-E-W- | | | | | | |
| | Aib-PyrimAla-Q-Dab- | | | | | | |
| | G-dProc3Bn4Br | | | | | | |
| 154 | Bip4CO2H-4Pal- | 0.011 | 2105.86607 | C105H117F2N23O23 | 2107.19 | 702.95 | [M + 3H]/3+ |
| | Phe4COOH-G-Proc4F- | | | | | | |
| | SbMeW4F-Aib-S-W- | | | | | | |
| | daMeD-PyrimAla-Q- | | | | | | |
| | Dab-G- | | | | | | |
| | dProc4CH24Pal | | | | | | |
| 155 | Bip4CO2H-AlaPent- | 0.011 | 2094.88647 | C105H120F2N22O23 | 2096.21 | 699.3 | [M + 3H]/3+ |
| | Phe4COOH-G-Proc4F- | | | | | | |
| | SbMeW4F-Aib-S-W- | | | | | | |
| | daMeD-PyrimAla-Q- | | | | | | |
| | Dab-G- | | | | | | |
| | dProc4CH24Pal | | | | | | |
| 156 | Bip4CO2H-4Pal- | 0.015 | 2092.83444 | C104H114F2N22O24 | 2094.15 | 698.6 | [M + 3H]/3+ |
| | Phe4COOH-G-Proc4F- | | | | | | |
| | SbMeW4F-Aib-S-W- | | | | | | |
| | daMeD-PyrimAla-Q-S- | | | | | | |
| | G-dProc4CH24Pal | | | | | | |
| 157 | Bip4CO2H-AlaPent- | 0.038 | 2079.85918 | C104H118FN21O25 | 2081.17 | 694.3 | [M + 3H]/3+ |
| | Phe4COOH-G- | | | | | | |
| | Proc4OH-SbMeW4F- | | | | | | |
| | Aib-S-W-daMeD- | | | | | | |
| | PyrimAla-Q-S-G- | | | | | | |
| | dProc4CH24Pal | | | | | | |
| 158 | Bip4CO2H-AlaPent- | 0.013 | 2081.85484 | C104H117F2N21O24 | 2083.16 | 694.95 | [M + 3H]/3+ |
| | Phe4COOH-G-Proc4F- | | | | | | |
| | SbMeW4F-Aib-S-W- | | | | | | |
| | daMeD-PyrimAla-Q-S- | | | | | | |
| | G-dProc4CH24Pal | | | | | | |
| 159 | Bip4CO2H-4Pal- | 0.013 | 2103.87041 | C105H118FN23O24 | 2105.2 | 702.3 | [M + 3H]/3+ |
| | Phe4COOH-G- | | | | | | |
| | Proc4OH-SbMeW4F- | | | | | | |
| | Aib-S-W-daMeD- | | | | | | |
| | PyrimAla-Q-Dab-G- | | | | | | |
| | dProc4CH24Pal | | | | | | |
| 160 | Bip4CO2H-AlaPent- | 0.023 | 2092.89081 | C105H121FN22O24 | 2094.22 | 698.65 | [M + 3H]/3+ |
| | Phe4COOH-G- | | | | | | |
| | Proc4OH-SbMeW4F- | | | | | | |
| | Aib-S-W-daMeD- | | | | | | |
| | PyrimAla-Q-Dab-G- | | | | | | |
| | dProc4CH24Pal | | | | | | |
| 161 | Bip4CO2H-4Pal- | 0.019 | 2090.83878 | C104H115FN22O25 | 2092.16 | 697.95 | [M + 3H]/3+ |
| | Phe4COOH-G- | | | | | | |
| | Proc4OH-SbMeW4F- | | | | | | |
| | Aib-S-W-daMeD- | | | | | | |
| | PyrimAla-Q-S-G- | | | | | | |
| | dProc4CH24Pal | | | | | | |
| 162 | Bip4CO2H-4Pal- | 0.05 | 2062.87149 | C103H116F2N24O21 | 2064.17 | 688.6 | [M + 3H]/3+ |
| | Phe4COOH-G-Proc4F- | | | | | | |
| | SbMeW4F-Aib-S- | | | | | | |
| | Trp4Az-Aib-PyrimAla- | | | | | | |
| | Q-Dab-G- | | | | | | |
| | dProc4CH24Pal | | | | | | |
| 163 | Bip4CO2H-cBuA- | 0.03 | 2038.89665 | C103H120F2N22O21 | 2040.19 | 680.65 | [M + 3H]/3+ |
| | Phe4COOH-G-Proc4F- | | | | | | |
| | SbMeW4F-Aib-S-W- | | | | | | |
| | Aib-PyrimAla-Q-Dab- | | | | | | |
| | G-dProc4CH24Pal | | | | | | |
| 164 | Bip4CO2H-cBuA- | 0.039 | 2080.89081 | C104H121FN22O24 | 2082.2 | 694.65 | [M + 3H]/3+ |
| | Phe4COOH-G- | | | | | | |
| | Proc4OH-SbMeW4F- | | | | | | |
| | Aib-S-W-daMeD- | | | | | | |
| | PyrimAla-Q-Dab-G- | | | | | | |
| | dProc4CH24Pal | | | | | | |
| 165 | Bip4CO2H-AlaPent- | 0.022 | 2173.99135 | C110H134F3N21O23 | 2175.36 | 1088.29 | [M + 2H]/2+ |
| | Phe4COOH-G-Proc4F- | | | | | | |
| | SbMeW4F-Aib-E-W- | | | | | | |
| | aMeK-AlaMph-Q- | | | | | | |
| | Dab-G-dProc4Bn4F | | | | | | |
| 166 | Bip4CO2H-AlaPent- | 0.039 | 2172.01209 | C111H136F3N21O22 | 2173.39 | 2173.38 | [M + H]+ |
| | Phe4COOH-G-Proc4F- | | | | | | |
| | SbMeW4F-Aib-E-W- | | | | | | |
| | aMeK-AlaPip4-Q-Dab- | | | | | | |
| | G-dProc4Bn4F | | | | | | |
| 167 | Bip4CO2H-AlaPent- | 0.013 | 2074.92294 | C105H125F3N20O22 | 2076.23 | 1038.2 | [M + 2H]/2+ |
| | Phe4COOH-G-Proc4F- | | | | | | |
| | SbMeW4F-Aib-S-W- | | | | | | |
| | Aib-AlaMph-Q-Dab- | | | | | | |
| | G-dProc4Bn4F | | | | | | |
| 168 | Bip4CO2H-AlaPent- | 0.018 | 2073.93892 | C105H126F3N21O21 | 2075.25 | 2075.42 | [M + H]+ |
| | Phe4COOH-G-Proc4F- | | | | | | |
| | SbMeW4F-Aib-S-W- | | | | | | |
| | Aib-AlaPiperaz-Q- | | | | | | |
| | Dab-G-dProc4Bn4F | | | | | | |
| 169 | Bip4CO2H-AlaPent- | 0.019 | 2072.94368 | C106H127F3N20O21 | 2074.26 | 2073.89 | [M + H]+ |
| | Phe4COOH-G-Proc4F- | | | | | | |
| | SbMeW4F-Aib-S-W- | | | | | | |
| | Aib-AlaPip4-Q-Dab- | | | | | | |
| | G-dProc4Bn4F | | | | | | |
| 170 | Bip4CO2H-AlaPent- | 0.015 | 2090.91786 | C105H125F3N20O23 | 2092.23 | 2091.57 | [M + H]+ |
| | Phe4COOH-G-Proc4F- | | | | | | |
| | SbMeW4F-Aib-S-W- | | | | | | |
| | daMeS-AlaMph-Q- | | | | | | |
| | Dab-G-dProc4Bn4F | | | | | | |
| 171 | Bip4CO2H-AlaPent- | 0.016 | 2089.93384 | C105H126F3N21O22 | 2091.25 | 2091.2 | [M + H]+ |
| | Phe4COOH-G-Proc4F- | | | | | | |
| | SbMeW4F-Aib-S-W- | | | | | | |
| | daMeS-AlaPiperaz-Q- | | | | | | |
| | Dab-G-dProc4Bn4F | | | | | | |
| 172 | Bip4CO2H-AlaPent- | 0.016 | 2088.93859 | C106H127F3N20O22 | 2090.26 | 2089.89 | [M + H]+ |
| | Phe4COOH-G-Proc4F- | | | | | | |
| | SbMeW4F-Aib-S-W- | | | | | | |
| | daMeS-AlaPip4-Q- | | | | | | |
| | Dab-G-dProc4Bn4F | | | | | | |
| 173 | Bip4CO2H-AlaPent- | 0.037 | 2118.94916 | C107H129F3N20O23 | 2120.28 | 2119.46 | [M + H]+ |
| | Phe4COOH-G-Proc4F- | | | | | | |
| | SbMeW4F-Aib-S-W- | | | | | | |
| | aMeK-AlaMph-Q-S-G- | | | | | | |
| | dProc4Bn4F | | | | | | |
| 174 | Bip4CO2H-AlaPent- | 0.025 | 2131.98079 | C108H132F3N21O22 | 2133.33 | 2132.76 | [M + H]+ |
| | Phe4COOH-G-Proc4F- | | | | | | |
| | SbMeW4F-Aib-S-W- | | | | | | |
| | aMeK-AlaMph-Q- | | | | | | |
| | Dab-G-dProc4Bn4F | | | | | | |
| 175 | Bip4CO2H-AlaPent- | 0.037 | 2117.96514 | C107H130F3N21O22 | 2119.3 | 2119.03 | [M + H]+ |
| | Phe4COOH-G-Proc4F- | | | | | | |
| | SbMeW4F-Aib-S-W- | | | | | | |
| | aMeK-AlaPiperaz-Q- | | | | | | |
| | S-G-dProc4Bn4F | | | | | | |
| 176 | Bip4CO2H-AlaPent- | 0.036 | 2084.92728 | C106H126F2N20O23 | 2086.25 | 2085.46 | [M + H]+ |
| | Phe4COOH-G-Proc4F- | | | | | | |
| | SbMeW4F-Aib-E-W- | | | | | | |
| | Aib-AlaPip4-Q-S-G- | | | | | | |
| | dProc4CH24Pal | | | | | | |
| 177 | Bip4CO2H-AlaPent- | 0.031 | 2102.90146 | C105H124F2N20O25 | 2104.22 | 2104.16 | [M + H]+ |
| | Phe4COOH-G-Proc4F- | | | | | | |
| | SbMeW4F-Aib-E-W- | | | | | | |
| | daMeS-AlaMph-Q-S- | | | | | | |
| | G-dProc4CH24Pal | | | | | | |
| 178 | Bip4CO2H-AlaPent- | 0.041 | 2100.92219 | C106H126F2N20O24 | 2102.25 | 1051.42 | [M + 2H]/2+ |
| | Phe4COOH-G-Proc4F- | | | | | | |
| | SbMeW4F-Aib-E-W- | | | | | | |
| | daMeS-AlaPip4-Q-S- | | | | | | |
| | G-dProc4CH24Pal | | | | | | |
| 179 | Bip4CO2H-AlaPent- | 0.029 | 2044.89598 | C103H122F2N20O23 | 2046.19 | 2046.17 | [M + H]+ |
| | Phe4COOH-G-Proc4F- | | | | | | |
| | SbMeW4F-Aib-S-W- | | | | | | |
| | Aib-AlaMph-Q-S-G- | | | | | | |
| | dProc4CH24Pal | | | | | | |
| 180 | Bip4CO2H-AlaPent- | 0.027 | 2042.91671 | C104H124F2N20O22 | 2044.21 | 1022.63 | [M + 2H]/2+ |
| | Phe4COOH-G-Proc4F- | | | | | | |
| | SbMeW4F-Aib-S-W- | | | | | | |
| | Aib-AlaPip4-Q-S-G- | | | | | | |
| | dProc4CH24Pal | | | | | | |
| 181 | Bip4CO2H-AlaPent- | 0.021 | 2058.91163 | C104H124F2N20O23 | 2060.21 | 2059.87 | [M + H]+ |
| | Phe4COOH-G-Proc4F- | | | | | | |
| | SbMeW4F-Aib-S-W- | | | | | | |
| | daMeS-AlaPip4-Q-S- | | | | | | |
| | G-dProc4CH24Pal | | | | | | |
| 182 | Bip4CO2H-AlaPent- | 0.049 | 2106.90646 | C106H123FN22O24 | 2108.24 | 1054.45 | [M + 2H]/2+ |
| | Phe4COOH-G- | | | | | | |
| | Proc4OH-SbMeW4F- | | | | | | |
| | Aib-alT-W-daMeD- | | | | | | |
| | PyrimAla-Q-Dab-G- | | | | | | |
| | dProc4CH24Pal | | | | | | |
| 183 | Phe4AcPip-4Pal- | 0.025 | 2084.92976 | C104H123F3N24O20 | 2086.23 | 696 | [M + 3H]/3+ |
| | Phe4COOH-G-Proc4F- | | | | | | |
| | SbMeW4F-Aib-S-W- | | | | | | |
| | Aib-PyrimAla-Q-Dab- | | | | | | |
| | G-dProc4Bn4F | | | | | | |
| 184 | Phe4AcPip-4Pal- | 0.014 | 2128.91959 | C105H123F3N24O22 | 2130.24 | 710.65 | [M + 3H]/3+ |
| | Phe4COOH-G-Proc4F- | | | | | | |
| | SbMeW4F-Aib-S-W- | | | | | | |
| | aMeD-PyrimAla-Q- | | | | | | |
| | Dab-G-dProc4Bn4F | | | | | | |
| 185 | Phe4Pyrim5CO2H- | 0.043 | 2050.89148 | C102H119FN24O22 | 2052.18 | 684.65 | [M + 3H]/3+ |
| | AlaPent-Phe4COOH- | | | | | | |
| | G-Proc4OH- | | | | | | |
| | SbMeW4F-Aib-S-W- | | | | | | |
| | Aib-PyrimAla-Q-Dab- | | | | | | |
| | G-dProc4CH24Pal | | | | | | |
| 186 | Phe4Pyrim5CO2H- | 0.015 | 2063.86674 | C102H115F2N25O21 | 2065.16 | 688.95 | [M + 3H]/3+ |
| | 4Pal-Phe4COOH-G- | | | | | | |
| | Proc4F-SbMeW4F- | | | | | | |
| | Aib-S-W-Aib- | | | | | | |
| | PyrimAla-Q-Dab-G- | | | | | | |
| | dProc4CH24Pal | | | | | | |
| 187 | Phe4Pyrim5CO2H- | 0.015 | 2107.85657 | C103H115F2N25O23 | 2109.17 | 703.6 | [M + 3H]/3+ |
| | 4Pal-Phe4COOH-G- | | | | | | |
| | Proc4F-SbMeW4F- | | | | | | |
| | Aib-S-W-aMeD- | | | | | | |
| | PyrimAla-Q-Dab-G- | | | | | | |
| | dProc4CH24Pal | | | | | | |
| 188 | Phe4Pyrim5CO2H- | 0.02 | 2096.87697 | C103H118F2N24O23 | 2098.18 | 699.95 | [M + 3H]/3+ |
| | AlaPent-Phe4COOH- | | | | | | |
| | G-Proc4F-SbMeW4F- | | | | | | |
| | Aib-S-W-aMeD- | | | | | | |
| | PyrimAla-Q-Dab-G- | | | | | | |
| | dProc4CH24Pal | | | | | | |
| 189 | Bip4CO2H-4Pal- | 0.016 | 2098.87016 | C105H120F2N20O25 | 2100.19 | 1050.45 | [M + 2H]/2+ |
| | Phe4COOH-G-Proc4F- | | | | | | |
| | SbMeW4F-Aib-S-W- | | | | | | |
| | aMeD-AlaTHP4-Q-S- | | | | | | |
| | G-dProc4CH24Pal | | | | | | |
| 190 | Bip4CO2H-AlaPent- | 0.019 | 2064.8959 | C104H121FN22O23 | 2066.21 | 689.3 | [M + 3H]/3+ |
| | Phe4COOH-G- | | | | | | |
| | Proc4OH-SbMeW4F- | | | | | | |
| | Aib-S-W-daMeS- | | | | | | |
| | PyrimAla-Q-Dab-G- | | | | | | |
| | dProc4CH24Pal | | | | | | |
| 191 | Bip4CO2H-AlaPent- | 0.021 | 2098.92653 | C106H127FN20O25 | 2100.26 | 1050.45 | [M + 2H]/2+ |
| | Phe4COOH-G- | | | | | | |
| | Proc4OH-SbMeW4F- | | | | | | |
| | Aib-S-W-aMeD- | | | | | | |
| | AlaTHP4-Q-Dab-G- | | | | | | |
| | dProc4CH24Pal | | | | | | |
| 192 | Bip4CO2H-AlaPent- | 0.021 | 2070.93161 | C105H127FN20O24 | 2072.25 | 1036.45 | [M + 2H]/2+ |
| | Phe4COOH-G- | | | | | | |
| | Proc4OH-SbMeW4F- | | | | | | |
| | Aib-S-W-daMeS- | | | | | | |
| | AlaTHP4-Q-Dab-G- | | | | | | |
| | dProc4CH24Pal | | | | | | |
| 193 | Bip4CO2H-AlaPent- | 0.011 | 2100.92219 | C106H126F2N20O24 | 2102.25 | 1051.45 | [M + 2H]/2+ |
| | Phe4COOH-G-Proc4F- | | | | | | |
| | SbMeW4F-Aib-S-W- | | | | | | |
| | aMeD-AlaTHP4-Q- | | | | | | |
| | Dab-G- | | | | | | |
| | dProc4CH24Pal | | | | | | |
| 194 | Bip4CO2H-4Pal- | 0.014 | 2059.88058 | C104H118FN23O22 | 2061.19 | 687.65 | [M + 3H]/3+ |
| | Phe4COOH-G- | | | | | | |
| | Proc4OH-SbMeW4F- | | | | | | |
| | Aib-S-W-Aib- | | | | | | |
| | PyrimAla-Q-Dab-G- | | | | | | |
| | dProc4CH24Pal | | | | | | |
| 195 | Bip4CO2H-4Pal- | 0.013 | 2075.8755 | C104H118FN23O23 | 2077.19 | 692.95 | [M + 3H]/3+ |
| | Phe4COOH-G- | | | | | | |
| | Proc4OH-SbMeW4F- | | | | | | |
| | Aib-S-W-daMeS- | | | | | | |
| | PyrimAla-Q-Dab-G- | | | | | | |
| | dProc4CH24Pal | | | | | | |
| 196 | Phe4AcPip-4Pal- | 0.038 | 2082.93409 | C104H124F2N24O21 | 2084.24 | 695.3 | [M + 3H]/3+ |
| | Phe4COOH-G- | | | | | | |
| | Proc4OH-SbMeW4F- | | | | | | |
| | Aib-S-W-Aib- | | | | | | |
| | PyrimAla-Q-Dab-G- | | | | | | |
| | dProc4Bn4F | | | | | | |
| 197 | Phe4Pyrim5CO2H- | 0.036 | 2061.87108 | C102H116FN25O22 | 2063.17 | 688.3 | [M + 3H]/3+ |
| | 4Pal-Phe4COOH-G- | | | | | | |
| | Proc4OH-SbMeW4F- | | | | | | |
| | Aib-S-W-Aib- | | | | | | |
| | PyrimAla-Q-Dab-G- | | | | | | |
| | dProc4CH24Pal | | | | | | |
| 198 | Phe4Pyrim5CO2H- | 0.024 | 2052.88714 | C102H118F2N24O21 | 2054.17 | 685.3 | [M + 3H]/3+ |
| | AlaPent-Phe4COOH- | | | | | | |
| | G-Proc4F-SbMcW4F- | | | | | | |
| | Aib-S-W-Aib- | | | | | | |
| | PyrimAla-Q-Dab-G- | | | | | | |
| | dProc4CH24Pal | | | | | | |
| 199 | Bip4CO2H-4Pal- | 0.032 | 2096.87449 | C105H121FN20O26 | 2098.2 | 1049.45 | [M + 2H]/2+ |
| | Phe4COOH-G- | | | | | | |
| | Proc4OH-SbMeW4F- | | | | | | |
| | Aib-S-W-aMeD- | | | | | | |
| | AlaTHP4-Q-S-G- | | | | | | |
| | dProc4CH24Pal | | | | | | |
| 200 | Bip4CO2H-4Pal- | 0.014 | 2111.90179 | C106H123F2N21O24 | 2113.23 | 704.95 | [M + 3H]/3+ |
| | Phe4COOH-G-Proc4F- | | | | | | |
| | SbMeW4F-Aib-S-W- | | | | | | |
| | aMeD-AlaTHP4-Q- | | | | | | |
| | Dab-G- | | | | | | |
| | dProc4CH24Pal | | | | | | |
| 201 | Bip4CO2H-4Pal- | 0.019 | 2109.90613 | C106H124FN21O25 | 2111.24 | 704.3 | [M + 3H]/3+ |
| | Phe4COOH-G- | | | | | | |
| | Proc4OH-SbMeW4F- | | | | | | |
| | Aib-S-W-aMeD- | | | | | | |
| | AlaTHP4-Q-Dab-G- | | | | | | |
| | dProc4CH24Pal | | | | | | |
| 202 | Bip4CO2H-AlaPent- | 0.013 | 2046.87524 | C102H120F2N20O24 | 2048.16 | 1024.45 | [M + 2H]/2+ |
| | Phe4COOH-G-Proc4F- | | | | | | |
| | SbMeW4F-Aib-S-W- | | | | | | |
| | aMeD-alT-Q-Dab-G- | | | | | | |
| | dProc4CH24Pal | | | | | | |
| 203 | Bip4CO2H-AlaPent- | 0.016 | 2044.89598 | C103H122F2N20O23 | 2046.19 | 1023.45 | [M + 2H]/2+ |
| | Phe4COOH-G-Proc4F- | | | | | | |
| | SbMeW4F-Aib-S-W- | | | | | | |
| | aMeD-V-Q-Dab-G- | | | | | | |
| | dProc4CH24Pal | | | | | | |
| 204 | Bip4CO2H-AlaPent- | 0.017 | 2072.92728 | C105H126F2N20O23 | 2074.24 | 1037.45 | [M + 2H]/2+ |
| | Phe4COOH-G-Proc4F- | | | | | | |
| | SbMeW4F-Aib-S-W- | | | | | | |
| | daMeS-AlaTHP4-Q- | | | | | | |
| | Dab-G- | | | | | | |
| | dProc4CH24Pal | | | | | | |
| 205 | Bip4CO2H-4Pal- | 0.028 | 2075.89189 | C105H119F2N23O21 | 2077.21 | 692.95 | [M + 3H]/3+ |
| | Phe4COOH-G-Proc4F- | | | | | | |
| | SbMeW4F-Aib-S-W- | | | | | | |
| | Aib-PyrimAla-Q-Dab- | | | | | | |
| | Sar-dProc4CH24Pal | | | | | | |
| 206 | Bip4CO2H-4Pal- | 0.022 | 2091.88681 | C105H119F2N23O22 | 2093.21 | 698.3 | [M + 3H]/3+ |
| | Phe4COOH-G-Proc4F- | | | | | | |
| | SbMeW4F-Aib-S-W- | | | | | | |
| | daMeS-PyrimAla-Q- | | | | | | |
| | Dab-Sar- | | | | | | |
| | dProc4CH24Pal | | | | | | |
| 207 | Bip4CO2H-4Pal- | 0.044 | 2095.92686 | C106H126FN21O24 | 2097.26 | 699.65 | [M + 3H]/3+ |
| | Phe4COOH-G- | | | | | | |
| | Proc4OH-SbMeW4F- | | | | | | |
| | Aib-S-W-daMeS- | | | | | | |
| | AlaTHP4-Q-Dab-Sar- | | | | | | |
| | dProc4CH24Pal | | | | | | |
| 208 | Bip4CO2H-4Pal- | 0.015 | 2125.91744 | C107H125F2N21O24 | 2127.26 | 709.65 | [M + 3H]/3+ |
| | Phe4COOH-G-Proc4F- | | | | | | |
| | SbMeW4F-Aib-S-W- | | | | | | |
| | aMeD-AlaTHP4-Q- | | | | | | |
| | Dab-Sar- | | | | | | |
| | dProc4CH24Pal | | | | | | |
| 209 | Bip4CO2H-4Pal- | 0.034 | 2117.88606 | C106H120FN23O24 | 2119.23 | 706.95 | [M + 3H]/3+ |
| | Phe4COOH-G- | | | | | | |
| | Proc4OH-SbMeW4F- | | | | | | |
| | Aib-S-W-aMeD- | | | | | | |
| | PyrimAla-Q-Dab-Sar- | | | | | | |
| | dProc4CH24Pal | | | | | | |
| 210 | Bip4CO2H-4Pal- | 0.029 | 2123.92178 | C107H126FN21O25 | 2125.27 | 708.95 | [M + 3H]/3+ |
| | Phe4COOH-G- | | | | | | |
| | Proc4OH-SbMeW4F- | | | | | | |
| | Aib-S-W-aMeD- | | | | | | |
| | AlaTHP4-Q-Dab-Sar- | | | | | | |
| | dProc4CH24Pal | | | | | | |
| 211 | Bip4CO2H-4Pal- | 0.019 | 2067.91196 | C105H123F2N21O22 | 2069.22 | 690.3 | [M + 3H]/3+ |
| | Phe4COOH-G-Proc4F- | | | | | | |
| | SbMeW4F-Aib-S-W- | | | | | | |
| | Aib-AlaTHP4-Q-Dab- | | | | | | |
| | G-dProc4CH24Pal | | | | | | |
| 212 | Bip4CO2H-4Pal- | 0.029 | 2065.9163 | C105H124FN21O23 | 2067.23 | 689.65 | [M + 3H]/3+ |
| | Phe4COOH-G- | | | | | | |
| | Proc4OH-SbMeW4F- | | | | | | |
| | Aib-S-W-Aib- | | | | | | |
| | AlaTHP4-Q-Dab-G- | | | | | | |
| | dProc4CH24Pal | | | | | | |
| 213 | Bip4CO2H-4Pal- | 0.021 | 2081.91121 | C105H124FN21O24 | 2083.23 | 694.95 | [M + 3H]/3+ |
| | Phe4COOH-G- | | | | | | |
| | Proc4OH-SbMeW4F- | | | | | | |
| | Aib-S-W-daMeS- | | | | | | |
| | AlaTHP4-Q-Dab-G- | | | | | | |
| | dProc4CH24Pal | | | | | | |
| 215 | Bip4CONH2-4Pal- | 0.01 | 2082.92286 | C105H124F2N22O22 | 2084.24 | 1042.46 | [M + 2H]/2+ |
| | Phe4COOH-G-Proc4F- | | | | | | |
| | SbMeW4F-Aib-S-W- | | | | | | |
| | daMeS-AlaTHP4-Q- | | | | | | |
| | Dab-G- | | | | | | |
| | dProc4CH24Pal | | | | | | |
| 216 | Bip4CONH2-AlaPent- | 0.008 | 2088.93859 | C106H127F3N20O22 | 2090.26 | 2089.50 | [M + H]+ |
| | Phe4COOH-G-Proc4F- | | | | | | |
| | SbMeW4F-Aib-S-W- | | | | | | |
| | daMeS-AlaTHP4-Q- | | | | | | |
| | Dab-G-dProc3Bn4F | | | | | | |
| 217 | Bip4CONH2-AlaPent- | 0.028 | 2098.92294 | C107H125F3N20O22 | 2100.25 | 2099.55 | [M + H]+ |
| | Phe4COOH-G-Proc4F- | | | | | | |
| | SbMeW4F-Aib-S-W- | | | | | | |
| | daMeN-AlaTHP4-Q- | | | | | | |
| | Dab-G-dProc3Bn4F | | | | | | |
| 218 | TyrEtNAc-4Pal- | 0.036 | 2055.93443 | C102H123F2N25O20 | 2057.22 | 686.72 | [M + 3H]/3+ |
| | Phe4CONH2-G- | | | | | | |
| | Proc4F-SbMeW4F- | | | | | | |
| | Aib-S-W-Aib- | | | | | | |
| | PyrimAla-Q-NMeDab- | | | | | | |
| | G-dProc4CH24Pal | | | | | | |
| 219 | Bip4CO2H-4Pal- | 0.018 | 2133.88614 | C108H121F2N21O24 | 2135.24 | 712.57 | [M + 3H]/3+ |
| | Phe4COOH-G-Proc4F- | | | | | | |
| | SbMeW4F-Aib-S-W- | | | | | | |
| | daMeD-hY-Q-Dab-G- | | | | | | |
| | dProc3CH24Pal | | | | | | |
| 220 | Bip4CO2H-4Pal- | 0.039 | 2089.89631 | C107H121F2N21O22 | 2091.23 | 2090.98 | [M + H]+ |
| | Phe4COOH-G-Proc4F- | | | | | | |
| | SbMeW4F-Aib-S-W- | | | | | | |
| | Aib-hY-Q-Dab-G- | | | | | | |
| | dProc3CH24Pal | | | | | | |
| 221 | Bip4CO2H-4Pal- | 0.02 | 2117.89123 | C108H121F2N21O23 | 2119.24 | 1059.96 | [M + 2H]/2+ |
| | Phe4COOH-G-Proc4F- | | | | | | |
| | SbMeW4F-Aib-S-W- | | | | | | |
| | daMeD-hF-Q-Dab-G- | | | | | | |
| | dProc3CH24Pal | | | | | | |
| 222 | Bip4CO2H-4Pal- | 0.02 | 2118.88647 | C107H120F2N22O23 | 2120.23 | 2119.95 | [M + H]+ |
| | Phe4COOH-G-Proc4F- | | | | | | |
| | SbMeW4F-Aib-S-W- | | | | | | |
| | daMeD-h4Pal-Q-Dab- | | | | | | |
| | G-dProc3CH24Pal | | | | | | |
| 223 | Bip4CO2H-4Pal- | 0.027 | 2147.87664 | C107H119F2N23O24 | 2149.23 | 2148.99 | [M + H]+ |
| | Phe4COOH-G-Proc4F- | | | | | | |
| | SbMeW4F-Aib-S-W- | | | | | | |
| | daMeD-h4Pal-Q- | | | | | | |
| | DapUrea-G- | | | | | | |
| | dProc3CH24Pal | | | | | | |
| 224 | Bip4CO2H-4Pal- | 0.03 | 2091.88681 | C105H119F2N23O22 | 2093.21 | 698.32 | [M + 3H]/3+ |
| | Phe4COOH-G-Proc4F- | | | | | | |
| | SbMeW4F-Aib-S-W- | | | | | | |
| | daMeSMe-PyrimAla- | | | | | | |
| | Q-Dab-G- | | | | | | |
| | dProc3CH24Pal | | | | | | |
| 225 | Bip4CO2H-4Pal- | 0.019 | 2139.8271 | C105H116ClF2N23O23 | 2141.63 | 1071.15 | [M + 2H]/2+ |
| | Phe4COOH-G-Proc4F- | | | | | | |
| | SbMeW4F-Aib-S- | | | | | | |
| | Trp4Cl-daMeD- | | | | | | |
| | PyrimAla-Q-Dab-G- | | | | | | |
| | dProc3CH24Pal | | | | | | |
| 226 | Bip4CO2H-4Pal- | 0.022 | 2168.81726 | C105H115ClF2N24O24 | 2170.63 | 723.82 | [M + 3H]/3+ |
| | Phe4COOH-G-Proc4F- | | | | | | |
| | SbMeW4F-Aib-S- | | | | | | |
| | Trp4Cl-daMeD- | | | | | | |
| | PyrimAla-Q-DapUrea- | | | | | | |
| | G-dProc3CH24Pal | | | | | | |
| 227 | Bip4CO2H-4Pal- | 0.014 | 2071.87049 | C103H119F2N21O24 | 2073.17 | 1037.00 | [M + 2H]/2+ |
| | Phe4COOH-G-Proc4F- | | | | | | |
| | SbMeW4F-Aib-S-W- | | | | | | |
| | daMeD-ThrOMe-Q- | | | | | | |
| | Dab-G- | | | | | | |
| | dProc3CH24Pal | | | | | | |
| 228 | Bip4CO2H-4Pal- | 0.021 | 2100.86065 | C103H118F2N22O25 | 2102.17 | 1051.60 | [M + 2H]/2+ |
| | Phe4COOH-G-Proc4F- | | | | | | |
| | SbMeW4F-Aib-S-W- | | | | | | |
| | daMeD-ThrOMe-Q- | | | | | | |
| | DapUrea-G- | | | | | | |
| | dProc3CH24Pal | | | | | | |
| 229 | Bip4CO2H-4Pal- | 0.023 | 2147.90179 | C109H123F2N21O24 | 2149.27 | 717.08 | [M + 3H]/3+ |
| | Phe4COOH-G-Proc4F- | | | | | | |
| | SbMeW4F-Aib-S-W- | | | | | | |
| | daMeD-ThrOBn-Q- | | | | | | |
| | Dab-G- | | | | | | |
| | dProc3CH24Pal | | | | | | |
| 230 | Bip4CO2H-4Pal- | 0.022 | 2134.85624 | C105H116F2N24O24 | 2136.19 | 1068.78 | [M + 2H]/2+ |
| | Phe4COOH-G-Proc4F- | | | | | | |
| | SbMeW4F-Aib-S-W- | | | | | | |
| | daMeD-PyrimAla-Q- | | | | | | |
| | DapUrea-G- | | | | | | |
| | dProc3CH24Pal | | | | | | |
| 231 | Bip4CO2H-4Pal- | 0.027 | 2055.87558 | C103H119F2N21O23 | 2057.17 | 2057.34 | [M + H]+ |
| | Phe4COOH-G-Proc4F- | | | | | | |
| | SbMeW4F-Aib-S-W- | | | | | | |
| | daMeD-Nva-Q-Dab-G- | | | | | | |
| | dProc3CH24Pal | | | | | | |
| 232 | Bip4CO2H-4Pal- | 0.022 | 2109.84731 | C103H116F5N21O23 | 2111.14 | 2111.11 | [M + H]+ |
| | Phe4COOH-G-Proc4F- | | | | | | |
| | SbMeW4F-Aib-S-W- | | | | | | |
| | daMeD-NvaF3-Q-Dab- | | | | | | |
| | G-dProc3CH24Pal | | | | | | |
| 233 | Bip4CO2H-4Pal- | 0.03 | 2061.87624 | C104H117F2N23O21 | 2063.18 | 1031.71 | [M + 2H]/2+ |
| | Phe4COOH-G-Proc4F- | | | | | | |
| | SbMeW4F-Aib-S-W- | | | | | | |
| | Aib-dPyrimA-Q-Dab- | | | | | | |
| | G-dProc3CH24Pal | | | | | | |
| 234 | Bip4CO2H-4Pal- | 0.024 | 2027.84428 | C101H115F2N21O23 | 2029.12 | 1014.75 | [M + 2H]/2+ |
| | Phe4COOH-G-Proc4F- | | | | | | |
| | SbMeW4F-Aib-S-W- | | | | | | |
| | Aib-dD-Q-Dab-G- | | | | | | |
| | dProc3CH24Pal | | | | | | |
| 235 | Bip4CO2H-4Pal- | 0.018 | 2119.88172 | C106H119F2N23O23 | 2121.22 | 1061.42 | [M + 2H]/2+ |
| | Phe4COOH-G-Proc4F- | | | | | | |
| | SbMeW4F-Aib-S-W- | | | | | | |
| | daMeD-PyrimAla-Q- | | | | | | |
| | NMeDab-G- | | | | | | |
| | dProc3CH24Pal | | | | | | |
| 236 | Bip4CO2H-4Pal- | 0.021 | 2121.86615 | C107H118F3N21O23 | 2123.2 | 2123.17 | [M + H]+ |
| | Phe4COOH-G-Proc4F- | | | | | | |
| | SbMeW4F-Aib-S-W- | | | | | | |
| | daMeD-dPhe4F-Q- | | | | | | |
| | Dab-G- | | | | | | |
| | dProc3CH24Pal | | | | | | |
| 237 | Bip4CO2H-4Pal- | 0.025 | 2109.92253 | C107H125F2N21O23 | 2111.26 | 1055.62 | [M + 2H]/2+ |
| | Phe4COOH-G-Proc4F- | | | | | | |
| | SbMeW4F-Aib-S-W- | | | | | | |
| | daMeD-dcha-Q-Dab- | | | | | | |
| | G-dProc3CH24Pal | | | | | | |
| 238 | Bip4CO2H-4Pal- | 0.024 | 2069.89123 | C104H121F2N21O23 | 2071.2 | 2070.87 | [M + H]+ |
| | Phe4COOH-G-Proc4F- | | | | | | |
| | SbMeW4F-Aib-S-W- | | | | | | |
| | daMeD-dL-Q-Dab-G- | | | | | | |
| | dProc3CH24Pal | | | | | | |
| 239 | Bip4CO2H-4Pal- | 0.02 | 2081.89123 | C105H121F2N21O23 | 2083.21 | 2083.36 | [M + H]+ |
| | Phe4COOH-G-Proc4F- | | | | | | |
| | SbMeW4F-Aib-S-W- | | | | | | |
| | daMeD-dcBuA-Q- | | | | | | |
| | Dab-G- | | | | | | |
| | dProc3CH24Pal | | | | | | |
| 240 | Bip4CO2H-4Pal- | 0.025 | 2104.87082 | C106H118F2N22O23 | 2106.2 | 2105.57 | [M + H]+ |
| | Phe4COOH-G-Proc4F- | | | | | | |
| | SbMeW4F-Aib-S-W- | | | | | | |
| | daMeD-d3Pal-Q-Dab- | | | | | | |
| | G-dProc3CH24Pal | | | | | | |
| 241 | Bip4CO2H-4Pal- | 0.018 | 2075.89189 | C105H119F2N23O21 | 2077.21 | 693.14 | [M + 3H]/3+ |
| | Phe4COOH-G-Proc4F- | | | | | | |
| | SbMeW4F-Aib-S-W- | | | | | | |
| | Aib-PyrimAla-Q- | | | | | | |
| | NMeDab-G- | | | | | | |
| | dProc3CH24Pal | | | | | | |
| 242 | Bip4CO2H-4Pal- | 0.019 | 2043.83919 | C101H115F2N21O24 | 2045.12 | 2045.05 | [M + H]+ |
| | Phe4COOH-G-Proc4F- | | | | | | |
| | SbMeW4F-Aib-S-W- | | | | | | |
| | daMeD-dS-Q-Dab-G- | | | | | | |
| | dProc4CH24Pal | | | | | | |
| 243 | Bip4CO2H-4Pal- | 0.022 | 2103.87558 | C107H119F2N21O23 | 2105.21 | 2105.00 | [M + H]+ |
| | Phe4COOH-G-Proc4F- | | | | | | |
| | SbMeW4F-Aib-S-W- | | | | | | |
| | daMeD-dF-Q-Dab-G- | | | | | | |
| | dProc4CH24Pal | | | | | | |
| 244 | Bip-4Pal-Phe4COOH- | 0.03 | 2069.90246 | C103H121F2N23O22 | 2071.2 | 2071.08 | [M + H]+ |
| | G-Proc4F-SbMeW4F- | | | | | | |
| | Aib-S-W-daMeD-dK- | | | | | | |
| | Q-DapUrea-G- | | | | | | |
| | dProc4CH24Pal | | | | | | |
| 245 | Bip-4Pal-Phe4COOH- | 0.027 | 2055.88681 | C102H119F2N23O22 | 2057.17 | 1029.05 | [M + 2H]/2+ |
| | G-Proc4F-SbMeW4F- | | | | | | |
| | Aib-S-W-daMeD- | | | | | | |
| | dOrn-Q-DapUrea-G- | | | | | | |
| | dProc4CH24Pal | | | | | | |
| 246 | Bip-4Pal-Phe4COOH- | 0.031 | 2027.85551 | C100H115F2N23O22 | 2029.12 | 1015.16 | [M + 2H]/2+ |
| | G-Proc4F-SbMeW4F- | | | | | | |
| | Aib-S-W-daMeD- | | | | | | |
| | dDap-Q-DapUrea-G- | | | | | | |
| | dProc4CH24Pal | | | | | | |
| 247 | Bip-4Pal-Phe4COOH- | 0.03 | 2041.87116 | C101H117F2N23O22 | 2043.15 | 1022.13 | [M + 2H]/2+ |
| | G-Proc4F-SbMeW4F- | | | | | | |
| | Aib-S-W-daMeD- | | | | | | |
| | dDab-Q-DapUrea-G- | | | | | | |
| | dProc4CH24Pal | | | | | | |
| 248 | Bip-4Pal-Phe4COOH- | 0.028 | 2012.881 | C101H118F2N22O21 | 2014.15 | 2014.29 | [M + H]+ |
| | G-Proc4F-SbMeW4F- | | | | | | |
| | Aib-S-W-daMeD- | | | | | | |
| | dDab-Q-Dab-G- | | | | | | |
| | dProc4CH24Pal | | | | | | |
| 249 | Bip-4Pal-Phe4COOH- | 0.016 | 2041.85993 | C102H117F2N21O23 | 2043.14 | 2042.60 | [M + H]+ |
| | G-Proc4F-SbMeW4F- | | | | | | |
| | Aib-S-W-daMeD-dE- | | | | | | |
| | Q-Dab-G- | | | | | | |
| | dProc4CH24Pal | | | | | | |
| 250 | Bip-4Pal-Phe4COOH- | 0.027 | 1997.8701 | C101H117F2N21O21 | 1999.13 | 1000.21 | [M + 2H]/2+ |
| | G-Proc4F-SbMeW4F- | | | | | | |
| | Aib-S-W-Aib-dE-Q- | | | | | | |
| | Dab-G- | | | | | | |
| | dProc4CH24Pal | | | | | | |
| 251 | Bip4CO2H-4Pal- | 0.013 | 2139.8271 | C105H116ClF2N23O23 | 2141.63 | 1071.15 | [M + 2H]/2+ |
| | Phe4COOH-G-Proc4F- | | | | | | |
| | SbMeW4F-Aib-S- | | | | | | |
| | Trp4Cl-daMeD- | | | | | | |
| | PyrimAla-Q-Dab-G- | | | | | | |
| | dProc4CH24Pal | | | | | | |
| 252 | Bip4CO2H-4Pal- | 0.013 | 2123.85665 | C105H116F3N23O23 | 2125.18 | 1062.60 | [M + 2H]/2+ |
| | Phe4COOH-G-Proc4F- | | | | | | |
| | SbMeW4F-Aib-S- | | | | | | |
| | Trp4F-daMeD- | | | | | | |
| | PyrimAla-Q-Dab-G- | | | | | | |
| | dProc4CH24Pal | | | | | | |
| 253 | Bip4CO2H-4Pal- | 0.013 | 2055.87558 | C103H119F2N21O23 | 2057.17 | 2056.90 | [M + H]+ |
| | Phe4COOH-G-Proc4F- | | | | | | |
| | SbMeW4F-Aib-S-W- | | | | | | |
| | daMeD-Nva-Q-Dab-G- | | | | | | |
| | dProc4CH24Pal | | | | | | |
| 254 | Bip4CO2H-4Pal- | 0.013 | 2071.87049 | C103H119F2N21O24 | 2073.17 | 2073.18 | [M + H]+ |
| | Phe4COOH-G-Proc4F- | | | | | | |
| | SbMeW4F-Aib-S-W- | | | | | | |
| | daMeD-alTOMe-Q- | | | | | | |
| | Dab-G- | | | | | | |
| | dProc4CH24Pal | | | | | | |
| 255 | Bip4CO2H-4Pal- | 0.011 | 2071.87049 | C103H119F2N21O24 | 2073.17 | 2072.58 | [M + H]+ |
| | Phe4COOH-G-Proc4F- | | | | | | |
| | SbMeW4F-Aib-S-W- | | | | | | |
| | daMeD-ThrOMe-Q- | | | | | | |
| | Dab-G- | | | | | | |
| | dProc4CH24Pal | | | | | | |
| 256 | Bip4CO2H-4Pal- | 0.012 | 2133.88614 | C108H121F2N21O24 | 2135.24 | 2134.78 | [M + H]+ |
| | Phe4COOH-G-Proc4F- | | | | | | |
| | SbMeW4F-Aib-S-W- | | | | | | |
| | daMeD-hY-Q-Dab-G- | | | | | | |
| | dProc4CH24Pal | | | | | | |
| 257 | Bip4CO2H-4Pal- | 0.021 | 2089.89631 | C107H121F2N21O22 | 2091.23 | 2090.98 | [M + H]+ |
| | Phe4COOH-G-Proc4F- | | | | | | |
| | SbMeW4F-Aib-S-W- | | | | | | |
| | daMeF-alT-Q-Dab-G- | | | | | | |
| | dProc4CH24Pal | | | | | | |
| 258 | Bip4CO2H-4Pal- | 0.02 | 2117.89123 | C108H121F2N21O23 | 2119.24 | 1060.09 | [M + 2H]/2+ |
| | Phe4COOH-G-Proc4F- | | | | | | |
| | SbMeW4F-Aib-S-W- | | | | | | |
| | daMeF-E-Q-Dab-G- | | | | | | |
| | dProc4CH24Pal | | | | | | |
| 259 | Bip4CO2H-4Pal- | 0.014 | 2103.87558 | C107H119F2N21O23 | 2105.21 | 2104.78 | [M + H]+ |
| | Phe4COOH-G-Proc4F- | | | | | | |
| | SbMeW4F-Aib-S-W- | | | | | | |
| | daMeF-D-Q-Dab-G- | | | | | | |
| | dProc4CH24Pal | | | | | | |
| 260 | Bip4CO2H-4Pal- | 0.013 | 2091.88681 | C105H119F2N23O22 | 2093.21 | 2092.90 | [M + H]+ |
| | Phe4COOH-G-Proc4F- | | | | | | |
| | SbMeW4F-Aib-S-W- | | | | | | |
| | daMeSMe-PyrimAla- | | | | | | |
| | Q-Dab-G- | | | | | | |
| | dProc4CH24Pal | | | | | | |
| 261 | Bip4CO2H-4Pal- | 0.017 | 2043.87558 | C102H119F2N21O23 | 2045.16 | 2044.89 | [M + H]+ |
| | Phe4COOH-G-Proc4F- | | | | | | |
| | SbMeW4F-Aib-S-W- | | | | | | |
| | daMeSMe-alT-Q-Dab- | | | | | | |
| | G-dProc4CH24Pal | | | | | | |
| 262 | Bip4CO2H-4Pal- | 0.02 | 2075.89189 | C105H119F2N23O21 | 2077.21 | 2076.90 | [M + H]+ |
| | Phe4COOH-G-Proc4F- | | | | | | |
| | SbMeW4F-Aib-S-W- | | | | | | |
| | Aib-PyrimAla-Q- | | | | | | |
| | NMeDab-G- | | | | | | |
| | dProc4CH24Pal | | | | | | |
| 263 | Bip4CO2H-4Pal- | 0.017 | 2137.86107 | C107H118F3N21O24 | 2139.2 | 2138.87 | [M + H]+ |
| | Phe4COOH-G-Proc4F- | | | | | | |
| | SbMeW4F-Aib-S-W- | | | | | | |
| | daMeD-sbOH4Pal-Q- | | | | | | |
| | Dab-G-dProc3Bn4F | | | | | | |
| 264 | Bip-4Pal-Phe4COOH- | 0.017 | 2059.88058 | C104H118FN23O22 | 2061.19 | 2060.89 | [M + H]+ |
| | G-Proc4OH- | | | | | | |
| | SbMeW4F-Aib-S-W- | | | | | | |
| | daMeD-PyrimAla-Q- | | | | | | |
| | Dab-G- | | | | | | |
| | dProc4CH24Pal | | | | | | |
| 265 | Bip-4Pal-Phe4COOH- | 0.022 | 1983.85445 | C100H115F2N21O21 | 1985.11 | 1984.86 | [M + H]+ |
| | G-Proc4F-SbMeW4F- | | | | | | |
| | Aib-S-W-Aib-dD-Q- | | | | | | |
| | Dab-G- | | | | | | |
| | dProc4CH24Pal | | | | | | |
| 266 | Bip4CO2H-4Pal- | 0.019 | 2075.84765 | C102H119F2N21O23S | 2077.22 | 2076.86 | [M + H]+ |
| | Phe4COOH-G-Proc4F- | | | | | | |
| | SbMeW4F-Aib-S-W- | | | | | | |
| | Aib-MO2-Q-Dab-G- | | | | | | |
| | dProc4CH24Pal | | | | | | |
| 267 | Bip4CO2H-4Pal- | 0.014 | 2104.88206 | C105H118F2N24O22 | 2106.2 | 2105.89 | [M + H]+ |
| | Phe4COOH-G-Proc4F- | | | | | | |
| | SbMeW4F-Aib-S-W- | | | | | | |
| | Aib- | | | | | | |
| | AlaPyrim4CONH2-Q- | | | | | | |
| | Dab-G- | | | | | | |
| | dProc4CH24Pal | | | | | | |
| 268 | Bip4CO2H-4Pal- | 0.014 | 2105.86607 | C105H117F2N23O23 | 2107.19 | 2106.88 | [M + H]+ |
| | Phe4COOH-G-Proc4F- | | | | | | |
| | SbMeW4F-Aib-S-W- | | | | | | |
| | Aib-AlaPyrim4COOH- | | | | | | |
| | Q-Dab-G- | | | | | | |
| | dProc4CH24Pal | | | | | | |
| 269 | Bip-4Pal-Phe4COOH- | 0.014 | 2061.87624 | C104H117F2N23O21 | 2063.18 | 2062.89 | [M + H]+ |
| | G-Proc4F-SbMeW4F- | | | | | | |
| | Aib-S-W-Aib- | | | | | | |
| | AlaPyrim4COOH-Q- | | | | | | |
| | Dab-G- | | | | | | |
| | dProc4CH24Pal | | | | | | |
| 270 | Bip4CO2H-4Pal- | 0.023 | 2090.86641 | C104H116F2N24O22 | 2092.18 | 2091.88 | [M + H]+ |
| | Phe4COOH-G-Proc4F- | | | | | | |
| | SbMeW4F-Aib-S-W- | | | | | | |
| | Aib-PyrimAla-Q- | | | | | | |
| | DapUrea-G- | | | | | | |
| | dProc4CH24Pal | | | | | | |
| 271 | Bip4CO2H-4Pal- | 0.023 | 2155.90802 | C108H124F3N21O24 | 2157.26 | 2157.12 | [M + H]+ |
| | Phe4COOH-G-Proc4F- | | | | | | |
| | sbMeW4F-Aib-E-W- | | | | | | |
| | Aib-AlaPiperazAc-Q- | | | | | | |
| | S-G-dProc4Bn4F | | | | | | |
| 272 | Bip4CO2H-4Pal- | 0.018 | 2093.87124 | C106H118F3N21O22 | 2095.19 | 2094.88 | [M + H]+ |
| | Phe4COOH-G-Proc4F- | | | | | | |
| | SbMeW4F-Aib-S-W- | | | | | | |
| | Aib-SbOH4Pal-Q-Dab- | | | | | | |
| | G-dProc4Bn4F | | | | | | |
| 273 | Bip4CO2H-4Pal- | 0.013 | 2093.87124 | C106H118F3N21O22 | 2095.19 | 2094.88 | [M + H]+ |
| | Phe4COOH-G-Proc4F- | | | | | | |
| | SbMeW4F-Aib-S-W- | | | | | | |
| | Aib-RbOH4Pal-Q- | | | | | | |
| | Dab-G-dProc4Bn4F | | | | | | |
| 274 | Bip4CO2H- | 0.026 | 2094.86649 | C105H117F3N22O22 | 2096.18 | 2095.88 | [M + H]+ |
| | RbOH4Pal- | | | | | | |
| | Phe4COOH-G-Proc4F- | | | | | | |
| | SbMeW4F-Aib-S-W- | | | | | | |
| | Aib-PyrimAla-Q-Dab- | | | | | | |
| | G-dProc4Bn4F | | | | | | |
| 275 | Bip4CO2H-4Pal- | 0.018 | 2094.86649 | C105H117F3N22O22 | 2096.18 | 2095.88 | [M + H]+ |
| | orbOHorPhe4COOH- | | | | | | |
| | G-Proc4F-SbMeW4F- | | | | | | |
| | Aib-S-W-Aib- | | | | | | |
| | PyrimAla-Q-Dab-G- | | | | | | |
| | dProc4Bn4F | | | | | | |
| 276 | Bip4CO2H-4Pal- | 0.017 | 2108.88214 | C106H119F3N22O22 | 2110.21 | 1055.81 | [M + 2H]/2+ |
| | Phe4COOH-G-Proc4F- | | | | | | |
| | sbMeW4F-Aib-E-W- | | | | | | |
| | Aib-H-Q-Dab-G- | | | | | | |
| | dProc4Bn4F | | | | | | |
| 277 | Phe4F-4Pal- | 0.023 | 2020.83085 | C99H112F4N22O21 | 2022.08 | 1012.65 | [M + 2H]/2+ |
| | Phe4COOH-G-Proc4F- | | | | | | |
| | SbMeW4F-Aib-S-W- | | | | | | |
| | daMeD-PyrimAla-Q- | | | | | | |
| | Dab-G-dProc3Bn4F | | | | | | |
| 278 | Y-4Pal-Phe4COOH-G- | 0.021 | 2018.83519 | C99H113F3N22O22 | 2020.09 | 1010.48 | [M + 2H]/2+ |
| | Proc4F-SbMeW4F- | | | | | | |
| | Aib-S-W-daMeD- | | | | | | |
| | PyrimAla-Q-Dab-G- | | | | | | |
| | dProc3Bn4F | | | | | | |
| 279 | Phe4ImMe-4Pal- | 0.013 | 2082.87772 | C103H117F3N24O21 | 2084.17 | 1042.72 | [M + 2H]/2+ |
| | Phe4COOH-G-Proc4F- | | | | | | |
| | SbMeW4F-Aib-S-W- | | | | | | |
| | daMeD-PyrimAla-Q- | | | | | | |
| | Dab-G-dProc3Bn4F | | | | | | |
| 280 | Phe42ImMe-4Pal- | 0.016 | 2082.87772 | C103H117F3N24O21 | 2084.17 | 1042.26 | [M + 2H]/2+ |
| | Phe4COOH-G-Proc4F- | | | | | | |
| | SbMeW4F-Aib-S-W- | | | | | | |
| | daMeD-PyrimAla-Q- | | | | | | |
| | Dab-G-dProc3Bn4F | | | | | | |
| 281 | Phe4PyrimOMe-4Pal- | 0.012 | 2110.87264 | C104H117F3N24O22 | 2112.18 | 1056.56 | [M + 2H]/2+ |
| | Phe4COOH-G-Proc4F- | | | | | | |
| | SbMeW4F-Aib-S-W- | | | | | | |
| | daMeD-PyrimAla-Q- | | | | | | |
| | Dab-G-dProc3Bn4F | | | | | | |
| 282 | Phe4Pyrim-4Pal- | 0.014 | 2080.86207 | C103H115F3N24O21 | 2082.16 | 694.21 | [M + 3H]/3+ |
| | Phe4COOH-G-Proc4F- | | | | | | |
| | SbMeW4F-Aib-S-W- | | | | | | |
| | daMeD-PyrimAla-Q- | | | | | | |
| | Dab-G-dProc3Bn4F | | | | | | |
| 283 | Phe4Pyr4-4Pal- | 0.016 | 2079.86682 | C104H116F3N23O21 | 2081.17 | 1040.99 | [M + 2H]/2+ |
| | Phe4COOH-G-Proc4F- | | | | | | |
| | SbMeW4F-Aib-S-W- | | | | | | |
| | daMeD-PyrimAla-Q- | | | | | | |
| | Dab-G-dProc3Bn4F | | | | | | |
| 284 | Bip4CO2H-AlaPent- | 0.015 | 2094.88647 | C105H120F2N22O23 | 2096.21 | 1048.61 | [M + 2H]/2+ |
| | Phe4COOH-G-Proc4F- | | | | | | |
| | SbMeW4F-Aib-S-W- | | | | | | |
| | daMeD-4PyrimAla-Q- | | | | | | |
| | Dab-G- | | | | | | |
| | dProc3CH24Pal | | | | | | |
| 285 | Bip4CO2H-AlaPent- | 0.013 | 2094.88647 | C105H120F2N22O23 | 2096.21 | 1048.56 | [M + 2H]/2+ |
| | Phe4COOH-G-Proc4F- | | | | | | |
| | SbMeW4F-Aib-S-W- | | | | | | |
| | daMeD-4PyrimAla-Q- | | | | | | |
| | Dab-G- | | | | | | |
| | dProc4CH24Pal | | | | | | |
| 286 | Bip4CO2H-4Pal- | 0.01 | 2129.89237 | C106H122F3N21O24 | 2131.22 | 2130.91 | [M + H]+ |
| | Phe4COOH-G-Proc4F- | | | | | | |
| | SbMeW4F-Aib-S-W- | | | | | | |
| | daMeD-AlaMph-Q- | | | | | | |
| | Dab-G-dProc3Bn4F | | | | | | |
| 287 | Bip4CO2H-4Pal- | 0.011 | 2128.90835 | C106H123F3N22O23 | 2130.24 | 2130.30 | [M + H]+ |
| | Phe4COOH-G-Proc4F- | | | | | | |
| | SbMeW4F-Aib-S-W- | | | | | | |
| | daMeD-AlaPiperaz-Q- | | | | | | |
| | Dab-G-dProc3Bn4F | | | | | | |
| 288 | Bip-4Pal-Phe4COOH- | 0.019 | 2006.87918 | C103H119FN20O22 | 2008.17 | 2007.88 | [M + H]+ |
| | G-Proc4F-SbMe1Nal- | | | | | | |
| | Aib-S-W-daMeD-alT- | | | | | | |
| | Q-Dab-G- | | | | | | |
| | dProc4CH24Pal | | | | | | |
| 289 | Bip-4Pal-Phe4COOH- | 0.015 | 2029.83546 | C101H117ClFN21O22 | 2031.59 | 2030.84 | [M + H]+ |
| | G-Proc4F-SbMeW4Cl- | | | | | | |
| | Aib-S-W-daMeD-alT- | | | | | | |
| | Q-Dab-G- | | | | | | |
| | dProc4CH24Pal | | | | | | |
| 290 | Bip-4Pal-Phe4COOH- | 0.018 | 2060.92613 | C107H125FN20O22 | 2062.26 | 2061.93 | [M + H]+ |
| | G-Proc4F-SbMe1Nal- | | | | | | |
| | Aib-S-W-daMeD- | | | | | | |
| | AlaTHP4-Q-Dab-G- | | | | | | |
| | dProc4CH24Pal | | | | | | |
| 291 | Bip-4Pal-Phe4COOH- | 0.018 | 2054.89042 | C106H119FN22O21 | 2056.21 | 2055.90 | [M + H]+ |
| | G-Proc4F-SbMe1Nal- | | | | | | |
| | Aib-S-W-daMeD- | | | | | | |
| | PyrimAla-Q-Dab-G- | | | | | | |
| | dProc4CH24Pal | | | | | | |
| 292 | Bip-4Pal-Phe4COOH- | 0.015 | 2077.84669 | C104H117ClFN23O21 | 2079.63 | 2078.85 | [M + H]+ |
| | G-Proc4F-SbMeW4Cl- | | | | | | |
| | Aib-S-W-daMeD- | | | | | | |
| | PyrimAla-Q-Dab-G- | | | | | | |
| | dProc4CH24Pal | | | | | | |
| 293 | Bip-4Pal-Phe4COOH- | 0.01 | 2013.86501 | C101H117F2N21O22 | 2015.13 | 2014.87 | [M + H]+ |
| | G-Proc4F-SbMeW4F- | | | | | | |
| | Aib-S-W-daMeD-alT- | | | | | | |
| | Q-Dab-G- | | | | | | |
| | dProc4CH24Pal | | | | | | |
| 294 | Bip-4Pal-Phe4COOH- | 0.014 | 2067.91196 | C105H123F2N21O22 | 2069.22 | 2068.91 | [M + H]+ |
| | G-Proc4F-SbMeW4F- | | | | | | |
| | Aib-S-W-daMeD- | | | | | | |
| | AlaTHP4-Q-Dab-G- | | | | | | |
| | dProc4CH24Pal | | | | | | |
| 295 | Bip4CONMe2-4Pal- | 0.012 | 2138.94907 | C108H128F2N22O23 | 2140.3 | 2139.95 | [M + H]+ |
| | Phe4COOH-G-Proc4F- | | | | | | |
| | SbMeW4F-Aib-S-W- | | | | | | |
| | daMeD-AlaTHP4-Q- | | | | | | |
| | Dab-G- | | | | | | |
| | dProc4CH24Pal | | | | | | |
| 296 | Bip4CONH2-4Pal- | 0.013 | 2110.91777 | C106H124F2N22O23 | 2112.25 | 2111.92 | [M + H]+ |
| | Phe4COOH-G-Proc4F- | | | | | | |
| | SbMeW4F-Aib-S-W- | | | | | | |
| | daMeD-AlaTHP4-Q- | | | | | | |
| | Dab-G- | | | | | | |
| | dProc4CH24Pal | | | | | | |
| 297 | Bip4CO2H-4Pal- | 0.022 | 2086.94675 | C106H127F3N21O21 | 2088.27 | 2088.05 | [M + H]+ |
| | Phe4COOH-G-Proc4F- | | | | | | |
| | sbMeW4F-Aib-S-W- | | | | | | |
| | Aib-OrnMe3-Q-Dab- | | | | | | |
| | G-dProc4Bn4F | | | | | | |
| 298 | Bip4CO2H-4Pal- | 0.015 | 2100.9624 | C107H129F3N21O21 | 2102.29 | 2102.43 | [M + H]+ |
| | Phe4COOH-G-Proc4F- | | | | | | |
| | sbMeW4F-Aib-S-W- | | | | | | |
| | Aib-LysMe3-Q-Dab- | | | | | | |
| | G-dProc4Bn4F | | | | | | |
| 299 | Phe4Br-4Pal- | 0.012 | 2080.75079 | C99H112BrF3N22O21 | 2082.98 | 1041.52 | [M + 2H]/2+ |
| | Phe4COOH-G-Proc4F- | | | | | | |
| | SbMeW4F-Aib-S-W- | | | | | | |
| | daMeD-PyrimAla-Q- | | | | | | |
| | Dab-G-dProc3Bn4F | | | | | | |
| 300 | AEFPiv-4Pal- | 0.013 | 2084.94974 | C104H126F2N24O21 | 2086.26 | 1043.84 | [M + 2H]/2+ |
| | Phe4COOH-G-Proc4F- | | | | | | |
| | SbMeW4F-Aib-S-W- | | | | | | |
| | Aib-PyrimAla-Q-Dab- | | | | | | |
| | G-dProc4CH24Pal | | | | | | |
| 301 | AEFcHex-4Pal- | 0.017 | 2110.96539 | C106H128F2N24O21 | 2112.3 | 2111.61 | [M + H]+ |
| | Phe4COOH-G-Proc4F- | | | | | | |
| | SbMeW4F-Aib-S-W- | | | | | | |
| | Aib-PyrimAla-Q-Dab- | | | | | | |
| | G-dProc4CH24Pal | | | | | | |
| 302 | AEF4AcPip4c-4Pal- | 0.017 | 2153.97121 | C107H129F2N25O22 | 2155.32 | 1078.19 | [M + 2H]/2+ |
| | Phe4COOH-G-Proc4F- | | | | | | |
| | SbMeW4F-Aib-S-W- | | | | | | |
| | Aib-PyrimAla-Q-Dab- | | | | | | |
| | G-dProc4CH24Pal | | | | | | |
| 303 | AEFPyrzCO-4Pal- | 0.016 | 2106.90894 | C104H120F2N26O21 | 2108.22 | 2108.38 | [M + H]+ |
| | Phe4COOH-G-Proc4F- | | | | | | |
| | SbMeW4F-Aib-S-W- | | | | | | |
| | Aib-PyrimAla-Q-Dab- | | | | | | |
| | G-dProc4CH24Pal | | | | | | |
| 304 | AEF2PyrimCO-4Pal- | 0.014 | 2106.90894 | C104H120F2N26O21 | 2108.22 | 1054.93 | [M + 2H]/2+ |
| | Phe4COOH-G-Proc4F- | | | | | | |
| | SbMeW4F-Aib-S-W- | | | | | | |
| | Aib-PyrimAla-Q-Dab- | | | | | | |
| | G-dProc4CH24Pal | | | | | | |
| 305 | AEF4PyrimCO-4Pal- | 0.016 | 2106.90894 | C104H120F2N26O21 | 2108.22 | 1054.69 | [M + 2H]/2+ |
| | Phe4COOH-G-Proc4F- | | | | | | |
| | SbMeW4F-Aib-S-W- | | | | | | |
| | Aib-PyrimAla-Q-Dab- | | | | | | |
| | G-dProc4CH24Pal | | | | | | |
| 306 | AEFBz4Piperaz-4Pal- | 0.02 | 2188.98719 | C110H130F2N26O21 | 2190.37 | 1095.43 | [M + 2H]/2+ |
| | Phe4COOH-G-Proc4F- | | | | | | |
| | SbMeW4F-Aib-S-W- | | | | | | |
| | Aib-PyrimAla-Q-Dab- | | | | | | |
| | G-dProc4CH24Pal | | | | | | |
| 307 | AEF4PyrCO-4Pal- | 0.018 | 2105.91369 | C105H121F2N25O21 | 2107.24 | 1054.24 | [M + 2H]/2+ |
| | Phe4COOH-G-Proc4F- | | | | | | |
| | SbMeW4F-Aib-S-W- | | | | | | |
| | Aib-PyrimAla-Q-Dab- | | | | | | |
| | G-dProc4CH24Pal | | | | | | |
| 308 | AEFBz-4Pal- | 0.013 | 2104.91844 | C106H122F2N24O21 | 2106.25 | 1053.57 | [M + 2H]/2+ |
| | Phe4COOH-G-Proc4F- | | | | | | |
| | SbMeW4F-Aib-S-W- | | | | | | |
| | Aib-PyrimAla-Q-Dab- | | | | | | |
| | G-dProc4CH24Pal | | | | | | |
| 309 | TyrEtNAc-4Pal- | 0.018 | 2116.88389 | C105H123ClF2N22O22 | 2118.68 | 1059.22 | [M + 2H]/2+ |
| | Phe4COOH-G-Proc4F- | | | | | | |
| | SbMeW4F-Aib-E-W- | | | | | | |
| | Aib-Phe3Cl-Q-Dab-G- | | | | | | |
| | dProc4CH24Pal | | | | | | |
| 310 | TyrEtNAc-4Pal- | 0.017 | 2098.91777 | C105H124F2N22O23 | 2100.24 | 1050.53 | [M + 2H]/2+ |
| | Phe4COOH-G-Proc4F- | | | | | | |
| | SbMeW4F-Aib-E-W- | | | | | | |
| | Aib-Y-Q-Dab-G- | | | | | | |
| | dProc4CH24Pal | | | | | | |
| 311 | Phe4tButyl-4Pal- | 0.017 | 2039.92828 | C103H123F2N23O20 | 2041.22 | 2040.95 | [M + H]+ |
| | Phe4COOH-G-Proc4F- | | | | | | |
| | SbMeW4F-Aib-E-W- | | | | | | |
| | Aib-PyrimAla-Q-Dab- | | | | | | |
| | G-dProc4CH24Pal | | | | | | |
| 312 | Bip4CO2H-4Pal- | 0.017 | 2120.88214 | C107H119F3N22O22 | 2122.22 | 2121.68 | [M + H]+ |
| | Phe4COOH-G-Proc4F- | | | | | | |
| | SbMeW4F-Aib-E-W- | | | | | | |
| | Aib-4PyrimAla-Q- | | | | | | |
| | Dab-G-dProc3Bn4F | | | | | | |
| 313 | Bip4CO2H-4Pal- | 0.015 | 2128.89712 | C107H123F3N20O24 | 2130.24 | 2129.80 | [M + H]+ |
| | Phe4COOH-G-Proc4F- | | | | | | |
| | SbMeW4F-Aib-S-W- | | | | | | |
| | daMeD-AlaTHP4-Q- | | | | | | |
| | Dab-G-dProc4Bn4F | | | | | | |
| 314 | Bip4CO2H-4Pal- | 0.02 | 2072.9311 | C105H125F3N21O21 | 2074.24 | 2074.05 | [M + H]+ |
| | Phe4COOH-G-Proc4F- | | | | | | |
| | sbMeW4F-Aib-S-W- | | | | | | |
| | Aib-DabMe3-Q-Dab- | | | | | | |
| | G-dProc4Bn4F | | | | | | |
| 315 | TyrEtNAc-4Pal- | 0.018 | 2087.91302 | C103H123F2N23O23 | 2089.22 | 2088.63 | [M + H]+ |
| | Phe4COOH-G-Proc4F- | | | | | | |
| | SbMeW4F-Aib-E-W- | | | | | | |
| | Aib-Ala3Isoxa5Me-Q- | | | | | | |
| | Dab-G- | | | | | | |
| | dProc4CH24Pal | | | | | | |
| 316 | TyrEtNAc-4Pal- | 0.02 | 2073.89737 | C102H121F2N23O23 | 2075.19 | 1038.16 | [M + 2H]/2+ |
| | Phe4COOH-G-Proc4F- | | | | | | |
| | SbMeW4F-Aib-E-W- | | | | | | |
| | Aib-Ala2Oxa-Q-Dab- | | | | | | |
| | G-dProc4CH24Pal | | | | | | |
| 317 | TyrEtNAc-4Pal- | 0.015 | 2072.91336 | C102H122F2N24O22 | 2074.2 | 1037.09 | [M + 2H]/2+ |
| | Phe4COOH-G-Proc4F- | | | | | | |
| | SbMeW4F-Aib-E-W- | | | | | | |
| | Aib-H-Q-Dab-G- | | | | | | |
| | dProc4CH24Pal | | | | | | |
| 318 | TyrEtNAc-4Pal- | 0.025 | 2097.93376 | C105H125F2N23O22 | 2099.25 | 700.07 | [M + 3H]/3+ |
| | Phe4COOH-G-Proc4F- | | | | | | |
| | SbMeW4F-Aib-E-W- | | | | | | |
| | Aib-h4Pal-Q-Dab-G- | | | | | | |
| | dProc4CH24Pal | | | | | | |
| 319 | Bip4CO2H-4Pal- | 0.017 | 2129.89237 | C106H122F3N21O24 | 2131.22 | 1066.30 | [M + 2H]/2+ |
| | Phe4COOH-G-Proc4F- | | | | | | |
| | SbMeW4F-Aib-S-W- | | | | | | |
| | daMeD-AlaMph-Q- | | | | | | |
| | Dab-G-dProc4Bn4F | | | | | | |
| 320 | Bip4CO2H-4Pal- | 0.015 | 2127.9131 | C107H124F3N21O23 | 2129.25 | 2129.40 | [M + H]+ |
| | Phe4COOH-G-Proc4F- | | | | | | |
| | SbMeW4F-Aib-S-W- | | | | | | |
| | daMeD-AlaPip4-Q- | | | | | | |
| | Dab-G-dProc4Bn4F | | | | | | |
| 321 | Bip4CO2H-4Pal- | 0.015 | 2122.8614 | C106H117F3N22O23 | 2124.19 | 1062.42 | [M + 2H]/2+ |
| | Phe4COOH-G-Proc4F- | | | | | | |
| | SbMeW4F-Aib-S-W- | | | | | | |
| | daMeD-AlaPyrz-Q- | | | | | | |
| | Dab-G-dProc4Bn4F | | | | | | |
| 322 | Bip4CO2H-4Pal- | 0.013 | 2122.8614 | C106H117F3N22O23 | 2124.19 | 2124.13 | [M + H]+ |
| | Phe4COOH-G-Proc4F- | | | | | | |
| | SbMeW4F-Aib-S-W- | | | | | | |
| | daMeD-4PyridaAla-Q- | | | | | | |
| | Dab-G-dProc4Bn4F | | | | | | |
| 323 | Bip4CO2H-4Pal- | 0.012 | 2121.86615 | C107H118F3N21O23 | 2123.2 | 1062.35 | [M + 2H]/2+ |
| | Phe4COOH-G-Proc4F- | | | | | | |
| | SbMeW4F-Aib-S-W- | | | | | | |
| | daMeD-3Pal-Q-Dab- | | | | | | |
| | G-dProc4Bn4F | | | | | | |
| 324 | Bip4CO2H-4Pal- | 0.013 | 2121.86615 | C107H118F3N21O23 | 2123.2 | 2123.06 | [M + H]+ |
| | Phe4COOH-G-Proc4F- | | | | | | |
| | SbMeW4F-Aib-S-W- | | | | | | |
| | daMeD-4Pal-Q-Dab- | | | | | | |
| | G-dProc4Bn4F | | | | | | |
| 325 | Bip4CO2H-4Pal- | 0.027 | 2059.88058 | C104H118FN23O22 | 2061.19 | 1031.37 | [M + 2H]/2+ |
| | Phe4COOH-G- | | | | | | |
| | Proc4OH-SbMeW4F- | | | | | | |
| | Aib-S-W-Aib- | | | | | | |
| | PyrimAla-Q-Dab-G- | | | | | | |
| | dProc3CH24Pal | | | | | | |
| 326 | Bip4CO2H-4Pal- | 0.011 | 2061.87624 | C104H117F2N23O21 | 2063.18 | 2063.36 | [M + H]+ |
| | Phe4COOH-G-Proc4F- | | | | | | |
| | SbMeW4F-Aib-S-W- | | | | | | |
| | Aib-PyrimAla-Q-Dab- | | | | | | |
| | G-dProc3CH24Pal | | | | | | |
| 327 | Bip4CO2H-4Pal- | 0.016 | 2172.87705 | C110H119F3N22O23 | 2174.25 | 2174.01 | [M + H]+ |
| | Phe4COOH-G-Proc4F- | | | | | | |
| | SbMeW4F-Aib-S-W- | | | | | | |
| | daMeD-PyrimAla-Q- | | | | | | |
| | Dab-G- | | | | | | |
| | dProc4CH21Naph4F | | | | | | |
| 328 | Bip4CO2H-4Pal- | 0.008 | 2105.86607 | C105H117F2N23O23 | 2107.19 | 2106.51 | [M + H]+ |
| | Phe4COOH-G-Proc4F- | | | | | | |
| | SbMeW4F-Aib-S-W- | | | | | | |
| | daMeD-PyrimAla-Q- | | | | | | |
| | Dab-G- | | | | | | |
| | dProc3CH24Pal | | | | | | |
| 329 | Bip4CO2H-4Pal- | 0.009 | 2138.83185 | C106H117ClF2N22O23 | 2140.65 | 2140.28 | [M + H]+ |
| | Phe4COOH-G-Proc4F- | | | | | | |
| | SbMeW4F-Aib-S-W- | | | | | | |
| | daMeD-PyrimAla-Q- | | | | | | |
| | Dab-G-dProc4Bn4Cl | | | | | | |
| 330 | Bip4CO2H-4Pal- | 0.008 | 2138.83185 | C106H117ClF2N22O23 | 2140.65 | 2139.87 | [M + H]+ |
| | Phe4COOH-G-Proc4F- | | | | | | |
| | SbMeW4F-Aib-S-W- | | | | | | |
| | daMeD-PyrimAla-Q- | | | | | | |
| | Dab-G-dProc3Bn4Cl | | | | | | |
| 331 | Bip4CO2H-4Pal- | 0.011 | 2122.8614 | C106H117F3N22O23 | 2124.19 | 2123.92 | [M + H]+ |
| | Phe4COOH-G-Proc4F- | | | | | | |
| | SbMeW4F-Aib-S-W- | | | | | | |
| | daMeD-PyrimAla-Q- | | | | | | |
| | Dab-G-dProc3Bn4F | | | | | | |
| 332 | Bip4CO2H-4Pal- | 0.013 | 2122.8614 | C106H117F3N22O23 | 2124.19 | 2124.04 | [M + H]+ |
| | Phe4COOH-G-Proc4F- | | | | | | |
| | SbMeW4F-Aib-S-W- | | | | | | |
| | daMeD-4PyrimAla-Q- | | | | | | |
| | Dab-G-dProc4Bn4F | | | | | | |
| 333 | Bip-4Pal-Phe4COOH- | 0.022 | 2061.87624 | C104H117F2N23O21 | 2063.18 | 2062.88 | [M + H]+ |
| | G-Proc4F-SbMeW4F- | | | | | | |
| | Aib-S-W-daMeD- | | | | | | |
| | PyrimAla-Q-Dab-G- | | | | | | |
| | dProc4CH24Pal | | | | | | |
| 334 | Phe4pcCCA-AlaPent- | 0.022 | 2052.92219 | C102H126F2N20O24 | 2054.21 | 2053.93 | [M + H]+ |
| | Phe4COOH-G-Proc4F- | | | | | | |
| | SbMeW4F-Aib-S-W- | | | | | | |
| | daMeD-alT-Q-Dab-G- | | | | | | |
| | dProc4CH24Pal | | | | | | |
| 335 | Phe4pcCCA-AlaPent- | 0.022 | 2100.93342 | C105H126F2N22O23 | 2102.25 | 2101.94 | [M + H]+ |
| | Phe4COOH-G-Proc4F- | | | | | | |
| | SbMeW4F-Aib-S-W- | | | | | | |
| | daMeD-PyrimAla-Q- | | | | | | |
| | Dab-G- | | | | | | |
| | dProc4CH24Pal | | | | | | |
| 336 | TyrEtNAc-4Pal- | 0.023 | 2082.92286 | C105H124F2N22O22 | 2084.24 | 1042.12 | [M + 2H]/2+ |
| | Phe4COOH-G-Proc4F- | | | | | | |
| | SbMeW4F-Aib-E-W- | | | | | | |
| | Aib-F-Q-Dab-G- | | | | | | |
| | dProc4CH24Pal | | | | | | |
| 337 | Bip4CO2H-4Pal- | 0.022 | 2071.87049 | C103H119F2N21O24 | 2073.17 | 2072.87 | [M + H]+ |
| | Phe4COOH-G-Proc4F- | | | | | | |
| | SbMeW4F-Aib-S-W- | | | | | | |
| | daMeD-alT-Q-Dab- | | | | | | |
| | Sar-dProc4CH24Pal | | | | | | |
| 338 | Bip4CONMe2-4Pal- | 0.018 | 2104.91844 | C106H122F2N24O21 | 2106.25 | 2105.93 | [M + H]+ |
| | Phe4COOH-G-Proc4F- | | | | | | |
| | SbMeW4F-Aib-S-W- | | | | | | |
| | daMeS-PyrimAla-Q- | | | | | | |
| | Dab-G- | | | | | | |
| | dProc4CH24Pal | | | | | | |
| 339 | Bip4CONH2-4Pal- | 0.024 | 2076.88714 | C104H118F2N24O21 | 2078.19 | 2077.90 | [M + H]+ |
| | Phe4COOH-G-Proc4F- | | | | | | |
| | SbMeW4F-Aib-S-W- | | | | | | |
| | daMeS-PyrimAla-Q- | | | | | | |
| | Dab-G- | | | | | | |
| | dProc4CH24Pal | | | | | | |
| 340 | Bip-4Pal-Phe4COOH- | 0.021 | 2033.88133 | C103H117F2N23O20 | 2035.17 | 2034.89 | [M + H]+ |
| | G-Proc4F-SbMeW4F- | | | | | | |
| | Aib-S-W-daMeS- | | | | | | |
| | PyrimAla-Q-Dab-G- | | | | | | |
| | dProc4CH24Pal | | | | | | |
| 341 | Bip4CONMe2- | 0.024 | 2121.93376 | C107H125F2N23O22 | 2123.28 | 2122.94 | [M + H]+ |
| | AlaPent-Phe4COOH- | | | | | | |
| | G-Proc4F-SbMeW4F- | | | | | | |
| | Aib-S-W-daMeD- | | | | | | |
| | PyrimAla-Q-Dab-G- | | | | | | |
| | dProc4CH24Pal | | | | | | |
| 342 | Bip4CONH2-AlaPent- | 0.022 | 2093.90246 | C105H121F2N23O22 | 2095.22 | 2094.91 | [M + H]+ |
| | Phe4COOH-G-Proc4F- | | | | | | |
| | SbMeW4F-Aib-S-W- | | | | | | |
| | daMeD-PyrimAla-Q- | | | | | | |
| | Dab-G- | | | | | | |
| | dProc4CH24Pal | | | | | | |
| 343 | Bip-AlaPent- | 0.026 | 2050.89665 | C104H120F2N22O21 | 2052.2 | 2051.90 | [M + H]+ |
| | Phe4COOH-G-Proc4F- | | | | | | |
| | SbMeW4F-Aib-S-W- | | | | | | |
| | daMeD-PyrimAla-Q- | | | | | | |
| | Dab-G- | | | | | | |
| | dProc4CH24Pal | | | | | | |
| 344 | Bip4Cl-4Pal- | 0.022 | 2095.83727 | C104H116ClF2N23O21 | 2097.63 | 2096.85 | [M + H]+ |
| | Phe4COOH-G-Proc4F- | | | | | | |
| | SbMeW4F-Aib-S-W- | | | | | | |
| | daMeD-PyrimAla-Q- | | | | | | |
| | Dab-G- | | | | | | |
| | dProc4CH24Pal | | | | | | |
| 345 | Bip4CO2H-4Pal- | 0.012 | 2120.88214 | C107H119F3N22O22 | 2122.22 | 2121.65 | [M + H]+ |
| | Phe4COOH-G-Proc4F- | | | | | | |
| | SbMeW4F-Aib-E-W- | | | | | | |
| | Aib-4PyrimAla-Q- | | | | | | |
| | Dab-G-dProc4Bn4F | | | | | | |
| 346 | Bip4CO2H-4Pal- | 0.01 | 2120.88214 | C107H119F3N22O22 | 2122.22 | 1061.14 | [M + 2H]/2+ |
| | Phe4COOH-G-Proc4F- | | | | | | |
| | SbMeW4F-Aib-E-W- | | | | | | |
| | Aib-PyrimAla-Q-Dab- | | | | | | |
| | G-dProc3Bn4F | | | | | | |
| 347 | Bip4CO2H-4Pal- | 0.025 | 2041.89631 | C103H121F2N21O22 | 2043.19 | 2042.93 | [M + H]+ |
| | Phe4COOH-G-Proc4F- | | | | | | |
| | SbMeW4F-Aib-S-W- | | | | | | |
| | daMeS-L-Q-Dab-G- | | | | | | |
| | dProc4CH24Pal | | | | | | |
| 348 | Bip4CO2H-AlaPent- | 0.018 | 2070.91163 | C105H124F2N20O23 | 2072.23 | 2071.92 | [M + H]+ |
| | Phe4COOH-G-Proc4F- | | | | | | |
| | SbMeW4F-Aib-S-W- | | | | | | |
| | daMeD-cBuA-Q-Dab- | | | | | | |
| | G-dProc4CH24Pal | | | | | | |
| 349 | Bip4CO2H-AlaPent- | 0.017 | 2058.91163 | C104H124F2N20O23 | 2060.21 | 2060.92 | [M + H]+ |
| | Phe4COOH-G-Proc4F- | | | | | | |
| | SbMeW4F-Aib-S-W- | | | | | | |
| | daMeD-L-Q-Dab-G- | | | | | | |
| | dProc4CH24Pal | | | | | | |
| 350 | Phe4AcPip-4Pal- | 0.02 | 2083.92934 | C103H123F2N25O21 | 2085.23 | 2084.93 | [M + H]+ |
| | Phe4COOH-G-Proc4F- | | | | | | |
| | SbMeW4F-Aib-S-W- | | | | | | |
| | daMeS-PyrimAla-Q- | | | | | | |
| | Dab-G- | | | | | | |
| | dProc4CH24Pal | | | | | | |
| 351 | Phe4Pyrim5CO2H- | 0.016 | 2113.89229 | C104H121F2N23O24 | 2115.21 | 2114.90 | [M + H]+ |
| | 4Pal-Phe4COOH-G- | | | | | | |
| | Proc4F-SbMeW4F- | | | | | | |
| | Aib-S-W-daMeD- | | | | | | |
| | AlaTHP4-Q-Dab-G- | | | | | | |
| | dProc4CH24Pal | | | | | | |
| 352 | Bip4CO2H-4Pal- | 0.023 | 2043.87558 | C102H119F2N21O23 | 2045.16 | 2044.88 | [M + H]+ |
| | Phe4COOH-G-Proc4F- | | | | | | |
| | SbMeW4F-Aib-alT-W- | | | | | | |
| | daMeS-alT-Q-Dab-G- | | | | | | |
| | dProc4CH24Pal | | | | | | |
| 353 | Bip4CO2H-4Pal- | 0.017 | 2125.91744 | C107H125F2N21O24 | 2127.26 | 2126.92 | [M + H]+ |
| | Phe4COOH-G-Proc4F- | | | | | | |
| | SbMeW4F-Aib-alT-W- | | | | | | |
| | daMeD-AlaTHP4-Q- | | | | | | |
| | Dab-G- | | | | | | |
| | dProc4CH24Pal | | | | | | |
| 354 | Bip4CO2H-4Pal- | 0.014 | 2084.83004 | C104H118ClFN20O24 | 2086.62 | 2085.84 | [M + H]+ |
| | Phe2Cl4COOH-G- | | | | | | |
| | Proc4F-SbMe1Nal- | | | | | | |
| | Aib-S-W-daMeD-alT- | | | | | | |
| | Q-Dab-G- | | | | | | |
| | dProc4CH24Pal | | | | | | |
| 355 | Bip4CO2H-4Pal- | 0.013 | 2107.78632 | C102H116Cl2FN21O24 | 2110.04 | 2108.80 | [M + H]+ |
| | Phe2Cl4COOH-G- | | | | | | |
| | Proc4F-SbMeW4Cl- | | | | | | |
| | Aib-S-W-daMeD-alT- | | | | | | |
| | Q-Dab-G- | | | | | | |
| | dProc4CH24Pal | | | | | | |
| 356 | Bip4CO2H-4Pal- | 0.011 | 2091.81587 | C102H116ClF2N21O24 | 2093.59 | 2092.82 | [M + H]+ |
| | Phe2Cl4COOH-G- | | | | | | |
| | Proc4F-SbMeW4F- | | | | | | |
| | Aib-S-W-daMeD-alT- | | | | | | |
| | Q-Dab-G- | | | | | | |
| | dProc4CH24Pal | | | | | | |
| 357 | Bip4CO2H-4Pal- | 0.017 | 2050.86901 | C104H119FN20O24 | 2052.18 | 2051.88 | [M + H]+ |
| | Phe4COOH-G-Proc4F- | | | | | | |
| | SbMe1Nal-Aib-S-W- | | | | | | |
| | daMeD-alT-Q-Dab-G- | | | | | | |
| | dProc4CH24Pal | | | | | | |
| 358 | Bip4CO2H-4Pal- | 0.013 | 2073.82529 | C102H117ClFN21O24 | 2075.6 | 2074.83 | [M + H]+ |
| | Phe4COOH-G-Proc4F- | | | | | | |
| | SbMeW4Cl-Aib-S-W- | | | | | | |
| | daMeD-alT-Q-Dab-G- | | | | | | |
| | dProc4CH24Pal | | | | | | |
| 359 | Bip4CO2H-4Pal- | 0.011 | 2057.85484 | C102H117F2N21O24 | 2059.14 | 2058.85 | [M + H]+ |
| | Phe4COOH-G-Proc4F- | | | | | | |
| | SbMeW4F-Aib-S-W- | | | | | | |
| | daMeD-alT-Q-Dab-G- | | | | | | |
| | dProc4CH24Pal | | | | | | |
| 360 | Phe4Pyrim5CO2H- | 0.019 | 2140.86749 | C106H122ClFN22O24 | 2142.69 | 2141.87 | [M + H]+ |
| | 4Pal-Phe2Cl4COOH- | | | | | | |
| | G-Proc4F-SbMe1Nal- | | | | | | |
| | Aib-S-W-daMeD- | | | | | | |
| | AlaTHP4-Q-Dab-G- | | | | | | |
| | dProc4CH24Pal | | | | | | |
| 361 | Phe4Pyrim5CO2H- | 0.022 | 2147.85332 | C104H120ClF2N23O24 | 2149.66 | 2148.86 | [M + H]+ |
| | 4Pal-Phe3Cl4COOH- | | | | | | |
| | G-Proc4F-SbMeW4F- | | | | | | |
| | Aib-S-W-daMeD- | | | | | | |
| | AlaTHP4-Q-Dab-G- | | | | | | |
| | dProc4CH24Pal | | | | | | |
| 362 | TyrEtNAc-4Pal- | 0.023 | 2108.89879 | C103H126ClFN22O24 | 2110.69 | 2109.91 | [M + H]+ |
| | Phe4COOH-G-Proc4F- | | | | | | |
| | SbMeW4Cl-Aib-S-W- | | | | | | |
| | daMeD-AlaTHP4-Q- | | | | | | |
| | Dab-G- | | | | | | |
| | dProc4CH24Pal | | | | | | |
| 363 | TyrEtNAc-4Pal- | 0.021 | 2119.90354 | C105H127ClFN21O24 | 2121.71 | 2119.91 | [M + H]+ |
| | Phe2Cl4COOH-G- | | | | | | |
| | Proc4F-SbMe1Nal- | | | | | | |
| | Aib-S-W-daMeD- | | | | | | |
| | AlaTHP4-Q-Dab-G- | | | | | | |
| | dProc4CH24Pal | | | | | | |
| 364 | Bip4CO2H-4Pal- | 0.015 | 2083.90688 | C105H123F2N21O23 | 2085.22 | 2084.91 | [M + H]+ |
| | Phe4COOH-G-Proc4F- | | | | | | |
| | SbMeW4F-Aib-S-W- | | | | | | |
| | daMeS-AlaTHP4-Q- | | | | | | |
| | Dab-G- | | | | | | |
| | dProc4CH24Pal | | | | | | |
| 365 | Phe4AcPip-4Pal- | 0.023 | 2101.89546 | C103H122ClF2N25O20 | 2103.68 | 2102.90 | [M + H]+ |
| | Phe2Cl4COOH-G- | | | | | | |
| | Proc4F-SbMeW4F- | | | | | | |
| | Aib-S-W-Aib- | | | | | | |
| | PyrimAla-Q-Dab-G- | | | | | | |
| | dProc4CH24Pal | | | | | | |
| 366 | Phe4AcPip-4Pal- | 0.021 | 2067.93443 | C103H123F2N25O20 | 2069.23 | 2068.94 | [M + H]+ |
| | Phe4COOH-G-Proc4F- | | | | | | |
| | SbMeW4F-Aib-S-W- | | | | | | |
| | Aib-PyrimAla-Q-Dab- | | | | | | |
| | G-dProc4CH24Pal | | | | | | |
| 367 | TyrEtNAc-4Pal- | 0.017 | 2076.86382 | C101H119ClF2N24O21 | 2078.62 | 2077.87 | [M + H]+ |
| | Phe2Cl4COOH-G- | | | | | | |
| | Proc4F-SbMeW4F- | | | | | | |
| | Aib-S-W-Aib- | | | | | | |
| | PyrimAla-Q-Dab-G- | | | | | | |
| | dProc4CH24Pal | | | | | | |
| 368 | TyrEtNAc-4Pal- | 0.022 | 2042.90279 | C101H120F2N24O21 | 2044.18 | 2043.90 | [M + H]+ |
| | Phe4COOH-G-Proc4F- | | | | | | |
| | SbMeW4F-Aib-S-W- | | | | | | |
| | Aib-PyrimAla-Q-Dab- | | | | | | |
| | G-dProc4CH24Pal | | | | | | |
| 369 | TyrEtNAc-4Pal- | 0.021 | 2103.88795 | C103H120F3N23O23 | 2105.19 | 1053.81 | [M + 2H]/2+ |
| | Phe4COOH-G-Proc4F- | | | | | | |
| | SbMeW4F-Aib-S-W- | | | | | | |
| | daMeD-PyrimAla-Q- | | | | | | |
| | Dab-G-dProc3Bn4F | | | | | | |
| 370 | TyrEtNAc-AlaPent- | 0.015 | 2098.94407 | C104H129F3N20O24 | 2100.25 | 1051.35 | [M + 2H]/2+ |
| | Phe4COOH-G-Proc4F- | | | | | | |
| | SbMeW4F-Aib-S-W- | | | | | | |
| | daMeD-AlaTHP4-Q- | | | | | | |
| | Dab-G-dProc3Bn4F | | | | | | |
| 371 | TyrEtNAc-AlaPent- | 0.014 | 2070.94916 | C103H129F3N20O23 | 2072.24 | 1037.02 | [M + 2H]/2+ |
| | Phe4COOH-G-Proc4F- | | | | | | |
| | SbMeW4F-Aib-S-W- | | | | | | |
| | daMeS-AlaTHP4-Q- | | | | | | |
| | Dab-G-dProc3Bn4F | | | | | | |
| 372 | TyrEtNAc-4Pal- | 0.009 | 2109.92367 | C104H126F3N21O24 | 2111.23 | 1056.71 | [M + 2H]/2+ |
| | Phe4COOH-G-Proc4F- | | | | | | |
| | SbMeW4F-Aib-S-W- | | | | | | |
| | daMeD-AlaTHP4-Q- | | | | | | |
| | Dab-G-dProc3Bn4F | | | | | | |
| 373 | TyrEtNAc-AlaPent- | 0.012 | 2097.96005 | C104H130F3N21O23 | 2099.27 | 1050.81 | [M + 2H]/2+ |
| | Phe4CONH2-G- | | | | | | |
| | Proc4F-SbMeW4F- | | | | | | |
| | Aib-S-W-daMeD- | | | | | | |
| | AlaTHP4-Q-Dab-G- | | | | | | |
| | dProc3Bn4F | | | | | | |
| 374 | TyrEtNAc-AlaPent- | 0.017 | 2081.94874 | C103H129F2N21O24 | 2083.25 | 1042.91 | [M + 2H]/2+ |
| | Phe4COOH-G-Proc4F- | | | | | | |
| | SbMeW4F-Aib-S-W- | | | | | | |
| | daMeD-AlaTHP4-Q- | | | | | | |
| | Dab-G- | | | | | | |
| | dProc4CH24Pal | | | | | | |
| 375 | TyrEtNAc-AlaPent- | 0.025 | 2053.95383 | C102H129F2N21O23 | 2055.24 | 1028.58 | [M + 2H]/2+ |
| | Phe4COOH-G-Proc4F- | | | | | | |
| | SbMeW4F-Aib-S-W- | | | | | | |
| | daMeS-AlaTHP4-Q- | | | | | | |
| | Dab-G- | | | | | | |
| | dProc4CH24Pal | | | | | | |
| 376 | TyrEtNAc-AlaPent- | 0.048 | 2080.96473 | C103H130F2N22O23 | 2082.26 | 1041.97 | [M + 2H]/2+ |
| | Phe4CONH2-G- | | | | | | |
| | Proc4F-SbMeW4F- | | | | | | |
| | Aib-S-W-daMeD- | | | | | | |
| | AlaTHP4-Q-Dab-G- | | | | | | |
| | dProc4CH24Pal | | | | | | |
| 377 | TyrEtNAc-4Pal- | 0.04 | 2091.94432 | C103H127F2N23O23 | 2093.25 | 1047.67 | [M + 2H]/2+ |
| | Phe4CONH2-G- | | | | | | |
| | Proc4F-SbMeW4F- | | | | | | |
| | Aib-S-W-daMeD- | | | | | | |
| | AlaTHP4-Q-Dab-G- | | | | | | |
| | dProc4CH24Pal | | | | | | |
| 378 | TyrEtNAc-4Pal- | 0.043 | 2063.94941 | C102H127F2N23O22 | 2065.24 | 1033.88 | [M + 2H]/2+ |
| | Phe4CONH2-G- | | | | | | |
| | Proc4F-SbMeW4F- | | | | | | |
| | Aib-S-W-daMeS- | | | | | | |
| | AlaTHP4-Q-Dab-G- | | | | | | |
| | dProc4CH24Pal | | | | | | |
| 379 | Bip4CONH2-4Pal- | 0.016 | 2121.87739 | C106H118F3N23O22 | 2123.21 | 1062.77 | [M + 2H]/2+ |
| | Phe4COOH-G-Proc4F- | | | | | | |
| | SbMeW4F-Aib-S-W- | | | | | | |
| | daMeD-PyrimAla-Q- | | | | | | |
| | Dab-G-dProc3Bn4F | | | | | | |
| 380 | Bip4CONH2-4Pal- | 0.018 | 2120.89337 | C106H119F3N24O21 | 2122.22 | 1061.94 | [M + 2H]/2+ |
| | Phe4CONH2-G- | | | | | | |
| | Proc4F-SbMeW4F- | | | | | | |
| | Aib-S-W-daMeD- | | | | | | |
| | PyrimAla-Q-Dab-G- | | | | | | |
| | dProc3Bn4F | | | | | | |
| 381 | TyrEtNAc-4Pal- | 0.041 | 2035.95449 | C101H127F2N23O21 | 2037.23 | 1019.17 | [M + 2H]/2+ |
| | Phe4CONH2-G- | | | | | | |
| | Proc4F-SbMeW4F- | | | | | | |
| | Aib-S-W-Aib- | | | | | | |
| | alThrOEt-Q-NMeDab- | | | | | | |
| | G-dProc4CH24Pal | | | | | | |
| 382 | TyrEtNAc-4Pal- | 0.048 | 2035.95449 | C101H127F2N23O21 | 2037.23 | 680.14 | [M + 3H]/3+ |
| | Phe4CONH2-G- | | | | | | |
| | Proc4F-SbMeW4F- | | | | | | |
| | Aib-S-W-Aib-ThrOEt- | | | | | | |
| | Q-NMeDab-G- | | | | | | |
| | dProc4CH24Pal | | | | | | |
| 383 | TyrEtNAc-4Pal- | 0.042 | 2071.92934 | C102H123F2N25O21 | 2073.22 | 1037.58 | [M + 2H]/2+ |
| | SbOHPhe4CONH2-G- | | | | | | |
| | Proc4F-SbMeW4F- | | | | | | |
| | Aib-S-W-Aib- | | | | | | |
| | PyrimAla-Q-NMeDab- | | | | | | |
| | G-dProc4CH24Pal | | | | | | |
| 384 | TyrEtNAc-4Pal- | 0.086 | 2041.91878 | C101H121F2N25O20 | 2043.19 | 2042.94 | [M + H]+ |
| | Phe4CONH2-G- | | | | | | |
| | Proc4F-SbMeW4F- | | | | | | |
| | Aib-S-W-Aib- | | | | | | |
| | PyrimAla-Q-Dab-G- | | | | | | |
| | dProc4CH24Pal | | | | | | |
| 385 | Phe44PyNH3-4Pal- | 0.023 | 2094.87772 | C104H117F3N24O21 | 2096.19 | 2096.37 | [M + H]+ |
| | Phe4COOH-G-Proc4F- | | | | | | |
| | SbMeW4F-Aib-S-W- | | | | | | |
| | daMcD-PyrimAla-Q- | | | | | | |
| | Dab-G-dProc3Bn4F | | | | | | |

1. A compound of Formula (I) 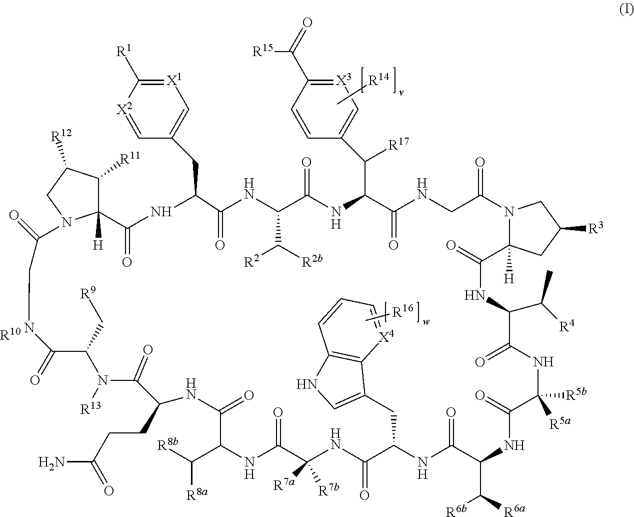
R¹ is R^{1e}—C(O)NH—CH₂CH₂—O—, C₁-C₄ alkyl, halo, or C¹;
R^{1e}— is:
(a) C₁-C₄ alkyl; or
(b) C^{Y1}; wherein C^{Y1} is:
(i) C₃-C₆ cycloalkyl;
(ii) phenyl; or
(iii) a 5- to 6-membered monocyclic heteroaryl, wherein said heteroaryl contains 1 to 2 heteroatoms independently selected from the group consisting of N, O, and S;
wherein C^{Y1} is unsubstituted or substituted by 1 to 3 R^{Y1} substituents selected from the group consisting of C₁-C₃ alkyl, halo, and piperazinyl;
C¹ is:
(i) a 5- to 6-membered monocyclic aryl or heteroaryl, wherein said heteroaryl contains 1 to 2 heteroatoms independently selected from the group consisting of N, O, and S;
(ii) a 3- to 6-membered monocyclic or a 5- to 8-membered bicyclic cycloalkyl; or
(iii) a 5- to 6-membered monocyclic, saturated heterocycloalkyl containing 1 to 2 heteroatoms independently selected from the group consisting of N, O, and S;
wherein C¹ is unsubstituted or substituted by 1 to 3 R^{C1} substituents independently selected from the group consisting of halo, C₁-C₃ alkyl, C₁-C₃ fluoroalkyl, carboxy, C₁-C₃ alkoxy, C₂-C₃ acyl, —C(O)NH₂, and —C(O)N(CH₃)₂;
R² is:
(i) a 5- to 6-membered monocyclic aryl or heteroaryl, wherein said heteroaryl contains 1 to 2 heteroatoms independently selected from the group consisting of N, O, and S; and
(ii) a 3- to 8-membered mono- or bicyclic cycloalkyl;
wherein R² is unsubstituted or substituted by 1 to 3 R^{2a} substituents independently selected from the group consisting of halo, amino, hydroxy, C₁-C₃ alkyl, C₁-C₃ fluoroalkyl, and C₁-C₃ alkoxy;
R^{2b} is H or hydroxy;
R³ is F or hydroxy;
R⁴ is:
(i) naphthyl; or
(ii) a 9- to 10-membered heteroaryl, wherein said heteroaryl contains 1 to 2 heteroatoms independently selected from the group consisting of N, O, and S;
wherein R⁴ is unsubstituted or substituted by 1 to 2 R^{4a} substituents independently selected from the group consisting of halo;
R^{5a} is H, C₁-C₃ alkyl, H₂N(CH₂)ₘ—, or HOCH₂—;
R^{5b} is H, C₁-C₃ alkyl, H₂N(CH₂)ₘ—, or HOCH₂—;
or, alternatively R^{5a} and R^{5b}, together with the carbon atom to which they attached, form a C₃-C₆ cycloalkyl or a 4- to 6-membered saturated heterocycloalkyl containing one N atom;
each of R^{6a} and R^{6b} is independently H, —(CH₂)ₙ₁CH₃, —(CH₂)ₙ₂—OH, or —(CH₂)ₙ₂CO₂H;
R^{7a} is H, C₁-C₃ alkyl, HOCH₂—, H₂N(CH₂)ₚ—, HO₂CCH₂—, H₂NC(O)CH₂—, CH₃OCH₂—, or PhCH₂—;
R^{7b} is H, C₁-C₃ alkyl, HOCH₂—, H₂N(CH₂)ₚ—, HO₂CCH₂—, H₂NC(O)CH₂—, CH₃₀CH₂—, or PhCH₂—;
or, alternatively R^{7a} and R^{7b}, together with the carbon atom to which they are attached, form a 4- to 6-membered saturated heterocycloalkyl containing one N atom;
R^{8a} is HO—(CH₂)_{q}—, CH₃—O—(CH₂)_{q}—, CH₃CH₂—O—(CH₂)_{q}—, PhCH₂—O—(CH₂)_{q}—, C₁-C₃ alkyl, C₁-C₃ fluoroalkyl, H₂N—(CH₂)ᵣ—, (CH₃)₃N—(CH₂)ᵣ—, H₂NC(NH)N(H)—(CH₂)ᵣ—, H₂NC(O)N(H)—(CH₂)ᵣ—, HO₂C—(CH₂)ᵣ—, (CH₃)SO₂—(CH₂)ᵣ—, C^{8a}, or C^{8a}—CH₂—;
wherein C^{8a} is:
(i) a 5- to 6-membered monocyclic aryl or heteroaryl, wherein said heteroaryl contains 1 to 2 heteroatoms independently selected from the group consisting of N, O, and S;
(ii) a 5- to 6-membered monocyclic, saturated heterocycloalkyl, wherein said heterocycloalkyl contains 1 to 2 heteroatoms independently selected from the group consisting of N, O, and S; or
(iii) a C₃-C₆ cycloalkyl;
wherein C^{8a} is unsubstituted or substituted by 1 to 3 R^{C8a} substituents independently selected from the group consisting of halo, amino, hydroxy, C₁-C₃ alkyl, C₁-C₃ fluoroalkyl, C₁-C₃ alkoxy, H₂N—(CH₂)ₛ—, H₂NC(O)—(CH₂)ₛ—, H₂C═CH—CH₂O—, and phenyl;
R^{8b} is H, methyl, or hydroxy;
R⁹ is HO—(CH₂)ₜ—, H₂N—(CH₂)ᵤ—, H₂NC(NH)N(H)—(CH₂)ᵤ—, H₂NC(O)N(H)—(CH₂)ᵤ—, or C⁹;
C⁹ is a 5- to 6-membered saturated heterocycloalkyl containing 1 to 2 heteroatoms independently selected from the group consisting of N, O and S;
wherein C⁹ is unsubstituted or substituted by 1 to 2 R^{C9} moieties independently selected from the group consisting of halo, C₁-C₃ alkyl, C₁-C₃ fluoroalkyl, and C₁-C₃ alkoxy;
R¹⁰ is H or methyl;
R¹¹ is H, —CH₂—C¹¹, or —CH₂—C¹¹—C^{a};
C¹¹ is:
(i) phenyl; or
(ii) a 5- to 6-membered monocyclic heteroaryl, wherein said heteroaryl contains 1 to 2 heteroatoms independently selected from the group consisting of N, O, and S;
wherein C¹¹ is unsubstituted or substituted by 1 to 3 R^{C11} substituents independently selected from the group consisting of halo, hydroxy, amino, C₁-C₃ alkyl, C₁-C₃ fluoroalkyl, carboxy, C₁-C₃ alkoxy, and C₂-C₃ acyl;
C^{a} is a 5- to 6-membered monocyclic heteroaryl, wherein said heteroaryl contains 1 to 2 heteroatoms independently selected from the group consisting of N, O, and S;
wherein C^{a} is unsubstituted or substituted by 1 to 3 R^{Ca} substituents independently selected from the group consisting of halo, hydroxy, amino, C₁-C₃ alkyl, C₁-C₃ fluoroalkyl, carboxy, C₁-C₃ alkoxy, and C₂-C₃ acyl;
R¹² is H or —CH₂C¹²;
C¹² is:
(i) phenyl; or
(ii) a 5- to 6-membered monocyclic heteroaryl, wherein said heteroaryl contains 1 to 2 heteroatoms independently selected from the group consisting of N, O, and S;
wherein C¹² is unsubstituted or substituted by 1 to 3 R^{C12} substituents independently selected from the group consisting of halo, hydroxy, amino, C₁-C₃ alkyl, C₁-C₃ fluoroalkyl, carboxy, C₁-C₃ alkoxy, and C₂-C₃ acyl;
R¹³ is H or methyl;
R¹⁴ is halo;
R¹⁵ is —OH or —NH₂;
R¹⁶ is halo, hydroxy, C₁-C₃ alkyl, C₁-C₃ fluoroalkyl, or C₁-C₃ alkoxy;
R¹⁷ is H, hydroxy, or methyl;
each occurrence of subscript m is independently 1, 2, 3, or 4;
subscript n1 is 0, 1, 2, or 3;
subscript n2 is 0, 1, or 2;
each occurrence of subscript p is independently 2, 3 or 4;
subscript q is 0, 1 or 2;
subscript r is 0, 1, 2, or 3;
each occurrence of subscript s is independently 1 or 2;
subscript t is 0, 1, or 2;
subscript u is 0, 1, 2, or 3;
subscript v is 0, 1 or 2;
subscript w is 0, 1, or 2;
X¹ and X² are independently C(H) or N; and
X³ and X⁴ are independently C(H), C(Cl), C(F) or N; or
a pharmaceutically acceptable salt thereof.

2. The compound of claim 1 or a pharmaceutically acceptable salt thereof, wherein the group 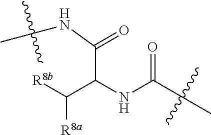
is

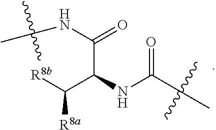

3. The compound of claim 1 or a pharmaceutically acceptable salt thereof, wherein the compound of Formula (I) has the Formula (IA)

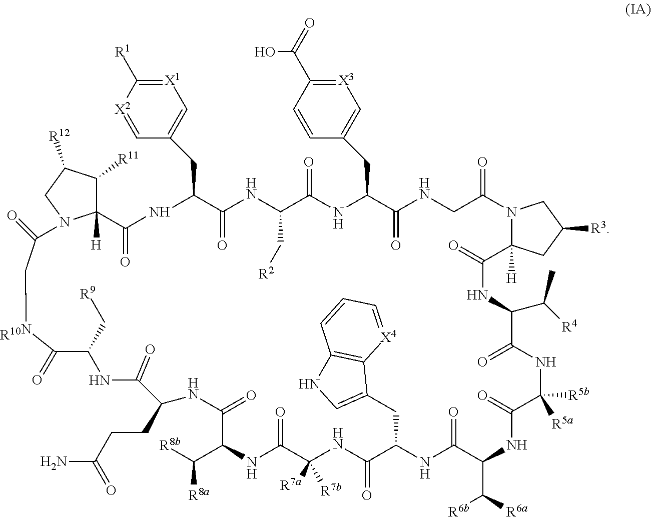

4. (canceled)

5. The compound of claim 1 or a pharmaceutically acceptable salt thereof, wherein
X¹ and X² are C(H); and
R¹ is:
C¹, wherein C¹ is phenyl or bicyclo[1.1.1]pentanyl;
wherein C¹ is substituted by carboxy; or
R^{1e}—C(O)NH—CH₂CH₂—O—,
wherein R^{1e} is C₁-C₄ alkyl.

6. The compound of claim 1 or a pharmaceutically acceptable salt thereof, wherein
X¹ and X² are C(H); and
R¹ is phenyl substituted by carboxy.

7. The compound of claim 1 or a pharmaceutically acceptable salt thereof, wherein R^{2b} is H.

8. The compound of claim 1 or a pharmaceutically acceptable salt thereof, wherein R² is the 5- to 6-membered monocyclic aryl or heteroaryl, unsubstituted or substituted by 1 to 3 R^{2a} substituents; and X³ is C(H).

9. The compound of claim 8 or a pharmaceutically acceptable salt thereof, wherein R² is unsubstituted or substituted pyridyl.

10. (canceled)

11. The compound of claim 1 or a pharmaceutically acceptable salt thereof, wherein R³ is fluoro.

12. The compound of claim 1 or a pharmaceutically acceptable salt thereof, wherein R⁴ is 4-fluoroindolyl.

13. The compound of claim 1 or a pharmaceutically acceptable salt thereof, wherein R^{5a} and R^{5b} bare methyl.

14. The compound of claim 1 or a pharmaceutically acceptable salt thereof, wherein:
R^{6a} is H, —(CH₂)ₙ₁CH₃, —(CH₂)ₙ₂—OH, or —(CH₂)ₙ₂CO₂H;
R^{6b} is H or methyl;
subscript n1 is 1, 2, or 3; and
subscript n2 is 0, 1, or 2.

15. (canceled)

16. The compound of claim 1 or a pharmaceutically acceptable salt thereof, wherein X⁴ is C(H).

17. The compound of claim 1 or a pharmaceutically acceptable salt thereof, wherein R^{7a} and R^{7b} are methyl.

18. The compound of claim 1 or a pharmaceutically acceptable salt thereof, wherein:
R^{8a} is phenyl, pyridyl pyrimidinyl, pyrazinyl, pyridazinyl, piperidinyl, tetrahydropyranyl, or morpholinyl, substituted or unsubstituted by 1 to 3 R^{C8a} substituents; and
R^{8b} is H.

19. The compound of claim 18 or a pharmaceutically acceptable salt thereof, wherein R^{8a} is unsubstituted pyridyl, pyrimidinyl or pyrazinyl.

20. The compound of claim 1 or a pharmaceutically acceptable salt thereof, wherein R⁹ is H₂N—(CH₂)ᵤ— and subscript u is 1 or 2.

21. The compound of claim 1 or a pharmaceutically acceptable salt thereof, wherein R¹⁰ is H.

22. (canceled)

23. The compound of claim 1 or a pharmaceutically acceptable salt thereof, wherein:
R¹¹ is:
—CH₂—C¹¹, wherein C¹¹ is phenyl, pyridyl, pyrimidinyl, or pyrazinyl, wherein C¹¹ is unsubstituted or substituted by 1 R^{C11}; or
—CH₂—C¹¹—C^{a}, wherein:
C¹¹ is phenyl, wherein C¹¹ is unsubstituted or substituted by 1 R^{C11}; and
C^{a} is pyridyl, pyrimidinyl, pyrazinyl, pyridazinyl, isoxazolyl, or imidazolyl; wherein C^{a} is unsubstituted or substituted by 1 R^{Ca}; and
R¹² is H.

24. The compound of claim 1 or a pharmaceutically acceptable salt thereof, wherein:
X¹ X², X³ and X⁴ are C(H);
R¹ is phenyl substituted by carboxy;
R² is the 5- to 6-membered monocyclic aryl or heteroaryl, unsubstituted or substituted by 1 to 3 R^{2a} substituents;
R³ is fluoro;
R⁴ is 4-fluoroindolyl;
R^{5a} and R^{5b} bare methyl;
R^{6a} is —OH or —CH₂CO₂H;
R^{6b} is H;
R^{7a} and R^{7b} are methyl;
R^{8a} is phenyl, pyridyl pyrimidinyl, pyrazinyl, pyridazinyl, piperidinyl, tetrahydropyranyl, or morpholinyl, unsubstituted or substituted by 1 to 3 R^{C8a} substituents;
R^{8b} is H
R⁹ is H₂N—(CH₂)ᵤ—;
R¹⁰ is H; and
subscript u is 1 or 2.

25-27. (canceled)

28. The compound of claim 1 or a pharmaceutically acceptable salt thereof, wherein the compound of Formula (I) has the Formula (IB)

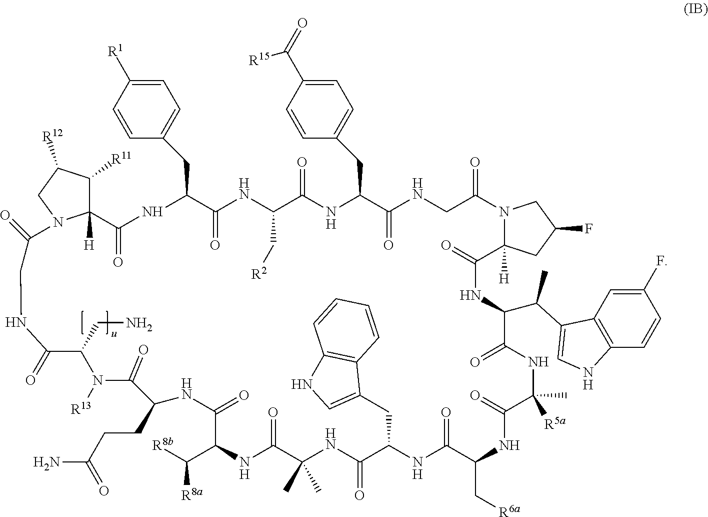

29. (canceled)

30. The compound of claim 1 selected from the group consisting of SEQ ID NOS: 1-213 and 215-385, or a pharmaceutically acceptable salt thereof.

31. The compound of claim 1 selected from the group consisting of (SEQ ID NOS 22, 29, 41, 42, 44, 48, 51, 67, 72, 99, 101, 164, 218, 264, 325, and 381 (respectively, in order of appearance): 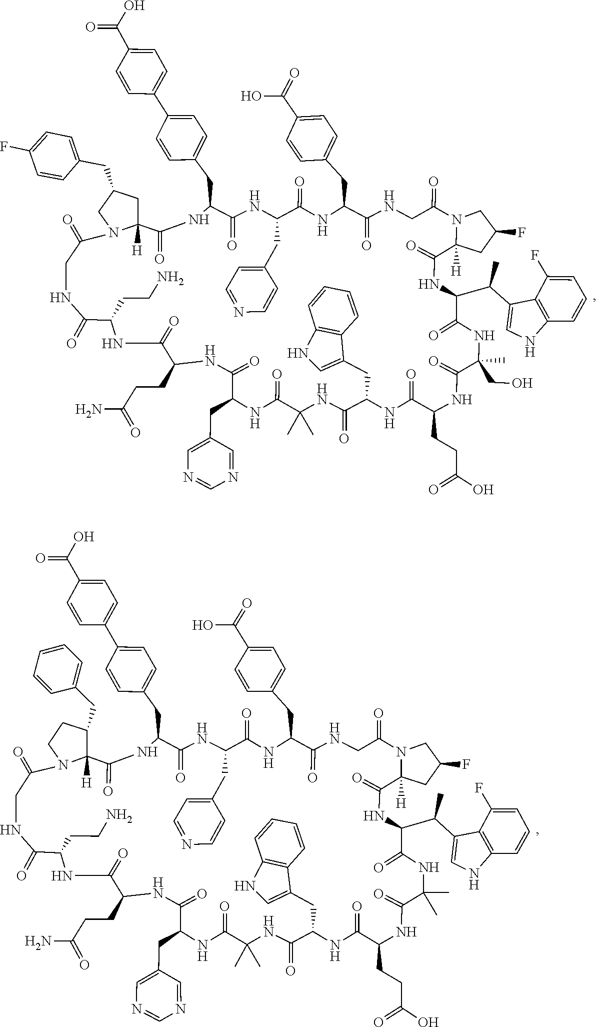 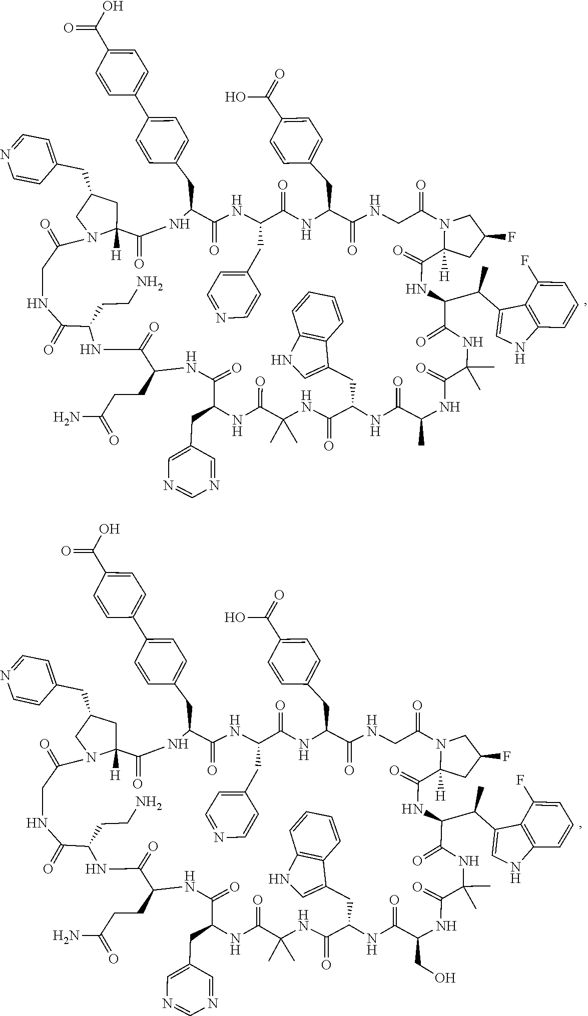 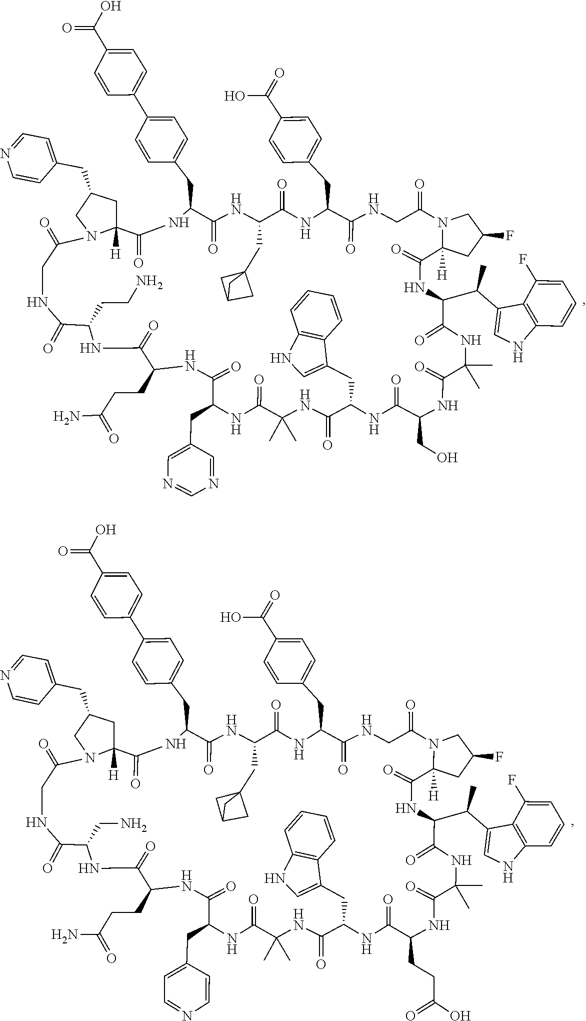 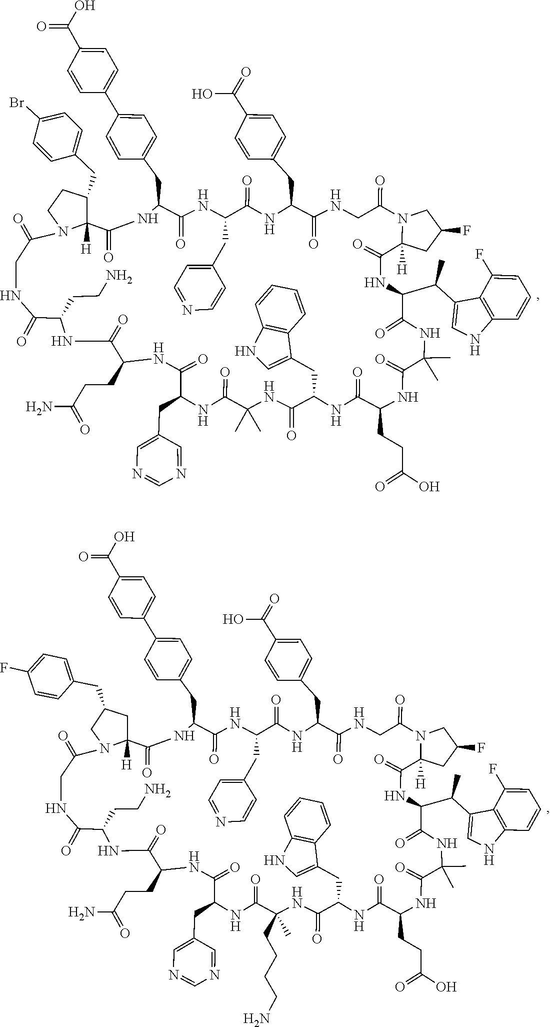 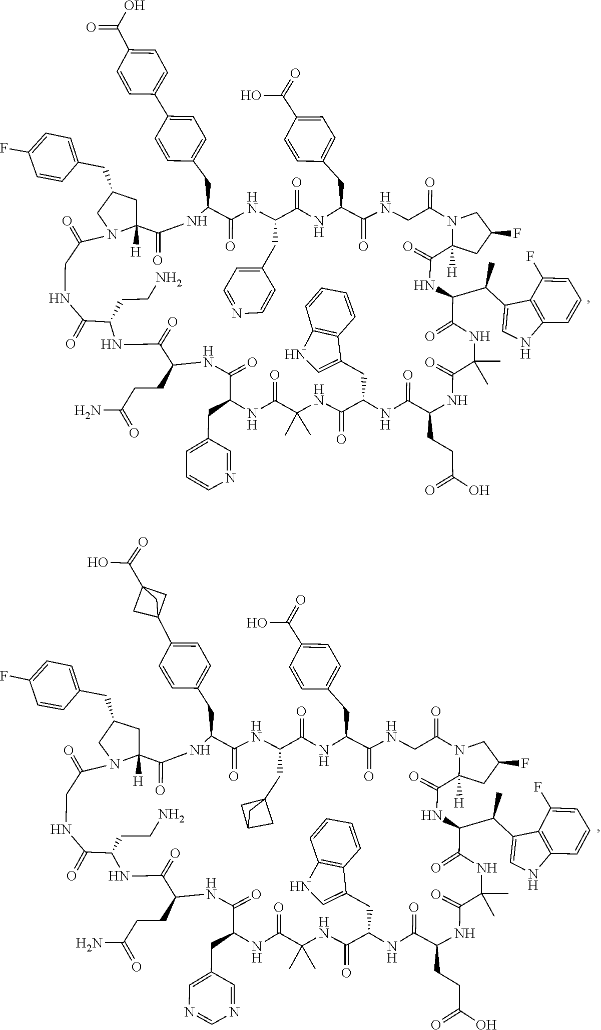 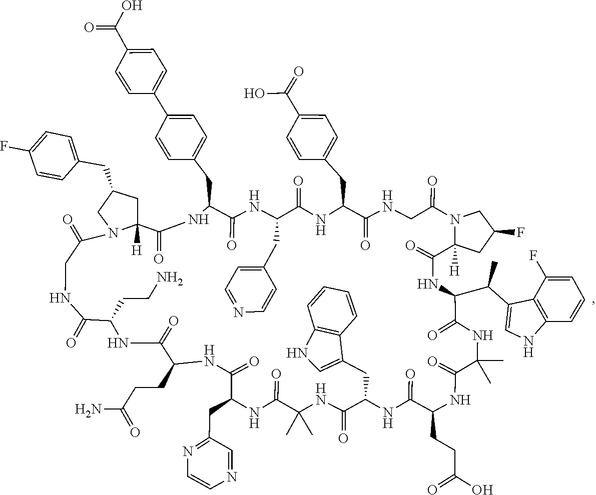 [SMILES: C[C@@H](c1c[nH]c2cccc(F)c12)[C@@H]1NC(=O)[C@@H]2C[C@H](F)CN2C(=O)CNC(=O)[C@H](Cc2ccc(C(=O)O)cc2)NC(=O)[C@H](Cc2ccncc2)NC(=O)[C@H](Cc2ccc(-c3ccc(C(=O)O)cc3)cc2)NC(=O)[C@H]2C[C@@H](Cc3ccc(F)cc3)CN2C(=O)CNC(=O)[C@H](CCN)NC(=O)[C@H](CCC(N)=O)NC(=O)[C@H](Cc2cnccn2)NC(=O)C(C)(C)NC(=O)[C@H](Cc2c[nH]c3ccccc23)NC(=O)[C@H](CCC(=O)O)NC(=O)C(C)(C)NC1=O] 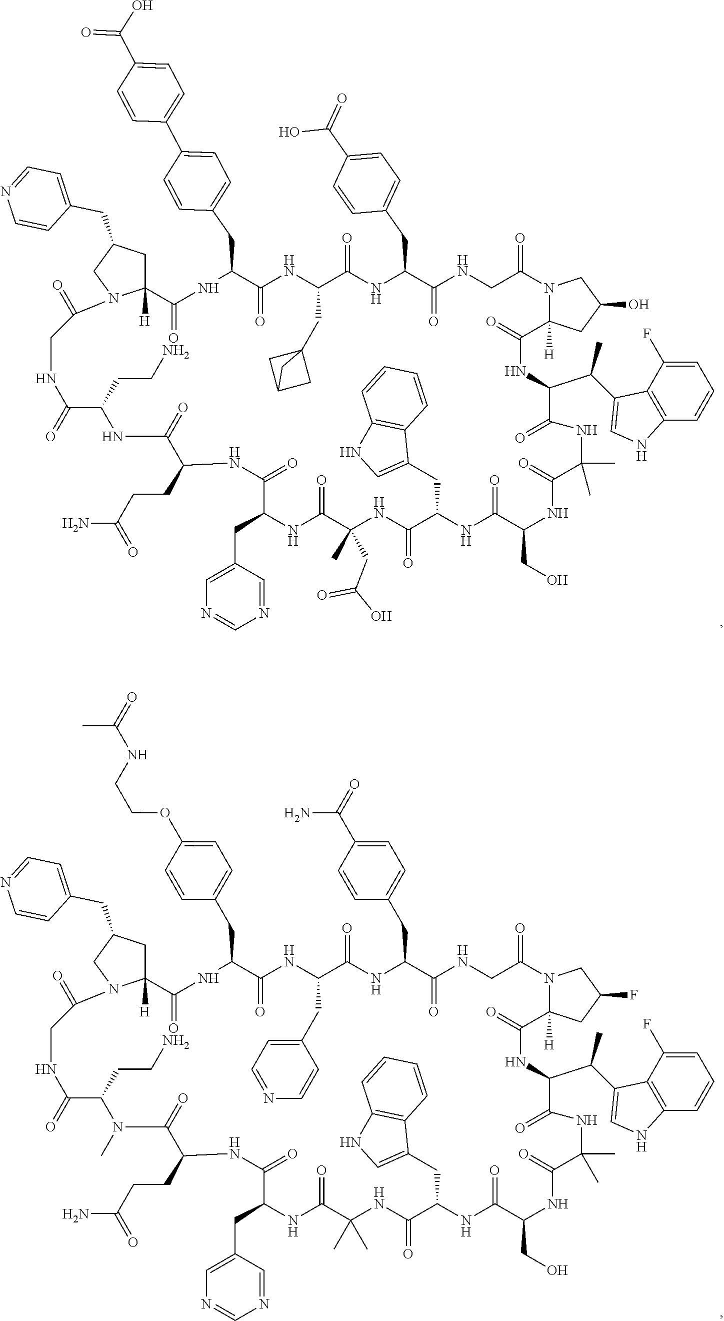 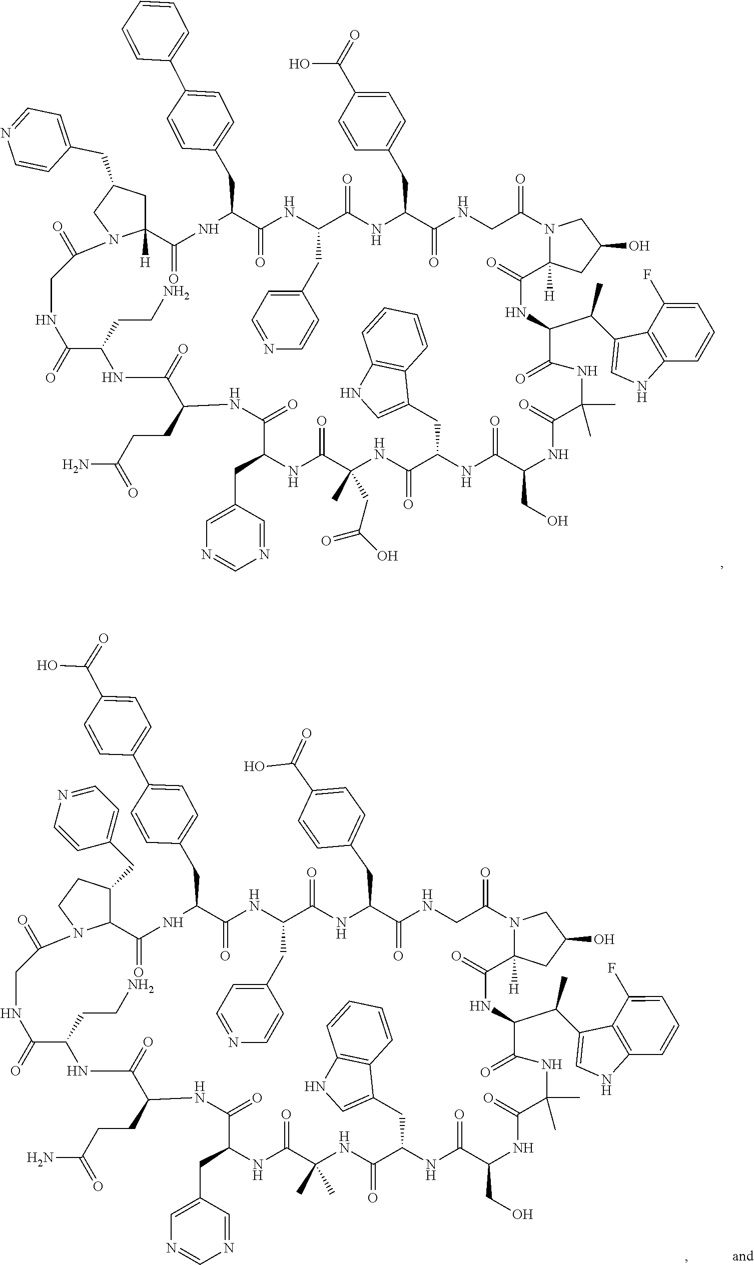 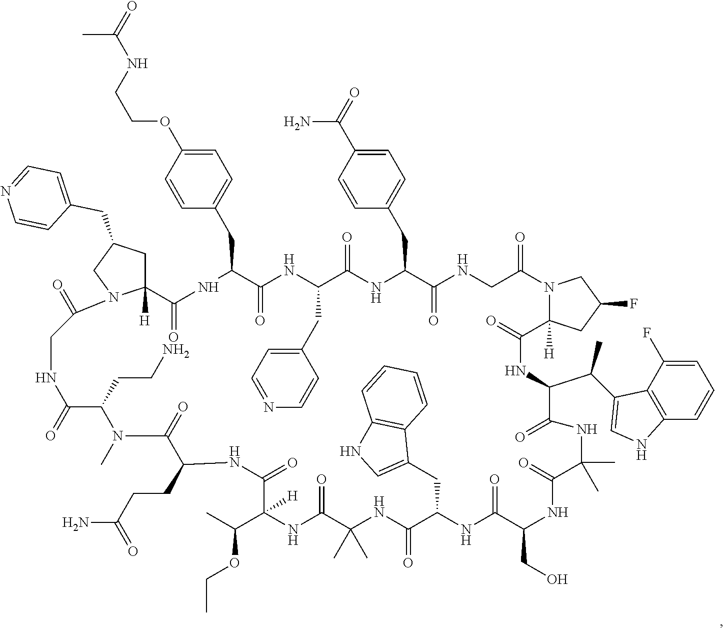 [SMILES: CCO[C@@H](C)[C@@H]1NC(=O)C(C)(C)NC(=O)[C@H](Cc2c[nH]c3ccccc23)NC(=O)[C@H](CO)NC(=O)C(C)(C)NC(=O)[C@H]([C@@H](C)c2c[nH]c3cccc(F)c23)NC(=O)[C@@H]2C[C@H](F)CN2C(=O)CNC(=O)[C@H](Cc2ccc(C(N)=O)cc2)NC(=O)[C@H](Cc2ccncc2)NC(=O)[C@H](Cc2ccc(OCCNC(C)=O)cc2)NC(=O)[C@H]2C[C@@H](Cc3ccncc3)CN2C(=O)CNC(=O)[C@H](CCN)N(C)C(=O)[C@H](CCC(N)=O)NC1=O]
or a pharmaceutically acceptable salt thereof.

32. A pharmaceutical composition comprising the compound of claim 1 or a pharmaceutically acceptable salt thereof, and a pharmaceutically acceptable carrier.

33. A method of treating atherosclerosis, comprising administering a therapeutically effective amount of the compound of claim 1 or a pharmaceutically acceptable salt thereof to a subject in need thereof.

34. A method of treating vascular inflammation, comprising administering a therapeutically effective amount of the compound of claim 1 or a pharmaceutically acceptable salt thereof to a subject in need thereof.

35. A method of treating an inflammatory disorder, comprising administering a therapeutically effective amount of the compound of claim 1 or a pharmaceutically acceptable salt thereof to a subject in need thereof.

36-39. (canceled)
